(19) United States
(12) Patent Application Publication
(10) Pub. No.: US 2022/0025039 A1
(43) Pub. Date: Jan. 27, 2022

(54) METHODS FOR TREATING DYSREGULATED LIPID METABOLISM

(71) Applicant: DENALI THERAPEUTICS INC., South San Francisco, CA (US)

(72) Inventors: Giuseppe ASTARITA, South San Francisco, CA (US); Gilbert DI PAOLO, South San Francisco, CA (US); Kai Lin LIN, South San Francisco, CA (US); Kathryn M. MONROE, South San Francisco, CA (US); Alicia A. NUGENT, South San Francisco, CA (US); Bettina VAN LENGERICH, South San Francisco, CA (US)

(73) Assignee: DENALI THERAPEUTICS INC., South San Francisco, CA (US)

(21) Appl. No.: 17/296,506

(22) PCT Filed: Nov. 26, 2019
(86) PCT No.: PCT/US2019/063427

§ 371 Date: May 24, 2021

Related U.S. Application Data
(60) Provisional application No. 62/890,506, filed on Aug. 22, 2019.
(60) Provisional application No. 62/817,955, filed on Mar. 13, 2019.
(60) Provisional application No. 62/771,456, filed on Nov. 26, 2018.

(51) Int. Cl. C07K 16/28; A61P 3/00

(52) U.S. Cl. CPC C07K 16/2803; A61K 2039/505; C07K 2317/75; A61P 3/00

(57) ABSTRACT

Certain embodiments described herein provide a method for treating dysregulated lipid metabolism and/or inflammation in a mammal in need thereof, comprising administering to the mammal an effective amount of an agonist anti-triggering receptor expressed on myeloid cells 2 (TREM2) antibody.

### CROSS-REFERENCE TO RELATED APPLICATIONS

This application claims priority to U.S. Provisional Application Ser. No. 62/771,456, filed Nov. 26, 2018, U.S. Provisional Application Ser. No. 62/817,955, filed Mar. 13, 2019 and U.S. Provisional Application Ser. No. 62/890,506, filed Aug. 22, 2019. The entire content of the applications referenced above are hereby incorporated by reference herein.

### BACKGROUND

Lipids are a large and diverse class of biomolecules that exert multiple biochemical functions, such as storing energy, signaling and acting as structural components of cell membranes and the myelin sheath. Lipid metabolism refers to the intracellular or extracellular synthesis and degradation of lipids, which includes the break-down or storage of fats for energy. Lipid dysregulation in myeloid cells, including neutrophils, monocytes, macrophages and microglia, has been shown to cause deleterious inflammatory responses as well as lipotoxicity in affected cells or tissues and mediate a large number of disease processes. While much research has been done to investigate lipid metabolism and its links to disease, additional work is needed to further elucidate the molecular mechanisms underlying lipid processing, lipid-associated inflammation and lipotoxicity, as well as to develop new therapies to correct lipid dysregulation and/or associated inflammatory responses in certain diseases and conditions.

### SUMMARY

Certain embodiments described herein provide a method for treating dysregulated lipid metabolism in a mammal in need thereof, comprising administering to the mammal an effective amount of an agonist anti-triggering receptor expressed on myeloid cells 2 (TREM2) antibody.

Certain embodiments described herein provide an agonist anti-TREM2 antibody for use in the treatment of dysregulated lipid metabolism in a mammal.

Certain embodiments described herein provide the use of an agonist anti-TREM2 antibody to prepare a medicament for treating dysregulated lipid metabolism in a mammal.

Certain embodiments described herein provide a method of reducing intracellular accumulation of one or more lipids in a cell, comprising contacting the cell with an effective amount of an agonist anti-TREM2 antibody.

Certain embodiments described herein provide an agonist anti-TREM2 antibody for use in reducing intracellular accumulation of one or more lipids in a cell.

Certain embodiments described herein provide the use of an agonist anti-TREM2 antibody to prepare a medicament for reducing intracellular accumulation of one or more lipids in a cell.

Certain embodiments described herein provide a method of treating Alzheimer's disease in a mammal in need thereof, the method comprising administering to the mammal an agonist anti-TREM2 antibody wherein the mammal has, or has been determined to have, dysregulated lipid metabolism.

Certain embodiments described herein provide an agonist anti-TREM2 antibody for use in the treatment of Alzheimer's disease in a mammal, wherein the mammal has, or has been determined to have, dysregulated lipid metabolism.

Certain embodiments described herein provide the use of an agonist anti-TREM2 antibody to prepare a medicament for treating Alzheimer's disease in a mammal, wherein the mammal has, or has been determined to have, dysregulated lipid metabolism.

Certain embodiments described herein provide a method of treating atherosclerosis in a mammal in need thereof, comprising administering to the mammal an effective amount of an agonist anti-TREM2 antibody.

Certain embodiments described herein provide an agonist anti-TREM2 antibody for use in the treatment of atherosclerosis in a mammal.

Certain embodiments described herein provide the use of an agonist anti-TREM2 antibody to prepare a medicament for treating atherosclerosis in a mammal.

Certain embodiments provide a method of treating inflammation in a mammal in need thereof, comprising administering to the mammal an effective amount of an agonist anti-TREM2 antibody.

Certain embodiments provide an agonist anti-TREM2 antibody for use in the treatment of inflammation in a mammal.

Certain embodiments provide the use of an agonist anti-TREM2 antibody to prepare a medicament for treating inflammation in a mammal.

### BRIEF DESCRIPTION OF THE FIGURES

FIGS. 1A-1C. Attenuated expression of genes implicated in lipid metabolism in Trem2 mutant mice with chronic demyelination induced by a cuprizone diet. (FIG. 1A) Venn diagram of number of differentially expressed genes in bulk microglia isolated from Trem2^{+/+}, Trem2^{+/-}, and Trem2^{-/-} mouse brain without treatment. (FIG. 1B) Venn diagram of number of differentially expressed genes in bulk microglia isolated from Trem2^{+/+}, Trem2^{+/-}, and Trem2^{-/-} mouse brain with chronic demyelination (12 weeks cuprizone treatment). (FIG. 1C) Log 2 fold expression changes in individual genes associated with lipid metabolism in Trem2^{+/+}, Trem2^{+/-}, and Trem2^{-/-} bulk microglia with control diet (left inset) vs. 5 or 12 weeks cuprizone treatment (right inset, top or bottom, respectively).

FIGS. 2A-2B. Attenuated expression of genes implicated in lipid metabolism in Trem2 knockout mice with chronic demyelination. (FIG. 2A) Microglia clusters of single cell RNA sequencing data from individually isolated Trem2^{+/+} control diet microglia (Trem2^{+/+} Ctrl) compared to isolated microglia from Trem2^{+/+}, Trem2^{+/-}, and Trem2^{-/-} mice with 12 week cuprizone treatment (Trem2^{+/+} CPZ, Trem2^{+/-} CPZ, Trem2^{-/-} CPZ). Left: % of all aggregated samples that are represented in each cluster; Right: Heatmap of the percent of each individual sample that is represented in the cluster. (FIG. 2B) Heatmap clustering displaying the ratio of up- and down-regulated top differentially expressed genes in clusters knn5, 8, and 10.

FIG. 3A-3F. Increased abundance of cholesteryl ester and myelin lipids in Trem2 knockout forebrain upon chronic demyelination. (FIG. 3A) Unchanged forebrain total free cholesterol levels in Trem2^{+/+}, Trem2^{+/-}, and Trem2^{-/-} mice with control or cuprizone diet. Increased (FIG. 3B) cholesteryl ester, (FIG. 3C) oxidized cholesteryl ester, (FIG. 3D) bis(monoacylglycero)phosphate (BMP), (FIG. 3E) triacylglyceride and (FIG. 3F) ganglioside levels noted in forebrain from Trem2^{-/-} mice with 12 week cuprizone diet compared to Trem2^{+/+}, Trem2^{+/-}, and Trem2′¹ mice with control diet or 5 week cuprizone, and Trem2^{+/+} and Trem2^{+/-} with 12 week cuprizone. *p<0.05, **p<0.01, ***p<0.001; two-way ANOVA, Tukey test; ***: comparison to +/+ control; +++: comparison between genotypes with 12 week CPZ. Lipids were quantified by LC/MS. (FIG. 3F) Data corresponding to the following conditions are shown from left to right for each ganglioside species: +/+ Control; +/+5 wk CPZ; +/+12 wk CPZ; +/− Control; +/−5 wk CPZ; +/−12 wk CPZ; −/− Control; −/−5 wk CPZ; and −/−12 wk CPZ.

FIG. 4A-4P. Increased abundance of cholesteryl ester and myelin-derived lipids in Trem2 knockout isolated microglia upon chronic demyelination. Increased (FIG. 4A) cholesteryl ester, (FIG. 4B) BMP, (FIG. 4C) hexosylceramide, and (FIG. 4D) galactosylceramide levels detected in microglia isolated from Trem2^{-/-} brain with 12 week cuprizone diet compared to Trem2^{+/+}, Trem2^{+/-}, and Trem2^{-/-} microglia with control diet or 5 week cuprizone, and Trem2^{+/+} and Trem2^{+/-} microglia with 12 week cuprizone. No changes in lipid levels of (FIG. 4E) cholesteryl ester, (FIG. 4F) BMP, (FIG. 4G) hexosylceramide, and (FIG. 4H) galactosylceramide were detected in astrocyte-enriched cell populations isolated from Trem2^{+/+}, Trem2^{+/-}, and Trem2^{-/-} brain with control or cuprizone diet. Increased (FIG. 4I) ceramide, (FIG. 4J) GM3, (FIG. 4K) phosphatidylglycerol, and (FIG. 4L) sulfatide levels detected in microglia isolated from Trem2^{-/-} brain with 12 week cuprizone diet compared to Trem2^{+/+}, Trem2^{+/-}, and Trem2^{-/-} microglia with control diet or 5 week cuprizone, and Trem2^{+/+} and Trem2^{+/-} microglia with 12 week cuprizone. No changes in lipid levels of (FIG. 4M) ceramide, (FIG. 4N) GM3, (FIG. 4O) phosphatidylglycerol, and (FIG. 4P) sulfatide were detected in astrocyte-enriched cell populations isolated from Trem2^{+/+}, Trem2^{+/-}, and Trem2^{-/-} brain with control or cuprizone diet. Lipids were quantified by LC/MS.

FIG. 5A-5B. TREM2 KO BMDM show increased neutral lipid staining upon treatment with oxidized low density lipoprotein (oxLDL). (FIG. 5A) Nile Red staining of cultured TREM2 WT or KO bone marrow-derived macrophages (BMDM) treated with either oxidized LDL (oxLDL, 50 ug/mL) or vehicle for 48h at 63× resolution. (FIG. 5B). Quantification of total spot area shows accumulation of neutral lipids in TREM2 KO, both to a small extent under vehicle condition, as well as a larger extent under lipid challenge (oxLDL) conditions. Data is shown as the mean and standard deviation of three technical replicates.

FIG. 6A-6E. TREM2 KO BMDM show increased lipid accumulation upon treatment with oxidized LDL. Increased levels of (FIG. 6A) cholesteryl esters, (FIG. 6B) ganglioside GM3, (FIG. 6C) triacylglycerides, and (FIG. 6D) hexosylceramide detected in cultured TREM2 KO BMDMs compared to WT BMDMs when dosed with 50 ug/mL oxLDL. (FIG. 6E) No changes in phosphatidylcholine were observed in the TREM2 KO compared to WT. Data is shown as the mean and standard deviation of three technical replicates, and all data is normalized to the average number of cells per well. Lipids were measured by LC/MS.

FIG. 7A-7G. TREM2 KO BMDM show increased lipid accumulation upon treatment with myelin. Cultured Trem2 KO BMDMs show greater accumulation of (FIG. 7A) cholesteryl esters, (FIG. 7B) oxidized cholesteryl esters, (FIG. 7C) diacylglycerides, (FIG. 7D) triacylglycerides, (FIG. 7E) hexosylceramides, (FIG. 7F) lactosylceramides, and (FIG. 7G) gangliosides, when treated with purified mouse brain myelin (25 ug/mL). Data is shown as the mean and S.E.M. of three technical replicates, and all data is normalized to the average number of cells per well. Lipids were measured by LC/MS.

FIG. 8A-8H. TREM2 KO induced pluripotent stem cell (iPSC)-derived human microglia show increased lipid accumulation upon treatment with myelin. TREM2 KO human iPSC-derived microglia show greater accumulation of (FIG. 8A) free cholesterol, (FIG. 8B) phosphatidylserine 38:4, (FIG. 8C) BMP 44:12, (FIG. 8D) lysophosphatidylcholine 16:0, (FIG. 8E) platelet activating factor, (FIG. 8F) cholesterol sulfate, (FIG. 8G) lysophosphatidylethanolamine and (FIG. 8H) plasmalogen phosphatidylethanolamine (PEp), when treated with myelin (25 ug/mL). Data is shown as the mean and standard deviation of three technical replicates, and all data is normalized to the average number of cells per well. Lipids were analyzed by LC/MS.

FIG. 9. Myelin-derived cholesterol is converted into cholesteryl ester via ACAT1 in BMDM. Free cholesterol and cholesteryl ester (CE) levels in BMDMs from wildtype mice dosed with or without myelin for 2 h, then extracted immediately after myelin uptake (TO), or following myelin washout and 2 h (T2) or 4 h (T4) chase. ACAT1 inhibitor was added during myelin uptake and maintained through 4 h washout (T4+ ACAT1 inhibitor). Lipids were measured by LC/MS.

FIG. 10. Recombinant human APOE3 improves the neutral lipid accumulation in Trem2 KO BMDM upon myelin treatment. Trem2 KO BMDMs accumulate more neutral lipid than WT BMDMs when treated for 24h with myelin debris (25 ug/mL), as quantified by Nile Red staining. This accumulation is improved by addition of recombinant human APOE3 (10 ug/mL) into the culture media.

FIG. 11A-11C. ACAT1 inhibition abolishes cholesteryl ester increase in iPSC-derived human microglia upon myelin treatment. (FIG. 11A) ACAT inhibition prevents accumulation of all cholesteryl ester species measured in both WT and Trem2 KO iPSC-derived human microglia treated with purified myelin. (FIG. 11B) Specific example of cholesteryl ester 22:6 is shown. (FIG. 11C) As a control, cholesterol levels are shown to be unaffected by the presence of ACAT1 inhibitor. Data is shown as the mean and standard deviation of three technical replicates, and all data is normalized to the average number of cells per well. Lipids were measured by LC/MS.

FIGS. 12A-12E. An agonist anti-TREM2 antibody decreases neutral lipid accumulation in iPSC-derived human microglia upon myelin treatment. (FIG. 12A) Nile Red images of iPSC-derived human microglia treated with either vehicle or myelin (50 ug/mL), then after 24h either with RSV control or an agonist anti-TREM2 antibody. (FIG. 12B) Spot quantification and (FIG. 12C) lipidomics of triacylglyceride show reduction of lipid accumulation upon treatment with TREM2 antibody compared to isotype control (RSV). Lipidomics data are normalized to the average number of cells per well. Triglycerides were measured by LC/MS. FIGS. 12D-12E include bar charts illustrating quantified levels of triacylglyceride lipid species (in iPSC microglia treated with myelin, followed by incubation with exemplary anti-TREM2 antibodies). FIG. 12E represents data for iPSC microglia for which a myelin washout step was included prior to incubation with the exemplary anti-TREM2 antibodies.

FIG. 13A-13B. (FIG. 13A) Effect of bexarotene on myelin storage in TREM2 KO BMDMs. Trem2 KO BMDMs accumulate more neutral lipid than WT BMDMs when treated for 48h with myelin debris (25 ug/mL), as quantified by Nile Red staining. This accumulation is reduced by co-treatment with bexarotene (10 uM). (FIG. 13B) An ACAT1 inhibitor and LXR agonist decrease cholesteryl ester (CE) levels in human iPSC-derived TREM2 KO microglia. Human iPSC-derived microglia were treated with myelin (25 ug/mL) in C+++ media for 48 hours together with no drug, the ACAT1 inhibitor K604 (500 nM) and the LXR agonist GW3965 (10 uM). CE levels were rescued by both drugs in WT and TREM2 KO cells, but the cholesterol increase seen in TREM2 KO cells was not rescued by the drugs. CE and cholesterol levels were measured in lysates by LC/MS. N=3 biological replicates.

FIG. 14A-14F. TREM2 deficiency prevents DAM conversion during chronic demyelination. (FIG. 14A) Log 2 fold expression changes in individual genes associated with lysosomal function in Trem2^{+/+}, Trem2^{+/-}, and Trem2^{-/-} bulk microglia with control diet (left inset) vs. 5 or 12 weeks cuprizone treatment (right inset, top or bottom, respectively). (FIG. 14B) Log 2 fold expression changes in individual genes associated with lipid metabolism in Trem2^{+/+}, Trem2^{+/-}, and Trem2^{-/-} bulk microglia with control diet (left inset) vs. 5 or 12 weeks cuprizone treatment (right inset, top or bottom, respectively). (FIG. 14C) Heatmap of bulk microglial expression changes (log 2 fold change) from 5 and 12 week control vs. CPZ treated Trem2^{+/+}, Trem2^{+/-}, and Trem2^{-/-} in the top 69 DAM genes downregulated (top) or upregulated (bottom) in 5XFAD compared to wildtype microglia from (Keren-Shaul, et al. (2017). Cell 169, 1276-1290 e1217). Camera, p<1×10⁻⁴¹. (FIGS. 14D-14F) Log 2 fold expression changes in individual (FIG. 14D) homeostatic, (FIG. 14E) Stage 1, and (FIG. 14F) Stage 2 DAM genes in Trem2^{+/+}, Trem2^{+/-}, and Trem2^{-/-} bulk microglia with control (left inset) or CPZ treatment (right inset) for 5 weeks (top) or 12 weeks (bottom).

FIG. 15A-15C. TREM2 deficiency prevents DAM conversion during chronic demyelination. (FIG. 15A) Log 2 fold change for Trem2+/+12 week control vs. CPZ treated mice (top) and Trem2−/− 12 week control vs. CPZ treated mice (bottom) for genes upregulated in DAM vs. homeostatic microglia from 5XFAD mice (see. Keren-Shaul, et al. (2017). Cell 169, 1276-1290 e1217)). (FIGS. 15B-15C) Microglia isolated from aged wildtype brain expressed damage-associated microglia features. (FIG. 15B) Comparison of t-statistics calculated from RNAseq profiles of microglia sorted from aged vs. young Trem2^{+/+} mice (x-axis) and aged vs. young Trem2^{-/-} mice (y-axis); n=7 mice per group. Genes are colored by membership within gene sets of interest. Homeostatic, DAM1 and DAM2 gene sets are as defined in (Keren-Shaul, et al. (2017). Cell 169, 1276-1290 e1217). Cholesterol metabolism-related gene set identified from a subset of differentially expressed genes from 12 week CPZ vs Control treated mice, which are a priori known to be related to cholesterol metabolism. (FIG. 15C) Heatmap of differentially expressed genes from the Trem2 cuprizone and aging cohorts that are upregulated in DAM2 microglia, as defined in (Keren-Shaul, et al. (2017). Cell 169, 1276-1290 e1217), or the cholesterol metabolism-related gene set as defined in (15B). Gene expression values are plotted as zero mean and unit variance.

FIG. 16A-16C. ScRNAseq confirms Trem2^{-/-} microglia exhibit attenuated transition to damage-associated microglia state upon demyelination. (FIG. 16A) Expression profiles for selected marker genes across the dataset, plotted as normalized counts per cell. Left legend denotes upregulated (up arrow) versus downregulated (down arrow) marker genes in indicated clusters. (FIG. 16B) Normalized count of unique molecular identifier (UMI) reads for individual marker genes that define a cluster, compared across expression in all clusters. Left legend denotes upregulated (up arrow) versus downregulated (down arrow) marker genes in indicated clusters. (FIG. 16C) Normalized expression score of upregulated marker genes from Cluster 8, compared across all other clusters. Dots represent individual cells shaded by cluster.

FIG. 17A-17F. TREM2 deficiency causes cholesteryl ester accumulation in the brain. (FIG. 17A) Neurofilament light chain (Nf-L) levels in plasma isolated from Trem2^{+/+}, Trem2^{+/-}, and Trem2^{-/-} mice with control, 5 week, or 12 week cuprizone (CPZ) diet. ***p<0.001, interaction p=0.06; two-way ANOVA, Tukey's test; n=7-16 mice per condition. (FIG. 17B) Neurofilament light chain (Nf-L) levels in plasma isolated from Trem2^{+/+} and Trem2^{-/-} mice at 2 months and 17 months (two-way ANOVA, FDR<0.05, interaction age-genotype p<0.05). (FIG. 17C) Heatmap comparison of lipids significantly altered with control vs. 5 week CPZ treatment. N=3 mice per condition; two-way ANOVA, FDR<0.05. (FIG. 17D) Heatmap of lipids significantly altered by genotype and/or 12 week CPZ treatment in Trem2^{+/+}, Trem2^{+/-}, and Trem2^{-/-} mouse forebrain. Two-way ANOVA, FDR<0.05; columns represent data from individual mice, n=3-7 mice per condition. (FIG. 17E) Concentration of cholesteryl ester (CE) species from extracted Trem2^{+/+}, Trem2^{+/-}, and Trem2^{-/-} mouse forebrain with control, 5 week, or 12 week CPZ diet. Data represent the mean±SEM and are presented in the log 10 scale. Two-way ANOVA, FDR<0.05; genotype-treatment interaction shown for indicated lipid species from Trem2^{-/-} on 12 week CPZ diet, as denoted by asterisks. *p<0.05, **p<0.01, ***p<0.001. Data corresponding to the following conditions are shown from left to right for each CE species: +/+ Control; +/+5 wk CPZ; +/+12 wk CPZ; +/− Control; +/−5 wk CPZ; +/−12 wk CPZ; −/− Control; −/−5 wk CPZ; and −/−12 wk CPZ. (FIG. 17F) Quantification of APP-positive puncta, APP-positive area, and APP intensity in the hippocampus of Trem2^{+/+} and Trem2^{-/-} mice after a control diet or 5 or 12 weeks of CPZ.

FIG. 18A-18M. TREM2 deficiency causes cholesteryl ester accumulation in isolated microglia. (FIG. 18A) Heatmap of lipids significantly altered by treatment and/or genotype in microglia isolated from Trem2^{+/+}, Trem2^{+/-}, and Trem2^{-/-} mouse brain upon control, 5 week, or 12 week cuprizone (CPZ) treatment. Two-way ANOVA, FDR<0.05; columns represent data from individual mice, n=6 mice per condition. (FIG. 18B) Heatmap comparison of lipids detected in cerebral spinal fluid (CSF) isolated from Trem2^{+/+}, Trem2^{+/-}, and Trem2^{-/-} mice upon control, 5 week, or 12 week CPZ treatment. Columns represent data from individual mice, n=5-6 mice per condition. As shown in FIGS. 18A-18B, certain lipidomic alterations are present in Trem2 knockout isolated microglia (FIG. 18A) upon chronic demyelination but are not present in CSF (FIG. 18B). (FIG. 18C) Heatmap comparison of lipids detected in astrocytes isolated from Trem2^{+/+}, Trem2^{+/-}, and Trem2^{-/-} mice upon control, 5 week, or 12 week CPZ treatment. Columns represent data from individual mice, n=5-6 mice per condition. Increased (FIG. 18D) cholesteryl ester levels were detected in microglia isolated from Trem2^{-/-} brain with 12 week cuprizone diet compared to Trem2^{+/+}, Trem2^{+/-}, and Trem2^{-/-} microglia with control diet or 5 week cuprizone, and Trem2^{+/+} and Trem2^{+/-} microglia with 12 week cuprizone. Generally, no changes in lipid levels of cholesteryl ester were detected in astrocyte-enriched cell populations (FIG. 18E) or CSF (FIG. 18F) isolated from Trem2^{+/+}, Trem2^{+/-}, and Trem2^{-/-} brain with control or cuprizone diet. Lipids were quantified by LC/MS. *p<0.05, **p<0.01, ***p<0.001; two-way ANOVA, Tukey test; ***: comparison to +/+ control; +++: comparison between genotypes with 12 week CPZ. (FIG. 18D-18F) Data corresponding to the following conditions are shown from left to right for each sterol species: +/+ Control; +/+5 wk CPZ; +/+12 wk CPZ; +/− Control; +/−5 wk CPZ; +/−12 wk CPZ; −/− Control; −/−5 wk CPZ; and −/−12 wk CPZ. (FIGS. 18G-18M) Concentrations of lipid species from sorted microglia, sorted astrocytes and CSF from Trem2^{+/+}, Trem2^{+/-}, and Trem2^{-/-} mouse brain with control, 5 week, or 12 week CPZ diet: (FIG. 18G) microglia BMP, (FIG. 18H) astrocyte BMP, (FIG. 18I) CSF sterols, (FIG. 18J) CSF sulfatides, (FIG. 18K) CSF BMP, (FIG. 18L) CSF hexosylceramides, and (FIG. 18M) CSF ceramides. Data represent the mean±SEM and are presented on a log 10 scale. Two-way ANOVA; genotype-treatment interaction shown for indicated lipid species from Trem2^{-/-} on a 12 week CPZ diet, as denoted by asterisks; ****p<0.0001. Data corresponding to the following conditions are shown from left to right for each species: +/+ Control; +/+5 wk CPZ; +/+12 wk CPZ; +/− Control; +/−5 wk CPZ; +/−12 wk CPZ; −/− Control; −/−5 wk CPZ; and −/−12 wk CPZ.

FIGS. 19A-19K. Myelin sulfatide binds TREM2 and promotes downstream signaling. (FIG. 19A) Phospho-SYK (pSYK) fold change in TREM2/DAP12-expressing stable HEK293 cells stimulated with liposomes composed from 30% of indicated test lipid and 70% phosphatidylcholine (PC), normalized to buffer control (dotted line) and compared with TREM2 agonist antibody and isotype control. N≥2 experimental replicates from ≥2 averaged technical replicates; SM: sphingomyelin, PE: phosphatidylethanolamine, PS: phosphatidylserine, PI: phosphatidylinositol, GalCer: galactosylceramide. *p<0.05, **p<0.01, ***p<0.001; two-way ANOVA, Sidak test. For each species, Trem2/DAP12 is shown on the left and DAP12 is on the right. (FIG. 19B) Liposome titration curve of pSYK fold changes in human macrophage cells from 2-4 donors upon stimulation with indicated test lipid, normalized to buffer control and compared with TREM2 agonist antibody and isotype control. (FIG. 19C) Liposome stimulation of indicated test lipid in human macrophage cells from 4-5 donors with liposomes only (stimulated) or liposomes with 3 μM recombinant TREM2- or TREM1-extracellular domain (ECD) protein normalized to buffer control (dotted line). *p<0.05; two-way ANOVA, Tukey test. For each species, the following conditions are shown from left to right: Stimulated; Trem2-ECD; and TREM1-ECD. (FIGS. 19D-19E) Surface plasmon resonance binding response of increasing concentrations of wildtype (dark shaded) and mutant (light shaded) R47H hTREM2 protein to (FIG. 19D) 30% sulfatide/70% PC or (FIG. 19E) 30% PS/70% PC 100 nm liposomes. (FIG. 19F) Intensity of vehicle or pHrodo-myelin (5 μg/mL) phagocytosis in Trem2^{+/+} and Trem2^{-/-} BMDM with 5 ng/mL M-CSF. Data represent 3 biological replicates ±SEM from 3 averaged technical replicates; *p<0.05 comparing Trem2^{+/+} and Trem2^{-/-} area under the curve, two-tailed t-test. (FIG. 19G) Intensity of vehicle or pHrodo-myelin (5 μg/mL) phagocytosis in Trem2^{+/+} and Trem2^{-/-} BMDM with 50 ng/mL M-CSF. Data represent 3 biological replicates ±SEM from 3 averaged technical replicates. (FIGS. 19H-19K) Surface plasmon resonance kinetic analysis of (FIGS. 19D-19E) sulfatide liposomes for (FIG. 19H) hTREM2 vs. (FIG. 19I) hTREM2 R47H mutant protein and (FIGS. 19J-19K) phosphatidylserine (PS) liposomes for (FIG. 19J) hTREM2 versus (FIG. 19K) hTREM2 R47H mutant protein. ka: association rate constant, kd: dissociation rate constant, KD: dissociation constant; s: second, M: molar.

FIG. 20A-20C. TREM2 KO BMDMs show sterol accumulation with myelin treatment. FIG. 20A depicts an increase in neutral lipid accumulation in Trem2 KO BMDMs treated with myelin (25 ug/ml) compared to WT BMDMs, as shown by Nile Red staining (left). Cells were imaged and Nile Red was quantified as total spot area (right). Data represent 5 biological replicates of 3 averaged technical replicates ±SEM; *p<0.05; one-tailed t-test for comparison between Trem2^{+/+} with myelin and Trem2^{-/-} with myelin. FIG. 20B shows that cholesteryl esters do not accumulate in the presence of the ACAT inhibitor in both WT and TREM2 KO BMDM dosed with myelin, indicating that the cholesteryl ester accumulation is ACAT-dependent. Cholesterol is shown as a control and is slightly elevated in Trem2 KO BMDM with myelin treatment and ACAT inhibition (FIG. 20C). (FIG. 20B) For each CE species, the following conditions are shown from left to right: +/+; −/−; +/+ myelin; −/− myelin; +/+ myelin/K604; −/− myelin/K604. (FIG. 20C) The bars from left to right represent: +/+; −/−; +/+ myelin; −/− myelin; +/+ myelin/K604; −/− myelin/K604.

FIG. 21A-21B. TREM2 deficiency-associated cholesteryl ester accumulation is rescued by an ACAT1 inhibitor and an LXR agonist in vitro. (FIGS. 21A-21B) Quantification of cholesteryl esters (CE), free cholesterol, triacylglycerols (TG), diacylglycerols (DG) and hexosylceramides (HexCer) from cultured Trem2^{+/+} and Trem2^{-/-} BMDM (FIG. 21B) and of sterols from cultured iPSC-derived microglia (iMG) treated with vehicle, myelin, or myelin with ACAT1 inhibitor K604 (500 nM) or LXR agonist GW (10 μM) for 48h. Differential abundance between experimental groups was identified by fitting the following ANOVA model for each lipid: log 10(abundance)˜treatment+ genotype+ challenge:genotype+ batch. Each plot shows the batch-adjusted mean and its 95% confidence interval for each group (N=3 biological replicates per group). Significant baseline differences between genotypes (main effect) are indicated as #p<0.05, ##p<0.01 and ###p<0.001. Significant drug treatment effects were identified within each genotype by performing a paired t-test on the log 10 transformed abundances comparing the Myelin/Myelin+ inhibitor groups (N=3 biological replicates per condition) and are indicated as * p<0.05 and ** p<0.01. (FIG. 21A) For each species, the following conditions are shown from left to right: vehicle; myelin; and myelin+K604. (FIG. 21B) For each species, the following conditions are shown from left to right: vehicle; myelin; myelin+K604; and myelin+GW.

FIGS. 22A-22G. TREM2 deficiency causes ACAT1-dependent cholesteryl ester accumulation in vitro. (FIG. 22A) Phospho-SYK (pSYK) fold change in TREM2/DAP12-expressing stable HEK293 cells stimulated with LDL or oxidized LDL (oxLDL) normalized to buffer control (dotted line) and compared with TREM2 agonist antibody and isotype control. ≥2 experimental replicates from 22 averaged technical replicates; ***p<0.001 by two-way ANOVA, Sidak's test. For each condition, Trem2/DAP12 is shown on the left and DAP12 is shown on the right. (FIG. 22B) Liposome titration curve of pSYK fold changes in human macrophage cells from 2-4 donors upon oxLDL stimulation, normalized to buffer control (dotted line) and compared with TREM2 agonist antibody and isotype control. (FIG. 22C) oxLDL stimulation of human macrophage cells from 4-5 donors with oxLDL only (stimulated) or oxLDL with 3 μM or 9 μM recombinant TREM2- or TREM1-extracellular domain (ECD) protein normalized to buffer control (dotted line). For each condition, the following are shown from left to right: Stimulated; Trem2-ECD; and Trem1-ECD. (FIG. 22D) Phospho-SYK (pSYK) fold change in Trem2^{+/+} (left) or Trem2^{-/-} (right) BMDM cells stimulated with oxLDL normalized to buffer control (dotted line) and compared with TREM2 agonist antibody and isotype control. (FIG. 22E) Quantification of total spot area of Nile Red staining in vehicle or 25 ug/ml oxLDL-treated Trem2^{+/+} and Trem2^{-/-} BMDM. Data represent 7 biological replicates from 3 averaged technical replicates ±SEM; *p<0.05; two-tailed t-test for comparison between Trem2^{+/+} with oxLDL and Trem2^{-/-} with oxLDL. (FIG. 22F) Average fluorescence intensity per cell in cultured Trem2^{+/+} and Trem2^{-/-} BMDM treated with vehicle or 50 ng/mL oxLDL over a 200 min timelapse. Data represent 3 biological replicates ±SEM from 3 averaged technical replicates. (FIG. 22G) Quantification of cholesteryl esters (CE), free cholesterol, triacylglycerols (TG), and hexosylceramides (HexCer) from cultured Trem2^{+/+} and Trem2^{-/-} BMDM treated with vehicle, oxLDL, or oxLDL with ACAT1 inhibitor K604 (500 nM) for 48h. Differential abundance between experimental groups was identified by fitting the following ANOVA model for each lipid: log 10(abundance)˜treatment+ genotype+ challenge:genotype+ batch. Each plot shows the batch-adjusted mean and its 95% confidence interval for each group (N=3 biological replicates per group). Significant baseline differences between genotypes (main effect) are indicated as #p<0.05, ##p<0.01 and ###p<0.001. Significant drug treatment effects were identified within each genotype by performing a paired t-test on the log 10 transformed abundances comparing the oxLDL/oxLDL+ inhibitor groups (N=3 biological replicates per condition) and are indicated as * p<0.05 and ** p<0.01. For each species, the following conditions are shown from left to right: vehicle; oxLDL; and oxLDL+K604.

FIGS. 23A-23D. TREM2 KO BMDMs show filipin stain accumulation with myelin treatment and an anti-TREM2 antibody reduces filipin stain in human iPSC-derived microglia. FIG. 23A depicts an increase in endolysosomal free cholesterol accumulation in Trem2 KO BMDMs treated with myelin compared to WT BMDMs, as shown by filipin staining. FIG. 23B shows the quantification of filipin fluorescence as total spot area. Data is shown as the mean and standard deviation of three technical replicates. FIG. 23C shows that iPSC microglia have a filipin stain reduction when treated with the TREM2 antibody in comparison to the RSV control. As a positive control cells were treated with the NPC1 inhibitor U18666A at 3 ug/mL. Representative images of endolysosomal free cholesterol stained by filipin. FIG. 23D shows the quantification of filipin fluorescence. Data is shown as the mean and standard deviation of 1-3 technical replicates.

FIG. 24. ApoE KO forebrains show accumulation of cholesteryl esters (CE) in the presence or absence of chronic demyelination. CE accumulation occurs in wildtype forebrain subjected to a 4 week-cuprizone diet compared to normal diet. CE accumulation is exacerbated in ApoE knockout mice on cuprizone versus normal diet. Abundance of acyl phosphatidylserine (Acyl PS) is increased with cuprizone treatment in both wildtype and ApoE knockout mice. *p<0.05, ***p<0.001; Two-way ANOVA, Dunnett's posthoc test, corrected for multiple comparisons. Lipids were quantified by liquid chromatography-mass spectrometry (LCMS). Animals were 6 months old, N=8 (3 male, 5 female) animals per group.

FIG. 25. ApoE KO forebrains show accumulation of multiple cholesteryl esters (CE) species in the presence or absence of chronic demyelination. CE species accumulation occurs in wildtype forebrain subjected to a 4 week-cuprizone diet compared to normal diet. CE species accumulation is exacerbated in ApoE knockout mice on cuprizone versus normal diet. *p<0.05, ***p<0.001; Two-way ANOVA, Dunnett's posthoc test, corrected for multiple comparisons. Lipids were quantified by LCMS. Animals were 6 months old, N=8 (3 male, 5 female) animals per group.

FIGS. 26A-26C. Increased levels of cholesteryl esters (CE) in microglia, astrocytes, and neurons isolated from ApoE KO mice upon chronic demyelination. Most CE species levels are higher in microglia (FIG. 26A) isolated from APOE KO mice versus wildtype (WT) brain, and 12 week-cuprizone diet increases levels in both groups compared to control diet. Astrocytes (FIG. 26B) from ApoE KO mice on 12 week-cuprizone diet show exacerbated accumulation of CE compared to ApoE KO mice on control diet, as well as WT mice on either cuprizone or control diet. Some species of CE, including CE(20:4) and CE(22:6), are increased in neurons (FIG. 26C) sorted from ApoE KO mice on either diet compared to WT mice on either diet. Lipids were quantified by LCMS. Animals were 6 months old, N=6 (3 male, 3 female) animals per group.

FIG. 27A-27H. APOE deficiency causes cholesteryl ester accumulation in the brain, sorted microglia and astrocytes, as well as CSF. (FIG. 27A) Heatmap of top 50 lipids altered by genotype and/or 12 week CPZ treatment in Apoe^{+/+} and Apoe^{-/-} mouse forebrain, ranked by p-value (one-way ANOVA). (FIG. 27B) Concentration of free cholesterol, cholesteryl ester (CE), BMP and triacylglycerides species from Apoe^{+/+} and Apoe^{-/-} mouse forebrain extracts with control or 12 week CPZ diet. (FIG. 27C) Heatmap of top 50 lipids altered by genotype and/or 12 week CPZ treatment in Apoe^{+/+} and Apoe^{-/-} sorted microglia, ranked by p-value (one-way ANOVA). (FIG. 27D) Concentration of free cholesterol, CE, hexosylceramide, ceramide, sulfatide and ganglioside species from Apoe^{+/+} and Apoe^{-/-} sorted microglia with control or 12 week CPZ diet. (FIG. 27E) Heatmap of top 50 lipids altered by genotype and/or 12 week CPZ treatment in Apoe^{+/+} and Apoe^{-/-} sorted astrocytes, ranked by p-value (one-way ANOVA). (FIG. 27F) Concentration of free cholesterol, CE, hexosylceramide, ceramide, sulfatide and ganglioside species from Apoe^{+/+} and Apoe^{-/-} sorted astrocytes with control or 12 week CPZ diet. (FIG. 27G) Heatmap of top 50 lipids altered by genotype and/or 12 week CPZ treatment from Apoe^{+/+} and Apoe^{-/-} CSF, ranked by p-value (one-way ANOVA). (FIG. 27H) Concentration of CE, sulfatide, ganglioside and phosphatidic acid species from Apoe^{+/+} and Apoe^{-/-} CSF with control or 12 week CPZ diet. For FIGS. 27B, 27D, 27F and 27H, data represent the mean±SEM (n=6) and are presented on a log 10 scale. Two-way ANOVA, FDR<0.05; genotype effects are indicated by hashtags and genotype-treatment interactions are indicated by asterisks. *p<0.05, ^{#}p<0.05, ^{##}p<0.01, ^{###}p<0.001, ^{####}p<0.0001. For each species, the following conditions are shown from left to right: Apoe +/+ control; Apoe +/+12 wk CPZ; Apoe −/− control; Apoe −/−12 wk CPZ.

FIG. 28A-28B. Increased abundance of cholesteryl esters (CE) in microglia and astrocytes derived from the brain of 5XFAD mice. CE levels are higher in microglia (FIG. 28A) and astrocytes (FIG. 28B) isolated from 5XFAD mice versus wild-type (WT) mice. Animals were 14 months old. N=4 animals per group.

FIG. 29A-29L. Increased inflammatory cytokine production in mouse TREM2 KO BMDM upon LPS stimulation and myelin treatment. WT and TREM2 KO BMDM were plated at 100,000 cells/well in 50 ng/mL mCSF, prior to treatment with vehicle or 25 ug/mL purified mouse myelin for 48h. For the last 16h of myelin treatment, cells were stimulated with either 0 or 10 ng/mL LPS. Cell culture media was collected and levels of (FIG. 29A) G-CSF, (FIG. 29B) INFy, (FIG. 29C) IL-12 (p40), (FIG. 29D) IL-12 (p70), (FIG. 29E) LIX (CXCL5), (FIG. 29F) MCP-1 (CCL2), (FIG. 29G) MIG (CXCL9), (FIG. 29H) IL-1a and (FIG. 29I) IL-1b were measured by quantitative immunoassay (Eve Technologies). Data represent mean±SEM, n=2 technical replicates.

FIG. 30A-30B. Increased IL-1β cytokine response in human iPSC-derived TREM2 KO microglia and attenuation of IL-1β mRNA response with an anti-TREM2 antibody. (FIG. 30A) WT and TREM2 KO iPSC-derived microglia were treated with LPS/ATP (1 ug/ml and 5 mM, respectively) for 4 hours to stimulate NLRP3 inflammasome activation, after which cell culture media was collected and levels of IL-1β were measured by quantitative immunoassay (Eve Technologies). **p<0.01; Two-way ANOVA, Tukey's posthoc test, corrected for multiple comparisons. Data represents mean±SEM, N=4 biological replicates. Within each grouping the following conditions are shown: control is shown on the left, LPS+ ATP is shown in the middle, and LPS+ ATP+ caspase 1 inhibitor VX-765 (InvivoGen) is shown in the right. (FIG. 30B) iPSC microglia were treated with 25 ug/mL myelin for 24 hours, then treated with a control antibody (anti-RSV) or an anti-TREM2 antibody for 48 hours. IL-1β mRNA levels were measured by qPCR and normalized to GAPDH. N=2 biological replicates.

FIG. 31A-31L TREM2 KO human iPSC-derived microglia (iMG) show differential regulation of lipid metabolism genes at baseline and upon treatment with myelin compared to TREM2 WT iMG. TREM2 KO iMG have higher levels of ABCA1 (FIG. 31A), ABCA7 (FIG. 31B), ABCG1 (FIG. 31C), and LDLR (FIG. 31K) mRNA compared to TREM2 WT iMG at both baseline (vehicle treatment) and upon 24h of 25 ug/mL purified myelin treatment. TREM2 KO iMG have lower levels of APOC1 (FIG. 31D), APOE (FIG. 31E), CH25H (FIG. 31F), FABP3 (FIG. 31G), FABP5 (FIG. 31H), LPL (FIG. 31I), OLR1 (FIG. 31J), and LIPA (FIG. 31L) mRNA compared to TREM2 WT iMG. N=4 biological replicates. Data shown as mean and S.E.M.

FIGS. 32A-32B. 48h treatment with 25 ug/mL purified myelin increases secreted APOE (FIG. 32A) and APOC1 (FIG. 32B) protein in both TREM2 KO and TREM2 WT human IPSC-derived microglia (iMG). Secreted APOE (FIG. 32A) and APOC1 (FIG. 32B) levels are decreased in TREM2 KO iMG under both vehicle and myelin-treated conditions compared to TREM2 WT iMG. N=3 technical replicates. Data shown as median and interquartile range. For each genotype grouping, “vehicle” is shown on the left and “myelin” is shown on the right.

### DETAILED DESCRIPTION

As described herein, it has been determined that a reduction in the functional levels of TREM2 results in dysregulation of lipid metabolism. When a comparable dysregulation is induced with a lipid challenge in cells expressing TREM2, it can be improved with agonist anti-TREM2 antibodies. Similarly, lipid dysregulation can be improved in cells that have reduced TREM2 activity by treatment with an ACAT1 inhibitor, apolipoprotein E (ApoE), an RXR agonist, an LXR agonist or a combination thereof (see. Examples). While RXR and LXR agonists and ACAT1 inhibitors have been used to improve lipid clearance, their mechanism of action targets a variety of cell types and may result in unwanted side effects. In contrast, TREM2 expression is restricted to cells of the myeloid lineage (e.g., microglia, dendritic cells and macrophages). Therefore, agonist anti-TREM2 antibodies may be used as a more targeted approach to facilitate lipid clearance for a variety of conditions. For example, a wide array of diseases and disorders have been associated with dysregulated lipid metabolism in myeloid lineage cells, including certain neurodegenerative disorders (e.g., Alzheimer's disease), atherosclerosis, diseases associated with metabolic syndrome and certain lysosomal storage disorders (e.g., Niemann-Pick disease type C (NPC)). Accordingly, as described herein, agonist anti-TREM2 antibodies may be used to treat dysregulated lipid metabolism in mammals having such conditions.

Further, it has also been shown that a reduction in the functional levels of TREM2 is pro-inflammatory (e.g., results in the upregulation of pro-inflammatory cytokines, including IL-1beta, which is a cytokine of the inflammasome pathway). Conversely, agonizing TREM2 with an antibody attenuates such inflammation (e.g., reduces the inflammasome response). Therefore, a variety of diseases and disorders associated with inflammation and the inflammasome response may also be treated with an agonist anti-TREM2 antibody.

The TREM2 gene encodes a type I transmembrane protein that is a member of the immunoglobulin (Ig) receptor superfamily. TREM2 was originally cloned as a cDNA encoding a TREM1 homologue (Bouchon, A et al., J Exp Med, 2001. 194(8): p. 1111-22). This receptor is a glycoprotein of about 40 kDa, which is reduced to 26 kDa after N-deglycosylation. The TREM2 gene encodes a 230 amino acid-length protein that includes an extracellular domain, a transmembrane region and a short cytoplasmic tail (see, UniProtKB Q9NZC2; NCBI Reference Sequence: NP_061838.1). The extracellular region, encoded by exon 2, is composed of a single type V Ig-SF domain, containing three potential N-glycosylation sites. The putative transmembrane region contains a charged lysine residue. The cytoplasmic tail of TREM2 lacks signaling motifs and is thought to signal through the signaling adaptor molecule DAP12/TYROBP and through DAP10. TREM2 is found on the surface of osteoclasts, immature dendritic cells, and macrophages. In the central nervous system, TREM2 is exclusively expressed in microglia.

Accordingly, certain embodiments disclosed herein provide a method for treating dysregulated lipid metabolism and/or inflammation in a mammal in need thereof (e.g., a human), comprising administering to the mammal an effective amount of an agonist anti-TREM2 antibody.

In certain embodiments, such a method may be used for treating dysregulated lipid metabolism. In certain embodiments, such a method may be used for treating inflammation (e.g., inflammation associated with dysregulated lipid metabolism). In certain embodiments, such a method may be used for treating both dysregulated lipid metabolism and inflammation.

In certain embodiments, cells expressing TREM2 in the mammal exhibit dysregulated lipid metabolism. In certain embodiments, the cells are microglial cells. In certain embodiments, the cells are macrophages.

As used herein the term “dysregulated lipid metabolism” refers to altered lipid metabolism in a cell/mammal as compared to a control (e.g., as compared to a healthy control mammal or a control animal that does not have dysregulated lipid metabolism, reduced TREM2 activity, reduced ApoE activity or an APOE ε4 allele). For example, dysregulated lipid metabolism may encompass altered levels (e.g., via increased formation or decreased degradation) or altered localization/storage of one or more classes or species of lipids. In certain embodiments, dysregulated lipid metabolism includes increased accumulation of one or more classes or species of lipids (e.g., intracellular or extracellular accumulation) as compared to a control.

In certain embodiments, the dysregulated lipid metabolism comprises increased intracellular accumulation of one or more lipids. In certain embodiments, one or more lipids accumulate intracellularly in microglial cells. In certain embodiments, one or more lipids accumulate intracellularly in macrophages. In certain embodiments, one or more lipids do not accumulate intracellularly in astrocytes.

In certain embodiments, the dysregulated lipid metabolism comprises increased extracellular accumulation of one or more lipids.

In certain embodiments, the one or more lipids are selected from the group consisting of cholesteryl esters, oxidized cholesteryl esters, bis(monoacylglycero)phosphate species (BMPs), diacylglycerides, triacylglycerides, hexosylceramides, galactosylceramides, lactosylceramides, sulfatides, gangliosides, phosphatidylserine 38:4, bis(monoacylglycero)phosphate 44:12, lysophosphatidylcholine 16:0, platelet activating factor, cholesterol sulfate, lysophosphatidylethanolamine, sphingomyelin (e.g., SMd18:1/18:0), phosphatidylglycerol (e.g., PG d16:0/18:1), phosphatidylethanolamine (e.g., PE38:6), and combinations thereof. In certain embodiments, the one or more lipids are selected from the group consisting of cholesteryl esters, oxidized cholesteryl esters, bis(monoacylglycero)phosphate species (BMPs), diacylglycerides, triacylglycerides, hexosylceramides, galactosylceramides, lactosylceramides, sulfatides, gangliosides, phosphatidylserine 38:4, bis(monoacylglycero)phosphate 44:12, lysophosphatidylcholine 16:0, platelet activating factor, cholesterol sulfate, lysophosphatidylethanolamine, and combinations thereof. In certain embodiments, the one or more lipids includes a lipid described herein, such as in the Figures or the Examples.

In certain embodiments, the one or more lipids includes a cholesteryl ester.

In certain embodiments, the one or more lipids includes oxidized metabolites of cholesteryl ester (e.g., CE oxoODE, CEHODE, CE HpODE, CE oxoHETE or CE HETE).

In certain embodiments, the agonist anti-TREM2 antibody reduces lipid accumulation (e.g., intracellular or extracellular accumulation). In certain embodiments, the agonist anti-TREM2 antibody reduces accumulation of a cholesteryl ester.

In certain embodiments, the lipid accumulation is reduced by at least about 5%, at least about 10%, at least about 20%, at least about 30%, at least about 40%, at least about 50%, at least about 60%, at least about 70%, at least about 80%, at least about 90%, at least about 95%, 96%, 97%, 98% or 99% as compared to a control. Lipid accumulation levels in a cell can be established by evaluating a sample (e.g., a sample comprising one or more cells) using an assay described herein or known in the art.

In certain embodiments, cells expressing TREM2 in the mammal exhibit inflammation or pro-inflammatory responses, such as the upregulation of pro-inflammatory cytokines. In certain embodiments, the inflammasome is upregulated in cells expressing TREM2. In certain embodiments, the cells are microglial cells. In certain embodiments, the cells are macrophages.

In certain embodiments, expression of at least one pro-inflammatory cytokine is upregulated in the mammal (e.g., in a TREM2 expressing cell, such as a macrophage or microglial cell). In certain embodiments, the at least one cytokine is selected from the group consisting of G-CSF, INFy, IL-12 (p40), IL-12 (p70), LIX (CXCL5), MCP-1 (CCL2), MIG (CXCL9), IL-1alpha, IL-1beta and IL-18. In certain embodiments, the at least one cytokine is associated with the inflammasome pathway (e.g., IL-1beta or IL-18). In certain embodiments, the at least one cytokine is IL-1beta.

In certain embodiments, the agonist anti-TREM2 antibody reduces pro-inflammatory responses (e.g., inflammasome responses) in the mammal. For example, in certain embodiments the expression of at least one pro-inflammatory cytokine is reduced (e.g., as compared to a control, such as a corresponding mammal that was not administered an agonist anti-TREM2 antibody). In certain embodiments, the at least one cytokine is selected from the group consisting of G-CSF, INFy, IL-12 (p40), IL-12 (p70), LIX (CXCL5), MCP-1 (CCL2), MIG (CXCL9), IL-1alpha, IL-1beta and IL-18. In certain embodiments, the at least one cytokine is associated with the inflammasome pathway (e.g., IL-1beta or IL-18). In certain embodiments, the at least one cytokine is IL-1beta. In certain embodiments, the expression of the at least one cytokine is reduced by at least about 5%, at least about 10%, at least about 20%, at least about 30%, at least about 40%, at least about 50%, at least about 60%, at least about 70%, at least about 80%, at least about 90%, at least about 95%, 96%, 97%, 98% or 99% as compared to a control. Cytokine expression levels in a cell/mammal can be established by evaluating a sample (e.g., a sample comprising one or more cells) using an assay described herein or known in the art. For example, the assay may evaluate RNA (e.g., mRNA) or protein expression levels (e.g., as compared to a control).

Certain embodiments described herein also provide a method of treating dysregulated lipid metabolism in a patient in need thereof, comprising:

1) obtaining or having obtained a biological sample from the patient;

2) analyzing the biological sample or having analyzed the sample to detect the presence dysregulated lipid metabolism, thereby diagnosing the patient as having dysregulated lipid metabolism; and

3) administering an effective amount of an agonist anti-TREM2 antibody to the diagnosed patient.

Certain embodiments described herein also provide a method of treating a patient with an agonist anti-TREM2 antibody, the method comprising:

1) obtaining or having obtained a biological sample from the patient;

2) analyzing the biological sample or having analyzed the sample to detect the presence dysregulated lipid metabolism, thereby diagnosing the patient as having dysregulated lipid metabolism; and

3) administering an effective amount of an agonist anti-TREM2 antibody to the diagnosed patient.

Reduced TREM2 Activity. Reduced ApoE Activity and ApoE4 Expression

In certain embodiments, a mammal treated using a method described herein has, or has been determined to have, normal TREM2 activity (e.g., as compared to a healthy control subject).

In certain other embodiments, a mammal treated using a method described herein has, or has been determined to have, reduced TREM2 activity. As used herein the term “reduced TREM2 activity” refers to a cell, or a mammal comprising such cells, that has reduced TREM2 function as compared to a control cell/mammal (e.g., a corresponding cell from a healthy subject). In certain embodiments, the reduced levels of functional protein may result from reduced expression of TREM2 (e.g., via inhibition of transcription, inhibition of RNA maturation, inhibition of RNA translation, altered post-translational modifications, or increased degradation of the RNA or protein) or reduced cell surface levels of TREM2 protein. In certain embodiments, the reduced levels of functional TREM2 are caused by loss or partial loss of function genetic mutations in the TREM2 gene (e.g., R47H, R62H, H157Y, Q33X, T66M or Y38C). In certain embodiments, the reduced levels of functional TREM2 are caused by reduced TREM2 protein levels. In certain embodiments, the reduced levels of functional TREM2 are caused by increased cleavage of the receptor by a disintegrin and metalloproteinase (ADAM) proteases (e.g., ADAM10 and ADAM17), which results in the release of soluble TREM2 (sTREM2) into the extracellular environment. In certain embodiments, the reduced TREM2 activity comprises reduced signaling.

The presence of reduced TREM2 activity in a cell/mammal can be established by evaluating a sample (e.g., a sample comprising one or more cells) using an assay described herein or known in the art. For example, the assay may evaluate RNA or protein expression levels, cell surface TREM2 protein levels or may examine TREM2 activity (e.g., signaling) (e.g., as compared to a control). In other embodiments, the assay may measure the levels of sTREM2 (e.g., as compared to a control). Other functional measures of TREM2 activity, such as reduced pSyk activity or class I PI 3-kinase activity as compared to control cells, can also be used be to identify cells or mammals that have reduced TREM2 activity.

In certain embodiments, the level of functional TREM2 in a sample is reduced by at least about 5%, at least about 10%, at least about 20%, at least about 30%, at least about 40%, at least about 50%, at least about 60%, at least about 70%, at least about 80%, at least about 90%, at least about 95%, 96%, 97%, 98% or 99% as compared to a control. In certain embodiments, the cell/mammal does not express functional TREM2.

In certain embodiments, the mammal has altered expression of one or more additional genes, such as a gene associated with lysosome functions or lipid metabolism (e.g., a gene described herein).

ApoE is a major cholesterol carrier that supports lipid transport and injury repair in the brain. In peripheral tissues, ApoE is primarily produced by the liver and macrophages, and mediates cholesterol metabolism in an isoform-dependent manner. The human APOE gene exists as three polymorphic alleles (e2, E3 and e4), which encode ApoE2, ApoE3 and ApoE4 (see. Genomic coordinates (GRCh38): 19:44,905,748-44,909,394; UniProtKB P02649). ApoE is composed of 299 amino acids and has a molecular mass of ˜34 kDa. Differences between the three ApoE isoforms are limited to amino acids 112 and 158, where either cysteine or arginine is present: ApoE2 (Cys112, Cys158), ApoE3 (Cys112, Arg158), and ApoE4 (Arg112, Arg158). The single amino acid differences at these two positions affect the structure of ApoE isoforms and influence their ability to bind lipids, receptors and AD.

In certain embodiments, a mammal treated using a method described herein has, or has been determined to have, normal ApoE activity (e.g., as compared to a healthy control subject).

In certain other embodiments, a mammal treated using a method described herein has, or has been determined to have, reduced ApoE activity. As used herein the term “reduced ApoE activity” refers to a cell, or a mammal comprising such cells, that has reduced ApoE function as compared to a control cell/mammal (e.g., a corresponding cell from a healthy subject). In certain embodiments, the reduced levels of functional protein may result from reduced expression of ApoE (e.g., via inhibition of transcription, inhibition of RNA maturation, inhibition of RNA translation, altered post-translational modifications, or increased degradation of the RNA or protein). In certain embodiments, the reduced levels of functional ApoE are caused by loss or partial loss of function genetic mutations or coding variants in the APOE gene. In certain embodiments, the reduced levels of functional ApoE are caused by reduced ApoE protein levels. In certain embodiments, the reduced levels of functional ApoE are caused by decreased ApoE secretion. In certain embodiments, the reduced levels of functional ApoE are caused by reduced intracellular or extracellular ApoE transport. In certain embodiments, the reduced levels of functional ApoE are caused by aberrant cellular trafficking, including decreased recycling to the plasma membrane, decreased retrograde transport from endolysosomes to the Golgi complex, decreased trafficking along the biosynthetic pathway. In certain embodiments, the reduced levels of functional ApoE are caused by reduced transport of ApoE cargoes, such as lipids. In certain embodiments, the reduced levels of functional ApoE are caused by reduced efflux of cellular lipids. In certain embodiments, the reduced levels of functional ApoE are caused by reduced anti-oxidant properties.

The presence of reduced ApoE activity in a cell/mammal can be established by evaluating a sample (e.g., a sample comprising one or more cells) using an assay described herein or known in the art. For example, the assay may evaluate RNA or protein expression levels or may examine ApoE activity (e.g., as compared to a control).

In certain embodiments, the level of functional ApoE in a sample is reduced by at least about 5%, at least about 10%, at least about 20%, at least about 30%, at least about 40%, at least about 50%, at least about 60%, at least about 70%, at least about 80%, at least about 90%, at least about 95%, 96%, 97%, 98% or 99% as compared to a control. In certain embodiments, the cell/mammal does not express functional ApoE.

In certain embodiments, a mammal treated using a method described herein does not have, or has been determined to not have, an APOE ε4 allele.

In certain other embodiments, a mammal treated using a method described herein has, or has been determined to have, an APOE ε4 allele. In certain embodiments, the mammal is heterozygous for the APOE ε4 allele. In certain embodiments, the mammal is homozygous for the APOE ε4 allele. An APOE ε4 allele may be detected in a sample (i.e., a sample comprising one or more cells from the mammal) using an assay described herein or using an assay known in the art. In certain embodiments, the assay is a genotyping assay, such as a sequencing assay.

As described herein, a mammal expressing ApoE4 may have dysregulated lipid metabolism and/or inflammation that can be treated with an agonist anti-TREM2 antibody. Thus, in certain embodiments, a mammal having an APOE ε4 allele may be treated using a method described herein.

Accordingly, certain embodiments disclosed herein also provide a method for treating dysregulated lipid metabolism in a mammal in need thereof, comprising administering to the mammal an effective amount of an agonist anti-TREM2 antibody, wherein the mammal has, or has been determined to have, reduced TREM2 activity, reduced ApoE activity and/or an APOE ε4 allele. In certain embodiments, the mammal has, or has been determined to have, reduced TREM2 activity. In certain embodiments, the mammal has, or has been determined to have, reduced ApoE activity. In certain embodiments, the mammal has, or has been determined to have, an APOE ε4 allele.

Certain embodiments also provide a method for treating inflammation in a mammal in need thereof, comprising administering to the mammal an effective amount of an agonist anti-TREM2 antibody, wherein the mammal has, or has been determined to have, reduced TREM2 activity.

Certain embodiments disclosed herein provide a method of treating dysregulated lipid metabolism in a patient in need thereof, comprising:

1) obtaining or having obtained a biological sample from the patient;

2) detecting or having detected reduced TREM2 activity, reduced ApoE activity or an APOE ε4 allele in the sample;

3) diagnosing the patient with dysregulated lipid metabolism when reduced TREM2 activity, reduced ApoE activity or an APOE ε4 allele is detected; and

4) administering an effective amount of an agonist anti-TREM2 antibody to the diagnosed patient.

In certain embodiments, the method comprises diagnosing the patient with dysregulated lipid metabolism when reduced TREM2 activity is detected. In certain embodiments, the method comprises diagnosing the patient with dysregulated lipid metabolism when reduced ApoE activity is detected. In certain embodiments, the method comprises diagnosing the patient with dysregulated lipid metabolism when an APOE ε4 allele is detected.

Certain embodiments disclosed herein provide a method of treating a patient with an agonist anti-TREM2 antibody, the method comprising:

1) obtaining or having obtained a biological sample from the patient;

2) analyzing the biological sample or having analyzed the sample to detect reduced TREM2 activity, reduced ApoE activity or an APOE ε4 allele, thereby diagnosing the patient as having dysregulated lipid metabolism; and

4) administering an effective amount of an agonist anti-TREM2 antibody to the diagnosed patient.

In certain embodiments, the method comprises analyzing the biological sample or having analyzed the sample to detect reduced TREM2 activity. In certain embodiments, the method comprises analyzing the biological sample or having analyzed the sample to detect reduced ApoE activity. In certain embodiments, the method comprises analyzing the biological sample or having analyzed the sample to detect an APOE ε4 allele.

### TREM2 Expressing Cells

As described herein, reduced TREM2 activity has been specifically shown to cause dysregulation of lipid metabolism in certain cell types (e.g., microglial cells and macrophages) but not in certain other cell types (e.g., astrocytes) (see, the Examples). Reduction of functional TREM2 also has been shown to cause an increase in pro-inflammatory responses.

Accordingly, certain embodiments disclosed herein provide a method of reducing intracellular accumulation of one or more lipids in a cell, comprising contacting the cell with an effective amount of an agonist anti-TREM2 antibody. Certain embodiments also provide a method of reducing the expression of at least one pro-inflammatory cytokine in a cell, comprising contacting the cell with an effective amount of an agonist anti-TREM2 antibody.

In certain embodiments, the cell expresses TREM2. In certain embodiments, the cell is a microglial cell. In certain embodiments, the cell is a macrophage.

In certain embodiments, the one or more lipids are selected from the group consisting of cholesteryl esters, oxidized cholesteryl esters, bis(monoacylglycero)phosphate species (BMPs), diacylglycerides, triacylglycerides, hexosylceramides, galactosylceramides, lactosylceramides, sulfatides, gangliosides, phosphatidylserine 38:4, bis(monoacylglycero)phosphate 44:12, lysophosphatidylcholine 16:0, platelet activating factor, cholesterol sulfate, lysophosphatidylethanolamine, sphingomyelin (e.g., SMd18:1/18:0), PG (e.g., PG d16:0/18:1), PE (e.g., PE38:6), and combinations thereof. In certain embodiments, the one or more lipids are selected from the group consisting of cholesteryl esters, oxidized cholesteryl esters, BMPs, diacylglycerides, triacylglycerides, hexosylceramides, galactosylceramides, lactosylceramides, sulfatides, gangliosides, phosphatidylserine 38:4, bis(monoacylglycero)phosphate 44:12, lysophosphatidylcholine 16:0, platelet activating factor, cholesterol sulfate, lysophosphatidylethanolamine, and combinations thereof. In certain embodiments, the one or more lipids includes a cholesteryl ester. In certain embodiments, the one or more lipids includes a lipid described herein.

In certain embodiments, the lipid accumulation is reduced by at least about 5%, at least about 10%, at least about 20%, at least about 30%, at least about 40%, at least about 50%, at least about 60%, at least about 70%, at least about 80%, at least about 90%, at least about 95%, 96%, 97%, 98% or 99% as compared to a control.

In certain embodiments, the at least one cytokine is selected from the group consisting of G-CSF, INFy, IL-12 (p40), IL-12 (p70), LIX (CXCL5), MCP-1 (CCL2), MIG (CXCL9), IL-1alpha, IL-1beta and IL-18. In certain embodiments, the at least one cytokine is associated with the inflammasome pathway (e.g., IL-1beta or IL-18). In certain embodiments, the at least one cytokine is IL-1beta.

In certain embodiments, the expression of the at least one cytokine is reduced by at least about 5%, at least about 10%, at least about 20%, at least about 30%, at least about 40%, at least about 50%, at least about 60%, at least about 70%, at least about 80%, at least about 90%, at least about 95%, 96%, 97%, 98% or 99% as compared to a control (e.g., a corresponding control cell that was not administered an agonist anti-TREM2 antibody).

In certain embodiments, the cell has, or has been determined to have, reduced TREM2 activity. In other embodiments, the cell has normal TREM2 activity.

In certain embodiments, the cell has, or has been determined to have, reduced ApoE activity (e.g., the cell has an APOE loss or partial loss of function mutation or coding variant).

In other embodiments, the cell has normal ApoE activity.

In certain embodiments, the cell expresses, or has been determined to express, ApoE4. In certain other embodiments, the cell does not express, or has been determined to not express, ApoE4.

In certain embodiments, the cell is contacted with an agonist anti-TREM2 antibody described herein (e.g., an agonist anti-TREM2 antibody described herein, such as MAB17291 or 78.18).

In certain embodiments, the cell is contacted with the agonist anti-TREM2 antibody, in vitro, in vivo or ex vivo. In certain embodiments, the cell is contacted with the agonist anti-TREM2 antibody in vitro. In certain embodiments, the cell is contacted with the agonist anti-TREM2 antibody in vivo. In certain embodiments, the cell is contacted with the agonist anti-TREM2 antibody ex vivo.

In certain embodiments, the cell is present in a mammal and is contacted with the agonist anti-TREM2 antibody in vivo. In such an embodiment, the cell may be contacted through administration of the antibody. In certain embodiments, the administration is systemic administration.

In certain embodiments, the mammal has inflammation associated with the intracellular lipid accumulation. In certain embodiments, the agonist anti-TREM2 antibody reduces the expression of at least one pro-inflammatory cytokine (e.g., a pro-inflammatory cytokine described herein, such as G-CSF, INFy, IL-12 (p40), IL-12 (p70), LIX (CXCL5), MCP-1 (CCL2), MIG (CXCL9), IL-1alpha, IL-1beta or IL-18). In certain embodiments, the at least one cytokine is IL-1beta.

In certain embodiments, the mammal has or is prone to developing a disease or condition described herein.

### Treatment of Certain Diseases or Conditions

As described in the Examples, TREM2 loss of function in macrophages and microglia results in the inability to process and metabolize lipids (e.g., cholesterol, cholesteryl esters (CE), triglycerides and sphingolipids). Further, it has been shown that accumulation of these lipids leads to pro-inflammatory responses (e.g., upregulation of pro-inflammatory cytokines, including IL-1beta, which is a cytokine of the inflammasome pathway). Conversely, agonizing TREM2 with an antibody decreases lipid burden and attenuates inflammation. Accordingly, as described herein, an agonist anti-TREM2 antibody may be used to correct lipid dysregulation and inflammatory responses in macrophages, microglia or other cell types expressing TREM2 and treat related diseases and disorders.

For example, increased lipid burden in microglial cells and associated inflammation are key features of a variety of neurodegenerative diseases, including Alzheimer's disease (AD). CE is known to accumulate in AD patient brain and AD mouse models (Astarita, et al. (2011). PLoS One 6, e24777; Chan, et al., (2012). J Biol Chem 287, 2678-2688; Morel, et al. (2013). Nat Commun 4, 2250; Shibuya, et al. (2015). Future Med Chem 7, 2451-2467) and LOAD-linked TREM2 variants result in a partial loss of function (Ulland, T. K., and Colonna, M. (2018). Nat Rev Neurol 14, 667-675). Thus, in light of the results described herein, enhancing TREM2 function may be beneficial in AD, in part by facilitating lipid clearance in microglia. Similarly, an agonist anti-TREM2 antibody may also be useful for reducing lipid burden and inflammation in other neurodegenerative disorders that feature these pathologies. Such diseases include, but are not limited to, Nasu-Hakola disease (NHD), Lewy body dementia, Parkinson's disease, retinal degeneration (e.g., macular degeneration), Huntington's disease, Frontotemporal Lobar Degeneration (FTD) and Amyotrophic Lateral Sclerosis (ALS).

As described in the Examples, TREM2 also plays a role in regulating lysosomal cholesterol and an agonist anti-TREM2 antibody was shown to reduce endolysosomal free cholesterol accumulation in cells having reduced TREM2 expression. Therefore, agonizing TREM2 may be useful to treat certain lysosomal storage disorders associated with cholesterol accumulation, such as Niemann-Pick disease (types A, B or C).

Additionally, TREM2 has been shown to be involved in the control of microglial gene expression and cholesterol transport upon chronic myelin phagocytosis, and failure to properly execute this program results in extensive neuronal damage in the brain (see, the Examples). These results indicate that increasing TREM2 activity may be neuroprotective (e.g., for aging) and may stimulate remyelination in certain neurodegenerative diseases, such as multiple sclerosis and vanishing white matter disease.

TREM2 is also expressed on a subset of macrophages outside of the CNS (e.g., in adipose tissue, the liver, skeletal muscle, and atherosclerotic lesions in arteries). TREM2 modulation may be used to alter lipid metabolism and inflammatory responses in these tissues to treat a variety of associated diseases. For example, metabolic syndrome, which comprises a series of conditions such as obesity, type 2 diabetes, atherosclerosis, alcoholic and non-alcoholic fatty liver disease, and alcoholic and non-alcoholic steatohepatitis, is typically associated with low grade, chronic (i.e., unresolved) inflammation in various tissues, including the adipose tissue, the liver and the skeletal muscle. Myeloid cells, such as monocytes and macrophages, are key mediators of these inflammatory responses, which may ultimately lead to insulin resistance, glucose intolerance and atherosclerosis. Central to the inflammatory response in these tissues is the interaction between myeloid cells and adipocytes or other fat-containing cells, such as hepatocytes, as well as the extent of lipid dysregulation in myeloid cells themselves. Accordingly, modulating lipid metabolism/inflammation with a TREM2 agonist may be useful to treat metabolic syndrome and conditions associated with metabolic syndrome. Additionally, rheumatoid arthritis (RA) is an autoimmune disease that causes chronic inflammation of the joints and is also associated with lipid dysregulation. These lipid anomalies increase the risk of developing various cardiovascular diseases. As such, modulating lipid metabolism/inflammation with a TREM2 agonist may be useful to treat RA.

A reduction in functional TREM2 also results in the upregulation of a variety of pro-inflammatory cytokines, including the IL-1beta inflammasome pathway associated cytokine. As described in the Examples, this inflammasome response was reduced by an agonist anti-TREM2 antibody, demonstrating its anti-inflammatory effect and its utility in treating diseases and conditions associated with inflammation (e.g., inflammasome related diseases and disorders). For example, administration of an anti-TREM2 antibody may be useful in treating diseases such as RA, gout, and certain bowel conditions (e.g., inflammatory bowel disease (IBD)).

Thus, in certain embodiments, a method disclosed herein may be used to treat mammal that has or is prone to developing Alzheimer's disease, NHD, Lewy body dementia, Parkinson's disease, retinal degeneration (e.g., macular degeneration), FTD, ALS or Huntington's disease. A method disclosed herein, may also be used to treat obesity, type 2 diabetes, alcoholic and non-alcoholic steatohepatitis, alcoholic and non-alcoholic fatty liver disease, atherosclerosis and other diseases associated with the metabolic syndrome. In certain embodiments, a method described herein is not used to treat non-alcoholic steatohepatitis. In certain embodiments, a method described herein may be used to treat a lysosomal storage disorder, such as Niemann-Pick disease type A, B or C. In certain other embodiments, a method described herein may be used to treat a disease associated with demyelination (e.g., multiple sclerosis or vanishing white matter disease). A method disclosed herein may be used to treat a mammal that has or is prone to developing inflammation or a disease or disorder associated with inflammation, such as an inflammasome related diseases and disorders. In certain embodiments, a method described herein may be used to treat rheumatoid arthritis (RA), gout, and certain bowel conditions (e.g., inflammatory bowel disease (IBD)). A method disclosed herein, may also be used to treat aging or an effect associated with aging. In certain embodiments, such a method reduces cellular aging and/or improves cellular function/activity. In certain embodiments, such a method increases the lifespan of a cell.

Treating lipid dysregulation and/or inflammation in a mammal having one or more of these conditions could alter the natural course of the disease (e.g., by preventing occurrence or recurrence of disease, alleviation of symptoms, diminishment of any direct or indirect pathological consequences of the disease, decreasing the rate of disease progression, amelioration or palliation of the disease state, or remission or improved prognosis).

As used herein, the term “prone to developing” refers to a mammal that is at an increased risk of developing the particular disease or condition (e.g., due to a genetic risk factor, such as expressing an ApoE4 isoform or a TREM2 mutation; due to a lifestyle choice, such as eating a diet high in fats; or due condition resulting from a combination of genetic and lifestyle factors, such as metabolic syndrome).

In certain embodiments, a mammal treated using a method described herein has NHD. In certain embodiments, the mammal is prone to developing NHD.

In certain embodiments, a mammal treated using a method described herein has Niemann Pick disease type C. In certain embodiments, the mammal is prone to developing Niemann Pick disease type C.

In certain embodiments, a mammal treated using a method described herein has Alzheimer's disease. In certain embodiments, the mammal is prone to developing Alzheimer's disease. Thus, certain embodiments disclosed herein also provide a method of treating Alzheimer's disease in a mammal in need thereof, the method comprising administering to the mammal an agonist anti-TREM2 antibody, wherein the mammal has, or has been determined to have, dysregulated lipid metabolism. In certain embodiments, the mammal has, or has been determined to have, dysregulated lipid metabolism in TREM2-expressing cells (e.g., microglial cells). In certain embodiments, the TREM2-expressing cells have, or have been determined to have, reduced TREM2 activity. In certain embodiments the dysregulated lipid metabolism comprises increased intracellular accumulation of one or more lipids described herein (e.g., a cholesteryl ester). In certain embodiments, the mammal has inflammation associated with dysregulated lipid metabolism (e.g., at least one pro-inflammatory cytokine is upregulated, such as a cytokine described herein (e.g., IL-1beta)).

In certain embodiments, a mammal treated using a method described herein has atherosclerosis. In certain embodiments, the mammal is prone to developing atherosclerosis. Thus, certain embodiments disclosed herein also provide a method of treating atherosclerosis in a mammal in need thereof, comprising administering to the mammal an effective amount of an agonist anti-TREM2 antibody described herein (e.g., MAB17291 or 78.18). In certain embodiments, the mammal has, or has been determined to have, dysregulated lipid metabolism. In certain embodiments, the dysregulated lipid metabolism comprises increased accumulation (e.g., intracellular or extracellular accumulation) of one or more lipids described herein. In certain embodiments, one or more lipids accumulate intracellularly in macrophages (e.g., macrophages that have, or that have been determined to have reduced TREM2 activity). In certain embodiments, the mammal has inflammation associated with the dysregulated lipid metabolism (e.g., at least one pro-inflammatory cytokine is upregulated, such as a cytokine described herein (e.g., IL-1beta)). In certain embodiments, the method further comprises administering a second therapeutic agent (e.g., therapeutic agent described herein). In certain embodiments, the second therapeutic agent is an agent useful for treating atherosclerosis. In certain embodiments, the second therapeutic agent is an LXR agonist or an RXR agonist as described herein or an ACAT1 inhibitor as described herein.

### Agonist Anti-TREM2 Antibodies

As described herein, in certain embodiments, an effective amount of an agonist anti-TREM2 antibody is administered to a mammal to treat dysregulation of lipid metabolism or a disease or condition associated with dysregulation of lipid metabolism, such as Alzheimer's disease or atherosclerosis. In certain other embodiments, an effective amount of an agonist anti-TREM2 antibody is administered to a mammal to treat inflammation or a disease or condition associated with inflammation.

As used herein, the term “TREM2 protein” refers to a triggering receptor expressed on myeloid cells 2 protein that is encoded by the gene Trem2. As used herein, a “TREM2 protein” refers to a native (i.e., wild-type) TREM2 protein of any vertebrate, such as but not limited to human, non-human primates (e.g., cynomolgus monkey), rodents (e.g., mice, rat), and other mammals. In some embodiments, a TREM2 protein is a human TREM2 protein having the sequence identified in UniprotKB accession number Q9NZC2.

As used herein, the term “TREM2” also includes protein variants and recombinant TREM2 or a fragment thereof.

As used herein, the term “anti-TREM2 antibody” refers to an antibody that specifically binds to a TREM2 protein (e.g., human TREM2).

As used herein, the term “agonist anti-TREM2 antibody” refers to an antibody that can bind to and activate TREM2 or increase at least one biological activity of TREM2.

Certain anti-TREM2 antibodies (e.g., agonist antibodies), and fragments thereof, are known in the art. For example, anti-TREM2 antibodies include, but are not limited to, MAB17291 (clone #237920, R&D Systems) and 78.18 (cat No. MCA4772; Bio-Rad). Antibodies to TREM2 have also been described, e.g., in Patent/Publication Nos. WO2016/023019, WO2017/062672, WO2017/058866, WO2018/195506, WO2019/118513, and WO2019/028292.

In certain embodiments, the agonist anti-TREM2 antibody, or fragment thereof, is MAB17291 or 78.18.

In one embodiment, the agonist anti-TREM2 antibody, or fragment thereof, specifically binds to TREM2 and increases its activity. In certain embodiments, the agonist anti-TREM2 antibody is a full-length antibody (for example, an IgG1 or IgG4 antibody). In certain embodiments, a fragment of an agonist anti-TREM2 antibody is used in the methods disclosed herein and comprises only an antigen-binding portion (for example, a Fab, F(ab′)₂ or scFv fragment). In certain embodiments, the agonist anti-TREM2 antibody, or fragment thereof, is modified to affect functionality, e.g., to eliminate residual effector functions (Reddy et al., 2000, J. Immunol. 164:1925-1933). Mutations that can eliminate effector include the “LALA” mutations (L234A/L235A mutations, numbered according the EU numbering scheme).

In certain embodiments, the agonist anti-TREM2 antibody is a monoclonal antibody, or fragment thereof. In certain embodiments, the agonist anti-TREM2 antibody is an isolated recombinant monoclonal antibody, or fragment thereof, that binds specifically to TREM2. In certain embodiments, the agonist anti-TREM2 antibody, or a fragment thereof, is a human antibody, or a fragment thereof. In certain embodiments, the antibodies are fully human.

In certain embodiments, the antibodies or antigen-binding fragments are bispecific comprising a first binding specificity to TREM2 and a second binding specificity for a second target epitope. The second target epitope may be another epitope on TREM2 or on a different protein.

As used herein, the term “Fc receptor” refers to the surface receptor protein found on immune cells including B lymphocytes, natural killer cells, macrophages, basophils, neutrophils, and mast cells, which has a binding specificity for the Fc region of an antibody. The term “Fc receptor” includes, but is not limited to, a Fcy receptor (e.g., FcyRI (CD64), FcyRI IA (CD32), FcyRII B (CD32), FcyRI IIA (CD16a), and FcyRIII B (CD16b)), Fca receptor (e.g., FcaRI or CD89) and Fcs receptor (e.g., FcsRI, and FcsRII (CD23)).

As used herein, the term “antibody” refers to a protein with an immunoglobulin fold that specifically binds to an antigen via its variable regions. The term encompasses intact polyclonal antibodies, intact monoclonal antibodies, single chain antibodies, multispecific antibodies such as bispecific antibodies, monospecific antibodies, monovalent antibodies, chimeric antibodies, humanized antibodies, and human antibodies. The term “antibody,” as used herein, also includes antibody fragments that retain binding specificity, including but not limited to Fab, F(ab′)₂, Fv, scFv, and bivalent scFv. Antibodies can contain light chains that are classified as either kappa or lambda. Antibodies can contain heavy chains that are classified as gamma, mu, alpha, delta, or epsilon, which in turn define the immunoglobulin classes, IgG, IgM, IgA, IgD and IgE, respectively.

An exemplary immunoglobulin (antibody) structural unit comprises a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms “variable light chain” (VL) and “variable heavy chain” (VH) refer to these light and heavy chains, respectively.

The term “variable region” or “variable domain” refers to a domain in an antibody heavy chain or light chain that is derived from a germline Variable (V) gene, Diversity (D) gene, or Joining (J) gene (and not derived from a Constant (Cμ and Cδ) gene segment), and that gives an antibody its specificity for binding to an antigen. Typically, an antibody variable region comprises four conserved “framework” regions interspersed with three hypervariable “complementarity determining regions.”

The term “complementarity determining region” or “CDR” refers to the three hypervariable regions in each chain that interrupt the four framework regions established by the light and heavy chain variable regions. The CDRs are primarily responsible for antibody binding to an epitope of an antigen. The CDRs of each chain are typically referred to as CDR1, CDR2, and CDR3, numbered sequentially starting from the N-terminus, and are also typically identified by the chain in which the particular CDR is located. Thus, a VH CDR3 or CDR-H3 is located in the variable region of the heavy chain of the antibody in which it is found, whereas a VL CDR1 or CDR-L1 is the CDR1 from the variable region of the light chain of the antibody in which it is found.

The “framework regions” or “FRs” of different light or heavy chains are relatively conserved within a species. The framework region of an antibody, that is the combined framework regions of the constituent light and heavy chains, serves to position and align the CDRs in three-dimensional space. Framework sequences can be obtained from public DNA databases or published references that include germline antibody gene sequences. For example, germline DNA sequences for human heavy and light chain variable region genes can be found in the “VBASE2” germline variable gene sequence database for human and mouse sequences.

The amino acid sequences of the CDRs and framework regions can be determined using various well-known definitions in the art, e.g., Kabat, Chothia, international ImMunoGeneTics database (IMGT), AbM, and observed antigen contacts (“Contact”). In some embodiments, CDRs are determined according to the Contact definition. See, MacCallum et al., J. Mol. Biol., 262:732-745 (1996). In some embodiments, CDRs are determined by a combination of Kabat, Chothia, and/or Contact CDR definitions.

The terms “antigen-binding portion” and “antigen-binding fragment” are used interchangeably herein and refer to one or more fragments of an antibody that retains the ability to specifically bind to an antigen (e.g., a TREM2 protein) via its variable region. Examples of antigen-binding fragments include, but are not limited to, a Fab fragment (a monovalent fragment consisting of the VL, VH, CL and CHI domains), F(ab′)₂ fragment (a bivalent fragment comprising two Fab fragments linked by a disulfide bridge at the hinge region), single chain Fv (scFv), disulfide-linked Fv (dsFv), complementarity determining regions (CDRs), a VL (light chain variable region), and a VH (heavy chain variable region).

The term “epitope” refers to the area or region of an antigen to which the CDRs of an antibody specifically binds and can include a few amino acids or portions of a few amino acids, e.g., 5 or 6, or more, e.g., 20 or more amino acids, or portions of those amino acids. For example, where the target is a protein, the epitope can be comprised of consecutive amino acids (e.g., a linear epitope), or amino acids from different parts of the protein that are brought into proximity by protein folding (e.g., a discontinuous or conformational epitope). In some embodiments, the epitope is phosphorylated at one amino acid (e.g., at a serine or threonine residue).

As used herein, the phrase “recognizes an epitope,” as used with reference to an anti-TREM2 antibody, means that the antibody CDRs interact with or specifically bind to the antigen (i.e., the TREM2 protein) at that epitope or a portion of the antigen containing that epitope.

As used herein, the term “multispecific antibody” refers to an antibody that comprises two or more different antigen-binding portions, in which each antigen-binding portion comprises a different variable region that recognizes a different antigen, or a fragment or portion of the antibody that binds to the two or more different antigens via its variable regions. As used herein, the term “bispecific antibody” refers to an antibody that comprises two different antigen-binding portions, in which each antigen-binding portion comprises a different variable region that recognizes a different antigen, or a fragment or portion of the antibody that binds to the two different antigens via its variable regions.

A “monoclonal antibody” refers to antibodies produced by a single clone of cells or a single cell line and consisting of or consisting essentially of antibody molecules that are identical in their primary amino acid sequence.

A “polyclonal antibody” refers to an antibody obtained from a heterogeneous population of antibodies in which different antibodies in the population bind to different epitopes of an antigen.

A “chimeric antibody” refers to an antibody molecule in which the constant region, or a portion thereof, is altered, replaced or exchanged so that the antigen-binding site (i.e., variable region, CDR, or portion thereof) is linked to a constant region of a different or altered class, effector function and/or species, or in which the variable region, or a portion thereof, is altered, replaced or exchanged with a variable region having a different or altered antigen specificity (e.g., CDR and framework regions from different species). In some embodiments, a chimeric antibody is a monoclonal antibody comprising a variable region from one source or species (e.g., mouse) and a constant region derived from a second source or species (e.g., human). Methods for producing chimeric antibodies are described in the art.

A “humanized antibody” is a chimeric antibody derived from a non-human source (e.g., murine) that contains minimal sequences derived from the non-human immunoglobulin outside the CDRs. In general, a humanized antibody will comprise at least one (e.g., two) antigen-binding variable domain(s), in which the CDR regions substantially correspond to those of the non-human immunoglobulin and the framework regions substantially correspond to those of a human immunoglobulin sequence. In some instances, certain framework region residues of a human immunoglobulin can be replaced with the corresponding residues from a non-human species to, e.g., improve specificity, affinity, and/or serum half-life. The humanized antibody can also comprise at least a portion of an immunoglobulin constant region (Fc), typically that of a human immunoglobulin sequence. Methods of antibody humanization are known in the art.

A “human antibody” or a “fully human antibody” is an antibody having human heavy chain and light chain sequences, typically derived from human germline genes. In some embodiments, the antibody is produced by a human cell, by a non-human animal that utilizes human antibody repertoires (e.g., transgenic mice that are genetically engineered to express human antibody sequences), or by phage display platforms.

The term “specifically binds” or “binds specifically to”, or the like, means that a binding molecule (e.g., an antibody or antigen-binding fragment thereof) is able to bind a target (e.g., an antigen such as TREM2), without substantially recognizing and binding other unrelated molecules present in a sample, such a biological sample. In some cases, specific binding can be characterized by an equilibrium dissociation constant of about 10⁻⁶, 10⁻⁷, or 10⁻⁸ M or less (a smaller K_{D} denotes a tighter binding). Methods for determining whether two molecules specifically bind are well known in the art and include, for example, equilibrium dialysis, surface plasmon resonance (e.g., BIACORE™), and the like. Moreover, multi-specific antibodies that bind to one domain in TREM2 and one or more additional antigens or a bi-specific that binds to two different regions of TREM2 are nonetheless considered antibodies that “specifically bind”, as used herein.

The term “high affinity” antibody refers to those mAbs having a binding affinity to TREM2, expressed as K_{D}, of at least 10⁻⁷ M, 10⁻⁸ M, 10⁻⁹M, 10⁻¹⁰ M, or 10⁻¹¹ M, as measured by surface plasmon resonance, e.g., BIACORE™ or solution-affinity ELISA.

Agonist anti-TREM2 antibodies, or fragments thereof, may be conjugated to a moiety such a ligand or a therapeutic moiety (“immunoconjugate”), such as a second agonist anti-TREM2 antibody, or an antibody to another antigen.

An “isolated antibody”, as used herein, is intended to refer to an antibody that is substantially free of other antibodies (Abs) having different antigenic specificities (e.g., an isolated antibody that specifically binds TREM2, or a fragment thereof, is substantially free of Abs that specifically bind antigens other than TREM2.

The term “surface plasmon resonance”, as used herein, refers to an optical phenomenon that allows for the analysis of real-time biomolecular interactions by detection of alterations in protein concentrations within a biosensor matrix, for example using the BIACORE™ system (Pharmacia Biosensor AB, Uppsala, Sweden and Piscataway, N.J.).

The term “K_{D}”, as used herein, is intended to refer to the equilibrium dissociation constant of a particular antibody-antigen interaction.

As applied to polypeptides, the term “substantial similarity” or “substantially similar” means that two peptide sequences, when optimally aligned, such as by the programs GAP or BESTFIT using default gap weights, share at least 80%, sequence identity, 90% sequence identity, or at least 95%, 98% or 99% sequence identity. Residue positions, which are not identical, may differ by conservative amino acid substitutions. A “conservative amino acid substitution” is one in which an amino acid residue is substituted by another amino acid residue having a side chain (R group) with similar chemical properties (e.g., charge or hydrophobicity). In general, a conservative amino acid substitution will not substantially change the functional properties of a protein. In cases where two or more amino acid sequences differ from each other by conservative substitutions, the percent or degree of similarity may be adjusted upwards to correct for the conservative nature of the substitution. Means for making this adjustment are well known to those of skill in the art. See, e.g., Pearson (1994) Methods Mol. Biol. 24: SOT-SSI, which is herein incorporated by reference. Examples of groups of amino acids that have side chains with similar chemical properties include 1) aliphatic side chains: glycine, alanine, valine, leucine and isoleucine; 2) aliphatic-hydroxyl side chains: serine and threonine; 3) amide-containing side chains: asparagine and glutamine; 4) aromatic side chains: phenylalanine, tyrosine, and tryptophan; 5) basic side chains: lysine, arginine, and histidine; 6) acidic side chains: aspartate and glutamate, and 7) sulfur-containing side chains: cysteine and methionine. Conservative amino acids substitution groups include: valine-leucine-isoleucine, phenylalanine-tyrosine, lysine-arginine, alanine-valine, glutamate-aspartate, and asparagine-glutamine. Alternatively, a conservative replacement is any change having a positive value in the PAM250 log-likelihood matrix disclosed in Gonnet et al. (1992) Science 256:1443 45, herein incorporated by reference. A “moderately conservative” replacement is any change having a nonnegative value in the PAM250 log-likelihood matrix.

Sequence similarity for polypeptides is typically measured using sequence analysis software. Protein analysis software matches similar sequences using measures of similarity assigned to various substitutions, deletions and other modifications, including conservative amino acid substitutions. For instance, GCG software contains programs such as GAP and BESTFIT which can be used with default parameters to determine sequence homology or sequence identity between closely related polypeptides, such as homologous polypeptides from different species of organisms or between a wild type protein and a mutein thereof. See, e.g., GCG Version 6.1. Polypeptide sequences also can be compared using FASTA with default or recommended parameters; a program in GCG Version 6.1. FASTA (e.g., FASTA2 and FAST A3) provides alignments and percent sequence identity of the regions of the best overlap between the query and search sequences (Pearson (2000) supra). Another algorithm when comparing a sequence disclosed herein to a database containing a large number of sequences from different organisms is the computer program BLAST, especially BLASTP or TBLASTN, using default parameters. See, e.g., Altschul et al. (1990) J. Mol. Biol. 215: 403-410 and (1997) Nucleic Acids Res. 25:3389-3402, each of which is herein incorporated by reference.

The antibodies and antigen-binding fragments, which may be used as disclosed herein, specifically bind to TREM2. The agonist anti-TREM2 antibodies may bind to TREM2 with high affinity or with low affinity. They may be used alone or as adjunct therapy with other therapeutic moieties or modalities known in the art (i.e., at least one additional therapeutic agent) for treating dysregulation of lipid metabolism and/or inflammation.

Certain agonist anti-TREM2 antibodies are capable of binding to and increasing the activity of TREM2, as determined by in vitro or in vivo assays. The ability of the antibodies to bind to and increase the activity of TREM2 may be measured using any standard method known to those skilled in the art, including binding assays or activity assays.

The antibodies specific for TREM2 may contain no additional labels or moieties, or they may contain an N-terminal or C-terminal label or moiety. In one embodiment, the label may be a radionuclide or a fluorescent dye. In certain embodiments, such labeled antibodies may be used in diagnostic assays.

Preparation of Anti-Human TREM2 Antibodies

Methods for generating human antibodies in transgenic mice are known in the art. Any such known methods can be used as disclosed herein to make human antibodies that specifically bind to TREM2.

An immunogen comprising any one of the following can be used to generate antibodies to TREM2, or fragments thereof. For example, the primary immunogen may be a full length TREM2 (see, UniProtKB Q9NZC2) or a recombinant form of TREM2 or modified human TREM2 fragments or modified cynomolgus TREM2 fragments. In certain embodiments, the primary immunogen may be followed by immunization with a secondary immunogen, or with an immunogenically active fragment of TREM2. Alternatively, TREM2 or a fragment thereof may be produced using standard biochemical techniques and modified and used as immunogen. The immunogen may be a biologically active and/or immunogenic fragment of TREM2 or DNA encoding the active fragment thereof.

In certain embodiments, the immunogen may be a peptide from the N terminal or C terminal end of TREM2. In one embodiment, the immunogen is a particular domain of TREM2. In some embodiments, the immunogen may be a recombinant TREM2 peptide expressed in E. coli or in any other eukaryotic or mammalian cells such as Chinese hamster ovary (CHO) cells. The peptides may be modified to include addition or substitution of certain residues for tagging or for purposes of conjugation to carrier molecules, such as, KLH. For example, a cysteine may be added at either the N terminal or C terminal end of a peptide, or a linker sequence may be added to prepare the peptide for conjugation to, for example, KLH for immunization.

Agonist anti-TREM2 antibodies Comprising Fc Variants

Agonist anti-TREM2 antibodies may comprise an Fc domain comprising one or more mutations, which enhance or diminish antibody binding to the FcRn receptor, e.g., at acidic pH as compared to neutral pH. For example, agonist anti-TREM2 antibodies may comprise a mutation in the C_{H}2 or a C_{H}3 region of the Fc domain, wherein the mutation(s) increases the affinity of the Fc domain to FcRn in an acidic environment (e.g., in an endosome where pH ranges from about 5.5 to about 6.0). Such mutations may result in an increase in serum half-life of the antibody when administered to an animal.

Biological Characteristics of Agonist Anti-TREM2 Antibodies, or Fragments Thereof

In general, the antibodies useful as disclosed herein function by binding to TREM2, and include agonist anti-TREM2 antibodies and antigen-binding fragments thereof that bind TREM2 molecules, e.g., with high affinity. For example, antibodies and antigen-binding fragments of antibodies that bind TREM2 (e.g., at 25° C. or at 37° C.) with a K_{D} of less than about 50 nM as measured by surface plasmon resonance may be used as disclosed herein. In certain embodiments, the antibodies or antigen-binding fragments thereof bind TREM2 with a K_{D} of less than about 40 nM, less than about 30 nM, less than about 20 nM, less than about 10 nM less than about 5 nM, less than about 2 nM or less than about 1 nM, as measured by surface plasmon resonance or a substantially similar assay. The antibodies or antigen-binding fragments thereof disclosed herein may also bind cynomolgus (Macaca fascicularis) TREM2 (e.g., at 25° C. or at 37° C.) with a K_{D} of less than about 35 nM as measured by surface plasmon resonance. In certain embodiments, the antibodies or antigen-binding fragments thereof bind cynomolgus TREM2 with a K_{D} of less than about 30 nM, less than about 20 nM, less than about 15 nM, less than about 10 nM, or less than about 5 nM, as measured by surface plasmon resonance or a substantially similar assay.

The methods and uses described herein also include the use of antibodies and antigen-binding fragments thereof that bind TREM2 with a dissociative half-life (t1/2) of greater than about 1.1 minutes as measured by surface plasmon resonance at 25° C. or 37° C., or a substantially similar assay. In certain embodiments, the antibodies or antigen-binding fragments bind TREM2 with a t1/2 of greater than about 5 minutes, greater than about 10 minutes, greater than about 30 minutes, greater than about 50 minutes, greater than about 60 minutes, greater than about 70 minutes, greater than about 80 minutes, greater than about 90 minutes, greater than about 100 minutes, greater than about 200 minutes, greater than about 300 minutes, greater than about 400 minutes, greater than about 500 minutes, greater than about 600 minutes, greater than about 700 minutes, greater than about 800 minutes, greater than about 900 minutes, greater than about 1000 minutes, or greater than about 1200 minutes, as measured by surface plasmon resonance at 25° C. or 37° C. (e.g., mAb-capture or antigen-capture format), or a substantially similar assay.

In some embodiments, the antibodies may bind to a particular domain of TREM2 or to a fragment of the domain. In some embodiments, the antibodies for use as disclosed herein may bind to more than one domain (cross-reactive antibodies).

In certain embodiments, antibodies for use as disclosed herein may be bi-specific antibodies. The bi-specific antibodies may bind one epitope in one domain and may also bind a second epitope in a different domain of TREM2. In certain embodiments, the bi-specific antibodies may bind two different epitopes in the same domain.

In one embodiment, the use of an isolated fully human monoclonal antibody or antigen-binding fragment thereof that binds to TREM2 is provided for.

### Species Selectivity and Species Cross-Reactivity

According to certain embodiments, an agonist anti-TREM2 antibody may bind to human TREM2 but not to TREM2 from other species. Alternatively, an agonist anti-TREM2 antibody may bind to human TREM2 and to TREM2 from one or more non-human species. For example, an agonist anti-TREM2 antibody may bind to human TREM2 and may bind or not bind, as the case may be, to one or more of mouse, rat, guinea pig, hamster, gerbil, pig, cat, dog, rabbit, goat, sheep, cow, horse, camel, cynomolgus, marmoset, rhesus or chimpanzee TREM2. In certain embodiments, an agonist anti-TREM2 antibody may bind to human and cynomolgus TREM2 with the same affinities or with different affinities, but do not bind to rat and mouse TREM2.

### Additional Therapeutic Agents

In certain embodiments, a method described herein further comprises administering one or more additional therapeutic agents (e.g., a second therapeutic agent).

The one or more additional therapeutic agents may be administered either simultaneously or sequentially with the agonist anti-TREM2 antibody. In certain embodiments, the one or more additional therapeutic agents are administered simultaneously with the agonist anti-TREM2 antibody. In certain embodiments, a pharmaceutical composition comprising the agonist anti-TREM2 antibody and the one or more additional therapeutic agents are administered. In certain embodiments, the agonist anti-TREM2 antibody and the one or more additional therapeutic agents are administered sequentially. In certain embodiments, the agonist anti-TREM2 antibody is administered before the one or more additional therapeutic agents. In certain embodiments, the one or more additional therapeutic agents are administered before the agonist anti-TREM2 antibody.

In certain embodiments, the additional therapeutic agent is an agent useful for treating Alzheimer's disease or atherosclerosis.

In certain embodiments, the additional therapeutic agent is an agent useful for treating inflammation.

In certain embodiments, the additional therapeutic agent is an LXR agonist. As described herein, an effective amount of an LXR agonist may be administered to a mammal to treat dysregulation of lipid metabolism or a disease or condition associated with such dysregulation. LXR is part of the superfamily of ligand dependent, nuclear receptor transcription factors. Oxidized derivatives of cholesterol (oxysterols) are the natural ligands of LXR and have the ability to both agonize and antagonize LXR activation. LXRα (encoded by NR1H3) is highly expressed in the liver, macrophages, and other highly metabolic tissues, whereas LXRβ (NR1H2) is ubiquitously expressed. Upon ligand activation, LXRs form a heterodimer with the RXR, and play a role in modulation of lipid metabolism and inflammatory signaling.

The term “LXR agonist” refers to an agent capable of activating, enhancing, increasing, or otherwise stimulating one or more functions of the target LXR. An agonist of LXR may induce any LXR activity, for example LXR-mediated signaling, either directly or indirectly. A LXR agonist, as used herein, may but is not required to bind an LXR, and may or may not interact directly with the LXR. An LXR agonist can specifically agonize LXRα, LXRβ or both. An LXR agonist may affect other receptors/pathways in addition to agonizing LXR.

LXR agonists include natural oxysterols, synthetic oxysterols, synthetic nonoxysterols, and natural nonoxysterols. Exemplary natural oxysterols include 20(S) hydroxycholesterol, 22(R) hydroxycholesterol, 24(S) hydroxycholesterol, 25-hydroxycholesterol, 24(S), 25 epoxycholesterol, and 27-hydroxycholesterol. Exemplary synthetic oxysterols include N,N-dimethyl-3.beta.-hydroxycholenamide (DMHCA). Exemplary synthetic nonoxysterols include N-(2,2,2-trifluoroethyl)-N-{4-[2,2,2-trifluoro-1-hydroxy-1-(trifluorometh-yl)ethyl]phenyl}benzene sulfonamide (TO901317; Tularik 0901317), [3-(3-(2-chloro-trifluoromethylbenzyl-2,2-diphenylethylamino)propoxy)phen-ylacetic acid] (GW3965), N-methyl-N-[4-(2,2,2-trifluoro-1-hydroxy-1-trifluoromethyl-1-ethyl)-pheny-1]-benzenesulfonamide (T0314407), 4,5-dihydro-1-(3-(3-trifluoromethyl-7-propyl-benzisoxazol-6-yloxy)propyl)-2,6-pyrimidinedione, 3-chloro-4-(3-(7-propyl-3-trifluoromethyl-6-(4,5)-isoxazolyl)propylthio)-phenyl acetic acid (F.sub.3-MethylAA), and acetyl-podocarpic dimer. Exemplary natural nonoxysterols include paxilline, desmosterol, and stigmasterol.

Other useful LXR agonists are disclosed, for example, in Published U.S. Patent Application Nos. 2006/0030612, 2005/0131014, 2005/0036992, 2005/0080111, 2003/0181420, 2003/0086923, 2003/0207898, 2004/0110947, 2004/0087632, 2005/0009837, 2004/0048920, and 2005/0123580; U.S. Pat. Nos. 6,316,503, 6,828,446, 6,822,120, and 6,900,244; WO2008036239; WO2001/41704; Menke J G et al., Endocrinology 143:2548-58 (2002); Joseph S B et al., Proc. Natl. Acad. Sci. USA 99:7604-09 (2002); Fu X et al., J. Biol. Chem. 276:38378-87 (2001); Schultz J R et al., Genes Dev. 14:2831-38 (2000); Sparrow C P et al., J. Biol. Chem. 277:10021-27 (2002); Yang C et al., J. Biol. Chem., Manuscript M603781200 (Jul. 20, 2006); Bramlett K S et al., J. Pharmacol. Exp. Ther. 307:291-96 (2003); Ondeyka J G et al., J. Antibiot (Tokyo) 58:559-65 (2005).

In certain embodiments, the LXR agonist is hypocholamide, T0901317, GW3965, IMB-808 or N,N-dimethyl-3beta-hydroxy-cholenamide (DMHCA). In certain embodiments, the LXR agonist is GW3965.

In certain other embodiments, the additional therapeutic agent is an RXR agonist. As described herein, an effective amount of an RXR agonist may also be administered to a mammal to treat dysregulation of lipid metabolism or a disease or condition associated with such dysregulation. RXR is a type of nuclear receptor that is activated by 9-cis retinoic acid and 9-cis-13,14-dihydro-retinoic acid. There are three RXR forms: RXR-alpha, RXR-beta, and RXR-gamma, encoded by the RXRA, RXRB, RXRG genes, respectively. RXR heterodimerizes with subfamily 1 nuclear receptors including CAR, FXR, LXR, PPAR, PXR, RAR, TR, and VDR. As with other type II nuclear receptors, the RXR heterodimer in the absence of ligand is bound to hormone response elements complexed with corepressor protein. Binding of agonist ligands to RXR results in dissociation of corepressor and recruitment of coactivator protein, which, in turn, promotes transcription of the downstream target gene into mRNA, and eventually protein.

The term “RXR agonist” refers to an agent capable of activating, enhancing, increasing, or otherwise stimulating one or more functions of the target RXR (e.g., increases the transcriptional regulation activity of RXR homo- and hetero-dimers). An agonist of RXR may induce any RXR activity, for example RXR-mediated signaling, either directly or indirectly. An RXR agonist, as used herein, may but is not required to bind an RXR, and may or may not interact directly with the RXR. An RXR agonist can specifically agonize RXRα, RXRβ, or RXRγ, or a combination thereof. An RXR agonist may affect other receptors/pathways in addition to agonizing RXR.

Certain RXR agonists and methods of synthesizing such agonists are known. For example, RXR agonists include, but are not limited to, those described in Boehm et al. J. Med. Chem. 38:3146 (1994), Boehm et al. J. Med. Chem. 37:2930 (1994), Antras et al., J. Biol. Chem. 266: 1157-61 (1991), Salazar-Olivo et al., Biochem. Biophys. Res. Commun. 204: 10 257-263 (1994), Safanova, Mol. Cell. Endocrin. 104:201 (1994), M. L. Dawson and W. H. Okamura, Chemistry and Biology of Synthetic Retinoids, Chapters 3, 8, 14 and 16, CRC Press, Inc., Florida (1990); M. L. Dawson and P. D. Hobbs, The Retinoids, Biology, Chemistry and Medicine, M. B. Spom et al., Eds. (2nd ed.), Raven Press, New York, N.Y., pp. 5-178 (1994); Liu et al., Tetrahedron, 40: 1931 (1984); Cancer Res., 43:5268 (1983); Eur. J. Med. Chem. 15:9 (1980); Allegretto et al., J. Bio. Chem., 270:23906 (1995); Bissonette et al., Mol. Cell. Bio., 15:5576(1995); Beard et al., J. Med. Chem., 38:2820 (1995), Koch et al., J. Med. Chem., 39:3229 (1996); U.S. Pat. Nos. 4,326,055; 4,578,498; 5,399,586; 5,466,861; 5,721,103; 5,780,676; 5,801,253; 5,830,959; 6,083,977; 6,131,050; U.S. 20160324874; US20180185342; US20180263939; US20180318241; WO 93/11755; WO 93/21146; WO 94/15902; WO94/23068; WO 95/04036; WO 96/20913; WO 20100105728; WO 2013056232; and WO 2013090616.

In certain embodiments, the RXR agonist is CD 3254, docosahexaenoic acid, fluorobexarotene, bexarotene (LGD1069), IRX4204, HX630, PA024, isotretinoin, retinoic acid, SR 11237, LG101506, LGD100268 or LGD100324. In certain embodiments, the RXR agonist is bexarotene.

In certain other embodiments, the additional therapeutic agent is an ACAT1 inhibitor. As described herein, an effective amount of an ACAT1 inhibitor may also be administered to a mammal to treat dysregulation of lipid metabolism or a disease or condition associated with such dysregulation.

The ACAT1 gene encodes mitochondrial acetyl-CoA acetyltransferase, a short-chain-length-specific thiolase (UniProtKB P24752). As used herein, an “ACAT1 inhibitor” includes any compound or treatment capable of inhibiting the expression and/or function of ACAT1, either directly or indirectly (e.g., inhibits transcription, RNA maturation, RNA translation, post-translational modification, or enzymatic activity). An ACAT1 inhibitor, as used herein, may but is not required to bind to ACAT1, and may or may not interact directly with the enzyme. In certain embodiments, the inhibitor detectably inhibits the expression level or biological activity of ACAT1 as measured, e.g., using an assay described herein or known in the art. In certain embodiments, the inhibitor inhibits the expression level or biological activity of ACAT1 by at least about 5%, at least about 10%, at least about 20%, at least about 30%, at least about 40%, at least about 50%, at least about 60%, at least about 70%, at least about 80%, or at least about 90%.

The inhibitor may be of natural or synthetic origin. For example, it may be a nucleic acid, a polypeptide, a protein, a peptide, or an organic compound. In one embodiment, the inhibitor is an siRNA, shRNA, a small molecule or an antibody.

In certain embodiments, the inhibitor is an antisense nucleic acid (e.g., siRNA or shRNA) capable of inhibiting transcription of ACAT1 or translation of the corresponding messenger RNA. An art worker can design an antisense nucleic acid using commercially available software and the gene sequence of ACAT1.

In certain embodiments, the inhibitor is a polypeptide, for example, an antibody against ACAT1, or a fragment or derivative thereof, such as a Fab fragment, a CDR region, or a single chain antibody.

The term “small molecule” includes organic molecules having a molecular weight of less than about 1000 amu. In one embodiment a small molecule can have a molecular weight of less than about 800 amu. In another embodiment a small molecule can have a molecular weight of less than about 500 amu.

Certain ACAT1 inhibitors are known in the art. For example, in certain embodiments, an ACAT1 inhibitor, is an ACAT1 inhibitor as described in US 2004/0038987, US 20140044757, US20170292128 or WO 2015/038585. In certain embodiments, the ACAT1 inhibitor is selected from the group consisting of avasimibe (CI-1011), pactimibe, purpactins, manassantin A, diphenylpyridazine derivatives, glisoprenin A, CP113, 818, K604, beauveriolide I, beauveriolide III, U18666A, TMP-153, YM750, GERI-BP002-A, Sandoz Sah 58-035, VULM 1457, Lovastatin, CI976, CL-283, 546, CI-999, E5324, YM17E, FR182980, ATR-101 (PD132301 or PD132301-2), F-1394, HL-004, F-12511 (eflucimibe), cinnamic acid derivatives, cinnamic derivative, Dup 128, RP-73163, pyripyropene C, FO-1289, AS-183, SPC-15549, FO-6979, Angekica, ginseng, Decursin, terpendole C, beauvericin, spylidone, pentacecilides, CL-283, 546, betulinic acid, shikonin derivatives, esculeogenin A, Wu-V-23, pyripyropene derivatives A, B, and D, glisoprenin B-D, saucemeol B, sespendole, diethyl pyrocarbonate, beauveriolide analogues, Acaterin, DL-melinamide, PD 138142-15, CL277, 082, EAB-309, Enniatin antibiotics, Epi-cochlioquinone A, FCE-27677, FR186485, FR190809, NTE-122, obovatol, panaxadiols, protopanaxadiols, polyacetylenes, SaH 57-118, AS-186, BW-447A, 447C88, T-2591, TEI-6522, TEI-6620, XP 767, XR 920, GERI-BP001, gomisin N, gypsetin, helminthosporol, TS-962, A 922500 (CAS 959122-11-3), N-[3-(4-hydroxyphenyl)-1-oxo-2-propenyl]-L-phenylalanine, methyl ester (CAS 615264-52-3), 3,4-dihydroxy Hydrocinnamic acid (L-Aspartic acid dibenzyl ester) amide (CAS 615264-62-5), CI-976, FR14523 (Fujisawa Pharmaceutical Co. Ltd.), F1394 (Fujirebio Inc.), isochromophilones, kudingosides, lateritin, naringenin, and combinations thereof. In certain embodiments, the ACAT1 inhibitor is CP-113,818, CI-1011 or K-604. In certain embodiments, the ACAT1 inhibitor is K-604.

In certain embodiments, the additional therapeutic agent is an agent described herein.

### Administration

A therapeutic agent (e.g., an agonist anti-TREM2 antibody or additional therapeutic agent) can be formulated as a pharmaceutical composition and administered to a mammalian host, such as a human patient in a variety of forms adapted to the chosen route of administration, i.e., orally or parenterally, by intravenous, intramuscular, topical or subcutaneous routes.

Thus, therapeutic agents may be systemically administered, e.g., orally (e.g., added to drinking water), in combination with a pharmaceutically acceptable vehicle such as an inert diluent or an assimilable edible carrier. They may be enclosed in hard or soft shell gelatin capsules, may be compressed into tablets, or may be incorporated directly with the food of the patient's diet. For oral therapeutic administration, the therapeutic agent may be combined with one or more excipients and used in the form of ingestible tablets, buccal tablets, troches, capsules, elixirs, suspensions, syrups, wafers, and the like. Such compositions and preparations generally contain at least 0.1% of the agent. The percentage of the compositions and preparations may, of course, be varied and may conveniently be between about 2 to about 60% of the weight of a given unit dosage form. The amount of agent in such therapeutically useful compositions is such that an effective dosage level will be obtained.

The tablets, troches, pills, capsules, and the like may also contain the following: binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring may be added. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. A syrup or elixir may contain the therapeutic agent, sucrose or fructose as a sweetening agent, methyl and propylparabens as preservatives, a dye and flavoring such as cherry or orange flavor. Of course, any material used in preparing any unit dosage form should be pharmaceutically acceptable and substantially non-toxic in the amounts employed. In addition, the therapeutic agent may be incorporated into sustained-release preparations and devices.

The therapeutic agent may also be administered intravenously or intraperitoneally by infusion or injection. Solutions of the therapeutic agent or its salts can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations contain a preservative to prevent the growth of microorganisms.

The pharmaceutical dosage forms suitable for injection or infusion can include sterile aqueous solutions or dispersions or sterile powders comprising the active ingredient which are adapted for the extemporaneous preparation of sterile injectable or infusible solutions or dispersions, optionally encapsulated in liposomes. In all cases, the ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. The prevention of the action of microorganisms can be brought about by various antibacterial and antifungal agents, for example, parabens, chlorobutanol, phenol, sorbic acid, thimerosal, and the like. In many cases, it can include isotonic agents, for example, sugars, buffers or sodium chloride. Prolonged absorption of the injectable compositions can be brought about by the use in the compositions of agents delaying absorption, for example, aluminum monostearate and gelatin.

Sterile injectable solutions are prepared by incorporating the therapeutic agent in the required amount in the appropriate solvent with various of the other ingredients enumerated above, as required, followed by filter sterilization. Sterile powders for the preparation of sterile injectable solutions can be prepared by vacuum drying and the freeze drying techniques, which yield a powder of the active ingredient plus any additional desired ingredient present in the previously sterile-filtered solutions.

For topical administration, the therapeutic agents may be applied in pure form, i.e., when they are liquids. However, it will generally be desirable to administer them to the skin as compositions or formulations, in combination with a dermatologically acceptable carrier, which may be a solid or a liquid.

Useful solid carriers include finely divided solids such as talc, clay, microcrystalline cellulose, silica, alumina and the like. Useful liquid carriers include water, alcohols or glycols or water-alcohol/glycol blends, in which the therapeutic agents can be dissolved or dispersed at effective levels, optionally with the aid of non-toxic surfactants. Adjuvants such as fragrances and additional antimicrobial agents can be added to optimize the properties for a given use. The resultant liquid compositions can be applied from absorbent pads, used to impregnate bandages and other dressings, or sprayed onto the affected area using pump-type or aerosol sprayers.

Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like, for application directly to the skin of the user.

Examples of useful dermatological compositions which can be used to deliver the therapeutic agents to the skin are known to the art; for example, see Jacquet et al. (U.S. Pat. No. 4,608,392), Geria (U.S. Pat. No. 4,992,478), Smith et al. (U.S. Pat. No. 4,559,157) and Wortzman (U.S. Pat. No. 4,820,508).

Useful dosages of the therapeutic agents can be determined by comparing their in vitro activity, and in vivo activity in animal models. Methods for the extrapolation of effective dosages in mice, and other animals, to humans are known to the art; for example, see U.S. Pat. No. 4,938,949.

The amount of the therapeutic agent, or an active salt or derivative thereof, required for use in treatment will vary not only with the particular salt selected but also with the route of administration, the nature of the condition being treated and the age and condition of the patient and will be ultimately at the discretion of the attendant physician or clinician.

The therapeutic agents may be conveniently formulated in unit dosage form. In one embodiment, a composition comprising a therapeutic agent formulated in such a unit dosage form can be used.

The desired dose may conveniently be presented in a single dose or as divided doses administered at appropriate intervals, for example, as two, three, four or more sub-doses per day. The sub-dose itself may be further divided, e.g., into a number of discrete loosely spaced administrations; such as multiple inhalations from an insufflator or by application of a plurality of drops into the eye.

In certain embodiments an agonist anti-TREM2 antibody is administered to a mammal. In certain embodiments, an additional therapeutic agent is further administered to the mammal (e.g., an RXR agonist, an LXR agonist, an ACAT1 inhibitor or other agents useful for treating lipid dysregulation/inflammation). When a combination of two or more of these agents is administered, they may be administered either simultaneously or sequentially. In certain embodiments, the two or more agents are administered sequentially. In certain embodiments, the two or more agents are administered simultaneously. In certain embodiments, a pharmaceutical composition comprising a combination of the two or more agents is administered. For example, in one embodiment a composition comprising an agonist anti-TREM2 antibody, at least one other therapeutic agent, and a pharmaceutically acceptable diluent or carrier, is provided for use in treating dysregulated lipid metabolism and/or inflammation.

Certain embodiments also provide is a kit comprising an agonist anti-TREM2, packaging material, and instructions for administering the agonist anti-TREM2 antibody to an animal to treat dysregulated lipid metabolism and/or inflammation. In certain embodiments, the kit further comprises at least one other therapeutic agent.

### Methods of Isolating Enriched CNS Cell Populations

Methods of isolating enriched populations of CNS cell types from brain tissue are provided herein (e.g., enriched populations of astrocytes or microglial cells).

Thus, certain embodiments provide a method of sorting populations of CNS cells from a tissue sample, comprising:

(a) contacting the tissue sample with: an anti-CD45 primary antibody, an anti-CD11b primary antibody and an anti-astrocyte cell surface antigen-2 (ACSA-2) primary antibody, wherein each primary antibody is uniquely labeled, to provide a labeled tissue sample; and

(b) sorting the cells in the labeled tissue sample by flow cytometry,

wherein the method provides distinct cell populations of astrocytes and microglial cells.

As used herein, the term “distinct cell population” refers to a physically separate population of cells that is enriched for a particular CNS cell type (e.g., neuronal or astrocytic).

Certain embodiments also provide a composition comprising a sorted distinct cell population isolated using a method described herein (e.g., a sorted microglial cell population or a sorted astrocytic cell population).

In certain embodiments, the microglial cell population is sorted based on the following marker profile: CD45^{low}/CD11b⁺/ACSA-2⁻.

In certain embodiments, the astrocyte population is sorted based on the following marker profile: CD45⁻/CD11b⁻/ACSA-2⁺.

Certain embodiments provide a collection of CNS cells comprising two physically separate cell populations, wherein the first cell population comprises an enriched population of CD45^{low}/CD11b⁺/ACSA-2⁻ cells and the second cell population comprises an enriched population CD45⁻/CD11b⁻/ACSA-2⁺ cells.

As described herein, a combination of an anti-CD45 primary antibody and an anti-CD11b antibody may be used to isolate an enriched population of microglial cells from other CNS cell types. CD45, also known as leukocyte common antigen (LCA) and protein tyrosine phosphatase receptor type C (PTPRC), is a cell surface antigen that is expressed in varying levels by most hematopoietic cells, with the exception of erythrocytes and platelets. CD45 is expressed at low levels in microglial cells, is not expressed by astrocytes, and is expressed at high levels in certain non-microglial, non-astrocytic cells, such as macrophages. Therefore, an anti-CD45 primary antibody may be used to label cells that express the CD45 cell surface marker (CD45⁺) and flow cytometry may be used to isolate labeled cells that express CD45 at a low level (e.g., CD45^{low} microglial cells). CD45^{low} cells may be identified and separated from CD45^{high} cells based on a cut-off reference value. For example, the reference value may be the amount of CD45 expression in a control cell or population of control cells, such as a known microglial cell(s) or in a known CD45^{high} cell. In some embodiments, the reference value is a range of values, e.g., when the reference values are obtained from a plurality of samples. Furthermore, the reference value can be presented as a single value (e.g., a measured abundance value, a mean value, or a median value) or a range of values, with or without a standard deviation or standard of error.

CD11b is an integrin family member that pairs with CD18 to form the CR3 heterodimer. CD11b is expressed on a variety of cell types, including macrophages and microglial cells, but is not express by astrocytes. Therefore, an anti-CD11b primary antibody may be used to label cells that express the CD11b cell surface marker and flow cytometry may be used to isolate labeled cells (e.g., CD11b⁺ microglial cells).

An anti-ACSA-2 antibody recognizes a glycosylated surface molecule expressed by astrocytes. In contrast, this surface molecule is not expressed by non-astrocytic cells in the CNS, such as neurons, oligodendrocytes, NG2⁺ cells, microglia, endothelial cells, leukocytes, or erythrocytes (Kantzer et al., 2017, Glia, 65:990-1004). Therefore, an anti-ACSA-2 primary antibody may be used to label cells that express the ACSA-2 cell surface marker and flow cytometry may be used to isolate labeled cells (e.g., ACSA-2⁺ astrocytic cells).

In certain embodiments, the primary antibodies are comprised within a composition, and the tissue sample is contacted with the composition (e.g., a composition comprising an anti-ACSA-2 antibody, an anti-CD11b antibody, and an anti-CD45 antibody). In certain embodiments, each primary antibody is uniquely labeled (i.e., each antibody within the composition comprises a different label) with a label suitable for sorting by flow cytometry (e.g., a fluorescent label). In certain embodiments, the composition further comprises a viability dye, which may be used to distinguish viable and non-viable cells by flow cytometry (e.g., Fixable Viability Stain BV510). In certain other embodiments, the viability dye is not comprised with the composition and the tissue sample is contacted with the viability dye simultaneously or sequentially with the composition.

In certain embodiments, the cells present within the tissue sample are dissociated prior to being contacted with the viability dye and/or composition.

In certain embodiments, the tissue sample is contacted with the composition under conditions suitable for the antibodies to bind to its corresponding marker and label the cells. In certain embodiments, the labeled tissue sample prior to being sorted by flow cytometry comprises labeled ACSA-2⁺ cells, labeled CD45⁺ cells, and labeled CD11b⁺ cells. In certain embodiments, the cells are further labeled with a viability dye.

In certain embodiments, the cells present within the tissue sample are sorted by flow cytometry into a population of non-viable cells and a population of viable cells (e.g., with a viability dye).

In certain embodiments, the cells present within the tissue sample are sorted by flow cytometry into a population of CD45⁺ cells and a population of CD45⁻ cells. In certain embodiments, the population of CD45⁺ cells are sorted by flow cytometry into a population of CD45^{low} cells and a population of CD45^{high} cells.

In certain embodiments, the cells present within the tissue sample are sorted by flow cytometry into a population of CD11b⁺ cells and a population of CD11b⁻ cells.

In certain embodiments, the cells present within the tissue sample are sorted by flow cytometry into a population of ACSA-2⁺ cells and a population of ACSA-2⁻ cells.

Labeled cells may be sorted by flow cytometry using any gating combination that results in isolated populations of CD45^{low}/CD11b⁺/ACSA-2⁻ microglial cells and isolated populations of CD45⁻/CD11b⁻/ACSA-2⁺ astrocytic cells. For example, in certain embodiments, the cells present within the tissue sample are sorted by flow cytometry into a population of non-viable cells and a population of viable cells (e.g., with a viability dye). In certain embodiments, the population of viable cells are sorted by flow cytometry into a population of CD11b⁺ cells and a population of CD11b⁻ cells. In certain embodiments, the population of CD11b⁺ cells is further sorted into a population of CD45⁺ cells and a population of CD45⁻ cells. In certain embodiments, the population of CD11b⁺/CD45⁺ cells are sorted by flow cytometry into a population of CD11b⁺/CD45^{low} cells and a population of CD11b⁺/CD45^{high} cells. In certain embodiments, the population of CD11b⁻ cells are sorted by flow cytometry into a population of ACSA-2⁺ cells and a population of ACSA-2⁻ cells. Such sorting results in a population of CD45^{low}/CD11b⁺/ACSA-2⁻ microglial cells and a population of CD45⁻/CD11b⁻/ACSA-2⁺ astrocytic cells.

In certain embodiments, the sorted population of enriched astrocytic cells (e.g., viable, CD45⁻, CD11b⁻, ACSA-2⁺ cells) comprises less than about 25%, 20%, 19%, 18%, 17%, 16%, 15%, 14%, 13%, 12%, 11%, 10%, 9%, 8%, 7%, 6%, 5%, 4%, 3%, 2%, 1% or less of non-astrocytic cells. In certain embodiments, the sorted population of enriched astrocytic cells does not contain non-astrocytic cells.

In certain embodiments, the sorted population of enriched microglial cells (e.g., viable, CD45^{low}, CD11b⁺, ACSA-2⁻ cells cells) comprises less than about 25%, 20%, 19%, 18%, 17%, 16%, 15%, 14%, 13%, 12%, 11%, 10%, 9%, 8%, 7%, 6%, 5%, 4%, 3%, 2%, 1% or less of non-microglial cells. In certain embodiments, the sorted population of enriched microglial cells (does not contain non-microglial cells.

In certain embodiments, one or more of the enriched cell populations are analyzed for quantification of a metabolic (e.g., lipid species) or nucleic acid species. In certain embodiments, the enriched astrocytic cell population is analyzed for quantification of a metabolic or nucleic acid species. In certain embodiments, the enriched microglial cell population is analyzed for quantification of a metabolic or nucleic acid species. In certain embodiments, the enriched astrocytic and microglial cell populations are analyzed for quantification of a metabolic or nucleic acid species. In certain embodiments, the one or more enriched cell populations are analyzed for quantification of a metabolic species. In certain embodiments, the one or more enriched cell populations are analyzed for quantification of more than one metabolic species (e.g., 2, 3, 4, 5, 10, 25, 50 or more). In certain embodiments, the one or more enriched cell populations are analyzed for quantification of a nucleic acid species. In certain embodiments, the one or more enriched cell populations are analyzed for quantification of more than one nucleic acid species (e.g., 2, 3, 4, 5, 10, 25, 50 or more). In certain embodiments, the one or more enriched cell populations are analyzed for quantification of a metabolic and a nucleic acid species. In certain embodiments, the one or more enriched cell populations are analyzed for quantification of more than one metabolic species and more than one nucleic acid species.

As used herein, the term metabolic species includes macromolecules such as lipid species. For example, in certain embodiments, the metabolic species is a lipid species, such as a lipid species described herein (e.g., a cholesteryl ester species). In certain embodiments, a combination of metabolic species is quantified, such as a combination of lipids described herein. Metabolic species may be quantified using methods known in the art. For example, a metabolic species may be quantified using a liquid chromatography mass spectrometry (LCMS) assay (see, e.g., the Examples).

A nucleic acid, may be e.g., RNA or DNA, such as genomic DNA, RNA transcribed from genomic DNA, or cDNA generated from RNA. In certain embodiments, the nucleic acid species is RNA. In certain embodiments, the nucleic acid species is DNA. In certain embodiments, the nucleic acid species is genomic DNA. Methods of quantifying nucleic acid species are known in the art. For example, such methods include, but are not limited to, polymerase chain reaction (PCR), including quantitative PCR (qPCR) and Real-Time Quantitative Reverse Transcription PCR (qRT-PCR); RNAseq; Northern blot analysis, expression microarray analysis; next generation sequencing (NGS); and fluorescence in situ hybridization (FISH). In certain embodiments, a nucleic acid species is quantified using an assay described herein.

In certain embodiments, one or more enriched cell populations are analyzed for quantification of an administered therapeutic agent. In certain embodiments, the enriched astrocytic cell population is analyzed for quantification of an administered therapeutic agent. In certain embodiments, the enriched microglial cell population is analyzed for quantification of an administered therapeutic agent. In certain embodiments, the enriched astrocytic and microglial cell populations are analyzed for quantification of an administered therapeutic agent. Methods for quantifying a therapeutic agent are known in the art and are described herein.

### Certain Techniques

As used herein, the phrase “physiological sample” is meant to refer to a biological sample obtained from a subject that contains protein, lipid, and/or nucleic acid. Thus, the sample may be evaluated at the nucleic acid, lipid, or protein level. In certain embodiments, the physiological sample comprises tissue, cerebrospinal fluid (CSF), urine, blood, serum, or plasma. In certain embodiments, the sample comprises tissue (e.g., comprises microglia). In certain embodiments, the sample comprises CSF. In certain embodiments, the sample comprises blood and/or plasma.

A biological sample, may be obtained using methods known to those skilled in the art. Biological samples may be obtained from vertebrate animals, and in particular, mammals. Variations in DNA, RNA or proteins (e.g., mutations, expression or localization) may be detected from a sample.

A nucleic acid, may be e.g., genomic DNA, RNA transcribed from genomic DNA, or cDNA generated from RNA. A nucleic acid or protein may be derived from a vertebrate, e.g., a mammal. A nucleic acid or protein is said to be “derived from” a particular source if it is obtained directly from that source or if it is a copy of a nucleic acid found in that source.

In certain embodiments, genomic DNA may be isolated from a biological sample and analyzed in a detection assay. In certain embodiments, mRNA is isolated from a biological sample and analyzed in a detection assay. In certain embodiments, mRNA isolated from the biological sample may be reverse transcribed to generate cDNA.

Variations in nucleic acids and amino acid sequences may be detected by certain methods known to those skilled in the art. Similarly, nucleic acid expression (e.g., mRNA expression) may be detected using methods known in the art. Such methods include, but are not limited to, polymerase chain reaction (PCR), including quantitative PCR (qPCR) and Real-Time Quantitative Reverse Transcription PCR (qRT-PCR); Northern blot analysis, expression microarray analysis; next generation sequencing (NGS); fluorescence in situ hybridization (FISH); DNA sequencing; primer extension assays, including allele-specific nucleotide incorporation assays and allele-specific primer extension assays (e.g., allele-specific PCR, allele-specific ligation chain reaction (LCR), and gap-LCR); allele-specific oligonucleotide hybridization assays (e.g., oligonucleotide ligation assays); cleavage protection assays in which protection from cleavage agents is used to detect mismatched bases in nucleic acid duplexes; analysis of MutS protein binding; electrophoretic analysis comparing the mobility of variant and wild type nucleic acid molecules; denaturing-gradient gel electrophoresis (DGGE, as in, e.g., Myers et al. (1985) Nature 313:495); analysis of RNase cleavage at mismatched base pairs; analysis of chemical or enzymatic cleavage of heteroduplex DNA; mass spectrometry (e.g., MALDI-TOF); genetic bit analysis (GBA); 5′ nuclease assays (e.g., TaqMan®); and assays employing molecular beacons.

In certain embodiments, protein expression may also be detected. Assays for detecting and measuring protein expression are known in the art and include, e.g., western blot analysis, immunofluorescence, immunohistochemistry (e.g., of tissue arrays), etc.

In certain embodiments, macrophages are evaluated using an assay known in the art or described herein. In certain embodiments, iPSCs are evaluated using an assay known in the art or described herein. In certain embodiments, microglial cells are evaluated using an assay known in the art or described herein. In certain embodiments, microglial cells differentiated from iPSCs are evaluated using an assay known in the art or described herein.

### Certain Definitions

The terms “control” or “control sample” refer to any sample appropriate to the detection technique employed. The control sample may contain the products of the detection technique employed or the material to be tested. Further, the controls may be positive or negative controls.

The term “gene” is used broadly to refer to any segment of nucleic acid associated with a biological function. Genes include coding sequences and/or the regulatory sequences required for their expression. For example, gene refers to a nucleic acid fragment that expresses mRNA, functional RNA, or a specific protein, including its regulatory sequences. Genes also include non-expressed DNA segments that, for example, form recognition sequences for other proteins. Genes can be obtained from a variety of sources, including cloning from a source of interest or synthesizing from known or predicted sequence information, and may include sequences designed to have desired parameters. In addition, a “gene” or a “recombinant gene” refers to a nucleic acid molecule comprising an open reading frame and including at least one exon and (optionally) an intron sequence. The term “intron” refers to a DNA sequence present in a given gene which is not translated into protein and is generally found between exons.

A “mutated gene” or “mutation” or “functional mutation” refers to an allelic form of a gene, which is capable of altering the phenotype of a subject having the mutated gene relative to a subject which does not have the mutated gene.

A “variant” of a molecule is a sequence that is substantially similar to the sequence of the native molecule. For nucleotide sequences, variants include those sequences that, because of the degeneracy of the genetic code, encode the identical amino acid sequence of the native protein. Naturally occurring allelic variants such as these can be identified with the use of well-known molecular biology techniques, as, for example, with polymerase chain reaction (PCR) and hybridization techniques. Variant nucleotide sequences also include synthetically derived nucleotide sequences, such as those generated, for example, by using site-directed mutagenesis that encode the native protein, as well as those that encode a polypeptide having amino acid substitutions. Generally, nucleotide sequence variants disclosed herein will have in at least one embodiment 40%, 50%, 60%, to 70%, e.g., 71%, 72%, 73%, 74%, 75%, 76%, 77%, 78%, to 79%, generally at least 80%, e.g., 81%-84%, at least 85%, e.g., 86%, 87%, 88%, 89%, 90%, 91%, 92%, 93%, 94%, 95%, 96%, 97%, to 98%, sequence identity to the native (endogenous) nucleotide sequence.

As used herein, the term “specifically hybridizes” or “specifically detects” in regards to nucleic acid, refers to the ability of a nucleic acid molecule to hybridize to at least approximately six consecutive nucleotides of a sample nucleic acid.

The terms “protein,” “peptide” and “polypeptide” are used interchangeably herein.

“Naturally occurring,” “native” or “wild type” is used to describe an object that can be found in nature as distinct from being artificially produced. For example, a nucleotide sequence present in an organism (including a virus), which can be isolated from a source in nature and which has not been intentionally modified in the laboratory, is naturally occurring. Furthermore, “wild-type” refers to the normal gene, or organism found in nature without any known mutation.

The following terms are used to describe the sequence relationships between two or more nucleic acids, polynucleotides or polypeptides: (a) “reference sequence,” (b) “comparison window,” (c) “sequence identity,” (d) “percentage of sequence identity,” and (e) “substantial identity.”

(a) As used herein, “reference sequence” is a defined sequence used as a basis for sequence comparison. A reference sequence may be a subset or the entirety of a specified sequence; for example, as a segment of a full length cDNA or gene sequence, or the complete cDNA or gene sequence.

(b) As used herein, “comparison window” makes reference to a contiguous and specified segment of a polynucleotide sequence, wherein the polynucleotide sequence in the comparison window may comprise additions or deletions (i.e., gaps) compared to the reference sequence (which does not comprise additions or deletions) for optimal alignment of the two sequences. Generally, the comparison window is at least 20 contiguous nucleotides in length, and optionally can be 30, 40, 50, 100, or longer. Those of skill in the art understand that to avoid a high similarity to a reference sequence due to inclusion of gaps in the polynucleotide sequence a gap penalty is typically introduced and is subtracted from the number of matches.

Methods of alignment of sequences for comparison are well known in the art. Thus, the determination of percent identity between any two sequences can be accomplished using a mathematical algorithm.

Computer implementations of these mathematical algorithms can be utilized for comparison of sequences to determine sequence identity. Such implementations include, but are not limited to: CLUSTAL in the PC/Gene program (available from Intelligenetics, Mountain View, Calif.); the ALIGN program (Version 2.0) and GAP, BESTFIT, BLAST, FASTA, and TFASTA in the Wisconsin Genetics Software Package, Version 8 (available from Genetics Computer Group (GCG), 575 Science Drive, Madison, Wis., USA). Alignments using these programs can be performed using the default parameters.

Software for performing BLAST analyses is publicly available through the National Center for Biotechnology Information (see the World Wide Web at ncbi.nlm.nih.gov). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold. These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when the cumulative alignment score falls off by the quantity X from its maximum achieved value, the cumulative score goes to zero or below due to the accumulation of one or more negative-scoring residue alignments, or the end of either sequence is reached.

In addition to calculating percent sequence identity, the BLAST algorithm also performs a statistical analysis of the similarity between two sequences. One measure of similarity provided by the BLAST algorithm is the smallest sum probability (P(N)), which provides an indication of the probability by which a match between two nucleotide or amino acid sequences would occur by chance. For example, a test nucleic acid sequence is considered similar to a reference sequence if the smallest sum probability in a comparison of the test nucleic acid sequence to the reference nucleic acid sequence is less than about 0.1, less than about 0.01, or even less than about 0.001.

To obtain gapped alignments for comparison purposes, Gapped BLAST (in BLAST 2.0) can be utilized. Alternatively, PSI-BLAST (in BLAST 2.0) can be used to perform an iterated search that detects distant relationships between molecules. When using BLAST, Gapped BLAST, PSI-BLAST, the default parameters of the respective programs (e.g., BLASTN for nucleotide sequences, BLASTX for proteins) can be used. The BLASTN program (for nucleotide sequences) uses as defaults a wordlength (W) of 11, an expectation (E) of 10, a cutoff of 100, M=5, N=−4, and a comparison of both strands. For amino acid sequences, the BLASTP program uses as defaults a wordlength (W) of 3, an expectation (E) of 10, and the BLOSUM62 scoring matrix. See the World Wide Web at ncbi.nlm.nih.gov. Alignment may also be performed manually by visual inspection.

Comparison of nucleotide sequences for determination of percent sequence identity to the promoter sequences disclosed herein can be made using the BlastN program (version 1.4.7 or later) with its default parameters or any equivalent program. By “equivalent program” is intended any sequence comparison program that, for any two sequences in question, generates an alignment having identical nucleotide or amino acid residue matches and an identical percent sequence identity when compared to the corresponding alignment generated by a BLAST program.

(c) As used herein, “sequence identity” or “identity” in the context of two nucleic acid or polypeptide sequences makes reference to a specified percentage of residues in the two sequences that are the same when aligned for maximum correspondence over a specified comparison window, as measured by sequence comparison algorithms or by visual inspection. When percentage of sequence identity is used in reference to proteins, it is recognized that residue positions that are not identical often differ by conservative amino acid substitutions, where amino acid residues are substituted for other amino acid residues with similar chemical properties (e.g., charge or hydrophobicity) and therefore do not change the functional properties of the molecule. When sequences differ in conservative substitutions, the percent sequence identity may be adjusted upwards to correct for the conservative nature of the substitution. Sequences that differ by such conservative substitutions are said to have “sequence similarity” or “similarity.” Means for making this adjustment are well known to those of skill in the art. Typically, this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., as implemented in the program PC/GENE (Intelligenetics, Mountain View, Calif.).

(d) As used herein, “percentage of sequence identity” means the value determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison, and multiplying the result by 100 to yield the percentage of sequence identity.

(e)(i) The term “substantial identity” of polynucleotide sequences means that a polynucleotide comprises a sequence that has at least 70%, 71%, 72%, 73%, 74%, 75%, 76%, 77%, 78%, or 79%; at least 80%, 81%, 82%, 83%, 84%, 85%, 86%, 87%, 88%, or 89%; at least 90%, 91%, 92%, 93%, or 94%; or even at least 95%, 96%, 97%, 98%, or 99% sequence identity, compared to a reference sequence using one of the alignment programs described using standard parameters. One of skill in the art will recognize that these values can be appropriately adjusted to determine corresponding identity of proteins encoded by two nucleotide sequences by taking into account codon degeneracy, amino acid similarity, reading frame positioning, and the like. Substantial identity of amino acid sequences for these purposes normally means sequence identity of at least 70%, or at least 80%, 90%, or even at least 95%.

Another indication that nucleotide sequences are substantially identical is if two molecules hybridize to each other under stringent conditions (see below). Generally, stringent conditions are selected to be about 5° C. lower than the thermal melting point (Tm) for the specific sequence at a defined ionic strength and pH. However, stringent conditions encompass temperatures in the range of about 1° C. to about 20° C., depending upon the desired degree of stringency as otherwise qualified herein. Nucleic acids that do not hybridize to each other under stringent conditions are still substantially identical if the polypeptides they encode are substantially identical. This may occur, e.g., when a copy of a nucleic acid is created using the maximum codon degeneracy permitted by the genetic code. One indication that two nucleic acid sequences are substantially identical is when the polypeptide encoded by the first nucleic acid is immunologically cross reactive with the polypeptide encoded by the second nucleic acid.

(e)(ii) The term “substantial identity” in the context of a peptide indicates that a peptide comprises a sequence with at least 70%, 71%, 72%, 73%, 74%, 75%, 76%, 77%, 78%, or 79%; at least 80%, 81%, 82%, 83%, 84%, 85%, 86%, 87%, 88%, or 89%; or at least 90%, 91%, 92%, 93%, or 94%; or even at least 95%, 96%, 97%, 98% or 99% sequence identity to the reference sequence over a specified comparison window. An indication that two peptide sequences are substantially identical is that one peptide is immunologically reactive with antibodies raised against the second peptide. Thus, a peptide is substantially identical to a second peptide, for example, where the two peptides differ only by a conservative substitution.

For sequence comparison, typically one sequence acts as a reference sequence to which test sequences are compared. When using a sequence comparison algorithm, test and reference sequences are input into a computer, subsequence coordinates are designated if necessary, and sequence algorithm program parameters are designated. The sequence comparison algorithm then calculates the percent sequence identity for the test sequence(s) relative to the reference sequence, based on the designated program parameters.

The term “RNA transcript” refers to the product resulting from RNA polymerase catalyzed transcription of a DNA sequence. When the RNA transcript is a perfect complementary copy of the DNA sequence, it is referred to as the primary transcript or it may be a RNA sequence derived from posttranscriptional processing of the primary transcript and is referred to as the mature RNA. “Messenger RNA” (mRNA) refers to the RNA that is without introns and that can be translated into protein by the cell. “cDNA” refers to a single- or a double-stranded DNA that is complementary to and derived from mRNA.

As used herein, “treatment” (and grammatical variations thereof such as “treat” or “treating”) refers to clinical intervention to alter the natural course of the individual being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, decreasing the rate of disease progression, amelioration or palliation of the disease state, and remission or improved prognosis.

The phrase “effective amount” means an amount of a compound described herein that (i) treats or prevents the particular disease, condition, or disorder, (ii) attenuates, ameliorates, or eliminates one or more symptoms of the particular disease, condition, or disorder, or (iii) prevents or delays the onset of one or more symptoms of the particular disease, condition, or disorder described herein.

A “therapeutically effective amount” of a substance/molecule disclosed herein may vary according to factors such as the disease state, age, sex, and weight of the individual, and the ability of the substance/molecule, to elicit a desired response in the individual. A therapeutically effective amount encompasses an amount in which any toxic or detrimental effects of the substance/molecule are outweighed by the therapeutically beneficial effects. A “prophylactically effective amount” refers to an amount effective, at dosages and for periods of time necessary, to achieve the desired prophylactic result. Typically, but not necessarily, since a prophylactic dose is used in subjects prior to or at an earlier stage of disease, the prophylactically effective amount would be less than the therapeutically effective amount.

The term “mammal” refers to any mammalian species such as a human, mouse, rat, dog, cat, hamster, guinea pig, rabbit, livestock, and the like.

The terms “obtaining a sample from a patient”, “obtained from a patient” and similar phrasing, is used to refer to obtaining the sample directly from the patient, as well as obtaining the sample indirectly from the patient through an intermediary individual (e.g., obtaining the sample from a courier who obtained the sample from a nurse who obtained the sample from the patient).

The term “enriched population” in the context of a cell composition means that population contains an amount of a specified cell type that is a substantially greater proportion than what is found in the tissue from which the cells are derived. The cells of the specified type may be enriched, relative to the natural tissue, by at least 10%, 25%, 50%, 75%, 100%, 150%, 200%, 500%, or 1000%. Alternatively, the cell population may contain at least 20%, 50%, 70%, 80%, 90%, or 95% of the specified cell type.

### CERTAIN EMBODIMENTS

Certain embodiments described herein are included below.

Embodiment 1. A method for treating dysregulated lipid metabolism in a mammal in need thereof, comprising administering to the mammal an effective amount of an agonist anti-triggering receptor expressed on myeloid cells 2 (TREM2) antibody.

Embodiment 2. The method of embodiment 1, wherein cells expressing TREM2 in the mammal exhibit dysregulated lipid metabolism.

Embodiment 3. The method of embodiment 2, wherein the cells are microglial cells or macrophages.

Embodiment 4. The method of any one of embodiments 1-3, wherein the mammal has, or has been determined to have, reduced TREM2 activity.

Embodiment 5. The method of embodiment 4, wherein the reduced TREM2 activity is caused by reduced TREM2 protein levels.

Embodiment 6. The method of embodiment 4, wherein the reduced TREM2 activity is caused by reduced cell surface protein levels.

Embodiment 7. The method of embodiment 4, wherein the reduced TREM2 activity is caused by a TREM2 loss or partial loss of function mutation.

Embodiment 8. The method of any one of embodiments 1-7, wherein the mammal has, or has been determined to have, reduced apolipoprotein E (ApoE) activity.

Embodiment 9. The method of embodiment 8, wherein the mammal has, or has been determined to have, an APOE loss of function or partial loss of function mutation or coding variant.

Embodiment 10. The method of any one of embodiments 1-7, wherein the mammal has, or has been determined to have, an APOE e4 allele.

Embodiment 11. The method of any one of embodiments 1-7, wherein the mammal does not have, or has been determined to not have, an APOE e4 allele.

Embodiment 12. The method of any one of embodiments 1-11, wherein the dysregulated lipid metabolism comprises increased accumulation of one or more lipids.

Embodiment 13. The method of embodiment 12, wherein the increased accumulation of the one or more lipids is intracellular.

Embodiment 14. The method of embodiment 13, wherein the one or more lipids accumulate intracellularly in microglial cells or macrophages.

Embodiment 15. The method of embodiment 12, wherein the increased accumulation of the one or more lipids is extracellular.

Embodiment 16 The method of any one of embodiments 12-15, wherein the one or more lipids are selected from the group consisting of cholesteryl esters, oxidized cholesteryl esters, bis(monoacylglycero)phosphate species (BMPs), diacylglycerides, triacylglycerides, hexosylceramides, galactosylceramides, lactosylceramides, sulfatides, gangliosides, phosphatidylserine 38:4, bis(monoacylglycero)phosphate 44:12, lysophosphatidylcholine 16:0, platelet activating factor, cholesterol sulfate, lysophosphatidylethanolamine, and combinations thereof.

Embodiment 17. The method of embodiment 16, wherein the one or more lipids includes a cholesteryl ester.

Embodiment 18. The method of any one of embodiments 1-17, wherein the mammal has inflammation associated with the dysregulated lipid metabolism.

Embodiment 19. The method of any one of embodiments 1-18, wherein the mammal has or is prone to developing Alzheimer's disease, Nasu-Hakola disease (NHD), Lewy body dementia, Parkinson's disease, retinal degeneration (e.g., macular degeneration), Huntington's disease, Frontotemporal Lobar Degeneration (FTD), Amyotrophic Lateral Sclerosis (ALS), Niemann-Pick disease type A, Niemann-Pick disease type B, Niemann-Pick disease type C, obesity, type 2 diabetes, alcoholic or non-alcoholic steatohepatitis, alcoholic or non-alcoholic fatty liver disease, multiple sclerosis, vanishing white matter disease, rheumatoid arthritis (RA) or atherosclerosis.

Embodiment 20. The method of any one of embodiments 1-18, wherein the mammal has or is prone to developing Alzheimer's disease, Nasu-Hakola disease (NHD), Lewy body dementia, Parkinson's disease, retinal degeneration, Huntington's disease, Frontotemporal Lobar Degeneration (FTD), Amyotrophic Lateral Sclerosis (ALS), Niemann-Pick disease type A, Niemann-Pick disease type B, Niemann-Pick disease type C, multiple sclerosis or vanishing white matter disease.

Embodiment 21. The method of any one of embodiments 1-18, wherein the mammal has or is prone to developing obesity, type 2 diabetes, alcoholic or non-alcoholic steatohepatitis, alcoholic or non-alcoholic fatty liver disease, rheumatoid arthritis (RA) or atherosclerosis.

Embodiment 22. The method of embodiment 19, wherein the mammal has or is prone to developing Alzheimer's disease.

Embodiment 23. The method of embodiment 19, wherein the mammal has or is prone to developing NHD.

Embodiment 24. The method of embodiment 19, wherein the mammal has or is prone to developing atherosclerosis.

Embodiment 25. The method of embodiment 19, wherein the mammal has or is prone to developing Niemann-Pick disease type C.

Embodiment 26 The method of any one of embodiments 1-25, wherein the agonist anti-TREM2 antibody is MAB17291 or 78.18.

Embodiment 27. The method of any one of embodiments 1-26, wherein the agonist anti-TREM2 antibody reduces lipid accumulation.

Embodiment 28. The method of embodiment 27, wherein the agonist anti-TREM2 antibody reduces accumulation of cholesteryl esters.

Embodiment 29. The method of embodiment 27 or 28, wherein the agonist anti-TREM2 antibody reduces intracellular lipid accumulation.

Embodiment 30. The method of embodiment 27 or 28, wherein the agonist anti-TREM2 antibody reduces extracellular lipid accumulation.

Embodiment 31. The method of any one of embodiments 1-30, wherein the administration reduces the expression of at least one pro-inflammatory cytokine.

Embodiment 32. The method of embodiment 31, wherein the at least one cytokine is selected from the group consisting of G-CSF, INFy, IL-12 (p40), IL-12 (p70), LIX (CXCL5), MCP-1 (CCL2), MIG (CXCL9), IL-1alpha, IL-1beta and IL-18.

Embodiment 33. The method of embodiment 32, wherein the at least one cytokine is IL-1beta.

Embodiment 34. The method of any one of embodiments 1-33, further comprising administering a second therapeutic agent.

Embodiment 35. The method of embodiment 34, wherein the second therapeutic agent is selected from the group consisting of an RXR agonist, an LXR agonist and an acetyl-CoA acetyltransferase 1 (ACAT1) inhibitor.

Embodiment 36 The method of embodiment 35, wherein the second therapeutic agent is an RXR agonist.

Embodiment 37. The method of embodiment 36, wherein the RXR agonist is bexarotene.

Embodiment 38. The method of embodiment 35, wherein the second therapeutic agent is an LXR agonist.

Embodiment 39. The method of embodiment 38, wherein the LXR agonist is GW3965.

Embodiment 40. The method of embodiment 35, wherein the second therapeutic agent is an acetyl-CoA acetyltransferase 1 (ACAT1) inhibitor.

Embodiment 41. The method of embodiment 40, wherein the ACAT1 inhibitor is CP-113,818, CI-1011 or K-604.

Embodiment 42. The method of embodiment 41, wherein the ACAT1 inhibitor is K-604.

Embodiment 43. The method of embodiment 34, wherein the second therapeutic agent is an agent useful for treating Alzheimer's disease or atherosclerosis.

Embodiment 44. A method of treating dysregulated lipid metabolism in a patient in need thereof, comprising:

1) obtaining or having obtained a biological sample from the patient;

2) detecting or having detected reduced TREM2 activity, reduced ApoE activity or an APOE ε4 allele in the sample;

3) diagnosing the patient with dysregulated lipid metabolism when reduced TREM2 activity, reduced ApoE activity or an APOE ε4 allele is detected; and

4) administering an effective amount of an agonist anti-TREM2 antibody to the diagnosed patient.

Embodiment 45. A method of treating a patient with an agonist anti-TREM2 antibody, the method comprising:

1) obtaining or having obtained a biological sample from the patient;

2) analyzing the biological sample or having analyzed the sample to detect the presence of reduced TREM2 activity, reduced ApoE activity or an APOE ε4 allele, thereby diagnosing the patient as having dysregulated lipid metabolism; and

3) administering an effective amount of an agonist anti-TREM2 antibody to the diagnosed patient.

Embodiment 46 An agonist anti-TREM2 antibody for use in the treatment of dysregulated lipid metabolism in a mammal.

Embodiment 47. The antibody for use as described in embodiment 46, wherein the mammal has, or has been determined to have, reduced TREM2 activity.

Embodiment 48. The antibody for use as described in embodiment 46 or 47, wherein the mammal has, or has been determined to have, reduced ApoE activity.

Embodiment 49. The antibody for use as described in embodiment 46 or 47, wherein the mammal has, or has been determined to have, an APOE loss or partial loss of function mutation or coding variant.

Embodiment 50. The antibody for use as described in embodiment 46 or 47, wherein the mammal has, or has been determined to have, an APOE ε4 allele.

Embodiment 51. The antibody for use as described in embodiment 46 or 47, wherein the mammal does not have, or has been determined to not have, an APOE ε4 allele.

Embodiment 52. The use of an agonist anti-TREM2 antibody to prepare a medicament for treating dysregulated lipid metabolism in a mammal.

Embodiment 53. The use of embodiment 52, wherein the mammal has, or has been determined to have, reduced TREM2 activity.

Embodiment 54. The use of embodiment 52 or 53, wherein the mammal has, or has been determined to have, reduced ApoE activity.

Embodiment 55. The use of embodiment 52 or 53, wherein the mammal has, or has been determined to have, an APOE loss or partial loss of function mutation or coding variant.

Embodiment 56 The use of embodiment 52 or 53, wherein the mammal has, or has been determined to have an APOE ε4 allele.

Embodiment 57. The use of embodiment 52 or 53, wherein the mammal does not have, or has been determined to not have, an APOE ε4 allele.

Embodiment 58. A method of reducing intracellular accumulation of one or more lipids in a cell, comprising contacting the cell with an effective amount of an agonist anti-TREM2 antibody.

Embodiment 59. The method of embodiment 58, wherein the cell is a microglial cell.

Embodiment 60. The method of embodiment 58, wherein the cell is a macrophage.

Embodiment 61. The method of any one of embodiments 58-60, wherein the one or more lipids are selected from the group consisting of cholesteryl esters, oxidized cholesteryl esters, BMPs, diacylglycerides, triacylglycerides, hexosylceramides, galactosylceramides, lactosylceramides, sulfatides, gangliosides, phosphatidylserine 38:4, bis(monoacylglycero)phosphate 44:12, lysophosphatidylcholine 16:0, platelet activating factor, cholesterol sulfate, lysophosphatidylethanolamine, and combinations thereof.

Embodiment 62. The method of embodiment 61, wherein the one or more lipids includes a cholesteryl ester.

Embodiment 63. The method of any one of embodiments 58-62, wherein the cell has, or has been determined to have, reduced TREM2 activity.

Embodiment 64. The method of any one of embodiments 58-63, wherein the cell has, or has been determined to have, reduced ApoE activity.

Embodiment 65. The method of any one of embodiments 58-63, wherein the cell has, or has been determined to have, an APOE loss or partial loss of function mutation or coding variant.

Embodiment 66 The method of any one of embodiments 58-63, wherein the cell expresses, or has been determined to express, ApoE4.

Embodiment 67. The method of any one of embodiments 58-63, wherein the cell does not express, or has been determined to not express, ApoE4.

Embodiment 68. The method of any one of embodiments 58-67, wherein the agonist anti-TREM2 antibody is MAB17291 or 78.18.

Embodiment 69. The method of any one of embodiments 58-68, wherein the cell is contacted with the agonist anti-TREM2 antibody in vitro, in vivo or ex vivo.

Embodiment 70. The method of any one of embodiments 58-68, wherein the cell is present in a mammal.

Embodiment 71. The method of any one of embodiments 58-68, wherein the cell is present in a mammal and is contacted with the agonist anti-TREM2 antibody in vivo.

Embodiment 72. The method of embodiment 70 or 71, wherein the mammal has inflammation associated with the intracellular lipid accumulation.

Embodiment 73. The method of embodiment 72, wherein the agonist anti-TREM2 antibody reduces the expression of at least one pro-inflammatory cytokine.

Embodiment 74. The method of embodiment 73, wherein the at least one cytokine is selected from the group consisting of G-CSF, INFy, IL-12 (p40), IL-12 (p70), LIX (CXCL5), MCP-1 (CCL2), MIG (CXCL9), IL-1alpha, IL-1beta and IL-18.

Embodiment 75. The method of embodiment 74, wherein the at least one cytokine is IL-1beta.

Embodiment 76 The method of any one of embodiments 70-75, wherein the mammal has or is prone to developing Alzheimer's disease, NHD, Lewy body dementia, Parkinson's disease, retinal degeneration (e.g., macular degeneration), Huntington's disease, FTD, ALS, Niemann-Pick disease type A, Niemann-Pick disease type B, Niemann-Pick disease type C, obesity, type 2 diabetes, alcoholic or non-alcoholic steatohepatitis, alcoholic or non-alcoholic fatty liver disease, multiple sclerosis, vanishing white matter disease, RA or atherosclerosis.

Embodiment 77. The method of embodiment 76, wherein the mammal has or is prone to developing NHD.

Embodiment 78. The method of embodiment 76, wherein the mammal has or is prone to developing atherosclerosis.

Embodiment 79. The method of embodiment 76, wherein the mammal has or is prone to developing Niemann-Pick disease type C.

Embodiment 80. The method of any one of embodiments 58-79, further comprising contacting the cell with a second therapeutic agent.

Embodiment 81. The method of embodiment 80, wherein the second therapeutic agent is an RXR agonist.

Embodiment 82. The method of embodiment 81, wherein the RXR agonist is bexarotene.

Embodiment 83. The method of embodiment 80, wherein the second therapeutic agent is an LXR agonist.

Embodiment 84. The method of embodiment 83, wherein the LXR agonist is GW3965.

Embodiment 85. The method of embodiment 80, wherein the second therapeutic agent is an ACAT1 inhibitor.

Embodiment 86 The method of embodiment 85, wherein the ACAT1 inhibitor is CP-113,818, CI-1011 or K-604.

Embodiment 87. The method of embodiment 86, wherein the ACAT1 inhibitor is K-604.

Embodiment 88. An agonist anti-TREM2 antibody for use in reducing intracellular accumulation of one or more lipids in a cell.

Embodiment 89. The use of an agonist anti-TREM2 antibody to prepare a medicament for reducing intracellular accumulation of one or more lipids in a cell.

Embodiment 90. A method of treating Alzheimer's disease in a mammal in need thereof, the method comprising administering to the mammal an agonist anti-TREM2 antibody, wherein the mammal has, or has been determined to have, dysregulated lipid metabolism.

Embodiment 91. The method of embodiment 90, wherein the mammal has, or has been determined to have, dysregulated lipid metabolism in TREM2 expressing cells.

Embodiment 92. A method of treating Alzheimer's disease in a mammal in need thereof, the method comprising administering to the mammal an agonist anti-TREM2 antibody, wherein the mammal has, or has been determined to have, dysregulated lipid metabolism in TREM2-expressing cells.

Embodiment 93. The method of any one of embodiments 91-92, wherein the TREM2-expressing cells are microglial cells.

Embodiment 94. The method of any one of embodiments 91-93, wherein the TREM2-expressing cells have, or have been determined to have, reduced TREM2 activity.

Embodiment 95. The method of any one of embodiments 90-94, wherein the dysregulated lipid metabolism comprises increased intracellular accumulation of one or more lipids.

Embodiment 96 The method of embodiment 95, wherein the one or more lipids are selected from the group consisting of cholesteryl esters, oxidized cholesteryl esters, BMPs, diacylglycerides, triacylglycerides, hexosylceramides, galactosylceramides, lactosylceramides, sulfatides, gangliosides, phosphatidylserine 38:4, bis(monoacylglycero)phosphate 44:12, lysophosphatidylcholine 16:0, platelet activating factor, cholesterol sulfate, lysophosphatidylethanolamine, and combinations thereof.

Embodiment 97. The method of embodiment 96, wherein the one or more lipids includes a cholesteryl ester.

Embodiment 98. The method of any one of embodiments 90-97, wherein the mammal has inflammation associated with the dysregulated lipid metabolism.

Embodiment 99. The method of embodiment 98, wherein the administration reduces the expression of at least one pro-inflammatory cytokine.

Embodiment 100. The method of embodiment 99, wherein the at least one cytokine is selected from the group consisting of G-CSF, INFy, IL-12 (p40), IL-12 (p70), LIX (CXCL5), MCP-1 (CCL2), MIG (CXCL9), IL-1alpha, IL-1beta and IL-18.

Embodiment 101. The method of embodiment 100, wherein the at least one cytokine is IL-1beta.

Embodiment 102. The method of any one of embodiments 90-101, wherein the agonist anti-TREM2 antibody is MAB17291 or 78.18.

Embodiment 103. The method of any one of embodiments 90-102, further comprising administering to the mammal a second therapeutic agent.

Embodiment 104. The method of embodiment 103, wherein the second therapeutic agent is an RXR agonist.

Embodiment 105. The method of embodiment 104, wherein the RXR agonist is bexarotene.

Embodiment 106. The method of embodiment 103, wherein the second therapeutic agent is an LXR agonist.

Embodiment 107. The method of embodiment 106, wherein the LXR agonist is GW3965.

Embodiment 108. The method of embodiment 103, wherein the second therapeutic agent is an ACAT1 inhibitor.

Embodiment 109. The method of embodiment 108, wherein the ACAT1 inhibitor is CP-113,818, CI-1011 or K-604.

Embodiment 110. The method of embodiment 109, wherein the ACAT1 inhibitor is K-604.

Embodiment 111. An agonist anti-TREM2 antibody for use in the treatment of Alzheimer's disease in a mammal, wherein the mammal has, or has been determined to have, dysregulated lipid metabolism.

Embodiment 112. An agonist anti-TREM2 antibody for use in the treatment of Alzheimer's disease in a mammal, wherein the mammal has, or has been determined to have, dysregulated lipid metabolism in TREM2-expressing cells.

Embodiment 113. The use of an agonist anti-TREM2 antibody to prepare a medicament for treating Alzheimer's disease in a mammal, wherein the mammal has, or has been determined to have, dysregulated lipid metabolism.

Embodiment 114. The use of an agonist anti-TREM2 antibody to prepare a medicament for treating Alzheimer's disease in a mammal, wherein the mammal has, or has been determined to have, dysregulated lipid metabolism in TREM2-expressing cells.

Embodiment 115. A method of treating atherosclerosis in a mammal in need thereof, comprising administering to the mammal an effective amount of an agonist anti-TREM2 antibody.

Embodiment 116. The method of embodiment 115, wherein the mammal has, or has been determined to have, dysregulated lipid metabolism.

Embodiment 117. The method of any one of embodiments 115-116, wherein the dysregulated lipid metabolism comprises increased accumulation of one or more lipids.

Embodiment 118. The method of embodiment 117, wherein the increased accumulation of the one or more lipids is intracellular.

Embodiment 119. The method of embodiment 117, wherein the one or more lipids accumulate intracellularly in macrophages.

Embodiment 120. The method of embodiment 119, wherein the macrophages have, or have been determined to have, reduced TREM2 activity.

Embodiment 121. The method of embodiment 117, wherein the increased accumulation of the one or more lipids is extracellular.

Embodiment 122. The method of any one of embodiments 117-121, wherein the one or more lipids are selected from the group consisting of cholesteryl esters, oxidized cholesteryl esters, BMPs, diacylglycerides, triacylglycerides, hexosylceramides, galactosylceramides, lactosylceramides, sulfatides, gangliosides, phosphatidylserine 38:4, bis(monoacylglycero)phosphate 44:12, lysophosphatidylcholine 16:0, platelet activating factor, cholesterol sulfate, lysophosphatidylethanolamine, and combinations thereof.

Embodiment 123. The method of embodiment 122, wherein the one or more lipids includes a cholesteryl ester.

Embodiment 124. The method of any one of embodiments 116-123, wherein the mammal has inflammation associated with the dysregulated lipid metabolism.

Embodiment 125. The method of embodiment 124, wherein the administration reduces the expression of at least one pro-inflammatory cytokine.

Embodiment 126. The method of embodiment 125, wherein the at least one cytokine is selected from the group consisting of G-CSF, INFy, IL-12 (p40), IL-12 (p70), LIX (CXCL5), MCP-1 (CCL2), MIG (CXCL9), IL-1alpha, IL-1beta and IL-18.

Embodiment 127. The method of embodiment 126, wherein the at least one cytokine is IL-1beta.

Embodiment 128. The method of any one of embodiments 115-127, wherein the agonist anti-TREM2 antibody is MAB17291 or 78.18.

Embodiment 129. The method of any one of embodiments 115-128, further comprising administering a second therapeutic agent.

Embodiment 130. The method of embodiment 129, wherein the second therapeutic agent is an agent useful for treating atherosclerosis.

Embodiment 131. The method of embodiment 129, wherein the second therapeutic agent is an RXR agonist, LXR agonist or ACAT1 inhibitor.

Embodiment 132. An agonist anti-TREM2 antibody for use in the treatment of atherosclerosis in a mammal.

Embodiment 133. The use of an agonist anti-TREM2 antibody to prepare a medicament for treating atherosclerosis in a mammal.

Embodiment 134. A method of treating inflammation in a mammal in need thereof, comprising administering to the mammal an effective amount of an agonist anti-TREM2 antibody.

Embodiment 135. The method of embodiment 134, wherein the administration reduces the expression of at least one pro-inflammatory cytokine.

Embodiment 136. The method of embodiment 135, wherein the at least one cytokine is associated with the inflammasome response.

Embodiment 137. The method of embodiment 135 or 136, wherein the at least one cytokine is selected from the group consisting of G-CSF, INFy, IL-12 (p40), IL-12 (p70), LIX (CXCL5), MCP-1 (CCL2), MIG (CXCL9), IL-1alpha, IL-1beta and IL-18.

Embodiment 138. The method of embodiment 137, wherein the at least one cytokine is IL-1beta.

Embodiment 139. The method of any one of embodiments 134-138, wherein the mammal has or is prone to developing an inflammasome related disease or disorder.

Embodiment 140. The method of any one of embodiments 134-138, wherein the mammal has or is prone to developing rheumatoid arthritis, gout, or inflammatory bowel disease (IBD).

Embodiment 141. The method of any one of embodiments 134-138, wherein the inflammation is associated with dysregulated lipid metabolism.

Embodiment 142. The method of embodiment 141, wherein administration of the agonist anti-TREM2 antibody reduces lipid accumulation.

Embodiment 143. The method of embodiment 141 or 142, wherein the mammal has or is prone to developing Alzheimer's disease, Nasu-Hakola disease (NHD), Lewy body dementia, Parkinson's disease, retinal degeneration (e.g., macular degeneration), Huntington's disease, Niemann-Pick disease type A, Niemann-Pick disease type B, Niemann-Pick disease type C, obesity, type 2 diabetes, alcoholic or non-alcoholic steatohepatitis, alcoholic or non-alcoholic fatty liver disease, multiple sclerosis, vanishing white matter disease, or atherosclerosis.

Embodiment 144. The method of embodiment 141 or 142, wherein the mammal has or is prone to developing Alzheimer's disease, Nasu-Hakola disease (NHD), Lewy body dementia, Parkinson's disease, retinal degeneration, Huntington's disease, Niemann-Pick disease type A, Niemann-Pick disease type B, Niemann-Pick disease type C, multiple sclerosis or vanishing white matter disease.

Embodiment 145. The method of embodiment 141 or 142, wherein the mammal has or is prone to developing obesity, type 2 diabetes, alcoholic or non-alcoholic steatohepatitis, alcoholic or non-alcoholic fatty liver disease or atherosclerosis.

Embodiment 146. The method of any one of embodiments 134-145, wherein the agonist anti-TREM2 antibody is MAB17291 or 78.18.

Embodiment 147. The method of any one of embodiments 134-146, further comprising administering a second therapeutic agent.

Embodiment 148. The method of embodiment 147, wherein the second therapeutic agent is an RXR agonist, LXR agonist or ACAT1 inhibitor.

Embodiment 149. An agonist anti-TREM2 antibody for use in the treatment of inflammation in a mammal.

Embodiment 150. The use of an agonist anti-TREM2 antibody to prepare a medicament for treating inflammation in a mammal.

Embodiment 151. The antibody of embodiment 149 or the use of embodiment 150, wherein the inflammation is associated with dysregulated lipid metabolism.

Embodiment 152. A method of sorting populations of CNS cells from a tissue sample, comprising:

(a) contacting the tissue sample with an anti-CD45 primary antibody, an anti-CD11b primary antibody and an anti-astrocyte cell surface antigen-2 (ACSA-2) primary antibody, wherein each primary antibody is uniquely labeled, to provide a labeled tissue sample; and

(b) sorting the cells in the labeled tissue sample by flow cytometry, wherein the method provides distinct cell populations of astrocytes and microglial cells.

Embodiment 153. The method of claim 152, wherein the anti-CD45 primary antibody, the anti-CD11b primary antibody and the anti-ACSA-2 primary antibody are present in a composition.

Embodiment 154. A method of sorting populations of CNS cells from a tissue sample, comprising:

(a) contacting the tissue sample with a composition comprising: an anti-CD45 primary antibody, an anti-CD11b primary antibody and an anti-astrocyte cell surface antigen-2 (ACSA-2) primary antibody, wherein each primary antibody is uniquely labeled, to provide a labeled tissue sample; and

(b) sorting the cells in the labeled tissue sample by flow cytometry, wherein the method provides distinct cell populations of astrocytes and microglial cells.

Embodiment 155. The method of embodiment 153 or 154, wherein the composition further comprises a viability dye.

Embodiment 156. The method of any one of embodiments 152-154, further comprising contacting the tissue sample with a viability dye.

Embodiment 157. The method of any one of embodiments 152-156, which provides a distinct population of microglial cells comprising less than about 20% non-microglial cells.

Embodiment 158. The method of any one of embodiments 152-156, which provides a distinct population of astrocytes comprising less than about 20% non-astrocytic cells.

Embodiment 159. The method of any one of embodiments 152-158, wherein the microglial cell population is sorted based on the following marker profile: CD45^{low}/CD11b⁺/ACSA-2⁻.

Embodiment 160. The method of any one of embodiments 152-159, wherein the astrocyte population is sorted based on the following marker profile: CD45⁻/CD11b⁻/ACSA-2⁺.

Embodiment 161. The method of any one of embodiments 152-160, wherein the distinct cell populations are analyzed for quantification of a metabolic or nucleic acid species.

Embodiment 162. The method of embodiment 161, wherein the metabolic species is a lipid species.

Embodiment 163. The method of embodiment 161, wherein the nucleic acid species is selected from RNA, DNA, and genomic DNA.

Embodiment 164. The method of any one of embodiments 152-160, wherein the distinct cell populations are analyzed for quantification of an administered therapeutic agent.

Embodiment 165. A composition comprising a distinct cell population isolated by a method described in any one of embodiments 152-160.

Embodiment 166. The composition of embodiment 165, wherein the distinct cell population is a microglial cell population.

Embodiment 167. The composition of embodiment 165, wherein the distinct cell population is an astrocytic cell population.

Embodiment 168. A collection of CNS cells comprising two physically separate cell populations, wherein the first cell population comprises an enriched population of CD45^{low}/CD11b⁺/ACSA-2⁻ cells and the second cell population comprises an enriched population CD45⁻/CD11b⁻/ACSA-2⁺ cells.

The following Examples are intended to be non-limiting.

### Example 1: Attenuated Expression of Genes Implicated in Lipid Metabolism in Trem2 Knockout Mice with Chronic Demyelination

This example describes mouse microglial gene expression analysis.

### Cuprizone Diet to Induce Demyelination in Mice

Trem2^{-/-} mice were purchased from the Jackson Laboratory (Stock #: 027197) and backcrossed to C57BU/6J mice to generate Trem2^{+/-} mice. Trem2^{+/-} mice were further intercrossed to generate three genotypes of littermates (Trem2^{+/+}, Trem2^{+/-} and Trem2) for this study. Mice around 9-11 months of age were used. Each genotype of mice was divided into two groups (6-11/group) with either a normal diet (Envigo TD.160766) or a cuprizone diet (0.2% cuprizone, Envigo TD.160765) treatment paradigm for 5 weeks or 12 weeks. The body weight of each animal was recorded weekly to monitor the effects of cuprizone.

Fluorescence Activated Cell Sorting (FACS) of Microglia, Astrocytes, and Other Cells from Mouse Brain

To prepare a single cell suspension for sorting CNS cells, mice were perfused with PBS, brains dissected and processed into a single cell suspension according to the manufacturers' protocol using the adult brain dissociation kit (Miltenyi Biotec 130-107-677). Cells were Fc blocked and stained for flow cytometric analysis with Fixable Viability Stain BV510 to exclude dead cells (BD Biosciences 564406), CD11b-BV421 (BD Biosciences 562605), CD45-APC (BD Biosciences 559864), and ACSA-2-PE (Miltenyi Biotec 130-102-365). Cells were washed twice with Hibernate A (BrainBits LLC) and strained through a 100 μm filter before sorting CD11b⁺ microglia and ACSA-2⁺ astrocytes on a FACS Aria III (BD Biosciences) with a 100 μm nozzle. Sorted cells were processed for downstream analysis including RNAseq, scRNAseq or lipidomics.

### FACS-RNAseq Analysis of Microglial Gene Expression

Live cells were sorted into CD11b⁺ microglia (100,000-120,000 cells) versus all other unstained cells (100,000-200,000 cells) and collected directly in RLT-plus buffer (Qiagen) with 1:100 beta-mercaptoethanol. RNA was extracted using the RNeasy Plus Micro Kit (Qiagen, 74034) and resuspended in 14 μl nuclease-free water. RNA quantity and quality were assessed with an RNA 6000 Pico chip (Agilent 5067-1513) on a 2100 Bioanalyzer (Agilent). RNA was processed using the QuantSeq 3 mRNA-Seq Library Prep Kit FWD for Illumina (Lexogen), following the ‘low-input’ protocol defined by the manufacturer. Barcoded samples were quantified using the NEBNext Library Quant Kit for Illumina (NEB, E7630S). All samples were pooled in equimolar ratios into one sequencing library, which was quantified on a Bioanalyzer with a High Sensitivity DNA chip (Agilent, 5067-4626). 50 bp single-end reads were generated in Illumina HiSeq 4000 lane at the UCSF Center for Advanced Technology.

Reads were aligned to the mouse genome version GRCm38_p6. A STAR index (Dobin, A et al., Bioinformatics, 2013. 29(1): p. 15-21; version 2.5.3a) was built with the -sjdbOverhang=50 argument. Splice junctions from Gencode gene models (release M17) were provided via the --sjdbGTFfile argument. STAR alignments were generated with the following parameters: -outFilterType BySJout, --quantMode TranscriptomeSAM, -outFilterlntronMotifs RemoveNoncanonicalUnannotated, --outSAMstrandField intronMotif, -outSAMattributes NH HI AS nM MD XS and -outSAMunmapped Within. Alignments were obtained with the following parameters: --readFilesCommand zcat -outFilterType BySJout --outFilterMultimapNmax 20 --alignSJoverhangMin 8 --alignSJDBoverhangMin 1 --outFilterMismatchNmax 999 --outFilterMismatchNoverLmax 0.6 -alignIntronMin 20 -alignIntronMax 1000000 -alignMatesGapMax 1000000 --quantMode GeneCounts --outSAMunmapped Within --outSAMattributes NH HI AS nM MD XS -outSAMstrandField intronMotif -outSAMtype BAM SortedByCoordinate -outBAMcompression 6. Gene level counts were obtained usingfeatureCounts from the subread package (Liao, Y et al., Nucleic Acids Res, 2013. 41(10): e108; version 1.6.2). Gene symbols and Entrez gene identifiers were mapped using Ensembl (version 91) via the biomaRt R package (Durinck, S et al., Nat Protoc, 2009. 4(8): p. 1184-91; version 2.34.2) using R (version 3.4.3).

To identify differentially expressed genes linear models were fit using the limma Bioconductor package (Liu, R et al., Nucleic Acids Res, 2015. 43: p. e97). Only genes with sufficiently large counts, as determined by edgeR's “filterByExpr” function were included in the statistical analysis. TMM scaling factors for each sample were calculated with the “calcNormFactors” function (Robinson, M D et al., Genome Biol, 2010. 11(3): p. R25). We estimated the mean-variance relationship of log 2 transformed counts and derived observation-level weights with the “voom” function from the limma Bioconductor package (Liu, R et al., Nucleic Acids Res, 2015. 43: p. e97). Linear models were fit with the “lmFit” and “eBayes” functions. Results were plotted using the ggplot2 R package (Wickham, H et al., ggplot2: Elegant Graphics for Data Analysis, 2016). Competitive gene set tests were performed using the “camera” algorithm from the limma R package (Wu, D et al., Nucleic Acids Res, 2012. 40(17): p. e133).

### Single Cell RNAseq Library Preparation and Analysis

Dissociated cells from (2) control diet Trem2^{+/+} hemibrains and (2) Trem2^{+/+}, (2) Trem2^{+/-}, and (2) Trem2^{-/-} 12-week cuprizone treated hemibrains were processed and stained as described above. 30,000 live CD11b+/CD45lo microglia were sorted from each hemibrain and (2) hemibrains per condition were pooled into PBS+0.5% BSA to generate 4 total sequencing groups. Microglia were counted and diluted to 500,000 cells/ml in 70 μl and viability was verified to be >70%. Single cell libraries were barcoded and prepared using Chromium Single Cell 3′ Library Kit with v2 chemistry (10× Genomics, product #120267) with a Chromium Controller (10× Genomics) at the Stanford Functional Genomics Facility. ScRNASeq libraries were sequenced using a NovaSeq S4 (sequencer) at the UCSF Center for Advanced Technology.

Four single cell datasets were generated. All quality control, filtering, and downstream analysis were performed using a combination of the DropletUtils (Lun, A T L et al., biorXiv, 2018. doi: 10.1101/234872), scater (McCarthy D J et al., Bioinformatics, 2017. 33(8): p. 1179-1186) and scran (Lun, A T L et al., Genome Biology, 2016. 17: p. 75) Bioconductor (v3.8) packages. Each experiment was independently quality controlled and filtered in the following manner: (i) droplets containing only ambient RNA (FDR <0.01) were identified using the “emptyDrops” function and removed; (ii) cells with low read counts, low gene counts, or high mitochondrial load were identified with the “isOutlier” function and removed. The remaining cells from each experiment were then combined into a universal atlas of cells for downstream analysis. Differences in sequencing depth per cell were accounted for using the “computeSumFactors” function, and normalized log counts for each gene were computed with the “normalize” function. An initial PCA and subsequent tSNE analysis further identified a group of cells from each sample whose only real distinguishing characteristic was a high mitochondrial load. This group of cells were deemed to be a technical artifact and removed to form a final, high quality cell atlas.

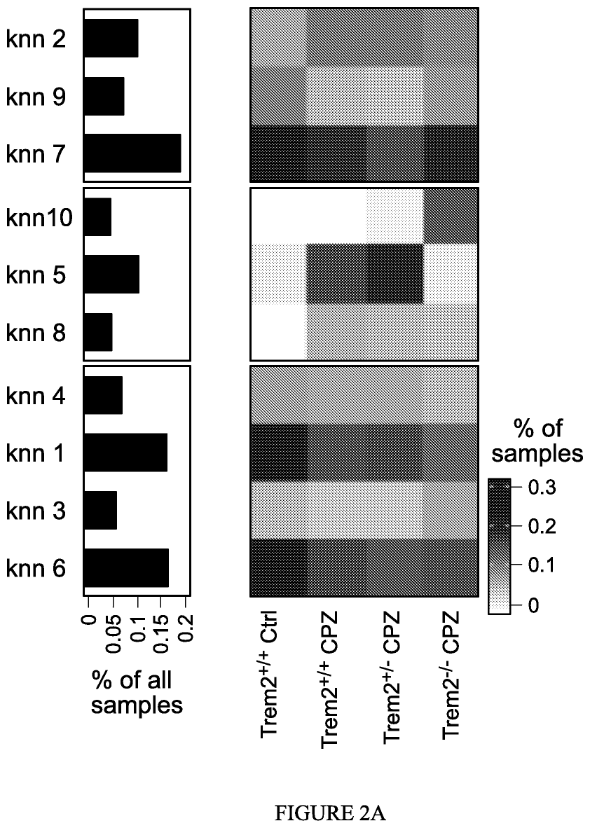

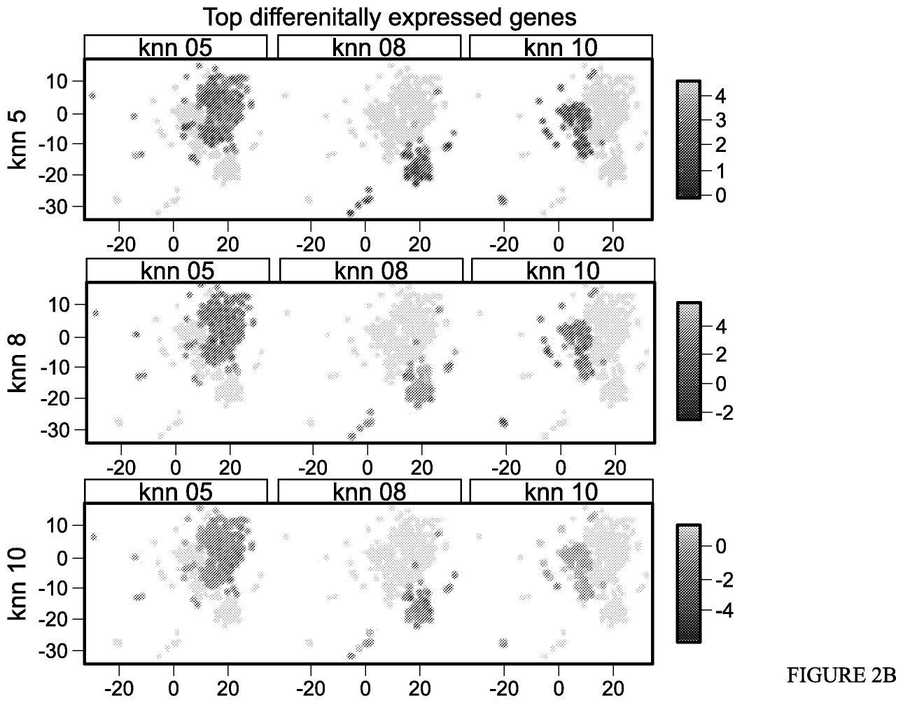

Ten clusters of microglial cells were identified in the atlas by first building a shared nearest-neighbor graph using the “buildSNNGraph” function (with k=6) followed by community detection using the Louvian method. The largest cluster contains 595 cells (knn_07) and the smallest contains 147 (knn_10). Clusters were characterized first by identifying the fraction of cells across the atlas that belong to each cluster (FIG. 2A, left), and subsequently by the fraction of cells per sample that belong to each cluster (FIG. 2A, right). Marker gene and gene set enrichment analyses were performed for each cluster under two scenarios: universal—each cluster was tested against the entire dataset; or restricted—the three interesting clusters of cells (knn_05, knn_05, and knn_10) were only tested against each other. Marker genes per cluster were identified by Wilcoxon Test using the “pairwiseWilcox” function and filtered using an FDR threshold of 0.001. Fold changes were reported per gene by taking the average of the log 2 fold change of the gene within the given cluster versus the each of the rest (as calculated by the “pairwiseTTests” function).

TABLE 1
Genes altered in microglial clusters knn 5, 8, and 10.

| Gene | Direction | Cluster | FDR | Log2 FC |
|---|---|---|---|---|
| Cd14 | up | knn_5 | 6.34E−04 | 1.448 |
| Cd74 | up | knn_5 | 1.33E−04 | 1.736 |
| Tmem176a | up | knn_5 | 5.07E−05 | 1.081 |
| Ptprc | up | knn_5 | 1.65E−06 | 0.720 |
| Bcl2a1a | up | knn_5 | 7.06E−10 | 0.777 |
| Spp1 | up | knn_5 | 6.67E−04 | 1.552 |
| Axl | up | knn_5 | 2.57E−07 | 0.804 |
| Ctsh | up | knn_5 | 1.97E−05 | 0.691 |
| Lpl | up | knn_5 | 3.78E−06 | 1.204 |
| Nfkbiz | up | knn_5 | 5.19E−04 | 0.703 |
| Tmsb4x | up | knn_8 | 7.43E−05 | 0.466 |
| Sh3bgrl3 | up | knn_8 | 5.86E−05 | 0.615 |
| Serf2 | up | knn_8 | 4.98E−04 | 0.453 |
| Alp5j2 | up | knn_8 | 7.54E−06 | 0.507 |
| Rpl35 | up | knn_8 | 1.85E−04 | 0.399 |
| Ppia | up | knn_8 | 3.04E−04 | 0.294 |
| Rpl6 | up | knn_8 | 1.52E−05 | 0.312 |
| Rpl23a | up | knn_8 | 3.07E−04 | 0.366 |
| Rpl36al | up | knn_8 | 2.59E−04 | 0.396 |
| Rpl38 | up | knn_8 | 9.22E−04 | 0.310 |
| Btg1 | down | knn_8 | 5.81E−23 | −1.783 |
| Ier5 | down | knn_8 | 1.35E−26 | −1.927 |
| Jun | down | knn_8 | 1.11E−25 | −2.494 |
| Dusp1 | down | knn_8 | 1.93E−29 | −2.539 |
| Rhob | down | knn_8 | 1.26E−30 | −1.911 |
| Zfp36 | down | knn_8 | 5.64E−27 | −2.166 |
| Malat1 | down | knn_8 | 1.03E−29 | −4.234 |
| Jund | down | knn_8 | 1.10E−30 | −2.471 |
| Btg2 | down | knn_8 | 1.21E−30 | −2.584 |
| Junb | down | knn_8 | 1.10E−30 | −2.954 |
| Ltc4s | up | knn_10 | 2.59E−10 | 1.069 |
| P2ry12 | up | knn_10 | 5.05E−10 | 1.123 |
| Sgk1 | up | knn_10 | 7.33E−04 | 1.278 |
| Serpine2 | up | knn_10 | 3.06E−08 | 0.886 |
| Arl4c | up | knn_10 | 2.55E−09 | 0.852 |
| Pmp22 | up | knn_10 | 1.02E−09 | 0.891 |
| Gpr34 | up | knn_10 | 7.32E−09 | 0.819 |
| Fcrls | up | knn_10 | 8.48E−06 | 0.713 |
| Sft2d1 | up | knn_10 | 2.03E−07 | 0.727 |
| Tmem86a | up | knn_10 | 9.81E−07 | 0.767 |
| Arpc1b | down | knn_10 | 1.08E−05 | −0.456 |
| Pfn1 | down | knn_10 | 3.42E−07 | −0.491 |
| Sh3bgrl3 | down | knn_10 | 2.73E−06 | −0.653 |
| Spp1 | down | knn_10 | 4.47E−06 | −1.792 |
| Fth1 | down | knn_10 | 1.03E−09 | −0.644 |
| Tmem176b | down | knn_10 | 5.97E−06 | −1.246 |
| Tmsb4x | down | knn_10 | 4.39E−17 | −0.628 |
| Tmsb10 | down | knn_10 | 2.64E−13 | −1.569 |
| Trem2 | down | knn_10 | 5.54E−09 | −1.322 |
| Cd52 | down | knn_10 | 1.40E−13 | −1.643 |

As shown in FIGS. 1A-C, FIGS. 2A-B, and Table 1, genes involved in lipid metabolism are upregulated in Trem2^{+/+} and Trem2^{+/-} murine microglia upon acute and chronic demyelination (5 and 12-week cuprizone treatment, respectively), but exhibit reduced upregulation in Trem2^{-/-} murine microglia with chronic and acute demyelination. There are few genotype differences between bulk isolated Trem2^{+/+}, Trem2^{+/-}, and Trem2^{-/-} murine microglia in mice with a control diet (FIG. 1A). Hundreds of genes are upregulated in bulk Trem2^{+/+} and Trem2^{+/-} murine microglia with chronic demyelination, but very few are upregulated in Trem2^{-/-} microglia (FIG. 1B). Of the genes that are upregulated in bulk Trem2^{+/+} and Trem2^{+/-}, but not Trem2^{-/-} microglia, many are implicated in lipid metabolism (FIG. 1C). FIG. 1C shows the log 2 fold change of gene expression in individual genes associated with lipid metabolism in Trem2^{+/+}, Trem2^{+/-}, and Trem2^{-/-} bulk microglia with control diet (left inset) versus 5 or 12 weeks cuprizone treatment (right inset, top or bottom, respectively). FIGS. 2A and 2B identify microglia clusters of single cell RNA sequencing data from individually isolated Trem2^{+/+} control diet microglia (Trem2^{+/+} Ctrl) compared to isolated microglia from Trem2^{+/+}, Trem2^{-/-} and Trem2^{-/-} mice with chronic demyelination (Trem2^{+/+} CPZ, Trem2^{+/-} CPZ, Trem2^{-/-} CPZ). Cluster knn 8 identifies a population of microglia with genes that are up- or down-regulated with cuprizone treatment, regardless of genotype. Cluster knn_5 identifies a population of microglia with genes that are upregulated with chronic demyelination in Trem2^{+/+} CPZ and Trem2^{+/-} CPZ mice, but not control or Trem2^{-/-} CPZ mice. Cluster knn_10 identifies a population of microglia with genes that are up- or down-regulated in Trem2 CPZ mice, but not Trem2^{+/+} CPZ, Trem2^{+/-} CPZ, or control mice. Table 1 lists the genes, log fold change and direction of change, and false discovery rate (FDR) identified in clusters knn 5, 8, and 10.

### Example 2. Increased Abundance of Cholesteryl Ester and Myelin Lipids in Trem2 Knockout Forebrain and Isolated Microglia Upon Chronic Demyelination

This example describes lipidomics of mouse forebrain and isolated microglia and astrocyte cell populations.

### Forebrain Lipid Extraction

Sagittal mouse hemibrains were flash frozen in liquid nitrogen after PBS perfusion and coronally cryosectioned at −20° C. with alternating 100 μm (lipidomics) or 20 μm (histology) widths using a Leica CM 1950 cryostat. Two 100 μm sections from matched forebrain regions containing the corpus callosum were placed in a 1.5 mL LoBind tube (Eppendorf) containing a 3 mm stainless steel bead (Qiagen) with 200 μl of LC-MS grade methanol containing internal standards. Tubes were lysed using a TissueLyser (Qiagen) 2×: 1 min, 25 Hz at 4° C. 20 μl of sample was removed for protein concentration measurements using the bicinchoninic acid (BCA) assay (Pierce, Rockford, Ill., USA). Lysate was spun for 20 min, 18,000×g at 4° C. Supernatant was transferred to glass LC-MS vials (Waters).

### FACS Lipid Extraction

Dissociated cells were stained according to above FACS protocols, except all staining buffers contained PBS+1% fatty acid-free BSA (Sigma). 400 μl LC-MS grade methanol containing internal standards was added to 2 mL lo-bind tubes (Eppendorf). After sorting, total volume was adjusted to 800 μl with deionized water (Milli-Q). Samples were vortexed 5 min, 2500 rpm at room temperature. 800 μl methyl tertiary-butyl ether (MTBE) was added and samples were vortexed 5 min, 2500 rpm at room temperature, then spun at 21000×g, 10 min, 4° C. 600 μl MTBE supernatant was transferred to glass LC-MS vial and dried under nitrogen gas. Samples were resuspended in 100 μl LC-MS grade methanol.

### Mass Spectrometry Analysis of Lipids

Lipid analyses were performed by liquid chromatography (Shimadzu Nexera X2 system, Shimadzu Scientific Instrument, Columbia, Md., USA) coupled to electrospray mass spectrometry (QTRAP 6500+, Sciex, Framingham, Mass., USA). For each analysis, 5 μL of sample was injected on a BEH C18 1.7 μm, 2.1×100 mm column (Waters Corporation, Milford, Mass., USA) using a flow rate of 0.25 mL/min at 55° C. For positive ionization mode, mobile phase A consisted of 60:40 acetonitrile/water (v/v) with 10 mM ammonium formate+0.1% formic acid; mobile phase B consisted of 90:10 isopropyl alcohol/acetonitrile (v/v) with 10 mM ammonium formate+0.1% formic acid. For negative ionization mode, mobile phase A consisted of 60:40 acetonitrile/water (v/v) with 10 mM ammonium acetate; mobile phase B consisted of 90:10 isopropyl alcohol/acetonitrile (v/v) with 10 mM ammonium acetate. The gradient was programmed as follows: 0.0-8.0 min from 45% B to 99% B, 8.0-9.0 min at 99% B, 9.0-9.1 min to 45% B, and 9.1-10.0 min at 45% B. Electrospray ionization was performed in either positive or negative ion mode applying the following settings: curtain gas at 30; collision gas was set at medium; ion spray voltage at 5500 (positive mode) or 4500 (negative mode); temperature at 250° C. (positive mode) or 600° C. (negative mode); ion source Gas 1 at 50; ion source Gas 2 at 60. Data acquisition was performed using Analyst 1.6.3 (Sciex) in multiple reaction monitoring mode (MRM), with the following parameters: dwell time (msec) and collision energy (CE) for each species reported in Table 2 (positive mode) or Table 3 (negative mode); declustering potential (DP) at 80; entrance potential (EP) at 10 (positive mode) or −10 (negative mode); and collision cell exit potential (CXP) at 12.5 (positive mode) or −12.5 (negative mode). Lipids were quantified using a mixture of non-endogenous internal standards as reported in Tables 2 and 3. Lipids were identified based on their retention times and MRM properties of commercially available reference standards (Avanti Polar Lipids, Birmingham, Ala., USA). Quantification was performed using MultiQuant 3.02 (Sciex). Metabolites were normalized to either total protein amount or cell number.

TABLE 2
LC-MS acquisition parameters for lipidomics assay in positive mode

| | | | Q1 | Q2 | Time |
|---|---|---|---|---|---|
| Lipid | Internal Std | RT | mass | mass | (msec) |
| Sphingosine d17:1 | N/A | 1.38 | 286.2 | 268.3 | 10 |
| Sphingosine | Sphingosine d17:1 | 1.56 | 300.2 | 282.2 | 10 |
| Sphinganine | Sphingosine d17:1 | 1.69 | 302.2 | 284.2 | 10 |
| Hexosyl sphingosine | Sphingosine d17:1 | 1.23 | 462.3 | 282.2 | 10 |
| Cer d18:1/17:0 | N/A | 5.83 | 552.4 | 264.3 | 5 |
| Cer d18:1/16:0 | Cer (d18:1/17:0) | 5.58 | 538.5 | 264.6 | 5 |
| Cer d18:1/18:0 | Cer (d18:1/17:0) | 6.05 | 566.6 | 264.4 | 5 |
| Cer d18:1/20:0 | Cer (d18:1/17:0) | 6.44 | 594.6 | 264.4 | 5 |
| Cer d18:1/22:0 | Cer (d18:1/17:0) | 6.78 | 622.6 | 264.4 | 5 |
| Cer d18:1/24:0 | Cer (d18:1/17:0) | 7.08 | 650.6 | 264.4 | 5 |
| Cer d18:1/24:1 | Cer (d18:1/17:0) | 6.74 | 648.6 | 264.4 | 5 |
| SM(d18:1(d9)/18:1) | N/A | 5.04 | 738.7 | 184.1 | 5 |
| SM d18:1/16:0 | SM(d18:1(d9)/18:1) | 5.02 | 703.6 | 184.1 | 5 |
| SM d18:1/18:0 | SM(d18:1(d9)/18:1) | 5.56 | 731.6 | 184.1 | 5 |
| SM d18:1/20:0 | SM(d18:1(d9)/18:1) | 6.01 | 759.6 | 184.1 | 5 |
| SM d18:1/22:0 | SM(d18:1(d9)/18:1) | 6.39 | 787.7 | 184.1 | 5 |
| SM d18:1/24:0 | SM(d18:1(d9)/18:1) | 6.73 | 815.7 | 184.1 | 5 |
| SM d18:1/24:1 | SM(d18:1(d9)/18:1) | 6.35 | 813.7 | 184.1 | 5 |
| GlcCer (d18:1/12:0) | N/A | 3.99 | 644.5 | 264.3 | 10 |
| HexCer d18:1/16:0 | GlcCer (d18:1/12:0) | 5.18 | 700.6 | 264.6 | 10 |
| HexCer d18:1/18:0 | GlcCer (d18:1/12:0) | 5.69 | 728.6 | 264.4 | 10 |
| HexCer d18:1/20:0 | GlcCer (d18:1/12:0) | 6.11 | 756.6 | 264.4 | 10 |
| HexCer d18:1/22:0 | GlcCer (d18:1/12:0) | 6.48 | 784.7 | 264.4 | 10 |
| HexCer d18:1/24:0 | GlcCer (d18:1/12:0) | 6.8 | 812.7 | 264.4 | 10 |
| HexCer d18:1/24:1 | GlcCer (d18:1/12:0) | 6.44 | 810.7 | 264.4 | 10 |
| LacCer d18:1/16:0 | GlcCer (d18:1/12:0) | 4.99 | 862.6 | 264.6 | 10 |
| LacCer d18:1/18:0 | GlcCer (d18:1/12:0) | 5.5 | 890.7 | 264.4 | 10 |
| LacCer d18:1/20:0 | GlcCer (d18:1/12:0) | 5.43 | 918.7 | 264.4 | 10 |
| LacCer d18:1/22:0 | GlcCer (d18:1/12:0) | 6.34 | 946.7 | 264.4 | 10 |
| LacCer d18:1/24:0 | GlcCer (d18:1/12:0) | 6.27 | 974.8 | 264.4 | 10 |
| LacCer d18:1/24:1 | GlcCer (d18:1/12:0) | 6.29 | 972.7 | 264.4 | 10 |
| LPC(18:1(d7)) | N/A | 1.85 | 529.3 | 184.1 | 5 |
| lysoPC 16:0 | LPC(18:1(d7)) | 1.81 | 496.3 | 184.1 | 5 |
| lysoPC 18:0 | LPC(18:1(d7)) | 2.34 | 524.3 | 184.1 | 5 |
| lysoPC 18:1 | LPC(18:1(d7)) | 1.86 | 522.3 | 184.1 | 5 |
| lysoPC 20:4 | LPC(18:1(d7)) | 1.48 | 544.3 | 184.1 | 5 |
| lysoPC 22:6 | LPC(18:1(d7)) | 1.41 | 568.3 | 184.1 | 5 |
| Lyso SM d18:1 | 18:1(d7)LPC | 1.41 | 465.5 | 184.1 | 10 |
| 15:0-18:1(d7)PC | N/A | 5.23 | 754.6 | 184.1 | 5 |
| PC 34:1 | 15:0-18:1(d7) PC | 5.5 | 760.6 | 184.1 | 5 |
| PC 34:2 | 15:0-18:1(d7) PC | 5.09 | 758.6 | 184.1 | 5 |
| PC 36:1 | 15:0-18:1(d7) PC | 5.95 | 788.6 | 184.1 | 5 |
| PC 36:2 | 15:0-18:1(d7) PC | 5.59 | 786.6 | 184.1 | 5 |
| PC 36:4 | 15:0-18:1(d7) PC | 4.67 | 782.6 | 184.1 | 5 |
| PC 38:1 | 15:0-18:1(d7) PC | 6.72 | 816.6 | 184.1 | 5 |
| PC 38:2 | 15:0-18:1(d7) PC | 6.35 | 814.6 | 184.1 | 5 |
| PC 38:4 | 15:0-18:1(d7) PC | 5.48 | 810.6 | 184.1 | 5 |
| PC 38:5 | 15:0-18:1(d7) PC | 4.98 | 808.6 | 184.1 | 5 |
| PC 38:6 | 15:0-18:1(d7) PC | 4.8 | 806.6 | 184.1 | 5 |
| PC 40:4 | 15:0-18:1(d7) PC | 5.92 | 838.6 | 184.1 | 5 |
| PC 40:5 | 15:0-18:1(d7) PC | 5.66 | 836.6 | 184.1 | 5 |
| PC 40:6 | 15:0-18:1(d7) PC | 5.31 | 834.6 | 184.1 | 5 |
| PC 40:7 | 15:0-18:1(d7) PC | 4.82 | 832.6 | 184.1 | 5 |
| PC 42:5 | 15:0-18:1(d7) PC | 5.82 | 864.6 | 184.1 | 5 |
| PC 42:6 | 15:0-18:1(d7) PC | 5.37 | 862.6 | 184.1 | 5 |
| PC 42:7 | 15:0-18:1(d7) PC | 5.3 | 860.6 | 184.1 | 5 |
| POVPC | 15:0-18:1(d7) PC | 1.79 | 594.5 | 184.1 | 10 |
| PGPC | LPC(18:1(d7)) | 1.85 | 610.2 | 184.1 | 10 |
| PC(16:0/9:0(CHO)) | LPC(18:1(d7)) | 2.05 | 650.3 | 184.1 | 10 |
| ALDO (PONPC) | | | | | |
| PC(16:0/9:0(COOH)) | LPC(18:1(d7)) | 1.98 | 666.4 | 184.1 | 10 |
| PAZPC | | | | | |
| KOOA-PC | LPC(18:1(d7)) | 2.36 | 648.3 | 184.1 | 10 |
| KOdiA-PC | LPC(18:1(d7)) | 1.54 | 664.4 | 184.1 | 10 |
| PAF 16:0 C2 | LPC(18:1(d7)) | 1.86 | 524.3 | 184.1 | 10 |
| 15:0-18:1(d7) PE | N/A | 5.38 | 711.6 | 570.5 | 5 |
| PE 34:1 | 15:0-18:1(d7) PE | 5.63 | 718.6 | 577.5 | 5 |
| PE 34:2 | 15:0-18:1(d7) PE | 5.22 | 716.6 | 575.5 | 5 |
| PE 36:1 | 15:0-18:1(d7) PE | 6.06 | 746.6 | 605.5 | 5 |
| PE 36:2 | 15:0-18:1(d7) PE | 5.72 | 744.6 | 603.5 | 5 |
| PE 36:4 | 15:0-18:1(d7) PE | 5.11 | 740.6 | 599.5 | 5 |
| PE 38:1 | 15:0-18:1(d7) PE | 6.39 | 774.6 | 633.5 | 5 |
| PE 38:2 | 15:0-18:1(d7) PE | 6.09 | 772.6 | 631.5 | 5 |
| PE 38:4 | 15:0-18:1(d7) PE | 5.61 | 768.6 | 627.5 | 5 |
| PE 38:5 | 15:0-18:1(d7) PE | 5.27 | 766.6 | 625.5 | 5 |
| PE 38:6 | 15:0-18:1(d7) PE | 4.93 | 764.6 | 623.5 | 5 |
| PE 38:7 | 15:0-18:1(d7) PE | 4.43 | 762.6 | 621.5 | 5 |
| PE 40:4 | 15:0-18:1(d7) PE | 5.91 | 796.6 | 655.5 | 5 |
| PE 40:5 | 15:0-18:1(d7) PE | 5.78 | 794.6 | 653.5 | 5 |
| PE 40:6 | 15:0-18:1(d7) PE | 5.45 | 792.6 | 651.5 | 5 |
| PE 40:7 | 15:0-18:1(d7) PE | 4.95 | 790.6 | 649.5 | 5 |
| PE 42:5 | 15:0-18:1(d7) PE | 6.38 | 822.6 | 681.5 | 5 |
| PE 42:6 | 15:0-18:1(d7) PE | 5.51 | 820.6 | 679.5 | 5 |
| d7-Cholesterol | N/A | 4.92 | 376.2 | 376.2 | 10 |
| Cholesterol | d7-Cholesterol | 4.95 | 369.3 | 369.3 | 10 |
| 18:1(d7) CE | N/A | 8.28 | 675.2 | 369.4 | 10 |
| CE 16:1 | 18:1(d7) CE | 8.05 | 640.6 | 369.3 | 10 |
| CE 18:1 | 18:1(d7) CE | 8.29 | 668.6 | 369.3 | 10 |
| CE 18:2 | 18:1(d7) CE | 8.07 | 666.6 | 369.3 | 10 |
| CE 20:1 | 18:1(d7) CE | 8.49 | 696.6 | 369.3 | 10 |
| CE 20:4 | 18:1(d7) CE | 7.94 | 690.6 | 369.3 | 10 |
| CE 20:5 | 18:1(d7) CE | 7.74 | 688.6 | 369.3 | 10 |
| CE 22:5 | 18:1(d7) CE | 7.93 | 716.6 | 369.3 | 10 |
| CE 22:6 | 18:1(d7) CE | 7.81 | 714.6 | 369.3 | 10 |
| d7-24 OH Cholesterol | N/A | 2.0 | 392.4 | 374.3 | 10 |
| 7 keto-cholesterol | 18:1(d7) CE | 3.5 | 401.3 | 383.3 | 10 |
| OH Cholesterol | d7-24 OH Cholesterol | 2.1 | 385.4 | 367.5 | 10 |
| 4-beta hydroxycholesterol | 18:1(d7) CE | 4.27 | 420.3 | 385.3 | 10 |
| 7 dehydrocholesterol | 18:1(d7) CE | 4.32 | 366.3 | 366.3 | 10 |
| CE oxoODE | 18:1(d7) CE | 7.09 | 680.6 | 369.2 | 10 |
| CE HODE | 18:1(d7) CE | 7.3 | 682.6 | 369.2 | 10 |
| CE HpODE | 18:1(d7) CE | 6.3 | 698.6 | 369.2 | 10 |
| CE oxoHETE | 18:1(d7) CE | 7.31 | 704.6 | 369.2 | 10 |
| CE HETE | 18:1(d7) CE | 7.27 | 706.6 | 369.2 | 10 |
| 15:0-18:1(d7)-15:0 TG | N/A | 7.93 | 829.4 | 523.5 | 8 |
| TG 50:2/16:1 | 15:0-18:1(d7)-15:0 TG | 7.93 | 848.7 | 577.4 | 8 |
| TG 52:4/18:1 | 15:0-18:1(d7)-15:0 TG | 7.73 | 872.7 | 573.4 | 8 |
| TG 52:5/18:1 | 15:0-18:1(d7)-15:0 TG | 7.55 | 870.6 | 571.3 | 8 |
| TG 52:3/18:1 | 15:0-18:1(d7)-15:0 TG | 7.94 | 874.7 | 575.4 | 8 |
| TG 54:1/18:0 | 15:0-18:1(d7)-15:0 TG | 8.47 | 906.8 | 605.5 | 8 |
| TG 54:2/18:0 | 15:0-18:1(d7)-15:0 TG | 8.3 | 904.7 | 603.4 | 8 |
| TG 54:3/18:0 | 15:0-18:1(d7)-15:0 TG | 8.13 | 902.7 | 601.4 | 8 |
| TG 54:4/18:1 | 15:0-18:1(d7)-15:0 TG | 7.93 | 900.7 | 601.4 | 8 |
| TG 52:5/20:4 | 15:0-18:1(d7)-15:0 TG | 7.66 | 870.6 | 549.3 | 8 |
| TG 54:4/20:4 | 15:0-18:1(d7)-15:0 TG | 8.08 | 900.6 | 579.3 | 8 |
| TG 54:5/20:4 | 15:0-18:1(d7)-15:0 TG | 7.87 | 898.6 | 577.3 | 8 |
| TG 54:6/20:4 | 15:0-18:1(d7)-15:0 TG | 7.68 | 896.6 | 575.3 | 8 |
| TG 54:7/20:4 | 15:0-18:1(d7)-15:0 TG | 7.46 | 894.6 | 573.3 | 8 |
| TG 56:3/18:1 | 15:0-18:1(d7)-15:0 TG | 8.27 | 930.8 | 631.5 | 8 |
| TG 56:4/20:4 | 15:0-18:1(d7)-15:0 TG | 8.26 | 928.8 | 607.5 | 8 |
| TG 56:5/20:4 | 15:0-18:1(d7)-15:0 TG | 8.06 | 926.7 | 605.4 | 8 |
| TG 56:6/20:4 | 15:0-18:1(d7)-15:0 TG | 7.87 | 924.7 | 603.4 | 8 |
| TG 56:7/20:4 | 15:0-18:1(d7)-15:0 TG | 7.67 | 922.7 | 601.4 | 8 |
| TG 56:8/20:4 | 15:0-18:1(d7)-15:0 TG | 7.47 | 920.7 | 599.4 | 8 |
| TG 56:9/20:4 | 15:0-18:1(d7)-15:0 TG | 7.27 | 918.6 | 597.3 | 8 |
| TG 58:5/20:4 | 15:0-18:1(d7)-15:0 TG | 8.24 | 954.7 | 633.4 | 8 |
| TG 58:6/20:4 | 15:0-18:1(d7)-15:0 TG | 8.04 | 952.7 | 631.4 | 8 |
| TG 58:7/20:4 | 15:0-18:1(d7)-15:0 TG | 7.86 | 950.7 | 629.4 | 8 |
| TG 58:8/22:6 | 15:0-18:1(d7)-15:0 TG | 7.76 | 948.7 | 603.4 | 8 |
| TG 58:9/22:6 | 15:0-18:1(d7)-15:0 TG | 7.57 | 946.7 | 601.4 | 8 |
| TG 60:7/22:6 | 15:0-18:1(d7)-15:0 TG | 8.15 | 978.7 | 633.4 | 8 |
| TG 60:8/22:6 | 15:0-18:1(d7)-15:0 TG | 7.95 | 976.7 | 631.4 | 8 |
| Sphingosine-1-phosphate | N/A | 1.41 | 366.3 | 250.3 | 10 |
| d17:1 | | | | | |
| Sphingosine-1-phosphate | Sphingosine-1-phosphate | 1.58 | 380.3 | 264.3 | 10 |
| | d17:1 | | | | |
| Sphinganine-1-phosphate | Sphingosine-1-phosphate | 1.68 | 382.3 | 266.3 | 10 |
| | d17:1 | | | | |
| 15:0-18:1(d7) DAG | N/A | 6.08 | 605.6 | 346.5 | 10 |
| DAG(16:0/18:0) | 15:0-18:1(d7) DAG | 6.64 | 614.4 | 313.2 | 10 |
| DAG(16:0/18:1) | 15:0-18:1(d7) DAG | 6.29 | 612.6 | 313.2 | 10 |
| DAG(18:0/18:1) | 15:0-18:1(d7) DAG | 6.65 | 640.4 | 341.3 | 10 |
| DAG(18:1/18:1) | 15:0-18:1(d7) DAG | 6.29 | 638.4 | 339.3 | 10 |
| DAG(16:0/20:4) | 15:0-18:1(d7) DAG | 5.8 | 634.5 | 313.3 | 10 |
| DAG(18:1/20:4) | 15:0-18:1(d7) DAG | 5.81 | 660.5 | 339.3 | 10 |
| DAG(18:0/20:4) | 15:0-18:1(d7) DAG | 6.21 | 662.5 | 341.3 | 10 |
| DAG(18:0/22:6) | 15:0-18:1(d7) DAG | 6.06 | 686.6 | 341.3 | 10 |
| DAG(18:1/22:6) | 15:0-18:1(d7) DAG | 5.65 | 684.6 | 339.3 | 10 |
| 18:1(d7) MAG | N/A | 2.5 | 381.3 | 272.5 | 10 |
| 20:4 MAG | 18:1(d7) MAG | 1.96 | 396.3 | 287.3 | 10 |
| 18:1 MAG | 18:1(d7) MAG | 2.56 | 374.3 | 265.3 | 10 |
| AEA | 18:1(d7) MAG | 2.1 | 348.3 | 62.1 | 10 |
| OEA | 18:1(d7) MAG | 2.57 | 326.3 | 62.1 | 10 |
| PEA | 18:1(d7) MAG | 2.22 | 300.3 | 62.1 | 10 |
| SM (d18:0/16:0) | SM(d18:1(d9)/18:1) | 5.1 | 710.6 | 184.2 | 10 |
| SM (d18:0/18:1) | SM(d18:1(d9)/18:1) | 5.6 | 736.6 | 184.2 | 10 |
| SM (d18:0/24:0) | SM(d18:1(d9)/18:1) | 6.75 | 822.7 | 184.2 | 10 |
| SM (d18:0/24:1) | SM(d18:1(d9)/18:1) | 6.4 | 820.7 | 184.2 | 10 |
| Cer (d18:0/16:0) | Cer (d18:1/17:0) | 5.72 | 540.6 | 522.3 | 10 |
| Cer (d18:0/18:0) | Cer (d18:1/17:0) | 6.03 | 568.7 | 550.4 | 10 |
| Cer (d18:0/24:0) | Cer (d18:1/17:0) | 7.16 | 652.9 | 634.4 | 10 |
| Cer (d18:0/24:1) | Cer (d18:1/17:0) | 6.85 | 650.9 | 632.4 | 10 |
| GB3 (d18:1/16:0) | GlcCer (d18:1/12:0) | 4.93 | 1024.6 | 520.5 | 10 |
| GB3 (d18:1/18:0) | GlcCer (d18:1/12:0) | 5.45 | 1052.6 | 548.6 | 10 |
| GB3 (d18:1/24:0) | GlcCer (d18:1/12:0) | 6.65 | 1136.8 | 632.6 | 10 |
| GB3 (d18:1/24:1) | GlcCer (d18:1/12:0) | 6.28 | 1134.8 | 630.6 | 10 |
| lysoPC 26:0 | LPC(18:1(d7)) | 4.93 | 636.5 | 104.1 | 10 |
| lysoPC 24:0 | LPC(18:1(d7)) | 4.31 | 608.5 | 184.1 | 10 |
| lysoPC 26:1 | LPC(18:1(d7)) | 4.25 | 634.5 | 104.1 | 10 |
| lysoPC 24:1 | LPC(18:1(d7)) | 3.63 | 606.5 | 184.1 | 10 |
| lysoPC 16:1 | LPC(18:1(d7)) | 1.36 | 494.5 | 184.1 | 10 |

TABLE 3
LC-MS acquisition parameters for Lipidomics Assay in negative mode

| | | | Q1 | Q2 | Time |
|---|---|---|---|---|---|
| Lipid | Internal Std | RT | mass | mass | (msec) |
| 15:0-18:1(d7) PA | | 5.37 | 666.5 | 241.3 | 10 |
| PA 34:1 | 15:0-18:1(d7) PA | 5.65 | 673.5 | 255.3 | 10 |
| PA 36:1 | 15:0-18:1(d7) PA | 6.12 | 701.5 | 283.3 | 10 |
| PA 36:2 | 15:0-18:1(d7) PA | 5.7 | 699.5 | 281.3 | 10 |
| PA 38:5 | 15:0-18:1(d7) PA | 5.1 | 721.5 | 281.3 | 10 |
| PA 38:4 | 15:0-18:1(d7) PA | 5.39 | 723.5 | 283.3 | 10 |
| PA 40:7 | 15:0-18:1(d7) PA | 5.71 | 745.5 | 281.3 | 10 |
| PA 40:6 | 15:0-18:1(d7) PA | 5.4 | 747.5 | 283.3 | 10 |
| 15:0-18:1(d7) PE | | 5.57 | 709.5 | 241.3 | 10 |
| PE 36:1p | 15:0-18:1(d7) PE | 6.08 | 728.6 | 283.3 | 10 |
| PE 36:2p | 15:0-18:1(d7) PE | 6.08 | 726.6 | 281.3 | 10 |
| PE 36:4p | 15:0-18:1(d7) PE | 5.56 | 722.6 | 303.3 | 10 |
| PE 38:4p | 15:0-18:1(d7) PE | 6.04 | 750.6 | 303.3 | 10 |
| PE 38:5p | 15:0-18:1(d7) PE | 5.55 | 748.6 | 303.3 | 10 |
| PE 38:6p | 15:0-18:1(d7) PE | 5.38 | 746.6 | 327.3 | 10 |
| PE 40:4p | 15:0-18:1(d7) PE | 5.49 | 778.6 | 303.3 | 10 |
| PE 40:5p | 15:0-18:1(d7) PE | 5.4 | 776.6 | 303.3 | 10 |
| PE 40:6p | 15:0-18:1(d7) PE | 5.89 | 774.6 | 327.3 | 10 |
| Sulfatide (d18:1/12:0) | | 3.41 | 722.5 | 96.7 | 10 |
| Sulfatide (d18:1/16:0) | Sulfatide (d18:1/12:0) | 4.66 | 778.5 | 96.7 | 10 |
| Sulfatide (d18:1/18:0) | Sulfatide (d18:1/12:0) | 5.21 | 806.6 | 96.7 | 10 |
| Sulfatide (d18:1/18:0h) | Sulfatide (d18:1/12:0) | 5.13 | 822.6 | 96.7 | 10 |
| Sulfatide (d18:1/24:0) | Sulfatide (d18:1/12:0) | 6.45 | 890.7 | 96.7 | 10 |
| Sulfatide (d18:1/24:0h) | Sulfatide (d18:1/12:0) | 6.39 | 906.7 | 96.7 | 10 |
| Sulfatide (d18:1/24:1) | Sulfatide (d18:1/12:0) | 6.08 | 888.7 | 96.7 | 10 |
| Sulfatide (d18:1/24:1h) | Sulfatide (d18:1/12:0) | 6.06 | 904.7 | 96.7 | 10 |
| GM3 d34:1 | 15:0-18:1(d7) PI | 4.63 | 1151.7 | 290.1 | 10 |
| GM3 d36:1 | 15:0-18:1(d7) PI | 5.18 | 1179.8 | 290.1 | 10 |
| GM3 d38:1 | 15:0-18:1(d7) PI | 5.66 | 1207.8 | 290.1 | 10 |
| GM3 d40:1 | 15:0-18:1(d7) PI | 6.09 | 1235.8 | 290.1 | 10 |
| GD3 d34:1 | 15:0-18:1(d7) PI | 4.35 | 720.9 | 290.1 | 10 |
| GD3 d36:1 | 15:0-18:1(d7) PI | 4.87 | 734.9 | 290.1 | 10 |
| GD3 d38:1 | 15:0-18:1(d7) PI | 5.42 | 748.9 | 290.1 | 10 |
| GD3 d40:1 | 15:0-18:1(d7) PI | 5.8 | 762.9 | 290.1 | 10 |
| GD3 d42:2 | 15:0-18:1(d7) PI | 5.7 | 775.9 | 290.1 | 10 |
| GD3 d42:1 | 15:0-18:1(d7) PI | 6.2 | 776 | 290.1 | 10 |
| GD1a/b d36:1 | 15:0-18:1(d7) PI | 4.6 | 917.5 | 290.1 | 10 |
| GD1a/b d38:1 | 15:0-18:1(d7) PI | 5.2 | 931.5 | 290.1 | 10 |
| GT1b d36:1 | 15:0-18:1(d7) PI | 4.4 | 1063 | 290.1 | 10 |
| GT1b d38:1 | 15:0-18:1(d7) PI | 5 | 1077 | 290.1 | 10 |
| GQ1b d36:1 | 15:0-18:1(d7) PI | 5.67 | 1208.6 | 290.1 | 10 |
| GQ1b d38:1 | 15:0-18:1(d7) PI | 5.89 | 1222.6 | 290.1 | 10 |
| 9-PAHSA | 15:0-18:1(d7) PI | 5.12 | 537.6 | 255 | 10 |
| 9-OAHSA | 15:0-18:1(d7) PI | 5.12 | 563.6 | 281 | 10 |
| 9-PAHPA | 15:0-18:1(d7) PI | 5.12 | 509.6 | 255 | 10 |
| 9-OAHOA | 15:0-18:1(d7) PI | 5.12 | 561.6 | 281 | 10 |
| 9-POAHSA | 15:0-18:1(d7) PI | 5.12 | 537.6 | 253 | 10 |
| 9-POAHPA | 15:0-18:1(d7) PI | 5.12 | 509.6 | 253 | 10 |
| BMP 28:0 | | 3.55 | 665.3 | 227.2 | 10 |
| BMP 40:8 | BMP 28:0 | 3.62 | 817.5 | 303.3 | 10 |
| BMP 44:12 | BMP 28:0 | 3.35 | 865.5 | 327.3 | 10 |
| BMP 36:2 | BMP 28:0 | 4.65 | 773.5 | 281.3 | 10 |
| AA d8 | | 2.39 | 311.3 | 311.3 | 10 |
| FFA(16:0) | AA d8 | 3.01 | 255.1 | 255.1 | 10 |
| FFA(16:1) | AA d8 | 2.47 | 253.1 | 253.1 | 10 |
| FFA(18:0) | AA d8 | 3.72 | 283.2 | 283.2 | 10 |
| FFA(18:1) | AA d8 | 3.05 | 281.2 | 281.2 | 10 |
| FFA(18:2) | AA d8 | 2.53 | 279.2 | 279.2 | 10 |
| FFA(18:3) | AA d8 | 2.22 | 277.2 | 277.2 | 10 |
| FFA(20:4) | AA d8 | 2.68 | 303.2 | 303.2 | 10 |
| FFA(20:5) | AA d8 | 2.4 | 301.2 | 301.2 | 10 |
| FFA(22:6) | AA d8 | 2.37 | 327.2 | 327.2 | 10 |
| 18:1(d7) pLPE | | 2.04 | 485.3 | 196.1 | 10 |
| lysoPEp C16:0 | 18:1(d7) pLPE | 2.21 | 436.3 | 196.1 | 10 |
| lysoPEp C18:0 | 18:1(d7) pLPE | 2.81 | 464.3 | 196.1 | 10 |
| lysoPEp C18:1 | 18:1(d7) pLPE | 2.25 | 462.3 | 196.1 | 10 |
| 18:1(d7) LPE | | 2.04 | 485.3 | 288.3 | 10 |
| LPE(16:0) | 18:1(d7) LPE | 2 | 452.3 | 255.3 | 10 |
| LPE(18:0) | 18:1(d7) LPE | 2.57 | 480.3 | 283.3 | 10 |
| LPE(18:1) | 18:1(d7) LPE | 2.06 | 478.3 | 281.3 | 10 |
| lysoPI 16:0 | 18:1(d7) LPE | 1.66 | 571.3 | 241.1 | 10 |
| lysoPI 18:0 | 18:1(d7) LPE | 2.13 | 599.3 | 241.1 | 10 |
| lysoPI 20:4 | 18:1(d7) LPE | 1.4 | 619.3 | 241.1 | 10 |
| LPS(17:1) | | 1.53 | 508.3 | 267.3 | 10 |
| LPS(16:0) | LPS(17:1) | 1.68 | 496.3 | 255.3 | 10 |
| LPS(18:0) | LPS(17:1) | 2.16 | 524.3 | 283.3 | 10 |
| LPS(18:1) | LPS(17:1) | 1.73 | 522.3 | 281.3 | 10 |
| LPS(20:4) | LPS(17:1) | 1.3 | 544.3 | 303.3 | 10 |
| LPS(22:6) | LPS(17:1) | 1.2 | 568.3 | 327.3 | 10 |
| LPG(16:0) | 18:1(d7) LPE | 1.6 | 483.3 | 255.3 | 10 |
| LPG(18:0) | 18:1(d7) LPE | 2.23 | 511.3 | 283.3 | 10 |
| LPG(18:1) | 18:1(d7) LPE | 1.78 | 509.3 | 281.3 | 10 |
| LPG(20:4) | 18:1(d7) LPE | 1.3 | 531.3 | 303.3 | 10 |
| LPA(16:0) | 18:1(d7) LPE | 1.68 | 409.3 | 255.3 | 10 |
| LPA(18:0) | 18:1(d7) LPE | 2.16 | 423.3 | 283.3 | 10 |
| LPA(18:1) | 18:1(d7) LPE | 1.73 | 421.3 | 281.3 | 10 |
| CL 58:0/14:0 | | 7.22 | 619.5 | 227.2 | 10 |
| CL 72:8 | CL 58:0/14:0 | 7.37 | 723.7 | 279.2 | 10 |
| Cholesterol Sulfate d7 | | 3.03 | 472.3 | 96.7 | 10 |
| Cholesterol Sulfate | Cholesterol Sulfate d7 | 3.06 | 465.3 | 96.7 | 10 |
| 15:0-18:1(d7) PG | | 5.01 | 740.5 | 241.3 | 10 |
| PG 32:0 | 15:0-18:1(d7) PG | 5.25 | 721.5 | 255.3 | 10 |
| PG 32:1 | 15:0-18:1(d7) PG | 5.58 | 719.5 | 255.3 | 10 |
| PG 34:0 | 15:0-18:1(d7) PG | 5.73 | 749.5 | 283.3 | 10 |
| PG 34:1 | 15:0-18:1(d7) PG | 5.24 | 747.5 | 255.3 | 10 |
| PG 34:2 | 15:0-18:1(d7) PG | 5.58 | 745.5 | 255.3 | 10 |
| PG 36:0 | 15:0-18:1(d7) PG | 6.15 | 777.5 | 283.3 | 10 |
| PG 36:1 | 15:0-18:1(d7) PG | 5.76 | 775.5 | 283.3 | 10 |
| PG 36:2 | 15:0-18:1(d7) PG | 5.25 | 773.5 | 281.3 | 10 |
| PG 38:4 | 15:0-18:1(d7) PG | 5.2 | 797.6 | 283.3 | 10 |
| 15:0-18:1(d7) PI | | 4.88 | 828.6 | 241.3 | 10 |
| PI 36:1 | 15:0-18:1(d7) PI | 5.64 | 863.6 | 283.3 | 10 |
| PI 36:2 | 15:0-18:1(d7) PI | 5.15 | 861.6 | 281.3 | 10 |
| PI 36:4 | 15:0-18:1(d7) PG | 4.63 | 857.6 | 255.3 | 10 |
| PI 38:4 | 15:0-18:1(d7) PG | 5.15 | 885.6 | 283.3 | 10 |
| PI 38:5 | 15:0-18:1(d7) PG | 4.65 | 883.6 | 281.3 | 10 |
| PI 38:6 | 15:0-18:1(d7) PG | 4.46 | 881.6 | 255.3 | 10 |
| PI 40:5 | 15:0-18:1(d7) PG | 5.01 | 911.6 | 283.3 | 10 |
| PI 40:6 | 15:0-18:1(d7) PG | 5.01 | 909.6 | 283.3 | 10 |
| PI 40:8 | 15:0-18:1(d7) PG | 4.06 | 905.6 | 303.3 | 10 |
| 15:0-18:1(d7)PS | | 5.03 | 753.5 | 241.3 | 10 |
| PS 36:0 | 15:0-18:1(d7) PS | 5.51 | 790.6 | 283.3 | 10 |
| PS 36:1 | 15:0-18:1(d7) PS | 5.75 | 788.6 | 283.3 | 10 |
| PS 38:5 | 15:0-18:1(d7) PS | 5.02 | 808.6 | 283.3 | 10 |
| PS 38:4 | 15:0-18:1(d7) PS | 5.25 | 810.6 | 283.3 | 10 |
| PS 38:6 | 15:0-18:1(d7) PS | 4.55 | 806.6 | 255.3 | 10 |
| PS 40:7 | 15:0-18:1(d7) PS | 4.56 | 832.6 | 281.3 | 10 |
| PS 40:6 | 15:0-18:1(d7) PS | 5.05 | 834.6 | 283.3 | 10 |
| PS 40:4 | 15:0-18:1(d7) PS | 5.77 | 838.6 | 303.3 | 10 |

Mass spectrometry analysis of GlcCer and GalCer

Glucosylceramiide (GlcCer), galatosylceramide (GalCer), glucosylsphigosine and galatosylsphigosine analyses were performed by liquid chromatography (Shimadzu Nexera X2 system, Shimadzu Scientific Instrument, Columbia, Md., USA) coupled to electrospray mass spectrometry (QTRAP 6500+, Sciex, Framingham, Mass., USA). For each analysis, 10 μL of sample was injected on a HALO HELIC 2.0 μm, 3.0×150 mm column (Advanced Materials Technology) using a flow rate of 0.45 ml/min at 45° C. Mobile phase A consisted of 92.5/5/2.5 ACN/IPA/H2O with 5 mM ammonium formate and 0.5% formic Acid. Mobile phase B consisted of 92.5/5/2.5 H2O/IPA/ACN with 5 mM ammonium formate and 0.5% formic acid. The gradient was programmed as follows: 0.0-3.1 min at 100% B, 3.2 min at 95% B, 5.7 min at 85% B, hold to 7.1 min at 85% B, drop to 0% B at 7.25 min and hold to 8.75 min, ramp back to 100% at 10.65 min and hold to 11 min. Electrospray ionization was performed in the positive-ion mode applying the following settings: curtain gas at 25; collision gas was set at medium; ion spray voltage at 5500; temperature at 350° C.; ion source Gas 1 at 55; ion source Gas 2 at 60. Data acquisition was performed using Analyst 1.6 (Sciex) in multiple reaction monitoring mode (MRM) with the following parameters: dwell time (msec) and collision energy (CE) for each species reported in Table 4; declustering potential (DP) at 45; entrance potential (EP) at 10; and collision cell exit potential (CXP) at 12.5. Lipids were quantified using a mixture of internal standards as reported in Table 4. Glucosylceramide and galactosylceramide were identified based on their retention times and MRM properties of commercially available reference standards (Avanti Polar Lipids, Birmingham, Ala., USA). Quantification was performed using MultiQuant 3.02 (Sciex). Metabolites were normalized to cell number.

TABLE 4
LC-MS acquisition parameters for GlcCer and GalCer Assay

| | | | Q1 | Q2 | Time |
|---|---|---|---|---|---|
| Lipid | Internal Std | RT | mass | mass | (msec) |
| GlcCer(d18:1, 16:0) | GlcCer(d18:1/18:0)-d5 | 2.33 | 700.6 | 264.3 | 50 |
| GlcCer(d18:1, 18:0) | GlcCer(d18:1/18:0)-d5 | 2.28 | 728.6 | 264.3 | 50 |
| GlcCer(d18:2, 18:0) | GlcCer(d18:1/18:0)-d5 | 2.27 | 726.6 | 262.3 | 50 |
| GlcCer(d18:1, 20:0) | GlcCer(d18:1/18:0)-d5 | 2.23 | 756.6 | 264.3 | 50 |
| GlcCer (d18:2, 20:0) | GlcCer(d18:1/18:0)-d5 | 2.22 | 754.6 | 262.3 | 50 |
| GlcCer (d18:1/22:0) | GlcCer(d18:1/18:0)-d5 | 2.19 | 784.6 | 264.3 | 50 |
| GlcCer (d18:1/22:1) | GlcCer(d18:1/18:0)-d5 | 2.2 | 782.6 | 264.3 | 50 |
| GlcCer (d18:2/22:0) | GlcCer(d18:1/18:0)-d5 | 2.18 | 782.6 | 262.3 | 50 |
| GlcCer (d18:1/24:1) | GlcCer(d18:1/18:0)-d5 | 2.17 | 810.6 | 264.3 | 50 |
| GlcCer (d18:1/24:0) | GlcCer(d18:1/18:0)-d5 | 2.15 | 812.7 | 264.3 | 50 |
| Glu-Sph 18:1 | Glu-Sph_d5 | 7.77 | 462.2 | 264.3 | 200 |
| GlcCer (d18:1/18:0)-d5 | | 2.27 | 733.6 | 269.3 | 7 |
| Glu-Sph_d5 | | 7.77 | 467.2 | 269.3 | 15 |
| GalCer (d18:1/16:0) | GlcCer(d18:1/18:0)-d5 | 2.5 | 700.5 | 264.3 | 50 |
| GalCer (d18:1/18:0) | GlcCer(d18:1/18:0)-d5 | 2.45 | 728.6 | 264.3 | 50 |
| GalCer (d18:2/18:0) | GlcCer(d18:1/18:0)-d5 | 2.45 | 726.6 | 262.3 | 50 |
| GalCer (d18:1/20:0) | GlcCer(d18:1/18:0)-d5 | 2.4 | 756.6 | 264.3 | 50 |
| GalCer (d18:2/20:0) | GlcCer(d18:1/18:0)-d5 | 2.39 | 754.6 | 262.3 | 50 |
| GalCer (d18:1/22:0) | GlcCer(d18:1/18:0)-d5 | 2.35 | 784.6 | 264.3 | 50 |
| GalCer (d18:1/22:1) | GlcCer(d18:1/18:0)-d5 | 2.56 | 782.6 | 264.3 | 50 |
| GalCer (d18:2/22:0) | GlcCer(d18:1/18:0)-d5 | 2.34 | 782.6 | 262.3 | 50 |
| GalCer (d18:1/24:1) | GlcCer(d18:1/18:0)-d5 | 2.31 | 810.7 | 264.3 | 50 |
| GalCer (d18:1/24:0) | GlcCer(d18:1/18:0)-d5 | 2.3 | 812.7 | 264.3 | 50 |
| Gal-Sph 18:1 | Glu-Sph_d5 | 7.87 | 462.2 | 282.3 | 200 |

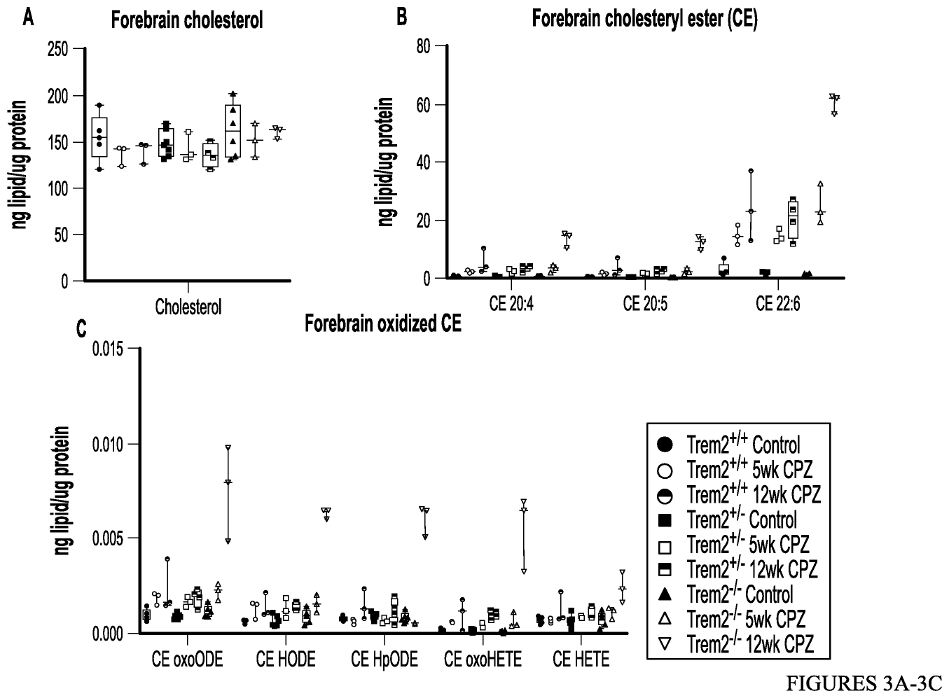

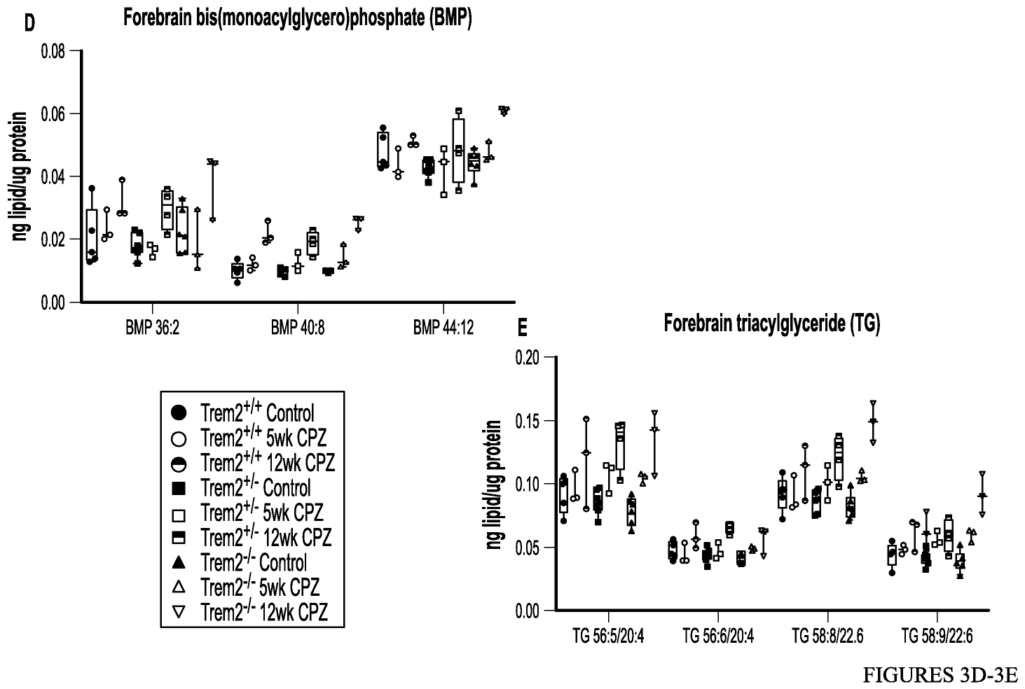

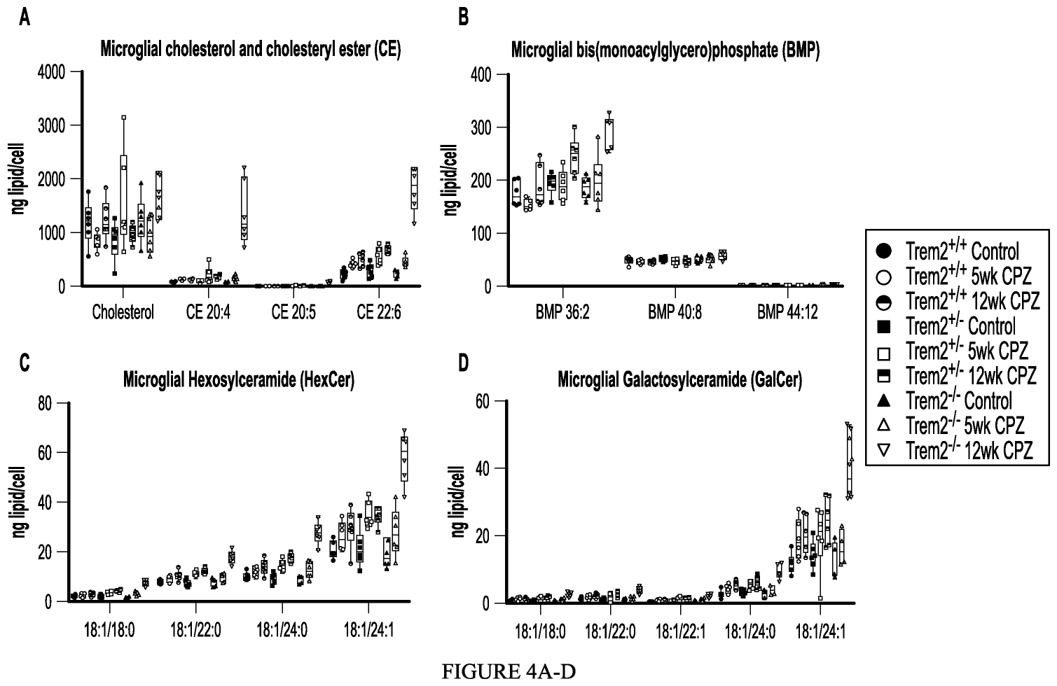

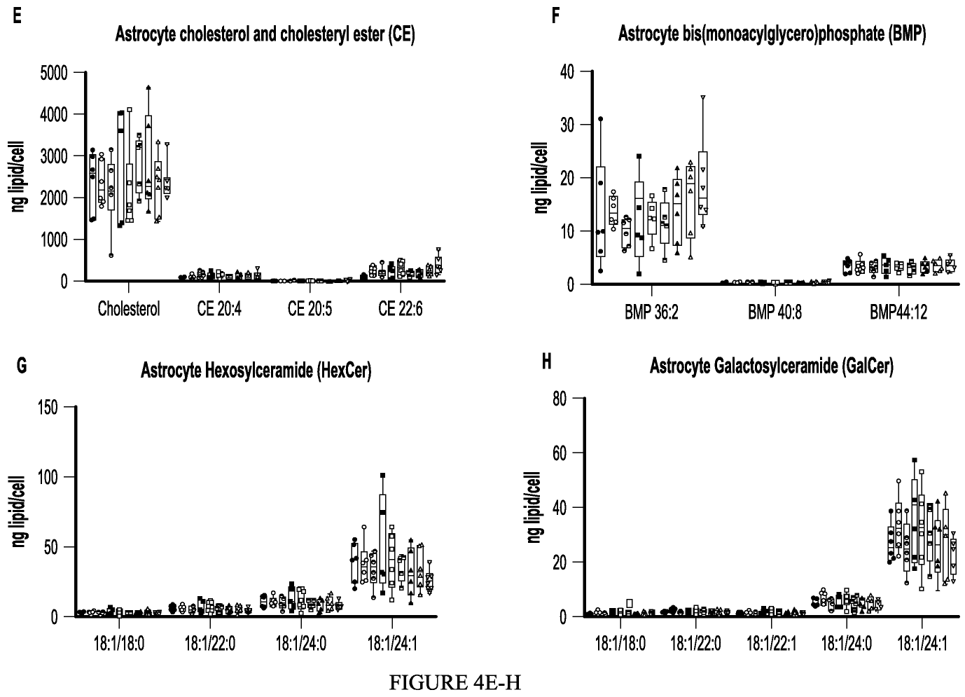

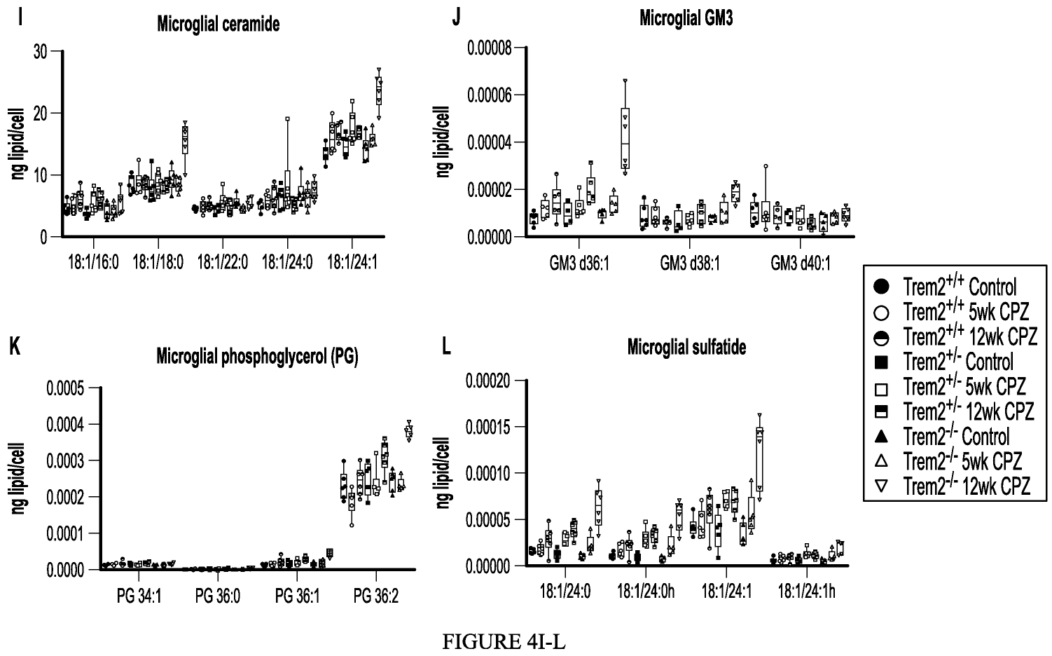

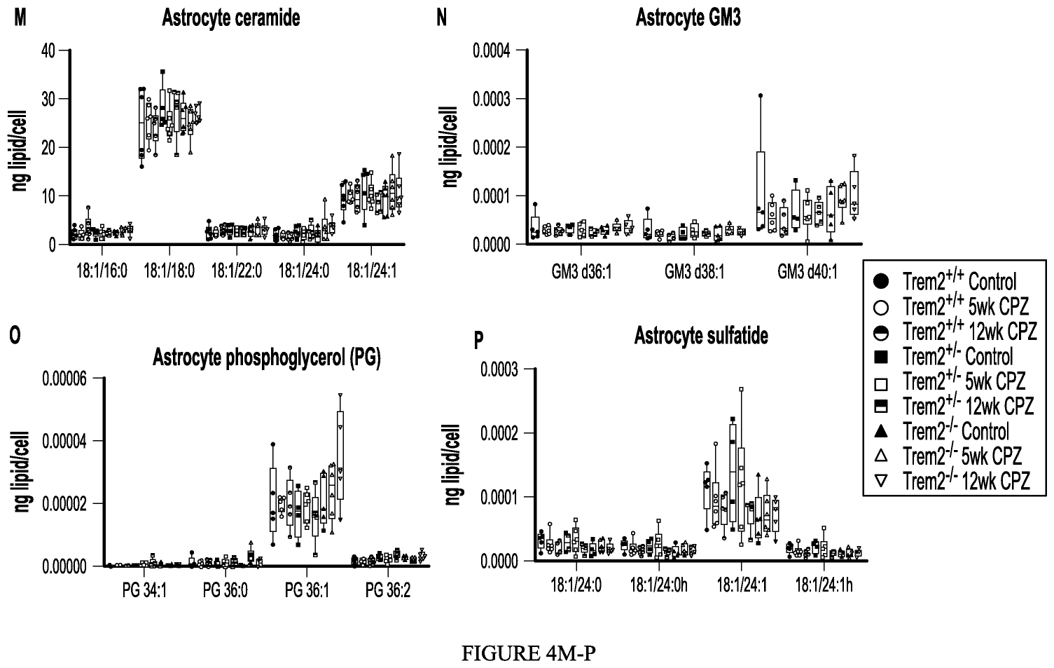

FIGS. 3A-F and FIGS. 4A-P highlight elevated cholesteryl ester and myelin-enriched lipids in forebrain and isolated microglia, but not astrocytes, from Trem2^{-/-} mice with chronic demyelination. In FIG. 3, forebrain total cholesterol levels do not change in Trem2^{+/+}, Trem2^{+/-}, and Trem2^{-/-} mice with control or cuprizone diet (FIG. 3A). Cholesteryl ester (FIG. 3B), oxidized cholesteryl ester (FIG. 3C), BMP (FIG. 3D), and triacylglyceride (FIG. 3E) levels increase in forebrain from Trem2^{-/-} mice with 12 week cuprizone diet compared to Trem2^{+/+}, Trem2^{+/-}, and Trem2^{-/-} mice with control diet or 5 week cuprizone, and Trem2^{+/+} and Trem2^{+/-} with 12 week cuprizone. GM3 d38:1 and GM3 d40:1 (FIG. 3F) levels increase in forebrain from Trem2^{-/-} mice with 12 week cuprizone diet compared to Trem2^{+/+}, Trem2^{+/-}, and Trem2^{-/-} mice with control diet or 5 week cuprizone, and Trem2^{+/+} and Trem2^{+/-} with 12 week cuprizone. In FIG. 4, microglia isolated from Trem2^{-/-} brain with 12 week cuprizone diet show increased cholesteryl ester (FIG. 4A), BMP (FIG. 4B), hexosylceramide (FIG. 4C), and galactosylceramide levels (FIG. 4D) compared to Trem2^{+/+}, Trem2^{+/-}, and Trem2^{-/-} microglia with control diet or 5 week cuprizone, and Trem2^{+/+} and Trem2^{+/-} microglia with 12 week cuprizone. No changes in lipid levels of cholesteryl ester (FIG. 4E), BMP (FIG. 4F), hexosylceramide (FIG. 4G), and galactosylceramide (FIG. 4H) were detected in astrocyte-enriched cell populations isolated from Trem2^{+/+}, Trem2^{+/-}, and Trem2 brain with control or cuprizone diet.

### Example 3. Increased Lipid Storage In Vitro in BMDMs Cultured from Trem2 KO Mice and iPSC Microglia

This example describes the lipid storage phenotype observed in Trem2 KO BMDMs cultured in vitro and treated with oxidized low-density lipoprotein (oxLDL) or myelin, both by immunocytochemistry and mass spectrometry analysis.

### Harvest and Culture of Mouse BMDMs

Mouse femur and tibia bones were dissected and briefly sterilized with 70% ethanol. The bones were washed twice with HBSS, then cracked in 10 mL HBSS by mortar and pestle. The cell suspension was filtered through a 70 μm cell strainer, spun at 300×g for 5 min, and supernatant was discarded. The cell pellet was resuspended in ACK Lysing Buffer (Thermo Fisher A1049201) for 4 min at room temperature. 10 mL RPMI-1640 (Thermo Fisher)+10% Hyclone FBS (GE Healthcare)+ Penicillin-Streptomycin (Thermo Fisher) was added to stop ACK lysis, then spun 300×g 5 min, and supernatant was discarded. Cells were resuspended in RPMI culture media with 50 ng/mL murine M-CSF (Life Technologies, PMC2044), counted and diluted to 1×10⁶ cells/mL, then plated on non-tissue culture treated petri dishes. Three days after seeding, fresh murine M-CSF (50 ng/mL) was added. Five days after seeding, cell culture media was aspirated and cells were washed once with PBS. Cells were resuspended in RPMI/FBS/Pen-Strep and harvested with a cell scraper. Cells were spun at 300×g for 5 min, supernatant was discarded, and cells were either diluted 1×10⁶ cells/mL for direct culture on tissue-culture treated plates, or frozen in RPMI/FBS/Pen-Strep+10% DMSO for later use.

iPSC Microglia Methods
Generation of Human iPSC-Derived Microglia

Summary of method: Hematopoietic progenitor cells are generated from wild type and knockout iPS cells (generation of knockout line protocol below) following manufacturer's instructions using a commercially available kit (StemCell Technologies cat #05310). On day 12 of hematopoietic stem cell differentiation, cells are positive for HSC markers CD34, CD43, and CD45, at which point floating and adherent cells are transferred into 6-well plate containing primary human astrocytes. Replated cells are co-cultured with astrocytes in Media C adapted from Pandya, H et al., Nat Neurosci, 2017. 20(5): p. 753-759 (IMDM, 10% Hyclone FBS, PenStrep, 20 ng/ml IL3, 20 ng/ml GM-CSF, 20 ng/ml M-CSF) for 14-21 days during which time progenitor cells are progressively removed and floating cells are predominantly (>80%) mature microglia. Mature microglia are transferred into homeostatic culture conditions adapted from Muffat, J et al., Nat Med, 2016. (11): p. 1358-1367 (MGdM media) for 3-7 days prior to assay.

### Generation of Stable Knockout Lines

A CRISPR-based approach was used with an RNP-based protocol with reagents from IDT (Alt-R system: https://www.idtdna.com/pages/products/crispr-genome-editing/alt-r-crispr-cas9-system) and NEB (Cas9 cat #M0646M) introduced via nucleofection using Lonza cat #V4XP-3032.

### Myelin Purification

Myelin was purified from wildtype C57Bl/6 mouse brain (Jackson Laboratories) using methods described in in Safaiyan, S et al., Nat Neurosci, 2016. 19(8): p. 995-8. Following purification, myelin was resuspended in PBS and adjusted to 1 mg/mL protein concentration using the DC Protein Assay Kit 2 (BioRad, 5000112).

BMDM were plated in RPMI/10% FBS/Pen-Strep at a density of 100,000 cells per well in tissue culture treated 96 well plates (CellCarrier, PerkinElmer) supplemented with 5 ng/mL mouse M-CSF. ACAT inhibitor K604 was prepared according to published protocols (US 2004/0038987 A1).

### In Vitro Lipid Storage Assay for Nile Red Staining, Filipin Staining or Lipidomics

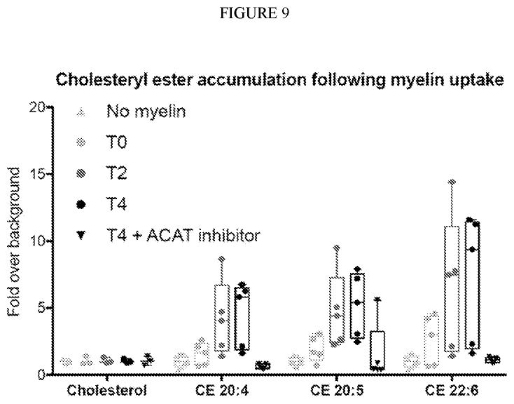

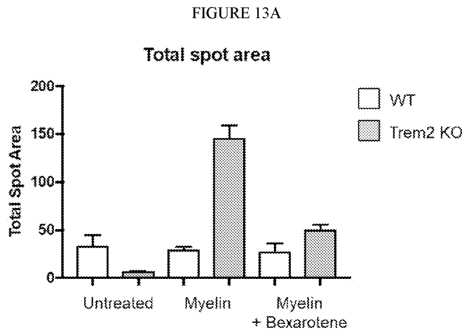

iPSC microglia (30,000/well) or BMDM (100,000/well), either WT or TREM2 KO, were plated on PDL-coated 96-well plates in their respective full serum media including 20 ng/mL mCSF. After 24 hrs at 37° C., purified myelin (isolated from mouse brain as described above, 5 μg/mL (2 hr uptake) or 25 μg/mL or 50 μg/mL (48-72 hr uptake) final concentration) or oxLDL (Thermo Fisher L34357, 50 μg/mL final concentration) was spiked into the wells. For experiments with oxLDL, a second addition of the same amount of oxLDL was spiked into the wells 24 hrs after the first addition. In experiments with ACAT inhibitor, 500 nM ACAT inhibitor K604 or vehicle control was spiked together with the first lipid dose. After 2 hrs (FIG. 9) or 48 hrs-72 hrs at 37° C. of lipid treatment, cells were collected or imaged. For myelin washout experiments, myelin was removed after the 24-hour incubation period and replaced with antibody-containing media for a subsequent 24-48 hours of incubation.

For LC-MS, cells were extracted according to the protocol below. For Nile Red imaging, the supernatant was removed, and cells were incubated at 37° C. for 30 min in live cell imaging buffer (Life Technologies, A14291DJ) containing 1 μM Nile Red (Thermo Fisher N1142) and 1 drop/mL of Nucblue (Thermo Fisher R37605). After the incubation, the staining solution was removed and the cells were fixed in 4% paraformaldehyde. The cells were then imaged using 568 and DAPI illumination settings on an Opera Phoenix high content confocal imager. For Filipin staining, the supernatant was removed and cells were fixed using 4% paraformaldehyde. Cells were washed with Cholesterol Detection Wash Buffer (Abcam ab133116) three times, for 5 min each. Filipin III was diluted 1:100 in Cholesterol Detection Assay Buffer (Abcam ab133116) and added to the cells for 30 min. Cells were washed with Wash Buffer two more times and imaged using DAPI illumination settings. Lipid and filipin spots were analyzed using a spot-finding algorithm on the Harmony software supplied with the instrument. FIGS. 5A-B depicts an increase in lipid accumulation in Trem2 KO BMDMs treated with oxLDL (50 μg/mL) for 48 hrs compared to WT BMDMs, as shown by Nile Red staining (FIG. 5A). Cells were imaged at 63× resolution and Nile Red was quantified as total spot area (FIG. 5B) using a spot-finding algorithm on the Harmony software.

FIG. 9 displays cholesterol and cholesteryl ester (CE) levels in bone-marrow differentiated macrophage from wildtype mice dosed with 5 μg/mL myelin for 2 hrs, then extracted immediately after myelin uptake (TO), or following myelin washout and 2 hrs (T2) or 4 hrs (T4) chase. ACAT inhibitor was added during myelin uptake and maintained through 4 hrs washout (T4+ ACAT inhibitor).

FIGS. 11A-C shows that cholesteryl esters do not accumulate in the presence of the ACAT inhibitor in both WT and TREM2 KO iPSC microglia dosed with myelin, indicating that the cholesteryl ester accumulation is ACAT-dependent. Cholesterol is shown as a control and is not affected by ACAT inhibition.

### Lipid Extraction Protocol for In Vitro Samples

Cells treated with either oxLDL or myelin as described above were then washed with PBS while being kept on ice. 70 μl of a 9:1 methanol:water solution containing internal standards was added to the cells in the 96 well plate. The plate was agitated on a shaker at 4 C and 1200 rpm for 20 min, then spun down for 5 min at 300×g. 50 μl of the supernatant was transferred to LC-MS vials and kept at −80 C until run on the instrument. Refer to Example 2 for mass spectrometry analysis protocol.

In FIGS. 6A-E, mass spectrometry analysis was performed on WT and Trem2 KO BMDMs treated with oxLDL for 48 hrs to characterize lipid species that accumulate intracellularly. OxLDL treatment increases lipid abundance in both WT and Trem2 KO BMDMs, but the increase of cholesteryl esters (FIG. 6A), gangliosides (FIG. 6B), triacylglycerides (FIG. 6C), and hexosylceramide (FIG. 6D) is exacerbated in Trem2 KO BMDMs.

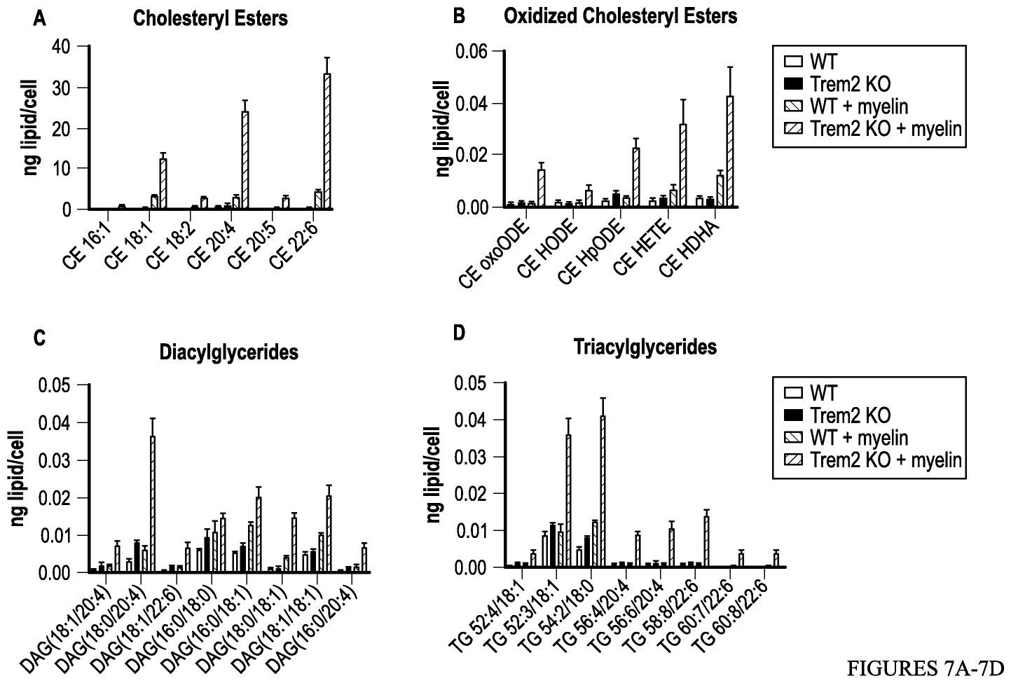

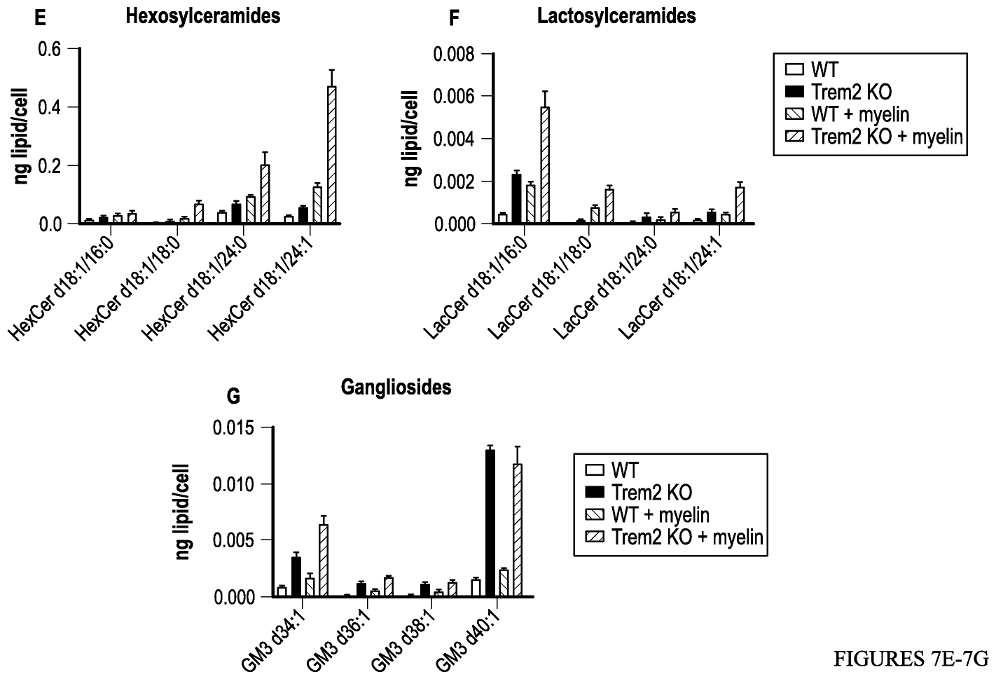

In FIGS. 7A-G, mass spectrometry analysis was performed on WT and Trem2 KO BMDMs treated with myelin for 48 hrs to characterize lipid species that accumulate intracellularly. Trem2 KO BMDMs show greater accumulation of cholesteryl esters (FIG. 7A), oxidized cholesteryl esters (FIG. 7B), diacylglycerides (FIG. 7C), triacylglycerides (FIG. 7D), hexosylceramides (FIG. 7E), lactosylceramides (FIG. 7F), and gangliosides (FIG. 7G) when treated with myelin compared to WT BMDMs.

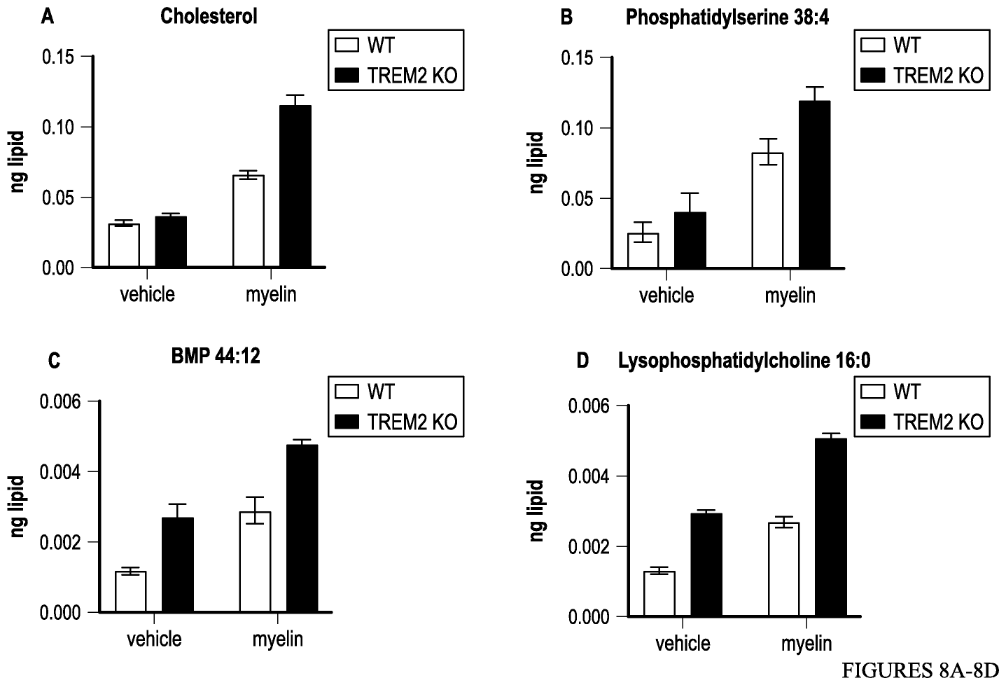

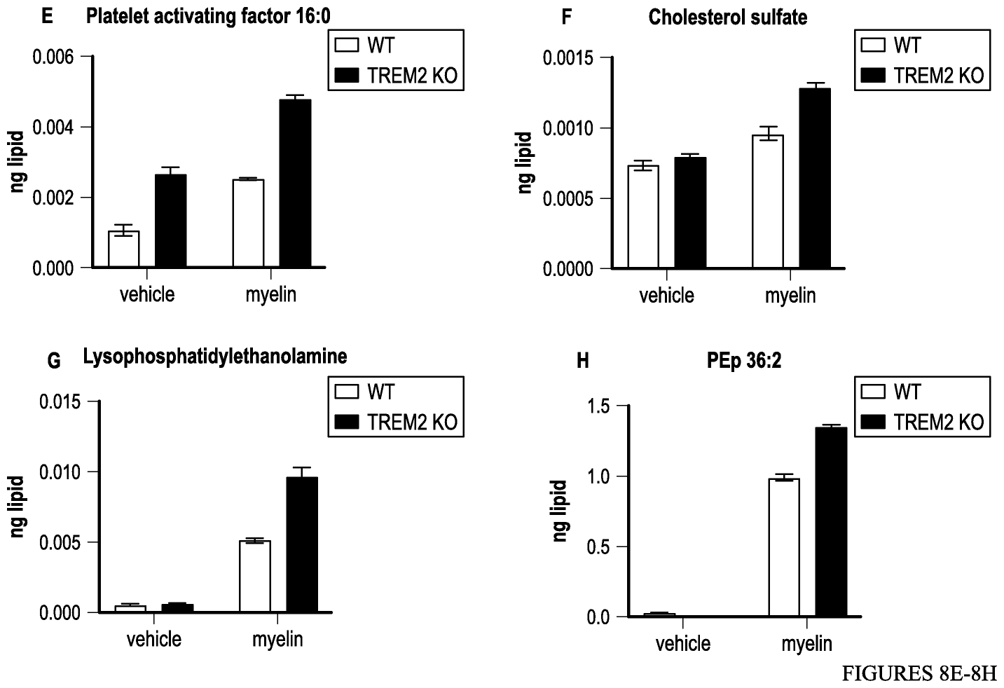

In FIGS. 8A-H, mass spectrometry analysis was performed on WT and TREM2 KO iPSC microglia treated with myelin (25 ug/mL) for 72 hrs to characterize lipid species that accumulate intracellularly. TREM2 KO iPSC show greater accumulation of cholesterol (FIG. 8A), phosphatidylserine 38:4 (FIG. 8B), bis(monoacylglycero)phosphate 44:12 (FIG. 8C), lysophosphatidylcholine 16:0 (FIG. 8D), platelet activating factor (FIG. 8E), cholesterol sulfate (FIG. 8F), and lysophosphatidylethanolamine (FIG. 8G).

FIGS. 23A and 23B show that Trem2 KO BMDMs accumulate more free cholesterol in the endolysosomal system than Trem2 WT BMDM, following treatment with myelin (25 ug/mL) and staining of free cholesterol with filipin.

FIGS. 23C and 23D show that an anti-TREM2 antibody reduces free cholesterol levels in human iPSC-derived microglia compared to a control antibody (anti-RSV). FIG. 23C shows the staining of free cholesterol with filipin in the various conditions and FIG. 23D shows the quantification of filipin puncta.

### Example 4. Improvement of Lipid Accumulation in iPSC Microglia with TREM2 Antibody or Exogenous APOE

iPSCs were generated and the BMDMs were harvested/cultured using methods similar to those of Example 3.

The protocols from Example 3 above for the in vitro lipid storage assay and Nile Red staining or lipidomics were modified as follows: After 24 hrs at 37° C. of lipid treatment, TREM2 antibody or RSV control was spiked into the wells to a final concentration of 100 nM. Cells were incubated for another 48 hrs at 37° C. before collecting or imaging cells. In experiments with APOE3, cells were treated for 24 hrs with myelin (25 μg/mL), then the media was exchanged for media containing 10 μg/mL APOE3. After 24 hrs, cells were collected for analysis. Lipidomics or Nile Red staining were done according to the above protocols.

FIG. 10 shows that Trem2 KO BMDMs accumulate more lipid than WT BMDMs when fed (24 hrs) with myelin, as quantified by total spot area of Nile Red staining. This accumulation is improved by the addition of exogenous human APOE3, which has been shown to mediate lipid efflux (PMID: 9541497, PMID: 10693931, PMID: 15485881).

FIGS. 12A-12C show that a TREM2 antibody can reduce the amount of myelin-induced lipid accumulation in human iPSC-derived WT microglia. This is shown by both Nile Red staining and triacylglyceride level measurements on LC-MS.

FIG. 12D illustrates levels of triacylglyceride (TAG) lipid species as detected by mass spectrometry in cell lysates of iPSC microglia cells treated with several different anti-TREM2 antibodies for 72 hours after a 24-hour myelin treatment. Anti-TREM2 antibodies A and B bind to the stalk region of TREM2, whereas anti-TREM2 antibodies C, D, and E bind to the IgV region of TREM2. FIG. 12E illustrates levels of TAG lipid species as detected by mass spectrometry in cell lysates of iPSC microglia cells which underwent myelin washout experiments with anti-TREM2 antibodies. LC/MS data generated in FIGS. 12D and 12E were normalized to myelin+ isotype control for each individual lipid species.

Lipid accumulation in iPSC microglia is induced by myelin treatment, which is reflected by an increase in neutral lipid staining (Nile Red) and by LC/MS for detection of specific lipid species in cellular lysates. The data illustrated in FIGS. 12A-12E collectively indicate that treatment of iPSC microglia cells post-myelin challenge with multiple anti-TREM2 antibodies reduced accumulation of lipid species, as indicated by the decrease of TAG lipid species levels, as measured by LC/MS. The reduction of lipid levels as a result of antibody treatment was observed at different timepoints ranging from 24 hours to 72 hours. To eliminate the possibility that the reduction in lipid levels is caused by blocking of lipid uptake, myelin washout experiments in which myelin was removed prior to anti-TREM2 antibody addition were carried out. FIG. 12E illustrates that anti-TREM2 antibodies also reduced lipid levels in iPSC microglia with myelin washout prior to antibody treatment relative to isotype control.

### Example 5. Effect of ACAT1 Inhibitor, Bexarotene, and GW3965 on Myelin or oxLDL Storage in TREM2 KO Cells

iPSC microglia (30,000/well) or BMDM (100,000/well), either WT or TREM2 KO, were plated on PDL-coated 96-well plates in their respective full serum media including 20 ng/mL mCSF. After 24 hrs at 37° C., purified myelin (isolated from mouse brain as described above, 5 μg/mL (2 hr uptake) or 25 μg/mL or 50 μg/mL (48-72 hr uptake) final concentration) or oxLDL (Thermo Fisher L34357, 50 μg/mL final concentration) was spiked into the wells. For experiments with oxLDL, a second addition of the same amount of oxLDL was spiked into the wells 24 hrs after the first addition. In experiments with ACAT inhibitor, 500 nM ACAT inhibitor K604 or vehicle control was spiked together with the first lipid dose. In experiments with bexarotene, 10 uM bexarotene or vehicle control was spiked together with purified myelin. In experiments with GW3965, 10 uM GW3965 or vehicle control was spiked together with purified myelin. After 2 hrs (FIG. 9) or 48 hrs-72 hrs at 37° C. of lipid treatment, cells were collected or imaged.

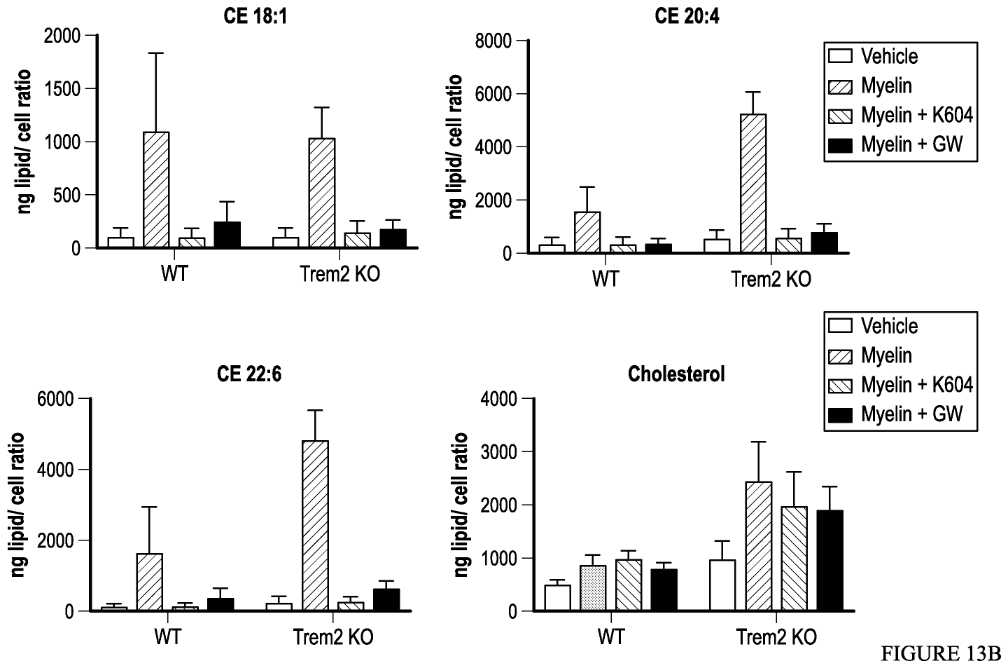

FIG. 13A shows Trem2 KO BMDMs accumulate more neutral lipid than WT BMDMs when treated for 48h with myelin debris (25 ug/mL), as quantified by Nile Red staining. This accumulation is reduced by co-treatment with bexarotene (10 uM).

FIG. 13B shows human iPSC-derived TREM2 KO microglia accumulate various cholesteryl ester (CE) species and that this accumulation is reduced by co-treatment with an ACAT inhibitor K604 (500 nM) and an LXR agonist GW3965 (10 μM).

### Example 6. Changed Expression of Genes Implicated in Lipid Metabolism and Lysosome Function in Trem2 Knockout Mice with Chronic Demyelination

This example describes mouse microglial gene expression analyses. In particular, these analyses demonstrate that 1) TREM2 deficiency prevents DAM conversion during chronic demyelination; 2) TREM2 deficiency blocks age-dependent conversion to damage-associated microglia states; and 3) Trem2^{-/-} microglia exhibit attenuated transition to a damage-associated microglia state upon demyelination, as shown by single cell RNAseq.

Generally, methods similar to those described in Example 1 were used, with the single cell RNAseq cluster and expression analysis performed as indicated below.

### Single Cell RNAseq Cluster and Expression Analysis

PCA was performed on the log 2 normalized gene expression matrix, and the top twenty-one principal components were retained. A shared nearest neighbor graph (Xu, C., and Su, Z. (2015). Bioinformatics 31, 1974-1980) was built over the data in PC-space followed by community detection using the Louvain method (Blondel, et al., (2008). Journal of Statistical Mechanics: Theory and Experiment 2008) to assign cells to one of eight clusters. Marker genes per cluster were identified by exhaustively performing pairwise Wilcoxon tests, as implemented in the scran package. Briefly, the expression level of each gene within a cluster was tested against each of the other seven clusters, individually. The seven resulting p-values were combined using Simes' method Simes, R. J. (1986). Biometrika 73, 751-754) to provide a final p-value for the gene's differential expression status per cluster, which were then adjusted to correct for multiple testing using the Benjamini-Hochberg method (Benjamini, Y., and Hochberg, Y. (1995). Journal of the Royal Statistical Society 57, 289-300). Effect sizes for each comparison are calculated as “overlap proportions” i.e. the probability that a cell selected at random within the source cluster has higher expression of gene X than a random cell in the query cluster. Overlap proportions (Wilcoxon effect sizes) are averaged over all pairwise comparisons to provide a final effect size for the gene within the cluster. Finally, marker genes per cluster were extracted by identifying the genes with an FDR<0.05 and averaged overlap proportions lower than 0.4 or greater than 0.6

### TREM2 Deficiency Prevents DAM Conversion During Chronic Demyelination

To characterize the effects of acute and chronic demyelination on Trem2-dependent gene expression in microglia, Trem2^{+/+}, Trem2^{+/-}, and Trem2^{-/-} mice were fed a 0.2% CPZ diet for either 5 or 12 weeks. CD11b⁺ microglia were isolated from hemibrain by FACS and transcriptome analysis was performed using RNAseq. The vast majority of CD11b⁺ cells were CD45^{low}. In fact, CD45^{high} cells represented less than 0.2% of the total live cells in the absence of CPZ for all three genotypes and less than 0.5% in the presence of CPZ diet, suggesting a minor infiltration of macrophages in this model. Principal Component analysis (PCA) showed that CPZ treatment induced transcriptional changes in microglial samples from Trem2^{+/+} and Trem2^{+/-} animals, whereas CPZ-challenged Trem2^{-/-} microglia clustered with those of untreated animals. Differential gene expression analysis failed to reveal marked genotype-dependent differences under normal diet conditions (see. Example 1, FIGS. 1A-B, absolute log 2 fold change >0.5, FDR<0.2). In contrast, applying the same thresholds to the effect of CPZ identified hundreds of significantly up- or down-regulated genes, with changes after 5 weeks and even stronger effects after 12 weeks (see, Example 1, FIGS. 1A-1B). Genotype-wise comparisons between 5 and 12 week CPZ vs. control diets confirmed that these gene expression changes were almost entirely restricted to microglia from Trem2^{+/-}+ and Trem2^{+/-} animals, while microglia from Trem2^{-/-} mice largely failed to respond to the myelin challenge.

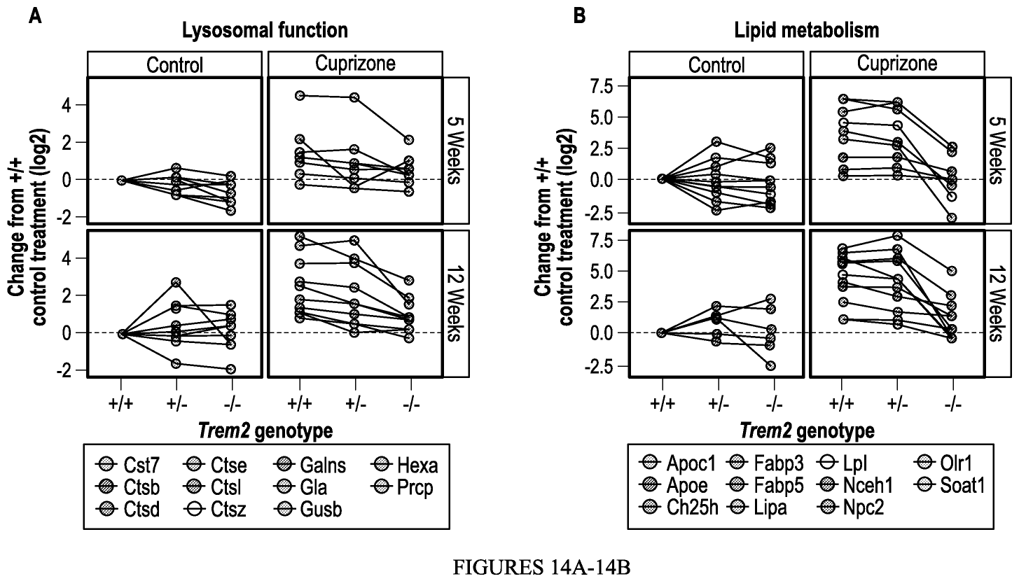

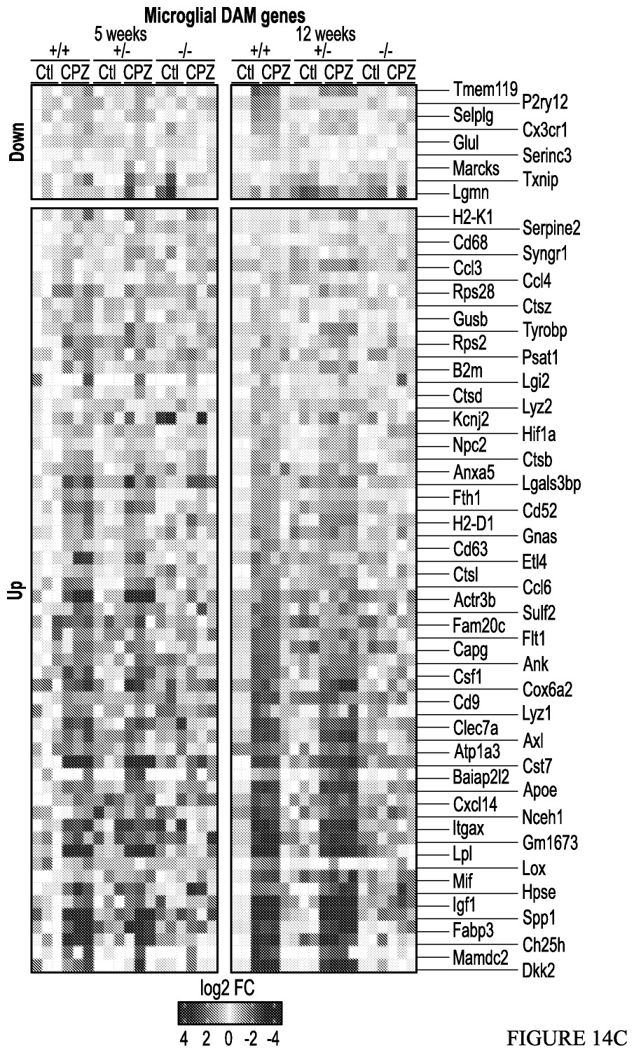

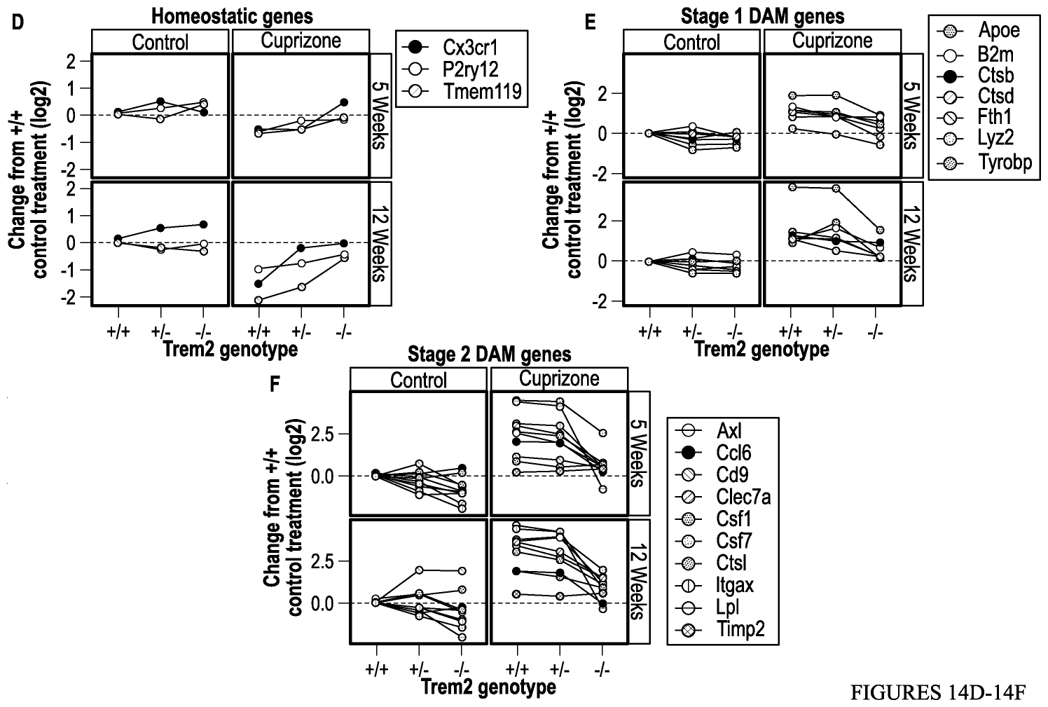

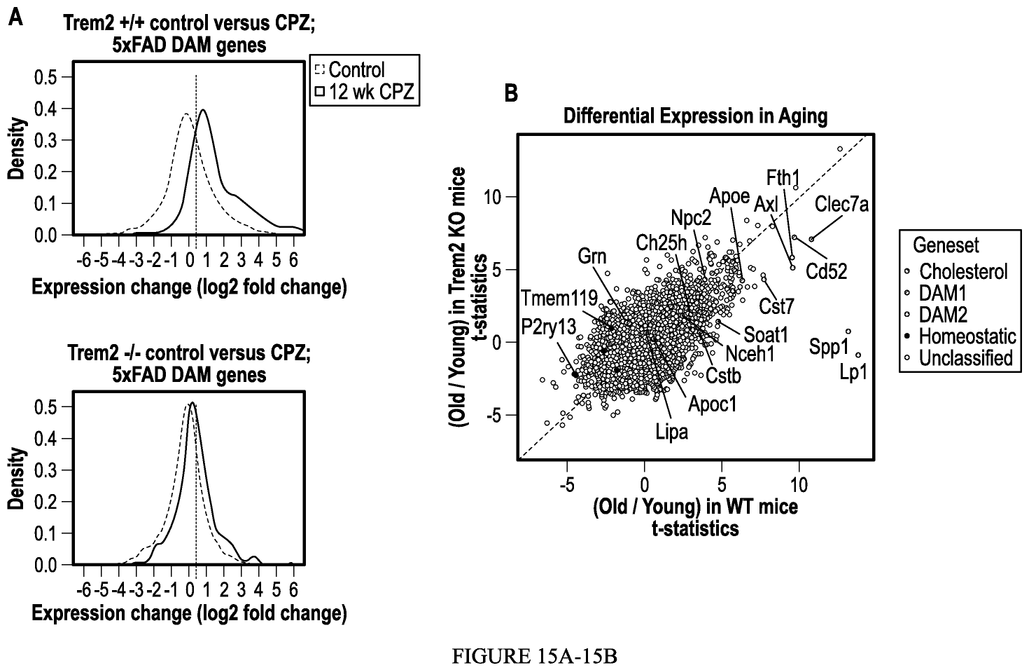

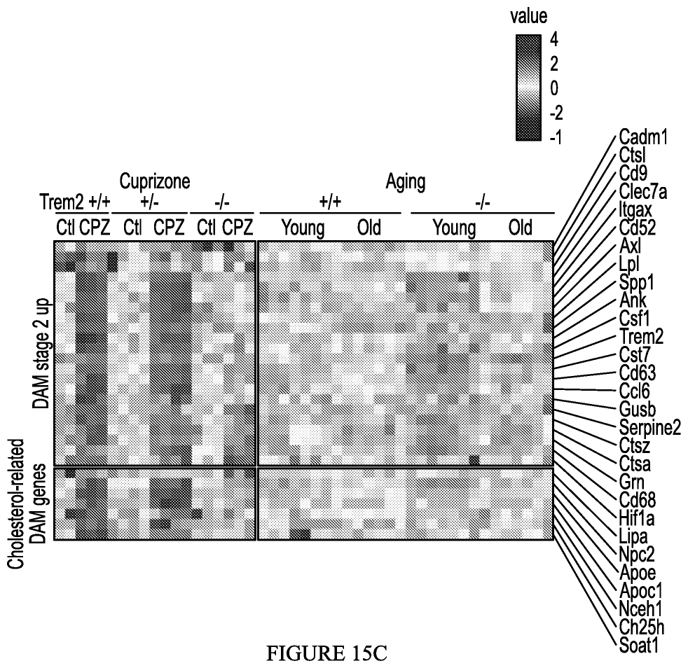

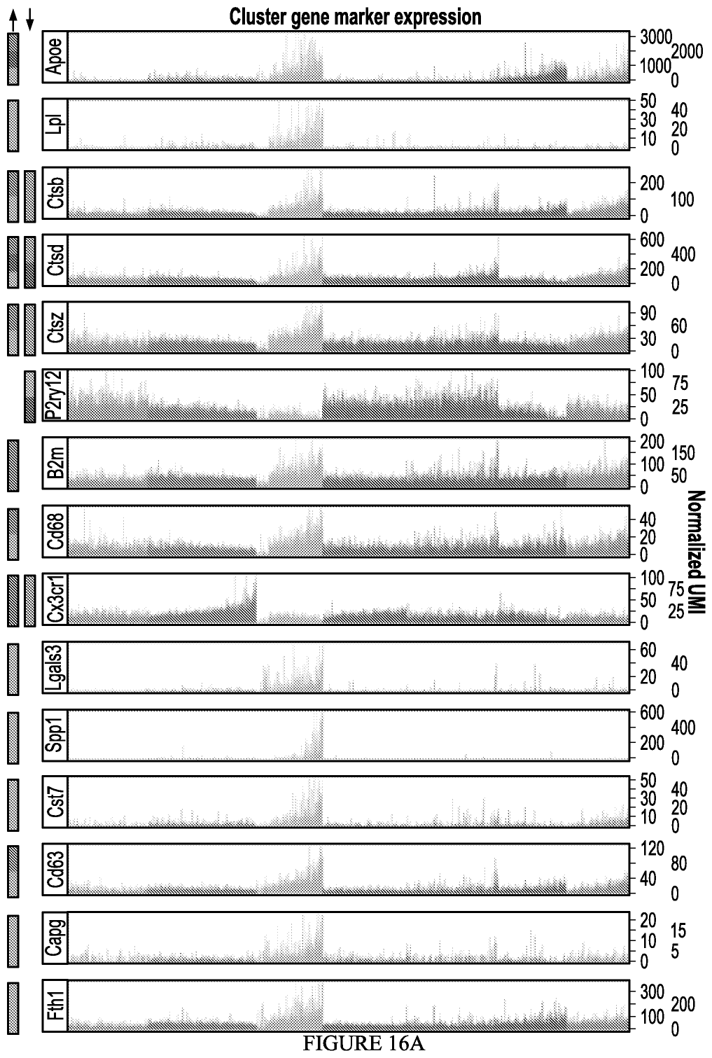

Using gene set analysis from the Reactome database (Fabregat, et al. (2018). Nucleic Acids Res 46, D649-D655), significant Trem2-dependent upregulation was detected of genes involved in lysosome and phagosome function, AD, oxidative phosphorylation and cholesterol metabolism. For example, as shown in FIGS. 14A-14B, genes involved in lysosomal function and lipid metabolism are upregulated in Trem2^{+/+} and Trem2^{+/-} murine microglia upon acute and chronic demyelination (5 and 12-week cuprizone treatment, respectively), but exhibit reduced upregulation in Trem2^{-/-} murine microglia with chronic and acute demyelination. FIG. 14A shows the log 2 fold change of gene expression in individual genes associated with lysosomal function in Trem2^{+/+}, Trem2^{+/-}, and Trem2^{-/-} bulk microglia with control diet (left inset) versus 5 or 12 weeks cuprizone treatment (right inset, top or bottom, respectively). FIG. 14B shows the log 2 fold change of gene expression in individual genes associated with lipid metabolism in Trem2^{+/+}, Trem2^{+/-}, and Trem2^{-/-} bulk microglia with control diet (left inset) versus 5 or 12 weeks cuprizone treatment (right inset, top or bottom, respectively). For example, key genes of the lysosome degradation pathway, such as Ctse and Ctsl, were upregulated 2-fold upon chronic demyelination in Trem2^{+/+} and Trem2^{+/-} microglia (FDR<0.05), but unchanged in Trem2^{-/-} microglia (FIG. 14A, interaction p-values ≤0.05). Several Trem2-genes appear to control multiple aspects of cholesterol transport and metabolism, including Ch25h, Lipa, Nceh1, Npc2 and Soat1 in addition to Apoe. Gene set enrichment also showed that previously described DAM genes (Keren-Shaul, et al. (2017). Cell 169, 1276-1290 e1217) were significantly upregulated in response to CPZ treatment in microglia from both Trem2^{+/+} and Trem2^{+/-} animals at both time points (FIG. 14C). This DAM-like response was attenuated in samples from Trem2^{-/-} animals (FIGS. 14C and 15C), which suggests that chronic CPZ demyelination induces microglial expression changes that reflect those observed in 5XFAD and SOD1 microglia (Keren-Shaul, et al. (2017). Cell 169, 1276-1290 e1217).

The microglia transition from homeostasis into DAM has been described as a two-step process: a TREM2-independent transition to an intermediate state (DAM stage 1), followed by a second, TREM2-dependent change (DAM stage 2) (Keren-Shaul et al. (2017) Cell 169, 1276-1290 e1217). Consistent with previous models, the homeostatic genes P2ry12 and Tmem119 were significantly downregulated in Trem2^{+/+} and Trem2^{+/-} (FDR<0.01) but not Trem2^{-/-} microglia in response to CPZ (FIG. 14D, genotype-diet interaction p-value <0.05). Reduced induction of stage 1 DAM genes such as Apoe (interaction p-value <0.001), Fth1 (interaction p-value <0.005), and Tyrobp (interaction p-value <0.1) in Trem2^{-/-} compared to Trem2^{+/-} and Trem2^{+/-} microglia following CPZ treatment was also observed (FIG. 14E). For example, the expression of Apoe was upregulated more than 8-fold by CPZ treatment of Trem2^{+/+} and Trem2^{+/-} animals after 12 weeks, but this response was attenuated in Trem2^{-/-} animals, similar to that observed for stage 2 DAM genes, such as Axl (interaction p-value <0.05), Cd9 (interaction p-value <0.001) or Csf1 (interaction p-value <0.1) (FIG. 14F). In summary, these data confirm TREM2 as a regulator of phagocytic clearance of myelin debris and point toward a role in endolysosomal processing and lipid metabolism, with a clear implication of cholesterol transport and metabolism. Additionally, they suggest CPZ chronic demyelination elicits a damage-associated microglia state, which fails to be initiated in Trem2^{-/-} microglia.

### TREM2 Deficiency Blocks Age-Dependent Conversion to Damage-Associated Microglia States

Gene expression studies have indicated that aged microglia acquire a DAM transcriptional state, suggesting that microglia may respond to age-induced parenchyma damage (Keren-Shaul, et al. (2017). Cell 169, 1276-1290 e1217). To test whether aged Trem2^{-/-} microglia are less competent at transitioning to damage-associated microglia states relative to wildtype microglia, gene expression analysis was performed on sorted microglia derived from young (2 month-old) and aged (15-17 month-old) wildtype and Trem2^{-/-} mice.

Based on the downregulation of a variety of homeostatic genes and upregulation of DAM 1 and DAM 2 genes, it was confirmed that microglia isolated from aged wildtype brain expressed damage-associated microglia features (FIGS. 15B and 15C). This response was largely attenuated in Trem2^{-/-} microglia, as observed in CPZ-treated Trem2^{-/-} microglia. The DAM 2 gene set was more profoundly affected than the DAM 1 gene set, as exemplified by the more striking upregulation of Lpl and Spp1 in aged wildtype microglia relative to aged Trem2^{-/-} microglia (FIGS. 15B and 15C). This is consistent with the Trem2-dependency of the DAM 2 profile exhibited in 5XFAD microglia (Keren-Shaul, et al. (2017). Cell 169, 1276-1290 e1217). However, the cholesterol metabolism-related gene module was not as profoundly affected in aged Trem2^{-/-} microglia as it was in CPZ-treated Trem2^{-/-} microglia relative to control microglia (FIGS. 15B and 15C). Together, these data suggest that the maladaptive functions observed in microglia derived from CPZ-challenged Trem2^{-/-} mice is also present in microglia from aged Trem2^{-/-} mice, although gene expression in the latter is not as profoundly altered when compared to the CPZ challenge.

### Trem2^{-/-} Microglia Exhibit Attenuated Transition to a Damage-Associated Microglia State Upon Demyelination, as Shown by Single Cell RNA Seq

The following experiments were designed to determine if 1) the population-based CPZ-induced transcriptional changes observed in the bulk RNAseq profiles are universal to all microglia; or 2) heterogeneous transcriptional responses occur at the single cell level. To address this question, scRNAseq was conducted on CD11b⁺/CD45^{low} microglia isolated from Trem2^{+/+} control brain compared to microglia from Trem2^{+/+}, Trem2^{+/-}, and Trem2^{-/-} mice with chronic demyelination.

These experiments produced data for 3,023 individual cells. In order to identify transcriptionally distinct sub-populations of microglia within these data, a shared nearest neighbor approach was used to perform unsupervised graph-based clustering over the single cell expression profiles (Xu, C., and Su, Z. (2015). Bioinformatics 31, 1974-1980). This identified eight sub-populations of microglia, each accounting for 2% to 19% of all analyzed cells. Quantification of cluster membership across groups identified two clusters (4 and 8) that were essentially absent in the Trem2^{+/+} controls (<3%) but exhibited strong treatment- and genotype-specific expansion and collapse. The expression profiles of the cells in Cluster 4 were only observed in microglia exposed to chronic demyelination with at least one functional copy of Trem2 (˜1% of Trem2^{+/+} controls; 16%-19% of Trem2^{+/+} CPZ and Trem2^{+/-} CPZ; ˜1.5% of Trem2^{-/-} CPZ). The expression profiles of the microglia in Cluster 8 were also largely absent in Trem2^{+/+} controls (<2.3%), their relative abundance increased mildly in the Trem2^{+/+} CPZ and Trem2^{+/-} CPZ mice (˜10%), and were most abundant in the Trem2^{-/-} CPZ mice (˜20%).

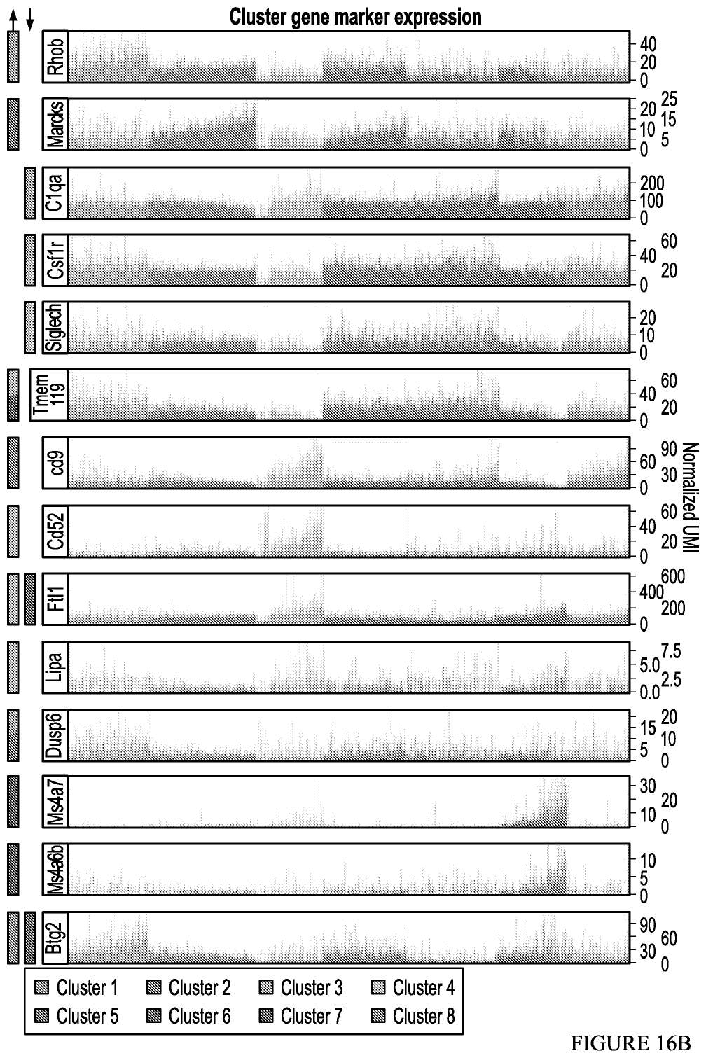

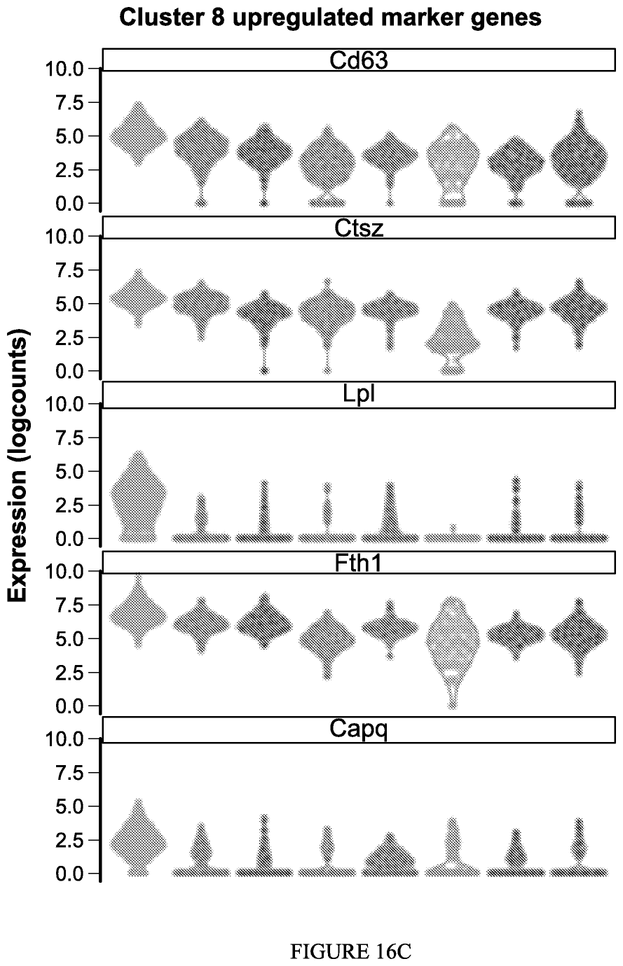

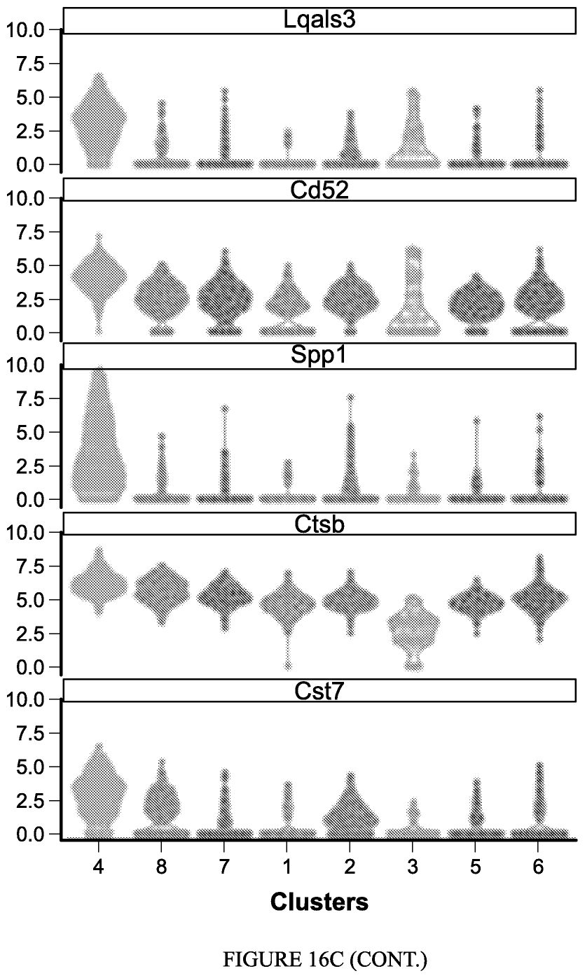

To further characterize the expression profiles within each cluster, a marker-gene analysis was performed to identify the genes within a given cluster that exhibited cluster-specific over- and under-expression with respect to the remaining seven clusters at an FDR<0.05. Relative expression of the top 15 up- and down-regulated genes per cluster confirms that these clusters are transcriptionally distinct, although it is rare to find genes that are exclusive in expression between the clusters. Consistent with the findings from the bulk microglial data, top upregulated marker genes in Trem2^{+/+} CPZ and Trem2^{+/-} CPZ-enriched Cluster 4 consisted of lysosomal genes, such as Ctsb, Ctsd, and Ctsz, as well as genes involved in lipid metabolism, such as Apoe and Lpl (FIG. 16A). Marker genes that were downregulated in Cluster 4 contained microglial homeostatic genes, such as P2ry12 and Tmem119, suggesting microglia in this cluster are in a more reactive state (FIGS. 16A and 16B). The top upregulated marker genes within Cluster 8 are similar to those upregulated Cluster 4, yet to a lesser degree (FIGS. 16A and 16C). Thus, upregulated marker genes in Cluster 8 similarly consisted of lysosome- and lipid metabolism-related genes, such as Ctsb. Ctsd. and Apoe (FIG. 16A). This data reinforces that Trem2^{-/-} CPZ microglia are not fully capable of upregulating transcription of lysosome- and lipid metabolism-related genes to enable proper conversion to reactive states in the presence of demyelination. However, not all microglia in the brain of Trem2^{+/+} and Trem2^{+/-} mice alter gene expression with chronic demyelination. Rather, approximately 20% of the total microglia population upregulate the above genes.

Consistent with the findings from the bulk microglial data, the transcriptional program found in the Trem2^{+/+} and Trem2^{+/-} CPZ microglial Cluster 4 was also largely comprised of upregulated marker genes that previously have been characterized in DAM 2 expression (Keren-Shaul, et al. (2017). Cell 169, 1276-1290 e1217), including Lgals3, Cd63, Spp1, Cst7, Cd68, Capg, and Fth1 (FIG. 16A). To characterize the degree to which Clusters 4 and 8 related to DAM stage 1 and 2 genes, a set of previously reported marker genes was used per DAM state to provide an aggregated DAM score for each cell. These scores were then averaged by cluster to summarize the degree to which each cluster resembles the given DAM state. Cluster 4 shows the highest enrichment for DAM 2 gene expression, followed by Cluster 8, suggesting Trem2^{+/+} CPZ and Trem2^{+/-} CPZ microglia exhibit a DAM 2-like transition in response to chronic demyelination that is greatly attenuated in Trem2^{-/-} CPZ microglia. Additionally, TREM2-independent DAM 1 genes were upregulated in Trem2^{+/+} CPZ, Trem2^{+/-} CPZ, and Trem2^{-/-} CPZ microglia, however Trem2^{+/+} CPZ and Trem2^{+/-} CPZ microglia exhibited higher expression of DAM 1 genes compared to Trem2^{-/-} CPZ microglia. Thus, upon chronic demyelination, a subset of Trem2^{-/-} microglia exhibit attenuated expression of certain DAM genes. These data demonstrate that Trem2^{-/-} CPZ microglia are not fully capable of upregulating transcription of lysosomal, lipid metabolism, and DAM genes, as in microglia with at least one functional copy of Trem2, to enable proper conversion to reactive states during chronic demyelination.

### Example 7. TREM2 Deficiency Causes Neuronal Damage During Chronic Demyelination

This example describes the effects of a TREM2 deficiency on neuronal damage during chromic demyelination.

### Cuprizone Diet to Induce Demyelination in Mice

Methods similar to those described in Example 1 were used for the demyelination protocol.

### Reagents

Primary antibodies: 1:100 Rabbit anti-beta-APP (Life Technologies, 512700); 1:1000 Mouse anti-SMI-32 (Millipore 559844). Secondary antibodies: goat anti-mouse-555, goat anti-mouse-647, goat anti-rabbit-555, and goat anti-rabbit-647 (Invitrogen).

### Immunofluorescence

Sagittal mouse hemibrains were flash frozen in liquid nitrogen after PBS perfusion and coronally cryosectioned at −200 C with alternating 100 μm (lipidomics) or 20 μm (histology) widths using a Leica CM 1950 cryostat. 10 consecutive histological slide sets representing rostral to caudal brain regions were collected for each hemibrain and were frozen at −800 C. Prior to staining, slides were thawed at room temperature until dry, then fixed with 4% paraformaldehyde (Electron Microscopy Sciences 15710) for 10 min. Slides were washed twice with PBS for 5 min, then blocked for 1 hr at room temperature with PBS+0.3% TritonX-100+5% Normal Goat Serum (Vector Labs S-1000). Primary antibodies were diluted in blocking buffer and added to slide overnight at 40 C. Slides were washed 3×15 min in PBS and incubated with secondary antibodies diluted 1:1000 in blocking buffer for 2 hr, room temperature. Slides were washed 3×15 min in PBS, dried at room temperature, and mounted with Fluoromount G (Southern Biotech 0100-01). Images of immunostained brain sections were captured in a Zeiss AxioScan automated slide imager with a 20× objective and Zeiss Cell Observer SD, then cropped to focus on the hippocampal area.

### Neurofilament Light Detection

Mouse blood was collected into EDTA tubes (Sarstedt 201341102) with a capillary tube (Sarstedt 201278100), spun at 15,000×g for 7 min at 40 C, and the top plasma layer was transferred to a 1.5 mL tube and stored at −800 C. Frozen plasma samples were thawed on ice and diluted 10 fold and run on a SR-X (Quanterix) using the Simoa NF-light advantage kit (Quanterix 103186) according to the manufacturer's protocol.

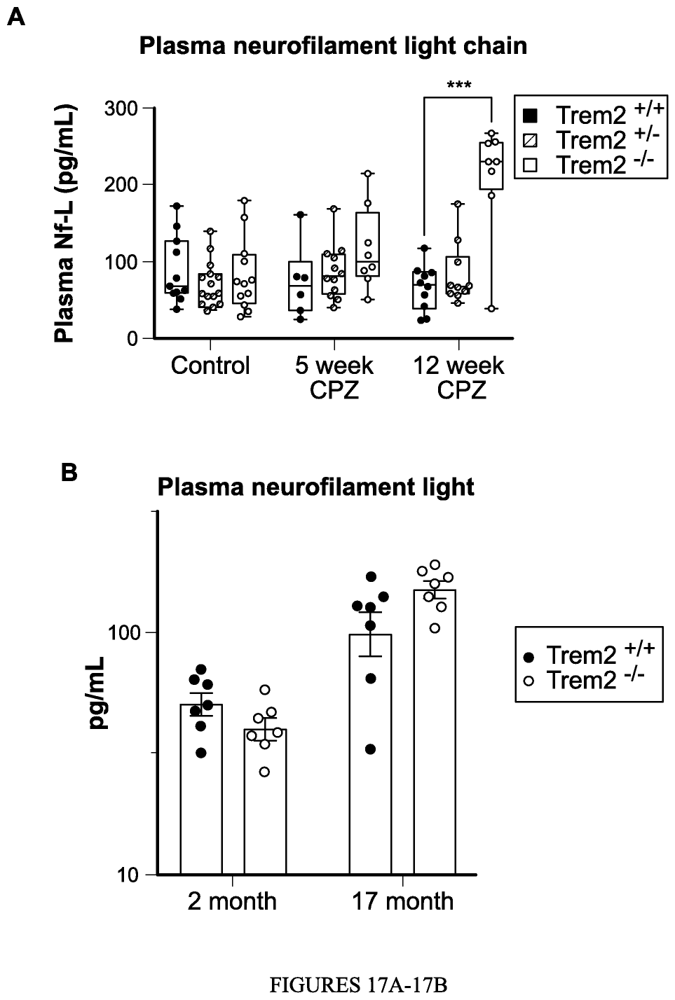

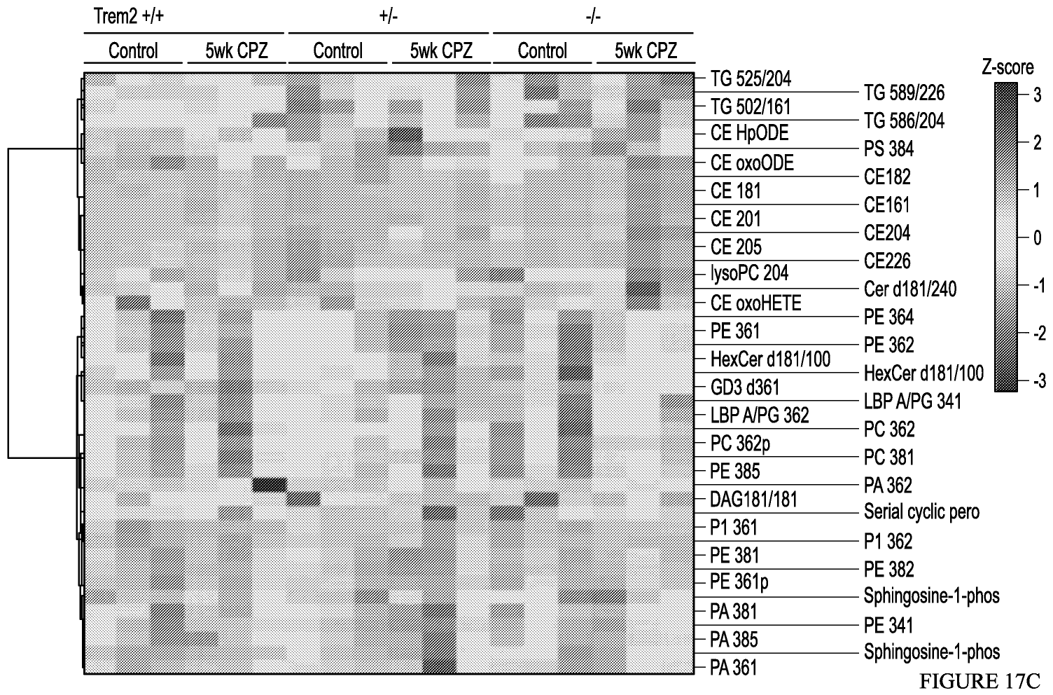

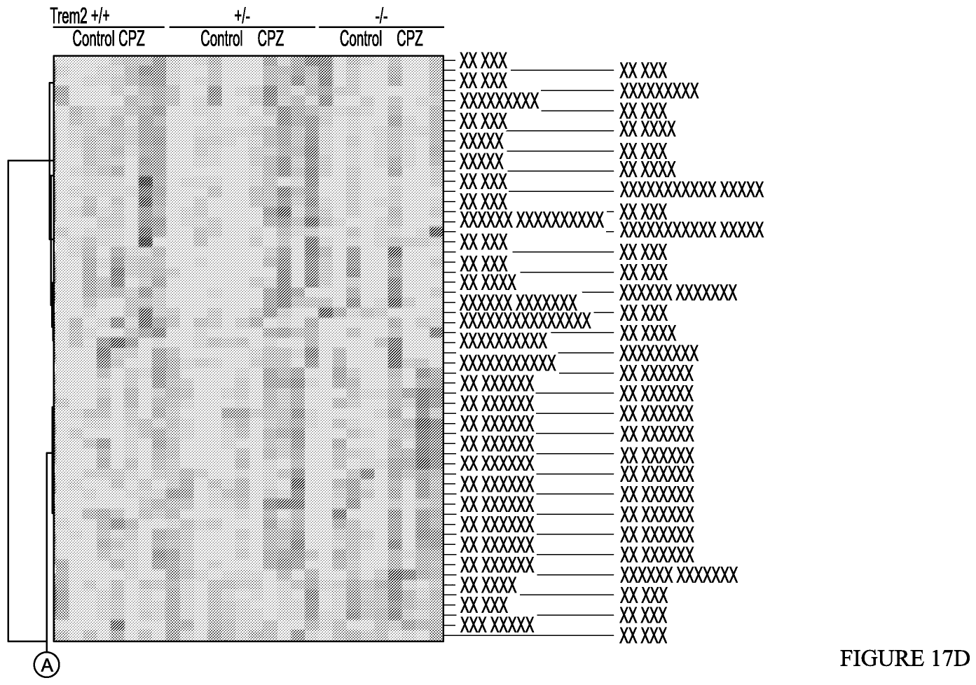

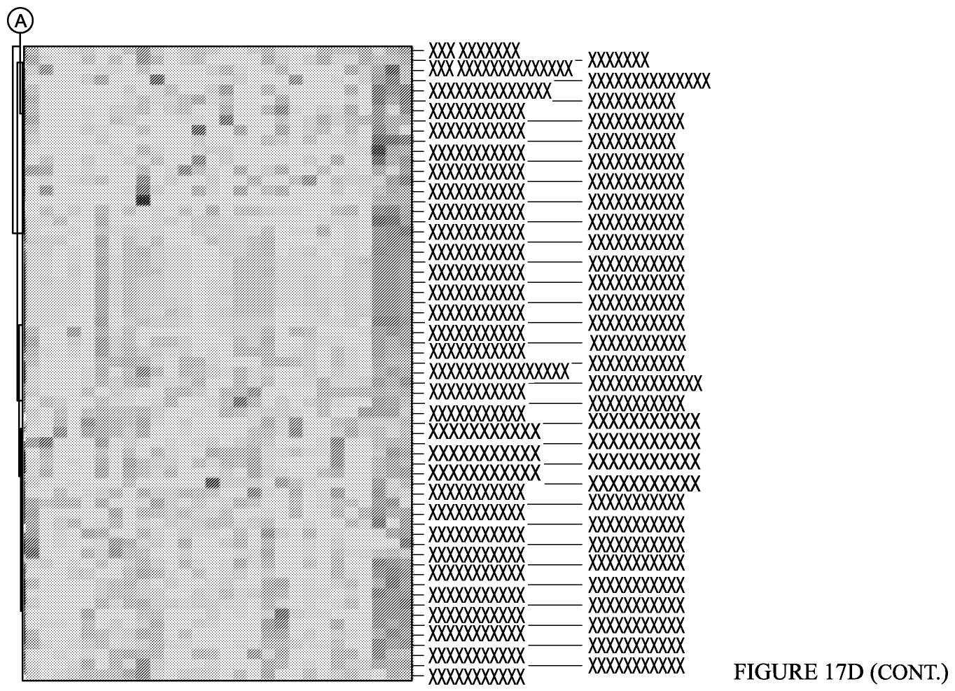

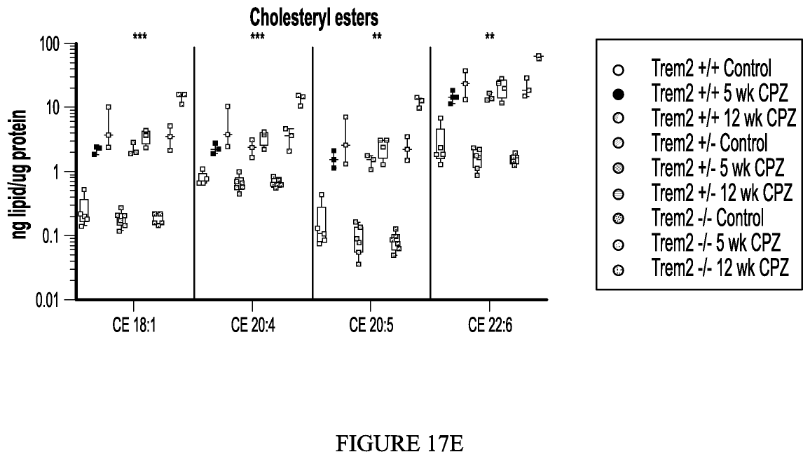

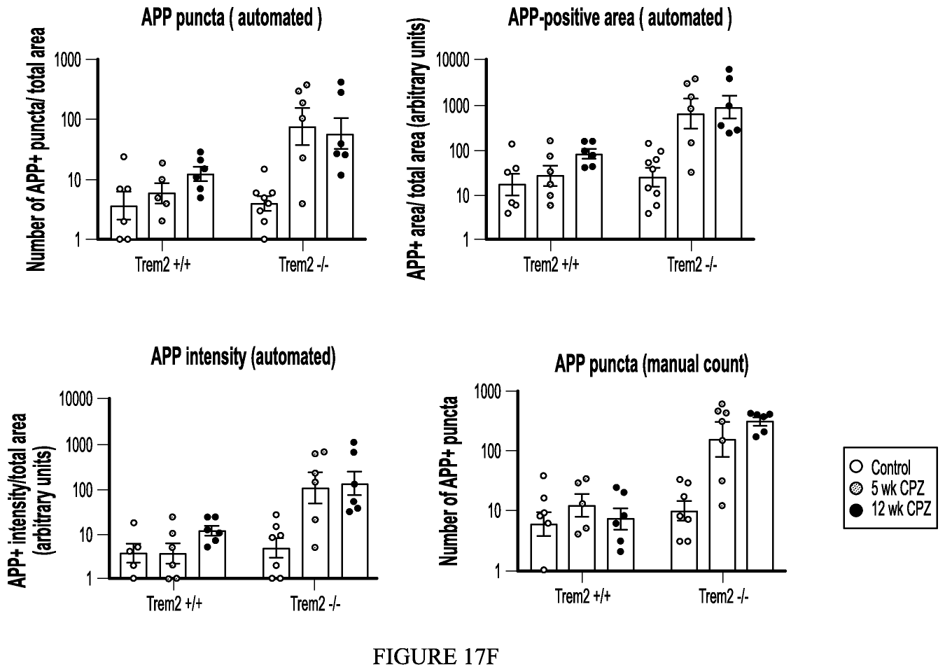

Accumulations of dystrophic APP-positive puncta were identified in the hippocampus and corpus callosum of Trem2^{-/-} mice after 5 or 12 weeks of CPZ compared to all other groups (FIG. 17F). APP puncta were the size of cell nuclei, but they did not co-localize with DAPI. Instead, the puncta were surrounded by or continuous with SMI32⁺ non-phosphorylated neurofilament staining, suggestive of dystrophic neurites. Quantification of APP-positive dystrophic neurite puncta, conducted in a semi-automated fashion or manually, showed a strong genotype effect as well as an interaction between genotype and CPZ treatment for the 5 and/or 12 week treatment, depending on the parameter quantified (puncta number, intensity or area) (FIG. 17F).

To confirm that chronic demyelination causes neuronal damage in the absence of TREM2, neurofilament-light chain (Nf-L) levels in plasma were also measured. Consistent with APP staining, Nf-L levels were unchanged in control or CPZ Trem2^{+/+} and Trem2^{+/-} plasma, but they were elevated in Trem2^{-/-} plasma upon chronic demyelination (FIG. 17A). In agreement with increased neuronal damage observed in the Trem2^{-/-} CNS with CPZ treatment, aged Trem2^{-/-} mice showed an increase in Nf-L levels in their plasma (FIG. 17B; two-way ANOVA, FDR<0.05, interaction age-genotype p<0.05). These data support the notion that TREM2 plays a neuroprotective role.

### Example 8. Lipidomic Alterations in Trem2 Knockout Mice with Chronic Demyelination

This example describes lipidomics of forebrain and isolated microglia, astrocytes and CSF from Trem2 knockout mice with chronic demyelination.

### Cuprizone Diet to Induce Demyelination in Mice

Methods similar to those described in Example 1 were used for the demyelination protocol.

### CSF Isolation

For CSF isolation, mice were anesthetized using 2.5% Avertin/tert-amyl alcohol. After sedation, a sagittal incision was made at the back of the animal's skull to expose the cisterna magna and a needle attached to a glass capillary tube was used to puncture the cisterna magna to collect CSF. CSF was transferred to 0.5 mL lo-bind tubes (Eppendorf) and spun at 12,000 rpm for 10 min, 4° C. 2 μL of supernatant was transferred to glass LCMS vials and 50 μL methanol containing internal standards was added before LCMS analyses.

FACS of Microglia, Astrocytes, and Other Cells from Mouse Brain

Methods similar to those described in Example 1 were used for brain dissociation and the FACS protocol.

### FACS Lipid Extraction and Mass Spectrometry Analysis

Lipid extraction and mass spectrometry of forebrain, microglia, astrocytes and CSF was performed using methods similar to those described in Example 2.

### TREM2 Deficiency Causes Cholesteryl Ester Accumulation in the Brain

As described in Example 6, expression of genes implicated in lipid metabolism is strongly induced upon chronic demyelination in wildtype but not Trem2^{-/-} microglia (FIGS. 14B and 16A), including six genes encoding for proteins directly involved in cholesterol metabolism, mediating extracellular transport (Apoe), hydrolysis of cholesteryl esters in lysosomes (Lipa), egress of unesterified cholesterol from lysosomes (Npc2), cholesteryl ester synthesis and storage in lipid droplets (Soat1), cholesteryl ester hydrolysis in lipid droplets (Nceh1), and 25-hydroxylation (Ch25h) (see, e.g., FIGS. 14B and 16A). Therefore, to test whether intracellular and extracellular cholesterol transport is defective in Trem2^{-/-} microglia after CPZ challenge, LCMS analysis of lipid extracts from coronal forebrain sections containing the corpus callosum was conducted.

No differences in the lipidomic profile of control Trem2^{+/+}, Trem2^{+/-}, and Trem2^{-/-} brain under control conditions were found (FIG. 17D). Upon acute demyelination (5 week CPZ), minimal changes were detected, with an enhancement in cholesteryl ester (CE) and oxidized forms of CE (oxCE) levels in all three genotypes (FIG. 17C). With chronic demyelination (12 week CPZ), CE and oxCE lipid species were significantly elevated (FIGS. 17D-17E; two-way ANOVA, FDR<0.05, interaction p<0.01 for 12 weeks CPZ; see also. Example 2, FIGS. 3A-3C). Further comparison revealed TREM2-deficient brain with chronic demyelination significantly accumulated CE species containing poly-unsaturated fatty acids, such as CE22:6 (docosahexaenoic acid, DHA) and to a lesser extent CE20:4 (arachidonic acid) compared to Trem2^{+/+} and Trem2^{+/-} 12 week CPZ brain (FIG. 17E; interaction p<0.0001, two-way ANOVA). CE22:6 showed the most striking increase in Trem2^{-/-} brain with chronic demyelination, upward of 38-fold compared to Trem2^{-/-} control brain and 2.5-fold compared to Trem2^{+/+}12 week CPZ brain (FIG. 17E). Likewise, oxCE species, previously only reported in atherosclerotic lesions (Choi, et al. (2017). Biochim Biophys Acta Mol Cell Biol Lipids 1862, 393-397; Hutchins, et al. (2011). J Lipid Res 52, 2070-2083), were significantly upregulated in Trem2^{-/-} brain with chronic demyelination compared to Trem2^{+/+} control brain, although they were found at much lower levels than CE (see, Example 2, FIG. 3C). Despite the significant accumulation of CE, cholesterol levels remained unaltered in the brain, consistent with the fact that it is present in larger amounts than CE (see, Example 2, FIG. 3A) (Martin, et al. (2014). EMBO Rep 15, 1036-1052). Levels of ganglioside GM3 were also increased in Trem2^{-/-} brain with chronic demyelination (see, Example 2, FIG. 3F), reminiscent of the endolysosomal defects seen in Niemann-Pick disease type C, a lysosomal storage disease (Bissig, C., and Gruenberg, J. (2013). Cold Spring Harb Perspect Biol 5, a016816; Zervas, et al. (2001). J Neuropathol Exp Neurol 60, 49-64). Other neutral lipids, such as TG, were also elevated in Trem2^{-/-} brain upon CPZ treatment relative to controls (see, Example 2, FIG. 3E).

Overall, these data indicate that chronic demyelination causes a profound alteration of cholesterol metabolism, as well as specific lipid alterations in TREM2-deficient brain.

TREM2 Deficiency Causes Cholesteryl Ester Accumulation in Microglia Isolated from CPZ Treated Mice

To investigate whether CE accumulation is primarily intracellular or extracellular, FACS-based lipidomic approaches were developed to measure lipid species in a cell type-specific manner. CSF was also collected and analyzed to assess circulating levels of lipids in the CNS.

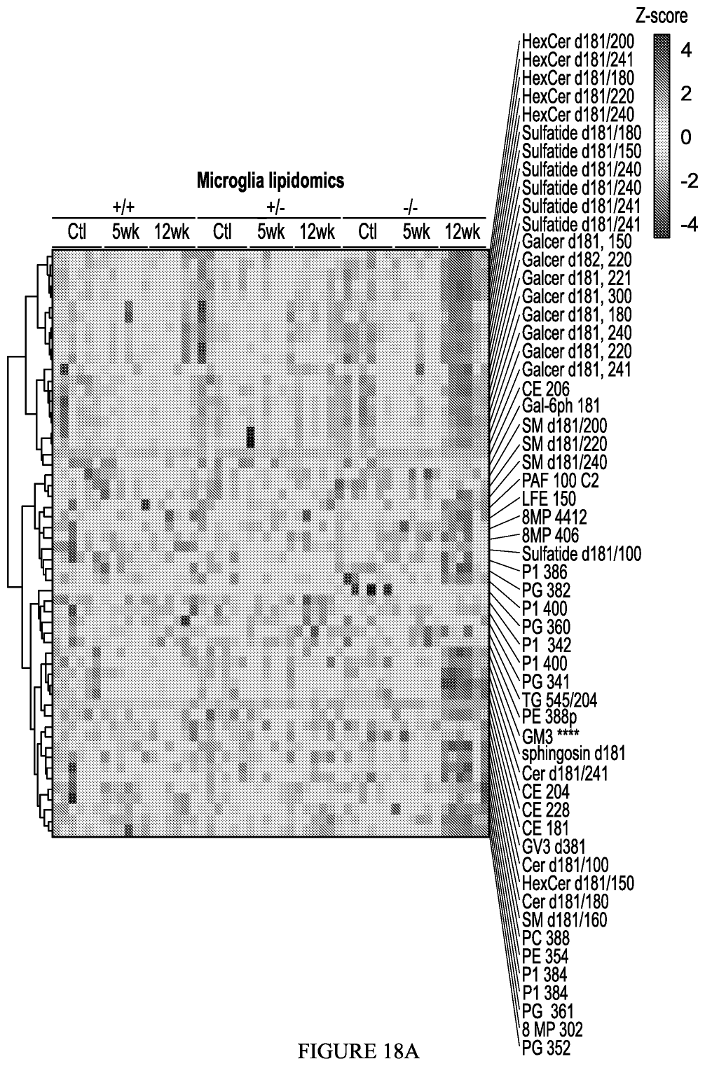

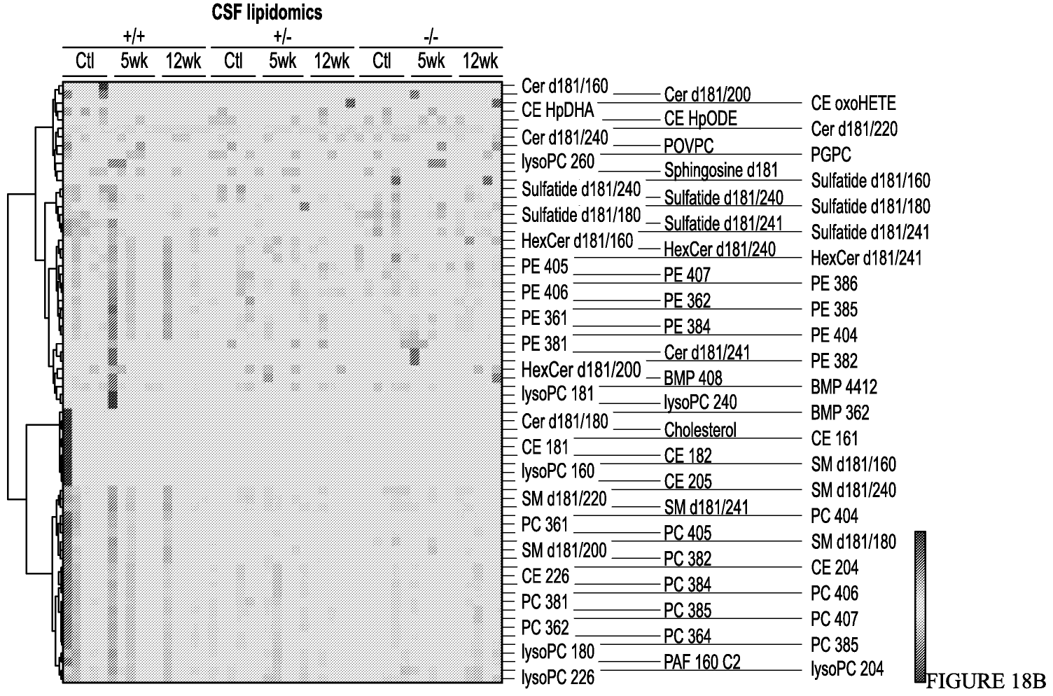

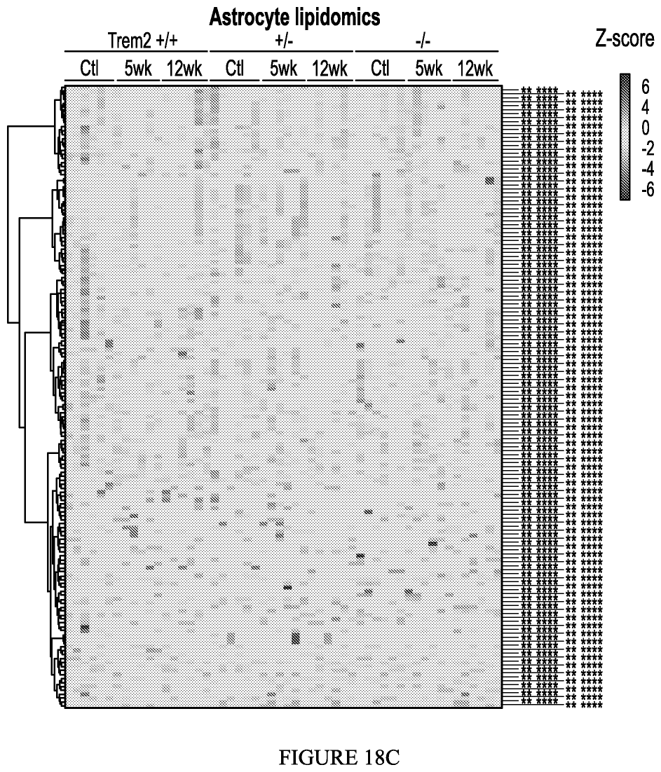

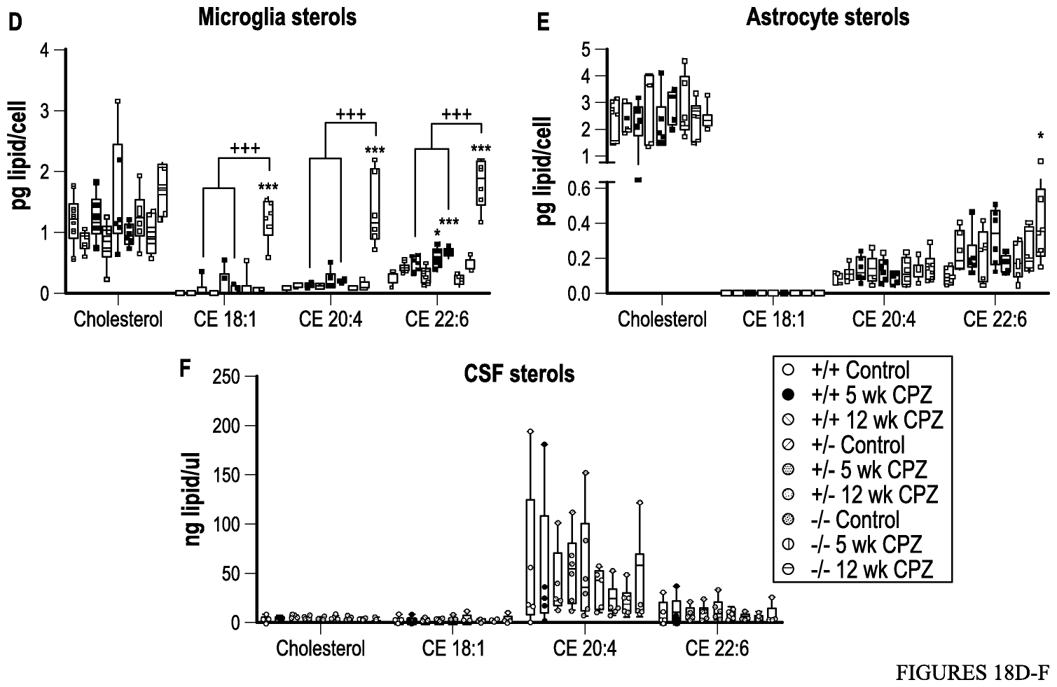

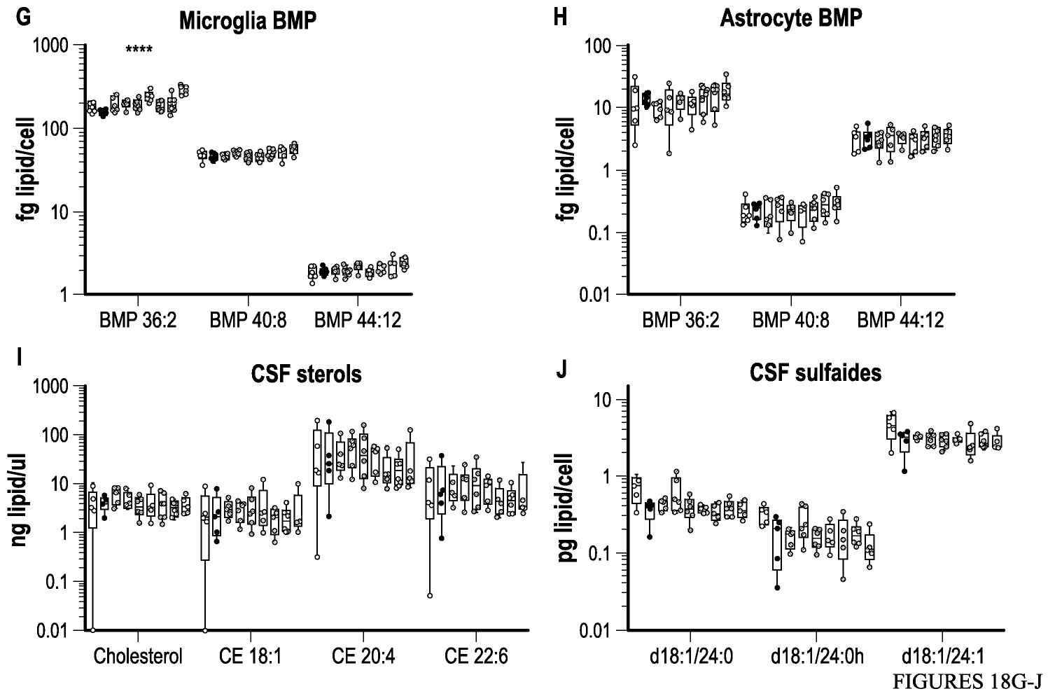

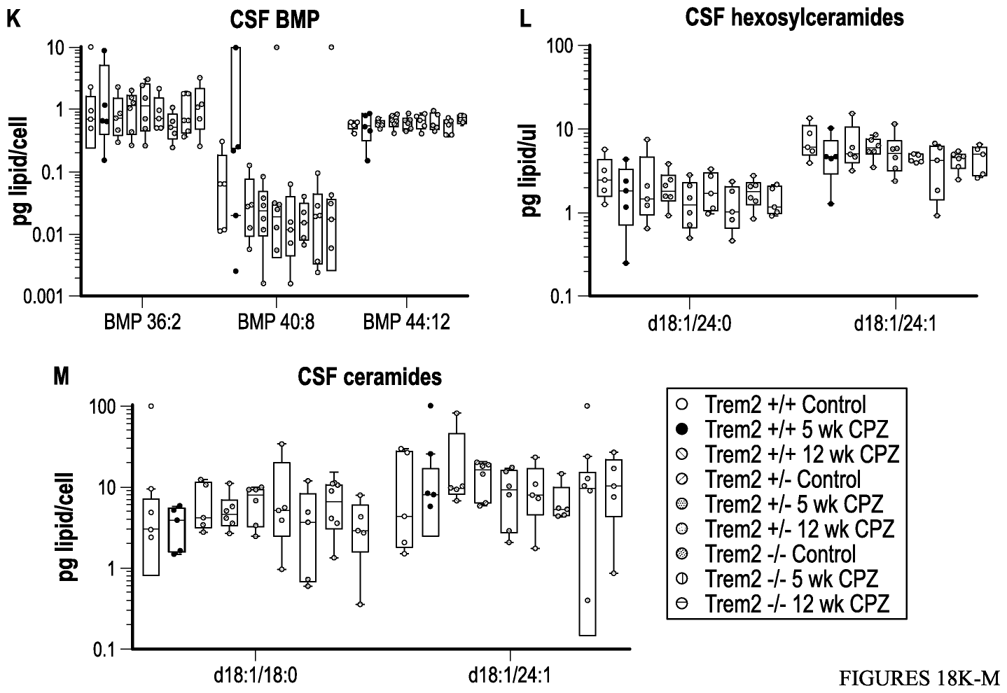

As observed in the whole tissue analysis from forebrain, chronic demyelination increased levels of certain lipid species in Trem2^{+/+}, Trem2^{+/-}, and Trem2^{-/-} microglia compared to untreated genotype controls. Trem2^{-/-} microglia exhibited dramatic increases in abundances of certain lipid species upon chronic demyelination compared to Trem2^{+/+} and Trem2^{+/-} microglia exposed to chronic demyelination, although there were no significant genotype-dependent effects in control microglia without CPZ treatment (FIG. 18A; two-way ANOVA, FDR<0.05). Strikingly, changes in the lipidomic profile upon chronic demyelination were unique to microglia, as the astrocyte-enriched population or CSF did not display any significant genotype- or CPZ treatment-specific alterations (FIGS. 18B and 18C; two-way ANOVA, FDR<0.05).

Increased (FIG. 18D) cholesteryl ester levels were detected in microglia isolated from Trem2^{-/-} brain with 12 week cuprizone diet compared to Trem2^{+/+}, Trem2^{+/-}, and Trem2^{-/-} microglia with control diet or 5 week cuprizone, and Trem2^{+/+} and Trem2^{+/-} microglia with 12 week cuprizone. Generally, no changes in lipid levels of cholesteryl ester were detected in astrocyte-enriched cell populations (FIG. 18E) or CSF (FIG. 18F) isolated from Trem2^{+/+}, Trem2^{+/-}, and Trem2^{-/-} brain with control or cuprizone diet. Lipids specifically increased in Trem2^{-/-} microglia vs. astrocytes upon chronic demyelination included some of the same species identified in the forebrain such as CE18:1, CE20:4, and CE22:6 (FIGS. 18D and 18E; interaction p<0.01 for 12 weeks CPZ, two-way ANOVA), which were elevated 8- to 43-fold in Trem2-microglia upon 12 week CPZ treatment compared to controls. Further confirming the accumulation of myelin debris in Trem2^{-/-} microglia upon demyelination, myelin-enriched lipids or metabolites thereof, and the ganglioside GM3 were uniquely enriched in Trem2^{-/-} microglia upon chronic demyelination but unchanged in astrocytes (see. Example 2, FIG. 4). This data suggests myelin lipids are able to be engulfed by Trem2^{-/-} microglia, but then fail to be properly metabolized and accumulate over time within microglia. Additionally, certain species of BMP, like BMP 36:2, were elevated in Trem2^{-/-} microglia upon 12 week CPZ treatment compared to controls (FIGS. 18G and 18H), potentially indicating lysosomal stress or dysfunction (Bissig, C., and Gruenberg, J. (2013). Cold Spring Harb Perspect Biol 5, a016816; Miranda, et al. (2018).

Nat Commun 9, 291). LCMS analysis of CSF from Trem2^{-/-} mice with chronic demyelination did not reveal any significant lipidomic differences from Trem2^{+/+}, Trem2^{+/-}, or Trem2^{-/-} CSF with or without CPZ treatment (FIGS. 18I-18M), suggesting that lipid accumulation observed in bulk forebrain tissue does not reflect extracellular accumulation, although changes in interstitial fluid were not ruled out.

These data indicate TREM2-deficient microglia are able to phagocytose myelin debris during demyelination but are unable to properly metabolize or mediate the efflux of myelin lipids.

### Example 9. Myelin Sulfatide Binds TREM2 and Promotes Downstream Signaling

This example describes the use of an in vitro system to more precisely delineate the mechanisms underlying the increased lipid accumulation in Trem2^{-/-} microglia. In particular, specific myelin lipids that bind to and signal through TREM2, which in turn may regulate the phagocytic clearance of myelin, were characterized.

### Liposome Preparation

70 molar percent DOPC (1,2-dioleoyl-sn-glycero-3-phosphocholine, Avanti Polar Lipids) and 30 molar percent of one test lipid were combined in chloroform in a glass vial and dried under a stream of N2 gas for 1-2h, or until completely dry. Test lipids included sulfatide (Avanti), POPS (Avanti), SM (Avanti), PI (Avanti), GalCer (Avanti), PE (Echelon Biosciences), and free cholesterol (Echelon Biosciences). The lipid mixture was re-suspended in HBSS (1-2 mg/mL final lipid concentration) and vortexed for 2-3 min. Subsequently, the lipid suspension was bath sonicated for 10 minutes. For surface plasmon resonance experiments, liposomes were extruded 10 times using an Avanti mini-extruder constructed with one 100 nm pore size membrane to form small unilamellar vesicles.

### TREM2/DAP12 and DAP12 HEK293 Stable Cell Lines

HEK293 cells were transfected with a pBudCE4.1 Mammalian Expression Vector (ThermoFisher) expressing wildtype human TREM2 and DAP12, and DAP12 alone. Stable expressing clones were selected and the cell surface TREM2 expression was evaluated by flow cytometry with APC-conjugated rat-anti-human/mouse-TREM2 monoclonal (R&D MAB17291). The highest wild type TREM2 expressing clone was selected for expansion. The clones stably expressing DAP12 were analyzed by Western blot.

pSYK AlphaLISA

Activation of TREM2-dependent pSYK signaling was measured using a commercial AlphaLISA assay (PerkinElmer). HEK293 cells: Two days before the experiment, HEK293 cells stably overexpressing TREM2 and DAP12 were plated at 40,000 cells/well on 96 well poly-D-lysine-coated plate. Differentiated human macrophage and BMDM were plated at 100,000 cells/well on tissue-culture treated 96-well plates. Cells were washed once with HBSS, then 50 μL of liposome mixture was added per well. For competition experiments, hTREM2-ECD or TREM1-his (Novoprotein Scientific) was incubated with liposomes for 1 hour at room temperature before adding to cells. 5 μg/mL human-specific mouse anti-TREM2 (Abnova) or 15 μg/mL mouse-specific sheep anti-TREM2 (R&D Systems) was added to each experiment as a positive control with side-by-side isotype controls, 5 μg/mL mouse IgG3 (R&D Systems) and 15 μg/mL polyclonal sheep IgG (R&D Systems), respectively. The cell plate was then transferred to a 37° C. incubator for 5 minutes. The liposome solution was discarded and 40 uL lysis buffer (Cell Signaling Technologies, CST). Lysate was incubated at 4 C for 30 min, then either frozen at −80 C or immediately carried forward to the alpha-LISA assay. Lysates were assayed using the standard protocol for the PerkinElmer pSYK AlphaLISA kit. 10 μL of lysate/well was transferred to a white opaque 384 well Optiplate (PerkinElmer). 5 μL of Acceptor Mix (containing the working solution of acceptor beads) was added per well followed by sealing of plates with foil seals and incubation 1 hour at room temperature. 5 μL of Donor Mix (containing the working solution of donor beads) was added to each well under reduced light conditions. Plates were again sealed and incubated 1 hour at room temperature. Plates were read using AlphaLISA settings on a PerkinElmer EnVision plate reader.

Recombinant Expression and Purification of His Tagged hTREM2 and hTREM2 R47H ECD

The ecto domain (residues 19-172) of TREM2 was sub cloned in the pRK vector with the secretion signal from mouse Ig kappa chain V-III, amino acids 1-20 at the N-terminal region and a 6×-His tag at the C-terminal region. Expi293F™ cells were transfected using the Expi293™ M Expression System Kit according to the manufacturer's instructions and the media supernatant was harvested 96 hr post transfection. Harvested media was supplemented with 1M imidazole pH 8.0 to a final concentration 10 mM, filtered, and loaded on to HisPur™ Ni-NTA Resin equilibrated with load buffer (20 mM Tris pH 8.0, 150 mM NaCl and 10 mM Imidazole). Nonspecifically bound proteins were washed with load buffer supplemented with 50 and 100 mM imidazole and TREM2 ecto domain was eluted with 20 mM Tris pH 8.0, 150 mM NaCl and 200 mM Imidazole. Eluted protein was pooled and subjected to size exclusion chromatography onto a HiLoad Superdex 75 16/600 column using 1×PBS as the running buffer. Elute fractions were analyzed by SDS PAGE and further characterized by analytical size exclusion chromatography and the intact protein mass determination.

### Lipid Binding Analysis Using Surface Plasmon Resonance (SPR)

The binding analysis was performed using Series S Sensor chip L1 and Biacore T200 instrument (GE Healthcare) at 25° C. Before coating with lipids, the sensor surfaces were washed with 1 minute injection of 40 μM 3-[(3cholamidopropyl)dimethylammonio]-1-propanesulfonate (CHAPS) and 40 uM of 0-octylglucoside at a flow rate of 30 μl/min. Residual detergent on the sensor surfaces was washed away by 30 second injection of 30% ethanol. 1 mg/ml Sulfatide/DOPC or PS/DOPC small unilamellar vesicles were injected for 15 minutes at 5 μl/min over the second flow cell. First flow cell was coated with DOPC and served as a reference surface. Loosely bound vesicles were washed away with two short pulses (15 s) of 10 mM NaOH at 30 μl/min followed by injection of 0.1 mg/ml bovine serum albumin for 3 minutes to block poorly-coated surface. Recombinant hTREM2-ECD or hTREM2-R74H-ECD proteins were diluted in PBS (0, 0.19, 0.56, 1.7, 5, and 15 μM) and injected over both flow cells for 60 seconds at 30 μl/min, and dissociation was monitored for additional 2 minutes. Between each measurement the lipids surface was regenerated by injection of 10 mM NaOH. Steady-state affinities were obtained by fitting the response at equilibrium against the concentration using Biacore™ T200 Evaluation Software v3.1

### Myelin Purification and Phagocytosis

Myelin was purified from wildtype C57Bl/6 mouse brain (Jackson Laboratories) using previously described methods (Safaiyan, et al. (2016). Nat Neurosci 19, 995-998). Following purification, myelin was resuspended in PBS and adjusted to 1 mg/mL protein concentration using the DC Protein Assay Kit 2 (BioRad, 5000112). Fractions of purified myelin were labeled using the pHrodo Red Microscale Labeling Kit (ThermoFisher, P35363) as per manufacturer recommendations. BMDM were plated in RPMI/10% FBS/Pen-Strep at a density of 100,000 cells per well in tissue culture treated 96 well plates (CellCarrier, PerkinElmer) supplemented with 5 ng/mL mouse M-CSF. As a negative control, 10 μM Cytochalasin D was added to cells 1 hr before myelin and retained throughout uptake assays. Cells were prestained with CellMask Deep Red Plasma Membrane Stain (1:5000, ThermoFisher C10046) and NucBlue Live ReadyProbes Reagent (2 drops per 1 mL, ThermoFisher R37605) in cell culture medium for 10 min, 370 C. PHrodo-myelin was diluted to 5 ug/mL in cell culture medium and bath sonicated for 1 min, then added to cells for 2-4 hr and imaged live (5% CO2, 37° C.) at 15-30 min intervals on an Opera Phenix HCS System (PerkinElmer). Individual cells were identified by nuclear and cell membrane stain, then pHrodo uptake intensity was quantified per cell per well using Harmony HCA Software (PerkinElmer).

### Results

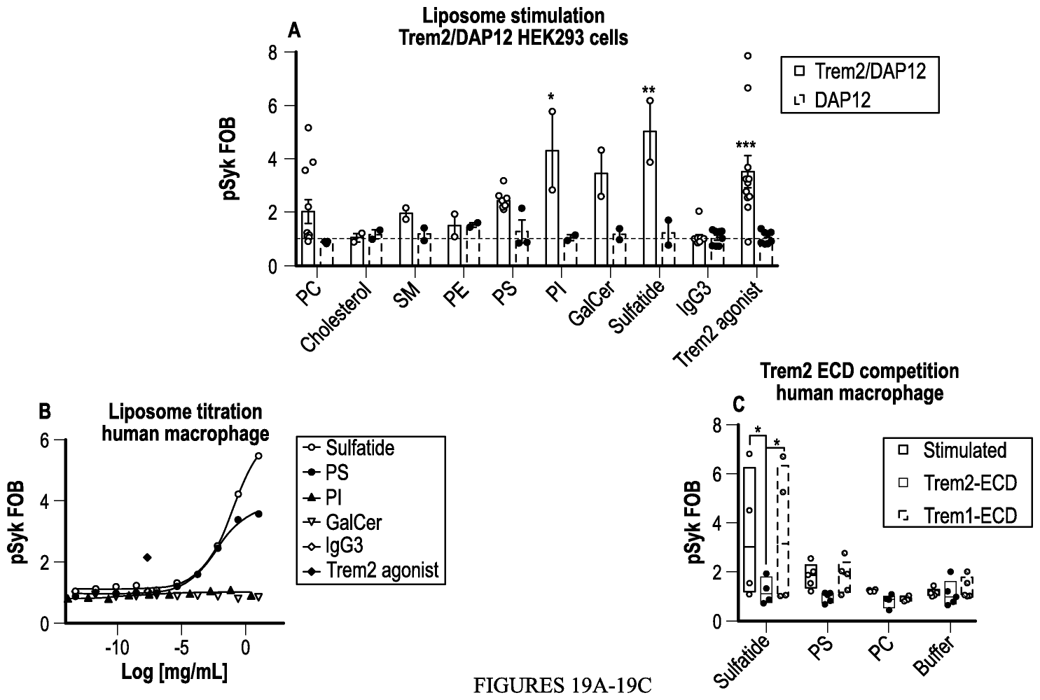

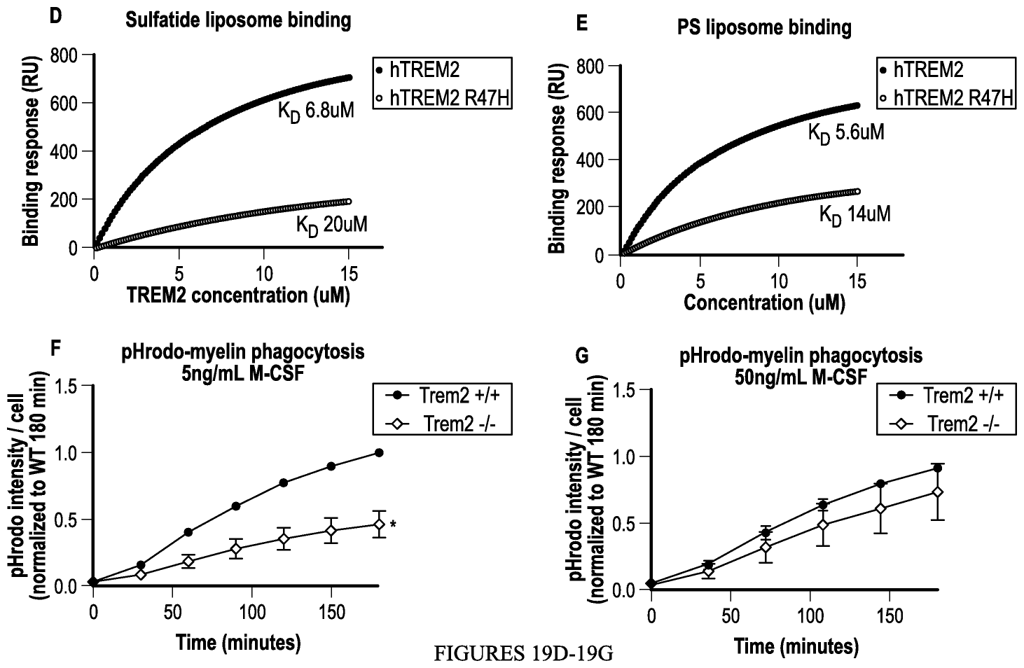

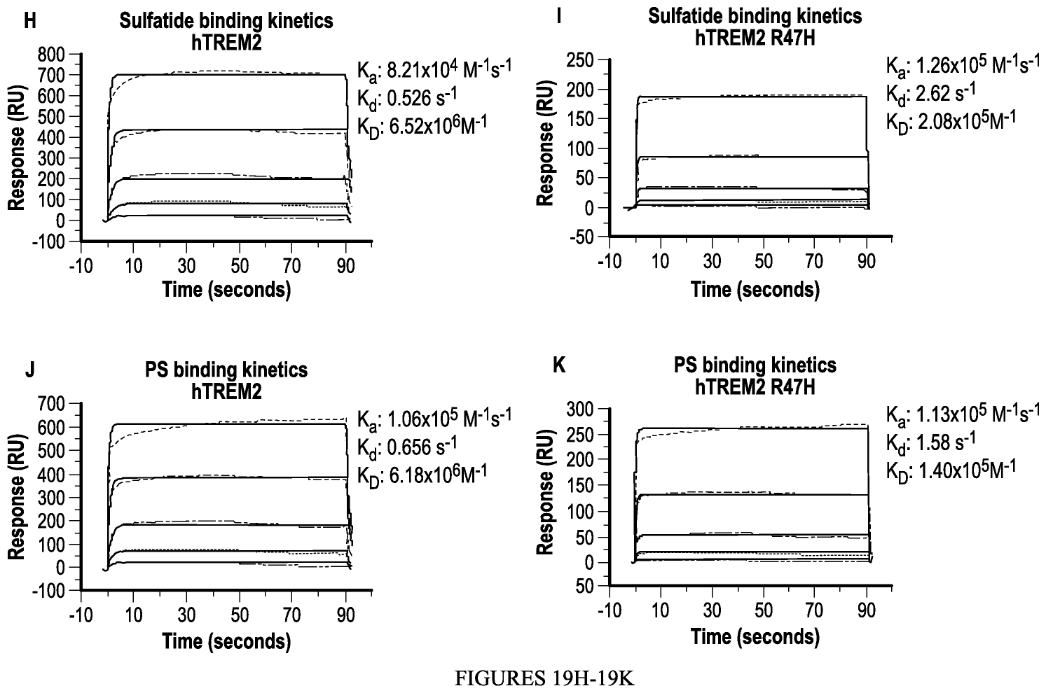

Human TREM2 was overexpressed in the presence or absence of human DAP12 in HEK293 cells. Downstream phospho-SYK (pSYK) levels were monitored to characterize the receptor activation by putative TREM2 lipid ligands found in myelin compared to PS, which is enriched on the surface of dead cells. Not all liposomes containing myelin candidate ligands increased pSYK levels in TREM2/DAP12 HEK293 cells. While PI and sulfatide significantly increased pSYK levels, none of the other lipids tested showed significant pSYK activation above DAP12-expressing cells or baseline buffer-stimulated controls (FIG. 19A).

To further characterize liposome-induced TREM2 signaling in a system with endogenous expression of TREM2/DAP12, human peripheral blood monocytes were differentiated into macrophages. Human macrophages exhibited liposome dose-dependent increases in pSYK levels for sulfatide and PS, but not PI or GalCer (FIG. 19B). Increased pSYK in response to liposomes containing sulfatide was reduced to baseline levels with addition of recombinant TREM2 but not TREM1 protein, confirming TREM2 binding and signaling specificity (FIG. 19C).

Certain TREM2 LOAD variants, including R47H, are thought to reduce TREM2 affinity to lipid ligands (Kober, et al. (2016). Elife 5; Ulland, T. K., and Colonna, M. (2018). Nat Rev Neurol 14, 667-675; Wang, et al. (2015). Cell 160, 1061-1071). Therefore, the binding affinity and kinetics of sulfatide and PS to the extracellular domain (ECD) of recombinant human wildtype TREM2 (hTREM2) and mutant R47H (hTREM2 R47H) protein was characterized through surface plasmon resonance (SPR) measurements. 30% sulfatide/70% PC and 30% PS/70% PC liposomes were coated onto a sensor chip and increasing concentrations of hTREM2 ECD protein were flowed over the chip to assess binding properties compared to 100% PC liposome baseline controls. hTREM2 exhibited similar binding affinity and response at the highest analyte concentration to sulfatide and PS liposomes, K_{D}=6.8 μM, Response Units (RU)=704 and K_{D}=5.6M, RU=631, respectively (FIGS. 19D and 19E). In comparison, hTREM2 R47H showed reduced affinity and lower binding response (i.e., RU) for sulfatide and PS liposomes, K_{D}=20 μM, RU=191 and K_{D}=14 μM, RU=267 respectively, suggesting ligand specificity (FIGS. 19D and 19E). Lower binding response is due to a faster off-rate of the interaction, which results in shorter residency of mutant TREM2 on the lipid surface. In the case of sulfatide, decreased affinity and response values observed with the R47H variant were accounted for by 5-fold (sulfatide) and 2.4-fold (PS) faster off-rates and relatively similar on-rates (FIGS. 19H-19K). This result demonstrates that sulfatide binds and signals via TREM2 and that the R47H LOAD variant is significantly impaired in its interaction with this lipid. Other myelin-enriched lipids did not appear to bind and signal via TREM2, including cholesterol, SM, PE, GalCer, and PI.

These studies demonstrating TREM2 binding to sulfatide lipid ligand suggests TREM2-deficient microglia may be impaired in myelin binding. To evaluate acute TREM2-dependent myelin uptake, Trem2^{+/+} and Trem2^{-/-} BMDM was treated with pHrodo-conjugated myelin. At low concentrations of M-CSF (5 ng/mL, a factor known to drive macrophage differentiation and survival), pHrodo-myelin phagocytosis was reduced in Trem2^{-/-} BMDM compared to Trem2^{+/+} (FIG. 19F). However, at high concentrations of M-CSF (50 ng/mL), pHrodo-myelin phagocytosis becomes more comparable for both genotypes (FIG. 19G). These data suggest that high levels of M-CSF may provide compensatory upregulation of phagocytic pathways in Trem2^{-/-} BMDM and reveal that the phagocytosis defects in TREM2-deficient cells can be context-dependent.

### Example 10. Increased Lipid Storage In Vitro in BMDMs Cultured from Trem2 KO Mice and in iPSC-Derived Microglia

This example describes the lipid storage phenotype observed in Trem2 KO BMDMs cultured in vitro and treated with myelin, both by immunocytochemistry and mass spectrometry analysis. The lipid storage phenotype was similarly evaluated in iPSC-derived microglia. Methods similar to those described in Examples 3 and 9 were used to perform the analysis.

Given that CE is a neutral lipid that preferentially accumulates in cytoplasmic lipid droplets, Trem2^{+/+} and Trem2^{-/-} BMDM were subjected to a treatment with 25 μg/mL myelin over 48 hr, and then stained with Nile red to assess neutral lipid storage with fluorescence microscopy. Cells were imaged and Nile Red was quantified as total spot area using a spot-finding algorithm on the Harmony software. To minimize genotype-specific differences in phagocytic uptake of myelin, these experiments were conducted in the presence of high M-CSF (50 μg/mL).

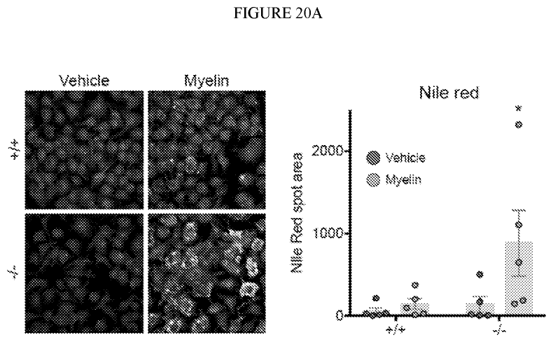

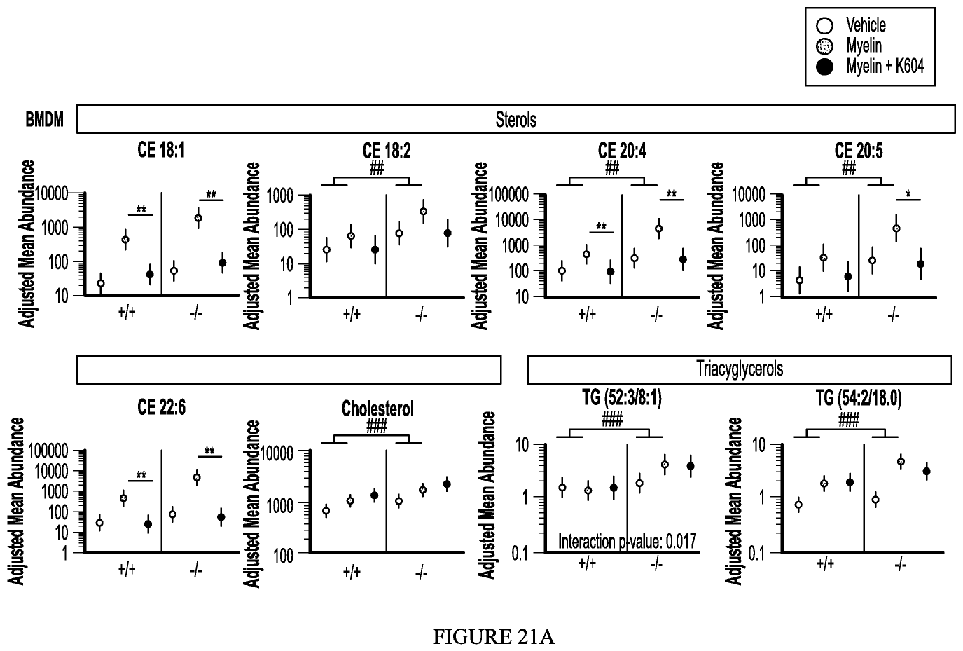

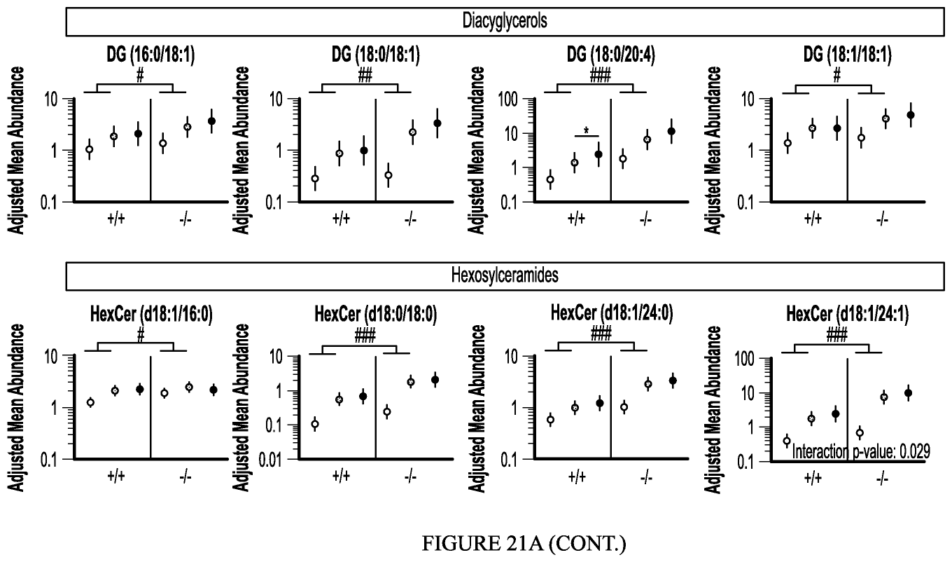

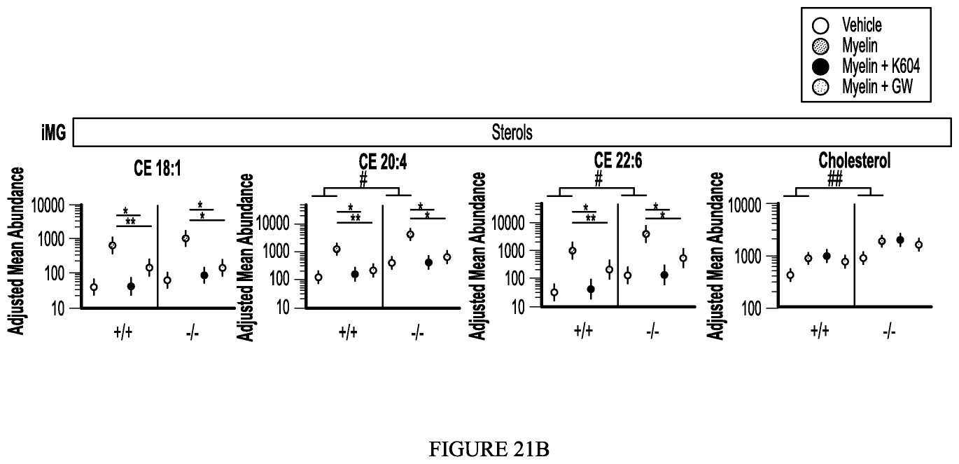

FIG. 20A depicts an increase in neutral lipid accumulation in Trem2 KO BMDMs treated with myelin compared to WT BMDMs, as shown by Nile Red staining. Subsequent lipidomic analysis revealed minimal genotype-specific lipid alterations in the absence of myelin treatment, but profound changes in the lipidome of Trem2^{-/-} BMDM with myelin treatment, including prominent genotype-specific accumulation of CE species CE18:2, CE20:4 and CE22:5 (FIG. 21A; two way ANOVA, p<0.01; see also. FIG. 20B). Free cholesterol and various species of triacylglycerols (TG), diacylgycerols (DG) and myelin-derived glycosphingolipid species (HexCer) also accumulated in Trem2^{-/-} BMDM (FIGS. 20C and 21A). Globally, these lipid changes were highly reminiscent of those observed in Trem2^{-/-} microglia in vivo after chronic CPZ-induced demyelination (see, Example 8, FIGS. 18A, 18D; and Example 2, FIGS. 4A-4P).

ACAT1 converts free cholesterol to CE in the endoplasmic reticulum. To determine the role of ACAT1 in the observed lipid accumulation in Trem2^{-/-} cells induced by myelin challenge, Trem2^{+/+} and Trem2^{-/-} BMDM were chronically treated for 48 hr with myelin and co-dosed with ACAT1 inhibitor (500 nM K604) (Ikenoya, et al. (2007). Atherosclerosis 191, 290-297). FIGS. 20B and 21A show that most cholesteryl esters do not accumulate in the presence of the ACAT inhibitor in both WT and TREM2 KO BMDM dosed with myelin, indicating that the cholesteryl ester accumulation is ACAT-dependent and myelin-derived cholesterol is indeed being stored as an esterified form in lipid droplets. Accumulation of other lipids in myelin-treated TREM2-deficient BMDM was not rescued by K604 (FIG. 21A), denoting the specificity of ACAT1 inhibition towards CE. Cholesterol is shown as a control and is slightly elevated in Trem2 KO BMDM with myelin and ACAT inhibition (FIGS. 20C and 21A).

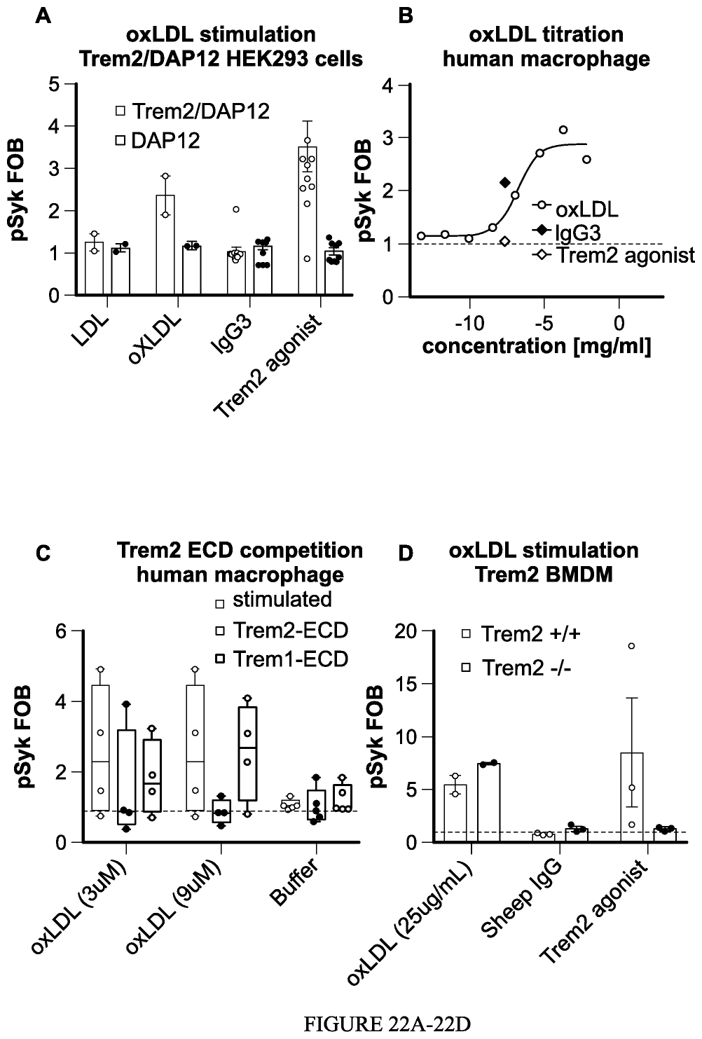

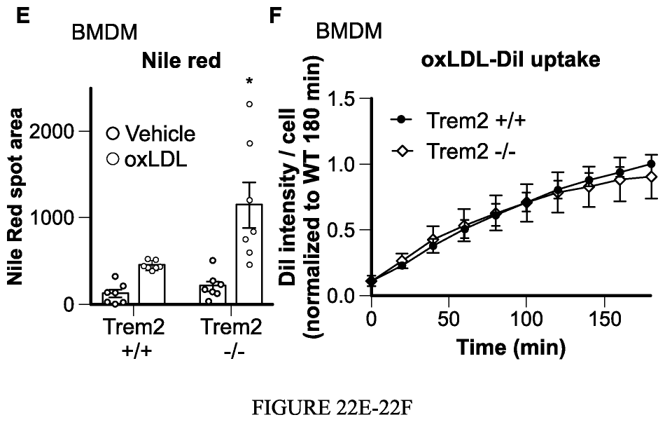

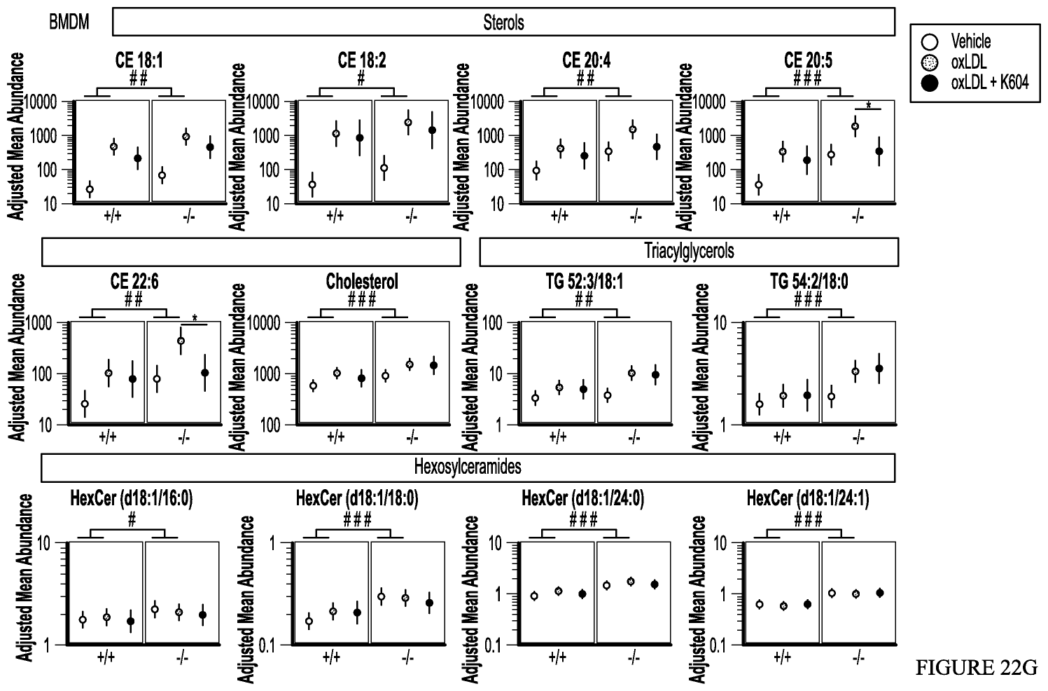

Trem2^{+/+} and Trem2^{-/-} BMDM were treated with oxidized LDL (oxLDL) to determine whether CE accumulation in Trem2^{-/-} BMDM was specific to myelin phagocytosis or if other physiologically-relevant uptake mechanisms could cause a similar effect. First, it was tested whether oxLDL could bind to and stimulate TREM2, using LDL as a control. Using a HEK293 cell line stably overexpressing human TREM2 in the presence or absence of human DAP12, it was determined that oxLDL stimulation trends toward increasing pSYK levels (FIG. 22A). Liposome titration in human macrophages revealed a dose-dependent increase in pSYK levels after stimulation with oxLDL (FIG. 22B). The increase was attenuated by pre-incubating oxLDL with high concentrations of recombinant hTREM2 ECD at 9 μM, but not at lower concentrations, such as those used in the liposome/hTREM2 competition experiments (3 μM) (FIG. 22C; see also. Example 9, FIG. 19C). This was corroborated by the fact that Trem2^{-/-} BMDM have similar pSYK levels to Trem2^{+/+} BMDM after acute stimulation with oxLDL (FIG. 22D). When treated chronically with 50 μg/mL oxLDL, Trem2^{-/-} BMDM exhibited an exacerbated accumulation of neutral lipids upon treatment, as shown by an increase in total spot area of Nile red staining (FIG. 22E; see also. Example 3, FIG. 5A) when compared to Trem2^{+/+} BMDM. This increase was not due to increased oxLDL uptake by Trem2^{-/-} BMDM, as indicated by comparable internalization of DiI-labeled oxLDL as Trem2^{+/+} cells (FIG. 22F). By LCMS, it was observed that certain species of CE and TG display an exacerbated increase in Trem2^{-/-} BMDM chronically treated with oxLDL (FIG. 22G). By contrast, HexCer, cholesterol, and DG did not display significant oxLDL-dependent lipidomic changes (FIG. 22G). K604 reduced levels of specific species of CE, such as CE20:5 and CE22:6, in Trem2^{-/-} BMDM upon oxLDL exposure, albeit less substantially than seen in myelin uptake experiments (FIG. 22G; Student's t-test, p<0.05), without increasing cholesterol levels or altering levels of other lipid families. These results indicate that in the oxLDL paradigm, and in contrast to the myelin paradigm, ACAT1 is only responsible for a fraction of CE accumulation in TREM2-deficient BMDM, suggesting that a pool of CE accumulates in organelles other than lipid droplets, likely lysosomes.

As shown for murine BMDM, iMG were capable of taking up pHrodo-myelin and TREM2 KO iMG showed a 22% decrease in myelin uptake relative to wildtype iMG after a 4 hr incubation (n=4 technical replicates). Despite the decrease in myelin phagocytosis, there was a genotype-specific increase in CE, particularly CE20:4 and CE22:6 species, as well as free cholesterol (FIG. 21B; two-way ANOVA, p<0.05 and p<0.01 for CE and free cholesterol, resp.). As in the case of murine BMDM, the CE increase, but not the free cholesterol increase, was abolished by co-treatment with K604, the ACAT1 inhibitor (FIG. 21B; Student's t-test, p<0.05).

To further delineate the molecular mechanisms underlying CE increase in TREM2^{-/-} iMG cells, iMG from both genotypes were treated with the LXR agonist, GW3695, which enhances the expression of the cholesterol efflux machinery, including ABCA1/ABCG1. This compound rescued the accumulation of all CE species measured in myelin-treated TREM2^{+/+} and TREM2^{-/-} iMG (FIG. 21B; Student's t-test, p<0.01 and p<0.05 for TREM2^{+/+} and TREM2^{-/-} iMG, respectively). These data suggest that TREM2 deficiency causes cholesterol efflux defects, leading to accumulation of an ACAT1 inhibitor-sensitive pool of CE in human iMG.

### Example 11. Cholesteryl Ester Accumulation in ApoE KO Brain, Sorted ApoE KO Microglia, Astrocytes and Neurons. And ApoE KO CSF

This example describes lipidomics of forebrain tissue, as well as of CSF, and isolated microglia, astrocytes and neurons from ApoE knockout mice with chronic demyelination. These experiments were performed in order to compare the phenotypes of Trem2 versus ApoE KO mice, given that the Trem2 KO microglia express much lower levels of ApoE.

### Cuprizone Diet to Induce Demyelination in Mice

Methods similar to those described in Example 1 were used for the demyelination protocol with cuprizone.

FACS of Microglia, Astrocytes, and Neurons from Mouse Brain

Generally, methods similar to those described in Example 1 were used for brain dissociation and the FACS protocol. To sort the neuronal, astrocyte and microglial cell populations, uniquely labeled antibodies that were specific for each cell type were used, along with Fixable Viability Stain BV510 to exclude dead cells.

### FACS Lipid Extraction and Mass Spectrometry Analysis

Lipid extraction and mass spectrometry of microglia, astrocytes and neurons were performed using methods similar to those described in Example 2.

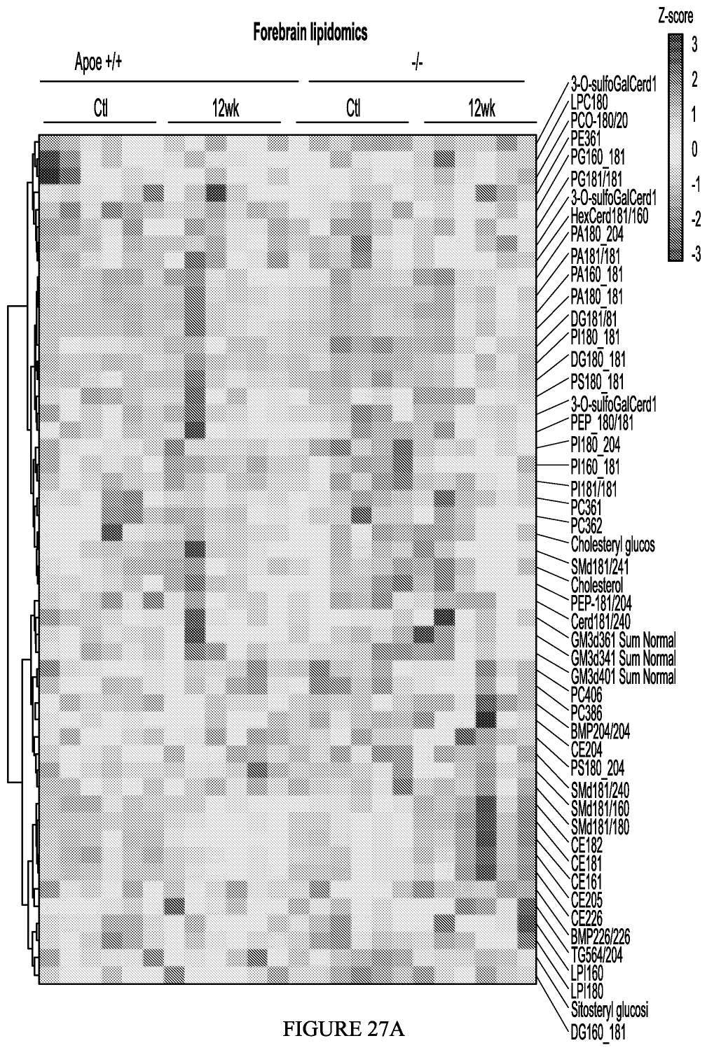

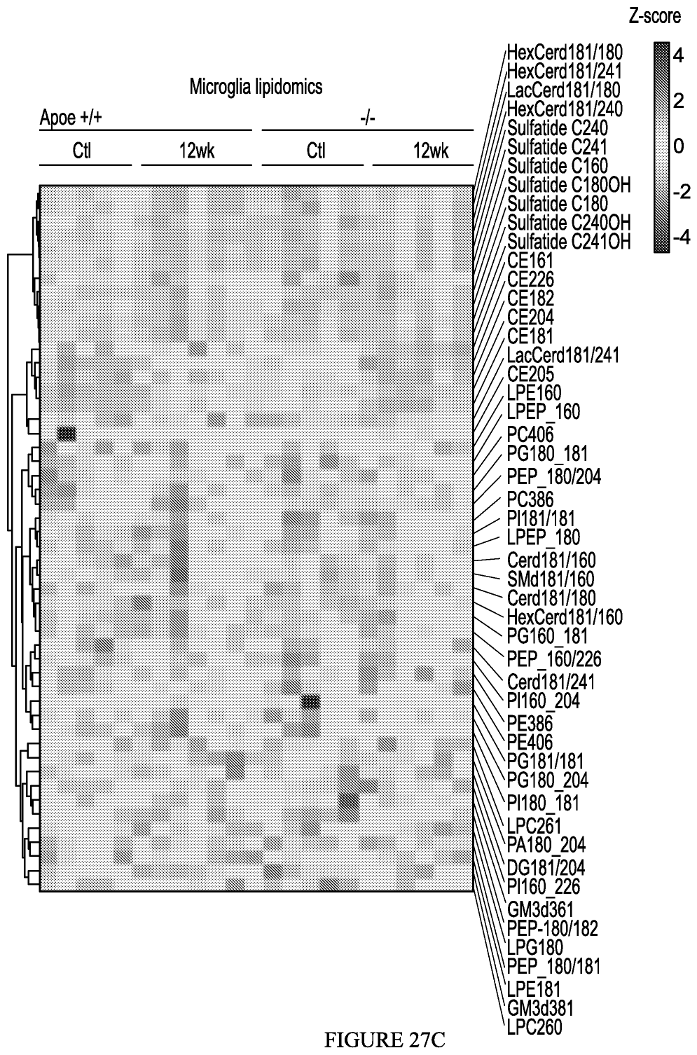

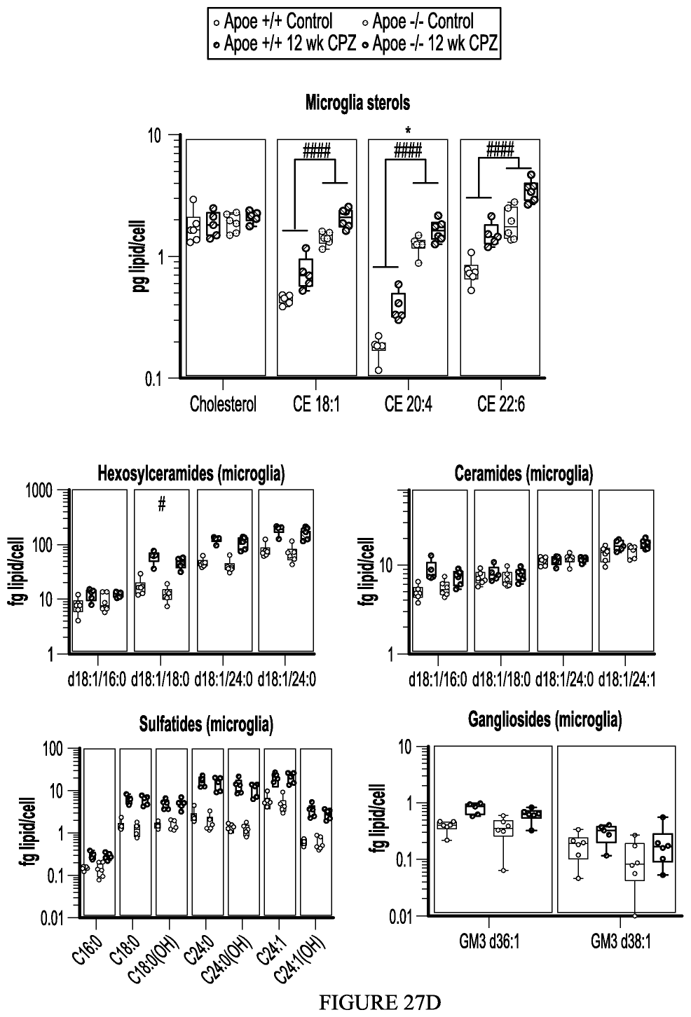

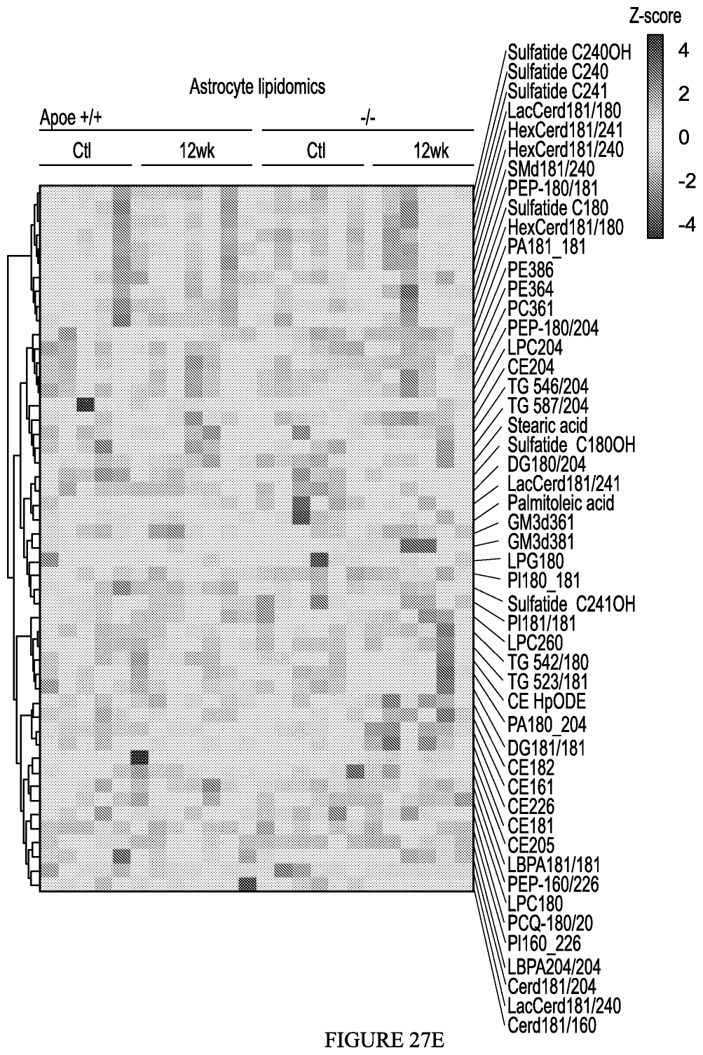

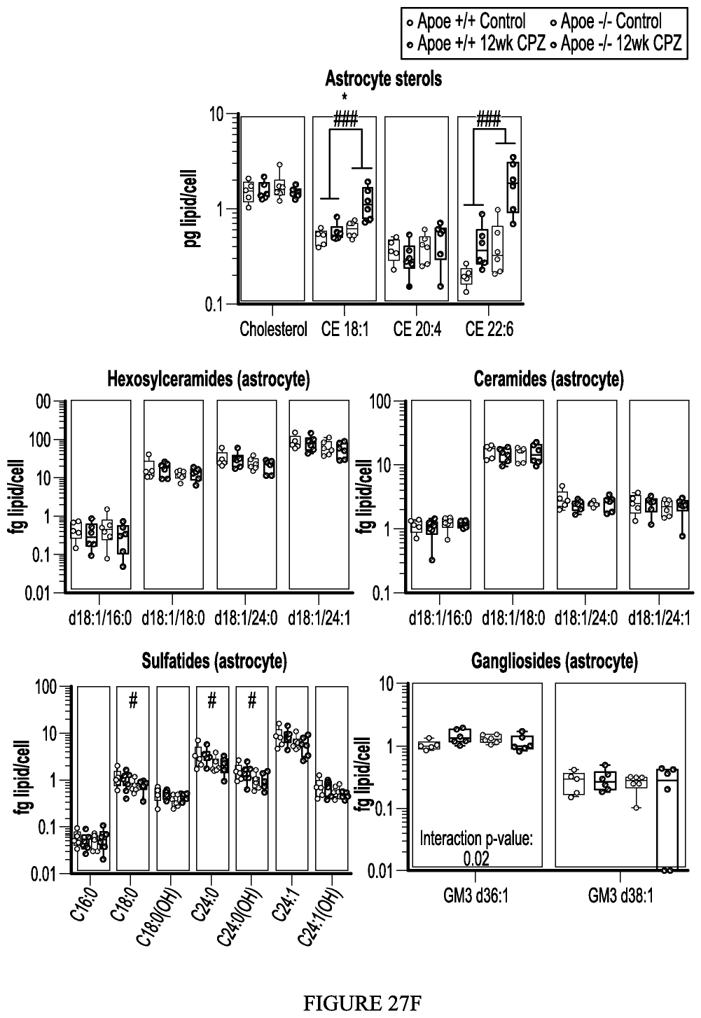

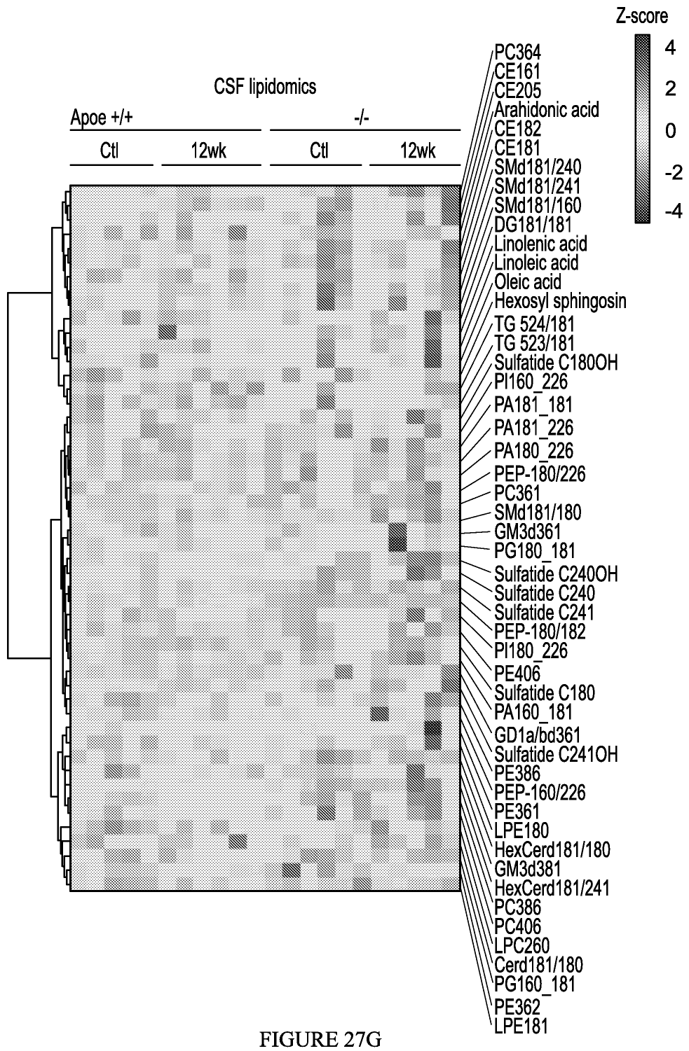

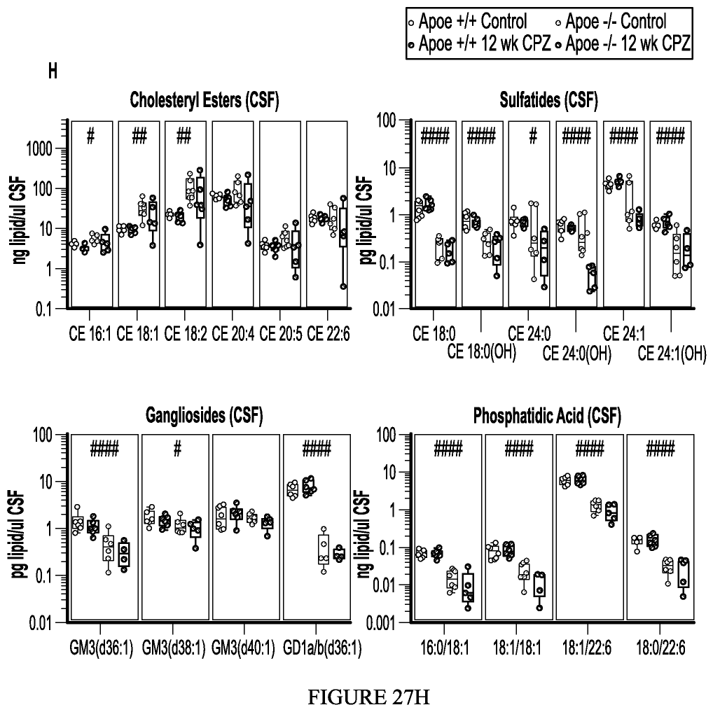

FIG. 24 shows total cholesteryl ester (CE) accumulation in ApoE KO forebrain in the presence or absence of demyelination induced by a 4 week-cuprizone diet. CE accumulated in the KO forebrain in the absence of demyelination and this accumulation was exacerbated by the cuprizone diet. Similarly, a lack of APOE and/or 12 week CPZ treatment generally caused a significant elevation of brain CE levels (FIG. 27A).

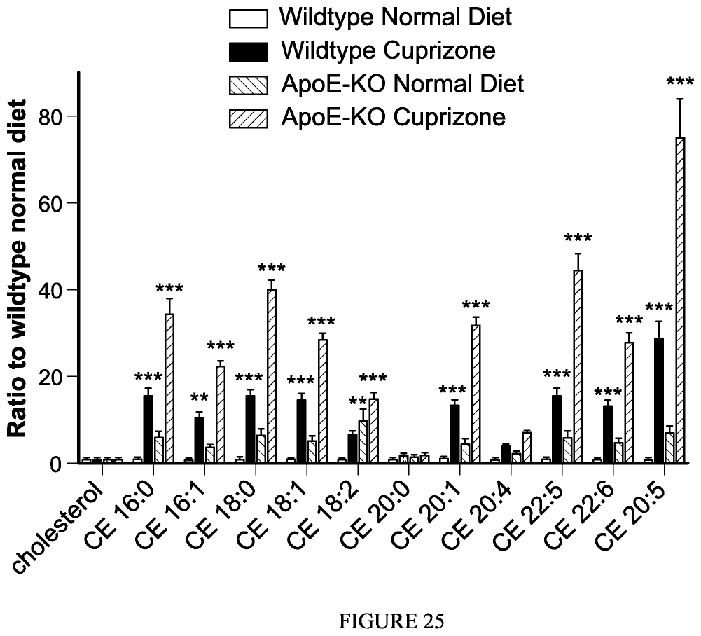

FIG. 25 shows accumulation of various molecular species of CE in the ApoE KO in the presence or absence of demyelination (4 week-CPZ diet compared to normal diet). Similarly, 12-week CPZ treatment led to a striking increase in CE18:1, 20:4 and 22:6 species levels (FIG. 27B; main effect from two-way ANOVA, FDR<0.05, p<0.001), and APOE deficiency significantly exacerbated the treatment effects for CE18:1 and CE20:4 (genotype-treatment interaction p<0.05). Levels of CE18:1 and CE22:6 were increased by 2.7- and 4-fold in Apoe ^{-/-} forebrain relative to wildtype forebrain with control diet. With the CPZ diet, fold-changes of CE18:1, CE20:4, and CE22:6 were 6.6, 1.4, and 6.7, respectively, compared to wildtype forebrain. In addition, levels of two BMP species (BMP40:4 and 44:12) were higher in the Apoe^{-/-} forebrain, consistent with lysosomal defects (FIG. 27B; p<0.05).

FIG. 26A shows that specific CE species, such as CE18:1, CE20:4 and CE22:6, accumulate in microglia isolated from ApoE KO brain with 12 week cuprizone diet compared to microglia isolated from ApoE WT brain, and to microglia isolated from ApoE KO brain with control diet (see also, FIGS. 27C-27D). FIG. 26B shows that specific CE species, such as CE18:1 and CE22:6, accumulate in astrocytes isolated from ApoE KO brain in the absence of demyelination (see also, FIGS. 27E-27F). These CE species accumulate more dramatically in the ApoE KO astrocytes with 12 week cuprizone diet compared to astrocytes isolated from ApoE WT brain with 12 week cuprizone diet, and to astrocytes isolated from ApoE KO brain with control diet. FIG. 26C shows that specific CE species, such as CE20:4 and CE22:6, accumulate in neurons isolated from ApoE KO brain in the absence of demyelination. These neuronal CE species are not affected by the cuprizone diet. No changes were found for free cholesterol in the forebrain or sorted glial cells (FIGS. 27B and 27D). Additionally, unlike in Trem2^{-/-} CSF, CEs were elevated in Apoe^{-/-} CSF, pointing to a widespread increase of these sterols in mutant brain (FIGS. 27G and 27H).

These data indicate that impaired cholesterol transport resulting from APOE deficiency causes massive accumulation of the storage form of cholesterol, i.e. CE, in the CNS, particularly in glial cells, which can also be detected in the CSF. The fact that Trem2 KO microglia, where APOE is downregulated, exhibit a similar CE storage suggest that this biochemical phenotype may originate from a defect in cholesterol transport.

### Example 12. Cholesteryl Ester Accumulation in Microglia and Astrocytes Isolated from 5XFAD Brain

This example describes lipidomics of microglia and astrocytes isolated from 5XFAD brain.

FACS of Microglia and Astrocytes from Mouse Brain

Methods similar to those described in Example 1 were used for brain dissociation and the FACS protocol.

### FACS Lipid Extraction and Mass Spectrometry Analysis

Lipid extraction and mass spectrometry of microglia and astrocytes were performed using methods similar to those described in Example 2.

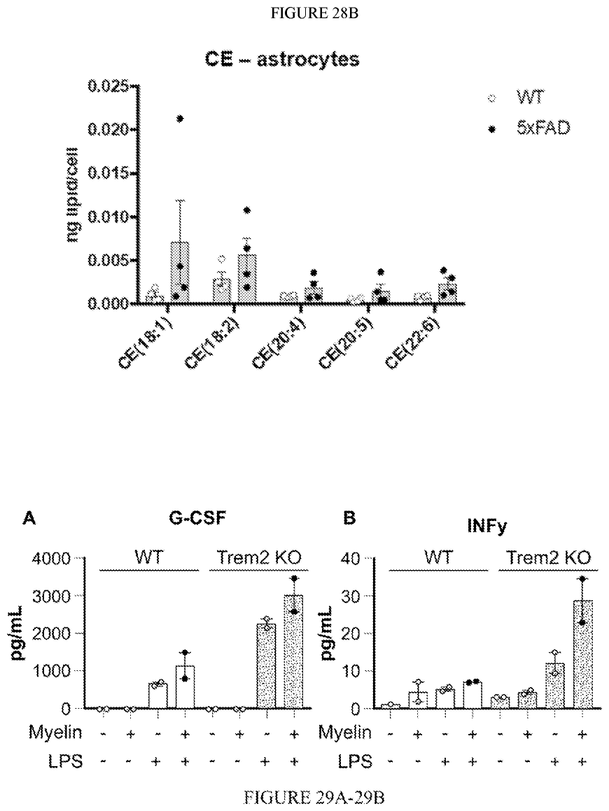

FIG. 28A shows increased levels of cholesteryl esters (CE) in microglia derived from the brain of 5XFAD mice relative to those derived from the brain of WT mice. Specific CE species, such as CE 18:1, CE20:4 and CE22:6, are higher in microglia derived from 5XFAD mice versus WT mice. FIG. 28B shows increased levels of cholesteryl esters (CE) in astrocytes derived from the brain of 5XFAD mice relative to those derived from the brain of WT mice. While most CE species are upregulated in 5XFAD astrocytes, it is to a lesser extent than in 5XFAD microglia. Animals were 14 months old. N=4 animals per group.

### Example 13. Increased Inflammatory Responses In Vitro in BMDMs Cultured from Trem2 KO Mice and in Human iPSC-Derived TREM2 KO Microglia and Anti-Inflammatory Effects of an Anti-TREM2 Antibody in Myelin-Treated Human iPSC-Derived TREM2 KO Microglia

Trem2 WT and Trem2 KO BMDMs were harvested/cultured using methods similar to those of Example 3. BMDMs were treated with either vehicle or purified mouse myelin and subsequently stimulated with lipopolysaccharide (LPS) to characterize the relationship between Trem2 genotype, lipid accumulation, and inflammatory cytokine secretion. Cells were plated at 100,000 cells per well and treated 24h later with either vehicle or 25 ug/mL myelin for 48h. For the last 16h of myelin treatment, either 0 or 10 ng/mL LPS was spiked into the wells. Cell culture media was collected, spun at 3000×g to remove debris, and frozen at −80° C. Cytokine levels were measured by quantitative immunoassay at Eve Technologies.

TREM2 WT and KO human iPSC-derived microglia (iMG) were plated at 30,000 cells/well on poly D-lysine-coated 96-well plates and cultured in homeostatic culture conditions by incubating in fully defined serum-free central nervous system cell culture media. Cells were treated with 50 μM Casp-1 inhibitor (InvivoGen, #VX-765) for 1h prior to LPS addition. Media was replaced with media containing 1 μg/ml LPS (InvivoGen). After 3 hours, cells were spiked with 5 mM ATP for 1 additional hour. 50 μl of culture media was then harvested, flash frozen, and assayed for IL-1β protein levels by quantitative immunoassay (Eve Technologies, Inc.). iMG were also treated with 25 ug/mL myelin for 24 hours, then treated with a control antibody (anti-RSV) or an anti-TREM2 antibody at 100 nM for 48 hours. IL-1β mRNA levels were measured by qPCR and normalized to GAPDH. N=2 biological replicates.

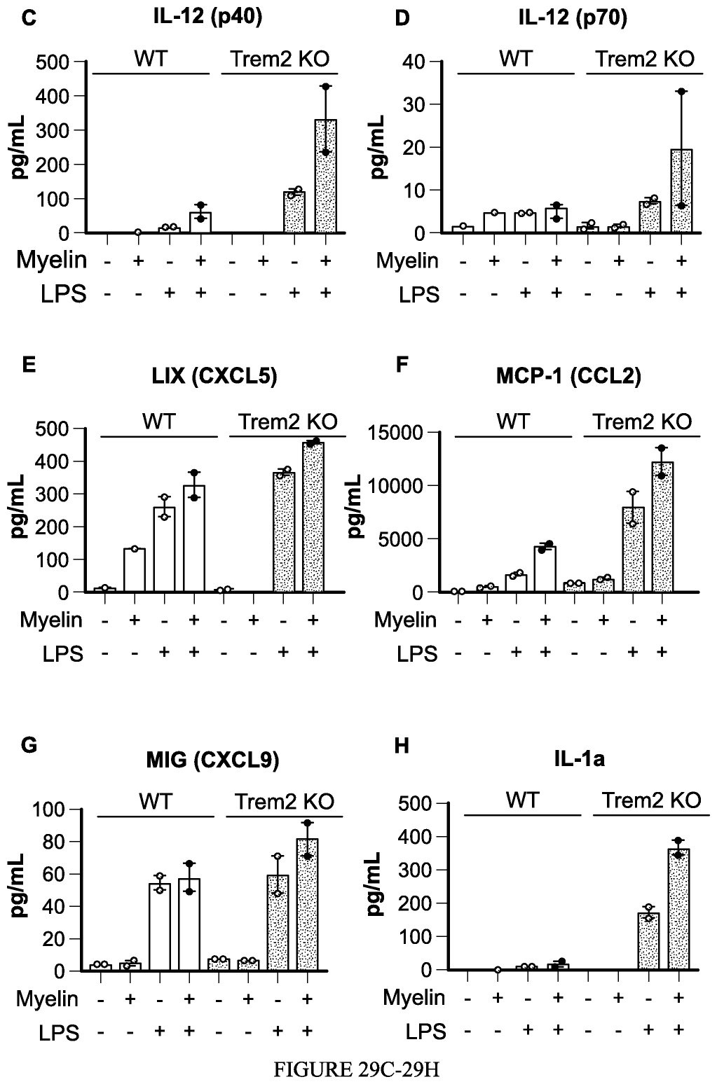

FIGS. 29A-29I show increased inflammatory cytokine production in Trem2 KO murine BMDM upon LPS stimulation (10 ng/mL) and myelin treatment. The following cytokines were increased in the Trem2 KO relative to Trem2 WT BMDMs (FIG. 29A) G-CSF, (FIG. 29B) INFy, (FIG. 29C) IL-12 (p40), (FIG. 29D) IL-12 (p70), (FIG. 29E) LIX (CXCL5), (FIG. 29F) MCP-1 (CCL2), (FIG. 29G) MIG (CXCL9), (FIG. 29H) IL-1a and (FIG. 29I) IL-1b were measured by quantitative immunoassay (Eve Technologies). Data represent mean±SEM, n=2 technical replicates.

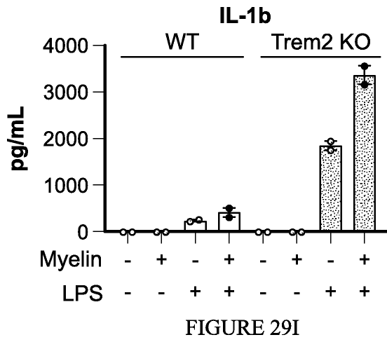

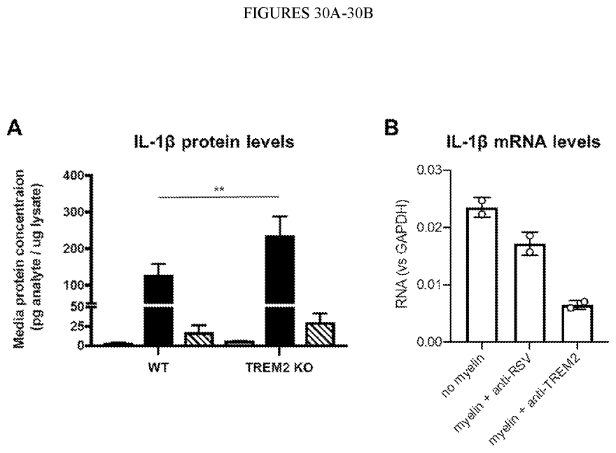

FIGS. 30A-30B show an increase in IL-1β cytokine response in human iPSC-derived TREM2 KO microglia and a decrease in IL-1β mRNA response with an anti-TREM2 antibody. FIG. 30A shows that TREM2 KO iPSC-derived microglia have an increased inflammasome response and IL-1β cytokine secretion after treatment of microglia with LPS and ATP. FIG. 30B shows that an anti-TREM2 antibody decreases IL-1β mRNA levels after treatment of microglia with myelin.

### Example 14. Differential Regulation of Lipid Metabolism Genes and Protein Secretion in Myelin-Treated Trem2 KO Human iPSC-Derived Microglia (MG)

This example describes gene expression and protein secretion analyses in WT and TREM2 KO human iPSC-derived microglia (iMG) treated with vehicle or myelin.

iPSC Microglia Methods

TREM2 WT and TREM2 KO human iMG were generated using methods similar to those of Example 3.

### Gene Expression Analysis

TREM2 WT and TREM2 KO human iPSC-derived microglia (iMG) were plated at 30,000 cells per well on poly D-lysine-coated 96-well plates. Cells were treated with vehicle or 25 ug/mL purified myelin for 24h and then lysed for collection of RNA. mRNA levels of select lipid metabolism genes were measured by qPCR and normalized to GAPDH.

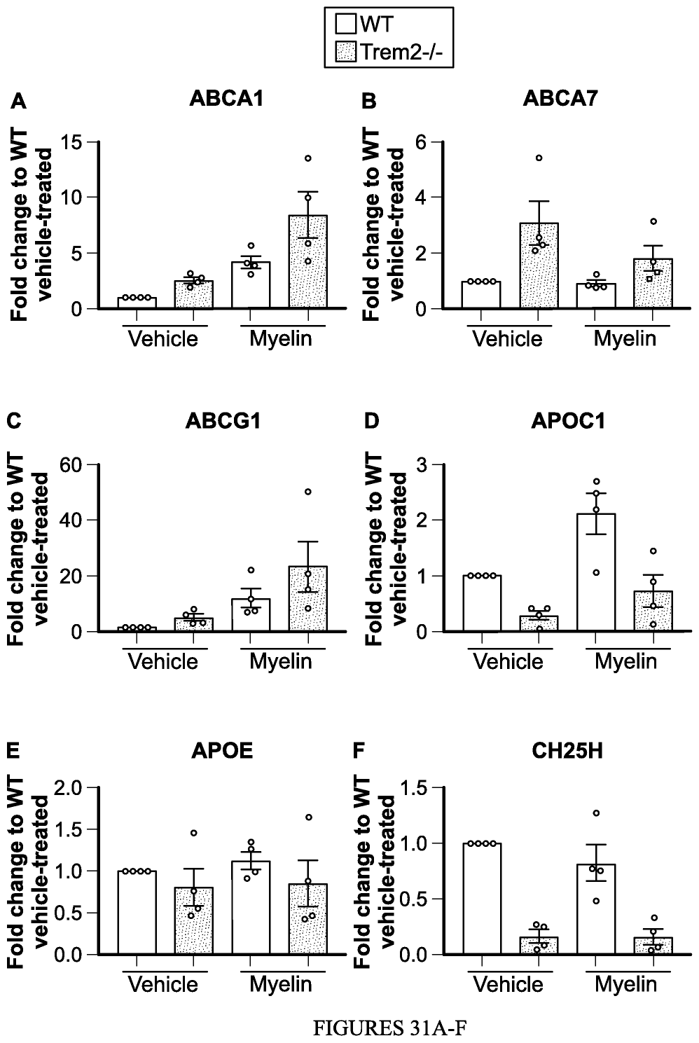

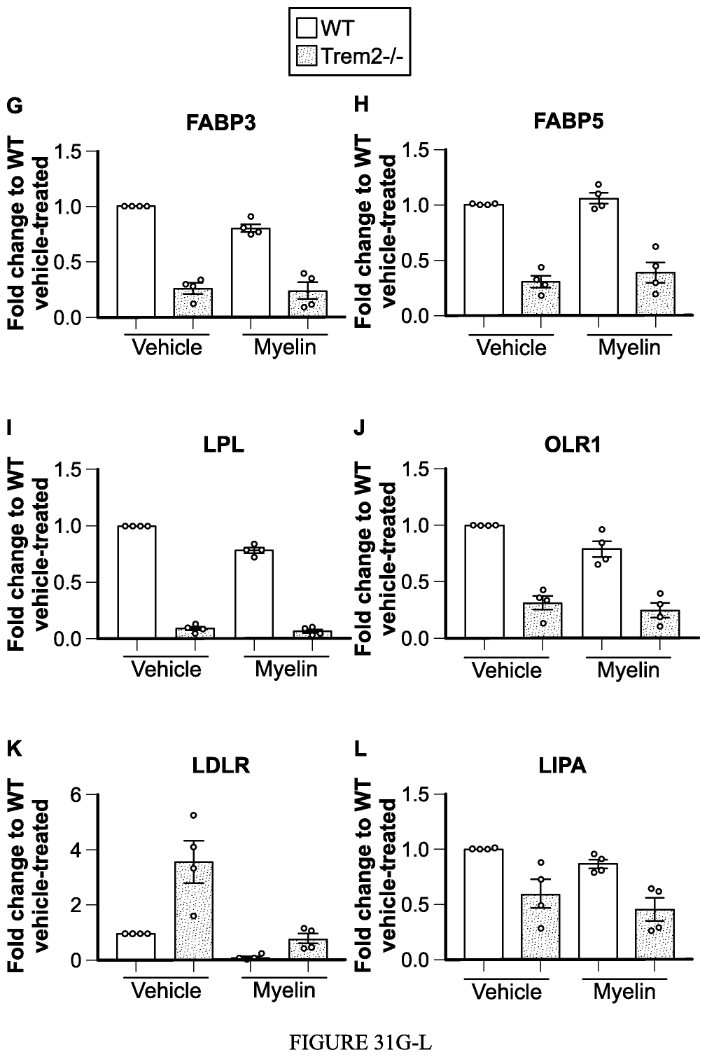

As shown in FIG. 31, ABCA1 (FIG. 31A) and ABCG1 (FIG. 31C) mRNA levels were increased in response to myelin in both TREM2 WT and TREM2 KO iMG, and higher in TREM2 KO iMG in vehicle and myelin-treated conditions compared to TREM2 WT iMG.

ABCA7 (FIG. 31B) and LDLR (FIG. 31K) mRNA were decreased in response to myelin, but higher in TREM2 KO compared to TREM2 WT iMG. APOC1 (FIG. 31D), APOE (FIG. 31E), CH25H (FIG. 31F), FABP3 (FIG. 31G), FABP5 (FIG. 31H), LPL (FIG. 31I), OLR1 (FIG. 31J), and LIPA (FIG. 1L) mRNA levels are lower in TREM2 KO compared to TREM2 WT iMG in both vehicle and myelin-treated conditions.

### Protein Secretion Analysis

TREM2 WT and TREM2 KO human iPSC-derived microglia (iMG) were plated at 30,000 cells per well on poly D-lysine-coated 96-well plates. Cells were treated with vehicle or 25 ug/mL purified myelin for 48h, and supernatant was subsequently collected for MSD analysis. Cells were lysed for BCA assay to determine protein concentration, and MSD data were normalized to these lysate concentrations.

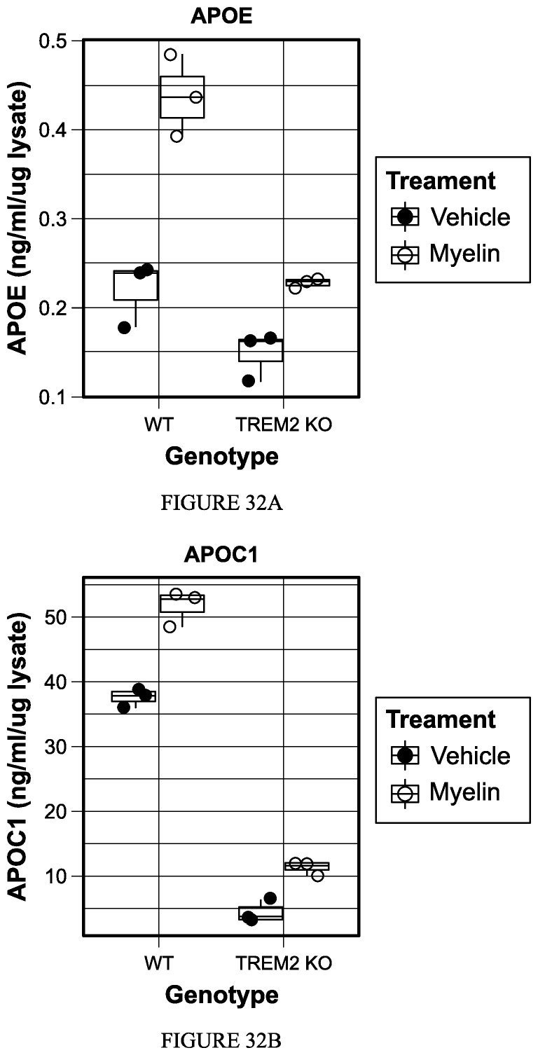

FIG. 32 shows that myelin increases secreted APOE (FIG. 32A) and APOC1 (FIG. 32B) protein in both TREM2 KO and TREM2 WT iMG, but APOE and APOC1 levels were lower in TREM2 KO cells in both conditions. These data further indicate that lack of TREM2 causes reduction of APOE function, consistent with the lipid accumulation observed in TREM2-deficient microglia. Additionally, the decreased levels of APOC1 suggest that reduced function of other apolipoproteins besides APOE contributes to the lipid phenotypes of TREM2-deficient microglia.

All publications, patents, and patent documents are incorporated by reference herein, as though individually incorporated by reference. The present disclosure has been described with reference to various specific and preferred embodiments and techniques. However, it should be understood that many variations and modifications may be made while remaining within the spirit and scope of the invention.

What is claimed is:

1. An agonist anti-triggering receptor expressed on myeloid cells 2 (TREM2) antibody for use in the treatment of dysregulated lipid metabolism in a mammal.

2. A method for treating dysregulated lipid metabolism in a mammal in need thereof, comprising administering to the mammal an effective amount of an agonist anti-TREM2 antibody.

3. The antibody or method of claim 1 or 2, wherein cells expressing TREM2 in the mammal exhibit dysregulated lipid metabolism.

4. The antibody or method of claim 3, wherein the cells are microglial cells or macrophages.

5. The antibody or method of any one of claims 1-4, wherein the mammal has, or has been determined to have, reduced TREM2 activity; and optionally, wherein the mammal has, or has been determined to have, reduced apolipoprotein E (ApoE) activity.

6. The antibody or method of any one of claims 1-5, wherein the dysregulated lipid metabolism comprises increased accumulation of one or more lipids.

7. The antibody or method of claim 6, wherein the one or more lipids are selected from the group consisting of cholesteryl esters, oxidized cholesteryl esters, bis(monoacylglycero)phosphate species (BMPs), diacylglycerides, triacylglycerides, hexosylceramides, galactosylceramides, lactosylceramides, sulfatides, gangliosides, phosphatidylserine 38:4, bis(monoacylglycero)phosphate 44:12, lysophosphatidylcholine 16:0, platelet activating factor, cholesterol sulfate, lysophosphatidylethanolamine, and combinations thereof.

8. The antibody or method of claim 7, wherein the one or more lipids includes a cholesteryl ester.

9. The antibody or method of any one of claims 1-8, wherein the mammal has or is prone to developing Alzheimer's disease, Nasu-Hakola disease (NHD), Lewy body dementia, Parkinson's disease, retinal degeneration, Huntington's disease, Frontotemporal Lobar Degeneration (FTD), Amyotrophic Lateral Sclerosis (ALS), Niemann-Pick disease type A, Niemann-Pick disease type B, Niemann-Pick disease type C, multiple sclerosis or vanishing white matter disease.

10. The antibody or method of any one of claims 1-8, wherein the mammal has or is prone to developing obesity, type 2 diabetes, alcoholic or non-alcoholic steatohepatitis, alcoholic or non-alcoholic fatty liver disease, rheumatoid arthritis (RA) or atherosclerosis.

11. The antibody or method of any one of claims 1-10, wherein the agonist anti-TREM2 antibody is MAB17291 or 78.18.

12. The method of any one of claims 2-11, wherein the administration reduces lipid accumulation, and optionally, reduces the expression of at least one pro-inflammatory cytokine selected from the group consisting of G-CSF, INFy, IL-12 (p40), IL-12 (p70), LIX (CXCL5), MCP-1 (CCL2), MIG (CXCL9), IL-1alpha, IL-1beta and IL-18.

13. The method of any one of claims 2-12, further comprising administering a second therapeutic agent selected from the group consisting of an RXR agonist, an LXR agonist and an acetyl-CoA acetyltransferase 1 (ACAT1) inhibitor.

14. The use of an agonist anti-TREM2 antibody to prepare a medicament for treating dysregulated lipid metabolism in a mammal.

15. An agonist anti-TREM2 antibody for use in reducing intracellular accumulation of one or more lipids in a cell.

16. A method of reducing intracellular accumulation of one or more lipids in a cell, comprising contacting the cell with an effective amount of an agonist anti-TREM2 antibody.

17. The antibody or method of claim 15 or 16, wherein the cell is a microglial cell or a macrophage.

18. The antibody or method of any one of claims 15-17, wherein the one or more lipids are selected from the group consisting of cholesteryl esters, oxidized cholesteryl esters, BMPs, diacylglycerides, triacylglycerides, hexosylceramides, galactosylceramides, lactosylceramides, sulfatides, gangliosides, phosphatidylserine 38:4, bis(monoacylglycero)phosphate 44:12, lysophosphatidylcholine 16:0, platelet activating factor, cholesterol sulfate, lysophosphatidylethanolamine, and combinations thereof.

19. The antibody or method of any one of claims 15-18, wherein the cell has, or has been determined to have, reduced TREM2 activity; and optionally, wherein the cell has, or has been determined to have, reduced ApoE activity.

20. The antibody or method of any one of claims 15-19, wherein the cell is present in a mammal.

21. The use of an agonist anti-TREM2 antibody to prepare a medicament for reducing intracellular accumulation of one or more lipids in a cell.

22. An agonist anti-TREM2 antibody for use in the treatment of Alzheimer's disease in a mammal, wherein the mammal has, or has been determined to have, dysregulated lipid metabolism.

23. A method of treating Alzheimer's disease in a mammal in need thereof, the method comprising administering to the mammal an agonist anti-TREM2 antibody, wherein the mammal has, or has been determined to have, dysregulated lipid metabolism.

24. The antibody or method of claim 22 or 23, wherein the mammal has, or has been determined to have, dysregulated lipid metabolism in TREM2 expressing cells.

25. The antibody or method of claim 24, wherein the TREM2-expressing cells have, or have been determined to have, reduced TREM2 activity.

26. The antibody or method of any one of claims 22-25, wherein the dysregulated lipid metabolism comprises increased intracellular accumulation of one or more lipids selected from the group consisting of cholesteryl esters, oxidized cholesteryl esters, BMPs, diacylglycerides, triacylglycerides, hexosylceramides, galactosylceramides, lactosylceramides, sulfatides, gangliosides, phosphatidylserine 38:4, bis(monoacylglycero)phosphate 44:12, lysophosphatidylcholine 16:0, platelet activating factor, cholesterol sulfate, lysophosphatidylethanolamine, and combinations thereof.

27. The use of an agonist anti-TREM2 antibody to prepare a medicament for treating Alzheimer's disease in a mammal, wherein the mammal has, or has been determined to have, dysregulated lipid metabolism.

28. An agonist anti-TREM2 antibody for use in the treatment of atherosclerosis in a mammal.

29. A method of treating atherosclerosis in a mammal in need thereof, comprising administering to the mammal an effective amount of an agonist anti-TREM2 antibody.

30. The antibody or method of claim 28 or 29, wherein the mammal has, or has been determined to have, dysregulated lipid metabolism, wherein the dysregulated lipid metabolism comprises increased accumulation of one or more lipids.

31. The antibody or method of claim 30, wherein the one or more lipids are selected from the group consisting of cholesteryl esters, oxidized cholesteryl esters, BMPs, diacylglycerides, triacylglycerides, hexosylceramides, galactosylceramides, lactosylceramides, sulfatides, gangliosides, phosphatidylserine 38:4, bis(monoacylglycero)phosphate 44:12, lysophosphatidylcholine 16:0, platelet activating factor, cholesterol sulfate, lysophosphatidylethanolamine, and combinations thereof.

32. The antibody or method of any one of claims 28-31, wherein macrophages in the mammal have, or have been determined to have, reduced TREM2 activity.

33. The use of an agonist anti-TREM2 antibody to prepare a medicament for treating atherosclerosis in a mammal.

34. An agonist anti-TREM2 antibody for use in the treatment of inflammation in a mammal.

35. A method of treating inflammation in a mammal in need thereof, comprising administering to the mammal an effective amount of an agonist anti-TREM2 antibody.

36. The method of claim 35, wherein the administration reduces the expression of at least one pro-inflammatory cytokine selected from the group consisting of G-CSF, INFy, IL-12 (p40), IL-12 (p70), LIX (CXCL5), MCP-1 (CCL2), MIG (CXCL9), IL-1alpha, IL-1beta and IL-18.

37. The antibody or method of any one of claims 34-36, wherein the mammal has or is prone to developing RA, gout, or inflammatory bowel disease (IBD).

38. The antibody or method of any one of claims 34-36, wherein the mammal has or is prone to developing Alzheimer's disease, Nasu-Hakola disease (NHD), Lewy body dementia, Parkinson's disease, retinal degeneration, Huntington's disease, Niemann-Pick disease type A, Niemann-Pick disease type B, Niemann-Pick disease type C, multiple sclerosis or vanishing white matter disease.

39. The antibody or method of any one of claims 34-36, wherein the mammal has or is prone to developing obesity, type 2 diabetes, alcoholic or non-alcoholic steatohepatitis, alcoholic or non-alcoholic fatty liver disease or atherosclerosis.

40. The use of an agonist anti-TREM2 antibody to prepare a medicament for treating inflammation in a mammal.

41. A method of sorting populations of CNS cells from a tissue sample, comprising:
(a) contacting the tissue sample with an anti-CD45 primary antibody, an anti-CD11b primary antibody and an anti-astrocyte cell surface antigen-2 (ACSA-2) primary antibody, wherein each primary antibody is uniquely labeled, to provide a labeled tissue sample; and
(b) sorting the cells in the labeled tissue sample by flow cytometry, wherein the method provides distinct cell populations of astrocytes and microglial cells.

42. A collection of CNS cells comprising two physically separate cell populations, wherein the first cell population comprises an enriched population of CD45^{low}/CD11b⁺/ACSA-2⁻ cells and the second cell population comprises an enriched population CD45⁻/CD11b⁻/ACSA-2⁺ cells.
